(19) United States
(12) Patent Application Publication
(10) Pub. No.: US 2023/0285556 A1
(43) Pub. Date: Sep. 14, 2023

(54) METHODS AND COMPOSITIONS FOR TREATING CANCER

(71) Applicant: Omeros Corporation, Seattle, WA (US)

(72) Inventors: George A. Gaitanaris, Seattle, WA (US); Marc A. Gavin, Seattle, WA (US); Robert H. Lemus, Seattle, WA (US); Caglar Cekic, San Marcos, CA (US); Mohammed Oukka, Newcastle, WA (US); Trupti Vardam-Kaur, Seattle, WA (US)

(21) Appl. No.: 17/931,398

(22) Filed: Sep. 12, 2022

Related U.S. Application Data
(60) Provisional application No. 63/243,621, filed on Sep. 13, 2021.
(60) Provisional application No. 63/352,558, filed on Jun. 15, 2022.

(51) Int. Cl. A61K 39/00; A61P 35/00; C12N 5/0783

(52) U.S. Cl. CPC A61K 39/4611; A61P 35/00; A61K 39/4632; A61K 39/4631; A61K 39/4622; A61K 39/464499; C12N 5/0638; C12N 2501/727; C12N 2501/2307; C12N 2501/2315; C12N 2501/01; A61K 2239/26

(57) ABSTRACT

Compounds, compositions, and methods for generating T cells with altered phenotype are disclosed. The phenotype-altered T cells have increased persistence, prolonged survival, and increased antitumor activity and are useful for treatment of cancers.

### CROSS-REFERENCE TO RELATED APPLICATIONS

This application claims the benefit of U.S. Provisional Application No. 63/243,621, filed Sep. 13, 2022, and claims the benefit of U.S. Provisional Application No. 63/352,558, filed Jun. 15, 2022, the disclosures of both of which are hereby incorporated by reference in their entirety.

### FIELD OF THE INVENTION

This disclosure is directed to methods and compositions for phenotypically modifying T cells by culturing the cells in a cell culture medium comprising an effective amount of a phenotype-altering agent (e.g., any combination of PKA inhibitor, an A2A adenosine receptor inhibitor, and a GPR174 inhibitor in combination with a p38 inhibitor, and/or a PI3K6 inhibitor, or combinations thereof) thereby stimulating alteration of a phenotype of at least a subpopulation of the cultured T cells.

### BACKGROUND

Adoptive cell therapy (ACT) using naturally occurring cancer antigen-reactive T cells or T cells with genetically engineered tumor specificity-such as chimeric antigen receptor-expressing T (CAR-T) cells-harnesses the body's own natural defenses to provide cellular therapies that offer the promise of minimal toxicities with the benefit of long-term immune protection. Despite multiple advances in the field of ACT, several obstacles to the successful use of ACT for the treatment of cancer and other diseases remain. For example, expansion of the numbers of T cells may produce T cells with a terminally differentiated phenotypes that have diminished antitumor activity and/or poor capacity for long-term persistence in vivo.

Accordingly, a need exists for compositions comprising T cells with improved therapeutic efficacy and improved methods of obtaining such isolated populations of T cells.

### BRIEF SUMMARY

The present disclosure is generally directed to compositions and methods related to treatment of diseases such as cancer, more specifically, by adoptive cell therapy (ACT).

In one aspect, the disclosure provides a method of treating a disease, the method comprising administering to a subject in need thereof a therapeutically effective amount of phenotype-altered T cells wherein the phenotype-altered T cells are prepared by a method comprising a step of culturing a population of T cells in vitro in the presence of a composition comprising a phenotype-altering agent selected from the group consisting of a protein kinase A (PKA) inhibitor, an A2A adenosine receptor inhibitor, a GPR174 inhibitor, and combinations thereof for a time sufficient to alter a phenotype of at least a subpopulation of the population of T cells. In some embodiments, the disease treatable by the methods of the disclosure is cancer.

Additionally, in another aspect, the disclosure provides a method for preparing an isolated population of T cells comprising a subpopulation of phenotype-altered T cells, wherein the method comprises culturing a population of T cells in vitro in the presence of a composition comprising a phenotype-altering agent selected from the group consisting of a protein kinase A (PKA) inhibitor, an A2A adenosine receptor inhibitor, a GPR174 inhibitor, and combinations thereof for a time sufficient to alter a phenotype of at least a subpopulation of the population of T cells.

Yet another aspect provides a population of T cells suitable for ACT, wherein the T cells have been cultured in the presence of a composition comprising at least one inhibitor selected from the group consisting of a protein kinase A (PKA) inhibitor, an A2A adenosine receptor inhibitor, a GPR174 inhibitor, and combinations thereof, optionally with a p38 inhibitor and/or with a PI3K6 inhibitor, for a period sufficient to alter a phenotype of at least a subpopulation of the T cells.

In the aspects described above, the cells with the altered phenotype have higher antitumor activity and/or capacity for long-term persistence in vivo compared to cells cultured without the presence of the phenotype-altering agents.

### BRIEF DESCRIPTION OF THE DRAWINGS

In the figures, identical reference numbers identify similar elements. The sizes and relative positions of elements in the figures are not necessarily drawn to scale and some of these elements may be enlarged and positioned to improved figure legibility. Further, the particular shapes of the elements as drawn are not intended to convey any information regarding the actual shape of the particular elements and have been solely selected for ease of recognition in the figures.

FIG. 1 graphically illustrates the impact of mouse OT-I CD8 T cell expansion in the presence or absence of exemplary GPR174 inhibitor (Compound 10) and A2A inhibitor (ZM-241385 or ZM) on IL-2 production following restimulation, as described in Example 1.

FIG. 2 shows the impact of human CD8 T cell expansion for 10 days in the presence or absence of exemplary GPR174 inhibitor (Compound 10) and A2A inhibitor (ZM-241385 or ZM) on IL-2 production following restimulation, as described in Example 2.

FIGS. 3A-3H show the T cell phenotypes that were enriched or reduced by the 10-day culture with the exemplary A2A inhibitor (ZM-241385 or ZM) and/or the exemplary GPR174 inhibitor, (Compound 10) as described in Example 2.

FIG. 4 graphically illustrates the impact of human CD8 T cell expansion—with IL-7 and IL-15 rather than IL-2—for 10 days in the presence or absence of exemplary GPR174 and A2A inhibitors (Compound 10 and ZM-241385 or ZM, respectively) on IL-2 production following restimulation, as described in Example 3.

FIG. 5 graphically illustrates the impact of human CD8 T cell expansion—with IL-7 and IL-15 rather than IL-2—for 10 days in the presence or absence of two exemplary GPR174 inhibitors (Compound 10 and Compound 49) and an A2A inhibitor (ZM-241385 or ZM) on IL-2 production following restimulation, as described in Example 3.

FIG. 6 graphically illustrates the production of IL-2 following restimulation of human CD8 T cells that had been cultured with the various exemplary small molecule inhibitors of GPR174, A2A, PKA, and EPAC, as described in Example 4.

FIGS. 7A-7B graphically illustrate the production of IL-2 following restimulation of human CD8 T cells that had been cultured with the indicated exemplary small molecule inhibitors of GPR174 and A2A (combined), PKA, or p38, and fold-increases in T cell number during the 10-day expansion with the compounds, as described in Example 5.

FIGS. 8A-8B graphically illustrate the same readouts shown in FIGS. 7A-7B for human CD8 T cells expanded in the presence of four exemplary inhibitors of cAMP signaling (PKA inhibitor Rp-8-Br-cAMPS, and EPAC inhibitors HJC-0197 and ESI-09), each with or without the p38 inhibitor doramapimod, as described in Example 5.

FIGS. 9A-9D graphically illustrate the impact of mouse OT-I CD8 T cell expansion in the presence or absence of exemplary PKA and p38 inhibitors, alone or combined, on cell growth and IL-2 production following restimulation on day 8 or day 10 of the expansion cultures, as described in Example 6.

FIGS. 10A-10C depict the expression of CD62L, TCF1/TCF7, CD39, CD69, PD-1, and CTLA-4 in OT-I cells after 8 days of culture with exemplary PKA and p38 inhibitors, alone or combined, as described in Example 6.

FIGS. 11A-11B graphically illustrates the tumor volume (FIG. 11A) and survival (FIG. 11B) of EG7 tumor-bearing mice following the transfer of OT-1 cells precultured with vehicle, a p38 inhibitor, a PKA inhibitor, or both compounds combined, as described in Example 7.

FIG. 12 is a schematic of an exemplary method of preparing a population of T cells of the disclosure.

FIGS. 13A-B graphically illustrate the effects of 8 combinatorial combinations of the p38 inhibitor doramapimod (Dora), the PKA inhibitor Rp-8-Br-cAMPS, and the PI3K6 inhibitor idelalisib-present during a 9-day expansion of mouse OT-I CD8 T cells-on IL-2 production following restimulation with EG7 cells in absence of the inhibitors, as described in Example 8.

FIG. 14 depicts the fold increase in OT-I CD8 T cell numbers during the 9-day expansion, as described in Example 8.

FIGS. 15A-B graphically illustrate the effects of combinatorial combinations of the p38 inhibitor doramapimod, either combined with GPR174 and A2A inhibitors Compound #10 and ZM-241385 respectively or PKA inhibitor Rp-8-Br-cAMPS, and the PI3K6 inhibitor idelalisib-present during a 10-day expansion of mouse OT-I CD8 T cells-on IL-2 production following restimulation with EG7 cells in absence of the inhibitors, as described in Example 9.

FIGS. 16A-B depict the fold increase in OT-I CD8 T cell numbers during the 10-day expansion, as described in Example 9.

FIG. 17A shows TCF1/TCF7 expression for 2 conditions, and FIGS. 17B and 17C show the % TCF1/TCF7⁺ cells for all 12 conditions of Example 9 following 10-day expansion of mouse OT-I CD8 T cells as described in Example 9.

FIG. 18A shows CD62L expression for 2 conditions and FIGS. 18B and 18C show the % CD62L⁺ cells for all 12 conditions following 10-day expansion of mouse OT-I CD8 T cells as described in Example 9.

FIG. 19A shows CD39 expression for 2 conditions, and FIGS. 19B and 19C show and the % CD39⁺ cells for all 12 conditions following 10-day expansion of mouse OT-I CD8 T cells as described in Example 9.

FIG. 20A shows CD69 expression for 2 conditions, and FIGS. 20B and 20C show the % CD69⁺ cells for all 12 conditions following 10-day expansion of mouse OT-I CD8 T cells as described in Example 9.

FIG. 21A shows CTLA-4 expression for 2 conditions, and FIGS. 21B and 21C show the % CTLA-4⁺ cells for all 12 conditions following 10-day expansion of mouse OT-I CD8 T cells as described in Example 9.

FIG. 22A shows PD-1 expression for 2 conditions, and FIGS. 22B and 22C show the % PD-1⁺ cells for all 12 conditions following 10-day expansion of mouse OT-I CD8 T cells as described in Example 9.

FIG. 23A shows TIM-3 expression for 2 conditions, and FIGS. 23B and 23C show the % TIM-3⁺ cells for all 12 conditions following 10-day expansion of mouse OT-I CD8 T cells as described in Example 9.

FIG. 24A shows CD103 expression for 2 conditions, and FIGS. 24B and 24C show the % CD103⁺ cells for all 12 conditions following 10-day expansion of mouse OT-I CD8 T cells as described in Example 9.

FIG. 25A shows CXCR3 expression for 2 conditions, and FIGS. 25B and 25C show the % CXCR3⁺ cells for all 12 conditions following 10-day expansion of mouse OT-I CD8 T cells as described in Example 9.

### DETAILED DESCRIPTION

In the following description, certain specific details are set forth in order to provide a thorough understanding of various embodiments of the disclosure. One of ordinary skill in the art will understand that embodiments of the disclosure may be practiced without these details.

The inventors discovered that protein kinase A (PKA) inhibitors, A2A adenosine receptor inhibitors, and/or GPR174 inhibitors, used alone or in combination with p38 inhibitors and/or PI3K6 inhibitors and/or combinations thereof, can advantageously modify the function and phenotype of cultured T cells. Specifically, in a non-limiting illustrative example, described below, T cells stimulated and grown in the presence of a PKA inhibitor retain a central memory phenotype and produce more IL-2 following re-stimulation in the absence of the inhibitor. The inventors further discovered that when the PKA inhibitor is combined with a p38 inhibitor and/or PI3K6 inhibitor, such treatment results in enhancement of IL-2 production, such as an additive, super additive, or synergistic enhancement. Thus, a significant enhancement of the efficacy of CAR-T and other adoptive T cell therapies can be achieved by culturing T cells in vitro in the presence of these inhibitors (e.g., PKA inhibitors, GPR174 inhibitors, A2A inhibitors, or combinations thereof with or without one or more of p38 and/or PI3K6 inhibitors) because the T cells cultured in this manner can survive longer, produce more IL-2, and more effectively reduce tumor burden when a therapeutically effective dose of such T cells is administered to a patient in need thereof, as compared to T cells prepared without the inhibitors.

Adoptive T cell therapies (ACTs) are emerging as effective and tractable cancer treatments; however, improving the persistence and phenotypic stability of transferred T cells in patients continues to be an area of intense research (See Grimes J M et al, Cellular therapy for the treatment of solid tumors, Transfusion and Apheresis Science, 60 (1):103056 (2021), Hou, A. J., Chen, L. C. & Chen, Y. Y. Navigating CAR-T cells through the solid-tumour microenvironment. Nat Rev Drug Discov 20, 531-550 (2021).) All ACTs incorporate a manufacturing process in which T cells are activated through their antigen receptor and expanded for several days or several weeks with T cell growth factors such as IL-2, IL-7, and IL-15, after which the cells are transferred into patients or cryopreserved for future use. Different types of ACT include, but are not limited to: 1) the isolation of cells enriched for natural-occurring tumor-reactive T cells (NTR-T cells), either from tumor biopsies or from patient blood based on specific cell-surface phenotypes, and activating with anti-CD3+anti-CD28 antibodies or with tumor-specific peptide antigens presented by antigen presenting cells, 2) the genetic modification, during the in vitro expansion, of patient T cells with introduced genes encoding a chimeric antigen receptor (CAR-T cells) or a native or chimeric T cell receptor (TCR-T cells) specific for the patient's tumor antigen, and 3) the same as (2) but with T cells from healthy individuals, or “universal donors”, whereby the T cells are expanded to large numbers and cryopreserved to administer “off-the-shelf” to multiple patients as needed. All three of these scenarios may also incorporate other genetic manipulations, such as gene deletions or insertions, to improve their survival and tumor killing activity in patients, as a common problem with ACT is the loss or inactivation of the transferred T cells. Because of the large number of proteins known to regulate T cell differentiation and function, multiple genetic modifications are being explored and may ultimately be required to engineer optimally effective ACT; however, the technical difficulties associated with genetically engineering various signaling pathways has generated new interest in simply altering T cell culture conditions to achieve similar outcomes. For example, T cells that display a memory rather than a terminally-differentiated phenotype will persist longer in patients, or in mice in experimental models, resulting in more effective tumor killing; and growing T cells with IL-7+IL-15 rather than IL-2, or with an inhibitor of the MAP kinase p38, facilitates this outcome. (See e.g., Chen, Gregory M. et al, Integrative bulk and single-cell profiling of pre-manufacture T-cell populations reveals factors mediating long-term persistence of CAR T-cell therapy, Cancer Discov Apr. 5, 2021; Krishna S. et al. Stem-like CD8 T cells mediate response of adoptive cell immunotherapy against human cancer. Science. 2020 Dec. 11; 370(6522):1328-1334; Yang Xu et al. Closely related T-memory stem cells correlate with in vivo expansion of CAR.CD19-T cells and are preserved by IL-7 and IL-15. Blood (2014) 123 (24): 3750-3759; Zhou, J., Jin, L., Wang, F. et al. Chimeric antigen receptor T (CAR-T) cells expanded with IL-7/IL-15 mediate superior antitumor effects. Protein Cell 10, 764-769 (2019); Gurusamy D, et al. Multi-phenotype CRISPR-Cas9 Screen Identifies p38 Kinase as a Target for Adoptive Immunotherapies. Cancer Cell. 2020; 37(6):818-833). Several phenotypic and functional characteristics of memory T cells can be measured after growing T cells in vitro to determine if certain reagents will generate T cells that are likely to persist in vivo and exhibit prolonged anti-tumor activity. These phenotypic characteristics include high expression of genes such as TCF7, CD62L, CCR7, and CD127, and low expression of other genes such as PD-1, CD39, and CD69. T cells with this desired memory phenotype produce more IL-2 upon in vitro restimulation compared to terminally differentiated effector T cells, indicating that they will undergo more rounds of autocrine IL-2-driven growth when they encounter tumor antigens in vivo.

One well-known negative regulator of T cell function is the cyclic AMP (cAMP)/protein kinase A (PKA) signaling pathway, which suppresses T cell responses, including production of IL-2 and interferon-γ. (Wehbi V L, Taskén K. Molecular Mechanisms for cAMP-Mediated Immunoregulation in T cells—Role of Anchored Protein Kinase A Signaling Units. Front Immunol. 2016; 7:222). Cyclic AMP is a “second-messenger” small molecule that is produced by G protein coupled receptors (GPCRs) that couple to Gαs and activate adenylyl cyclases to produce cAMP, which in turn binds the regulatory (R) subunit of PKA resulting in the release of active PKA catalytic (C) subunit for subsequent phosphorylation of substrates in various subcellular compartments. Cyclic AMP also activates a separate signaling protein termed EPAC. Gas-coupled GPCRs expressed on T cells include the prostaglandin receptors EP2 and EP4, the adenosine receptors A2A and A2B, GPR174, a receptor activated by lysophosphatidylserine (lysoPS), and the acidic pH sensor GPR65. Typical T cell cultures can contain high levels of adenosine and lysoPS and can also become acidic, leading to elevated cAMP signaling that may influence T cell phenotype. While suppressive effects of cAMP signaling on acute T cell responses following TCR or CD3/CD28 ligation have been described, the effects on T cell phenotypes following several days of cytokine-driven growth have not been explored until the present disclosure (See Mastelic-Gavillet, B., Navarro Rodrigo, B., Décombaz, L. et al. Adenosine mediates functional and metabolic suppression of peripheral and tumor-infiltrating CD8+ T cells. J. Immunotherapy Cancer 7, 257 (2019)).

The present disclosure is generally directed at methods and compositions for treating cancer. Namely, the present disclosure describes methods of manufacturing of therapeutic T cells comprising the step of contacting a population of T cells with one or more phenotype-altering agents (e.g., PKA inhibitors, GPR174 inhibitors, A2A inhibitors, or combinations thereof with one or more of p38 and/or PI3Kδ inhibitors) thereby modifying the phenotype of the T-cells. As used herein, the terms “T cell manufacturing,” “method of manufacturing T cells,” “method of generating T cells” or comparable terms refer to the process of producing a therapeutic composition of T cells, which manufacturing methods can comprise one or more of, or all of the following steps: harvesting, stimulation, activation, and expansion.

Surprisingly, the present inventors have identified that modification of T cells according to the methods described herein results in synergistic enhancement of the cells' anti-cancer and anti-tumor immune properties. Specifically, as described herein, the inventors discovered that inhibition of the cAMP/PKA pathway surprisingly promotes the expansion of memory-phenotype T cells that are more effective at reducing tumor growth. As further shown herein, combination of one or more PKA inhibitors, GPR174 inhibitors, A2A inhibitors, or combinations thereof with one or more p38 inhibitors and/or PI3Kδ inhibitors synergistically expanded these outcomes, resulting in a greater proportion of memory T cells capable of high-IL-2 production and markedly improved tumor killing in cancer models.

Accordingly, the present disclosure generally relates to in vivo and/or in vitro methods of inhibiting cancer and/or tumor growth and composition comprising therapeutic T cells. In some embodiments, T cells produced by the methods disclosed herein are administered to a mammalian subject, e.g., a human, a non-human primate, a dog, a cat, a horse, a sheep, a goat, a cow, a rabbit or a rodent. In some embodiments, the mammalian subject is a human. In some embodiments, the subject is a dog.

In one aspect, the disclosure provides a method for treating a disease, the method comprising administering to a subject in need thereof a therapeutically effective amount of phenotype-altered T cells wherein the phenotype-altered T cells are prepared by a method comprising the step of culturing a population of T cells in vitro or ex vivo in the presence of a phenotype-altering composition comprising one or more phenotype-altering agents for a time sufficient to alter a phenotype of at least a subpopulation of the population of T cells. As used herein, a “phenotype-altering agent” is an agent, such as a small molecule, a peptide, cocktail of peptides, an antibody or a fragment thereof, or a nucleic acid, that can alter the phenotype of at least a portion of a population of T cells when the T cells are cultured in the presence of the agent. In the embodiments of the methods and compositions of the disclosure, the phenotype-altering agent comprises an agent selected from the group consisting of a protein kinase A (PKA) inhibitor, an A2A adenosine receptor inhibitor, a GPR174 inhibitor, and combinations thereof. In some embodiments, the phenotype-altering composition further comprises a p38 inhibitor and/or a PI3Kδ inhibitor in addition to the one or more agents selected from the group consisting of a protein kinase A (PKA) inhibitor, an A2A adenosine receptor inhibitor, and a GPR174 inhibitor. In some embodiments of the methods and compositions disclosed herein, the PKA inhibitor is a PKA-RI or PKA-RII inhibitor, or a competitive antagonist of cAMP binding to PKA-RI or RII. Exemplary suitable p38 inhibitors, PI3Kδ inhibitors, protein kinase A (PKA) inhibitors, A2A adenosine receptor inhibitors, and GPR174 inhibitors are described in further details below.

Preferably, in the methods and compositions of the disclosure, the phenotype-altering agents are exogenous agents. As used herein, an “exogenous agent” is a small molecule or biomolecule that is not being produced by the cell (e.g., a T cell). Typically, in the methods of treatment of the disclosure, the phenotype-altering agent is removed from the cell culture prior to administration of the T cells to the subject, so that the phenotype-altering agent is not co-administered with the T cells.

T cells suitable to be used in the methods of the disclosure include autologous T cells and allogenic T cells. In some embodiments, the T cells are not genetically modified. For example, in some embodiments using adoptive T cell therapy approach (ACT), T cells can be taken from the patient, stimulated with the suitable tumor antigen and grown, and then administered the patient. These tumor-specific T cells are selected to expand by the in vitro stimulation.

In some embodiments, the population of T cells comprises genetically modified T cells. As used herein, the term “genetically engineered” or “genetically modified” refers to the addition of extra genetic material in the form of DNA or RNA into the total genetic material comprised in a cell or a deletion of a gene or a portion of a gene from the total genetic material comprised in a cell. In some embodiments, the genetically modified T cells comprise a deletion of a gene or a portion of a gene, for example, a gene encoding a checkpoint molecule such as PD-1 or a negative signaling molecule. In some embodiments, the genetically modified T cells comprise an exogenous nucleic acid, such as an exogenous nucleic acid encoding a T Cell Receptor (TCR), an exogenous nucleic acid encoding a Chimeric Antigen Receptor (CAR), or a combination thereof. In some embodiments, the T cells can be genetically modified to express a chimeric cytokine receptor such as those described in Oda S. et al, A Fas-4-1BB fusion protein converts a death to a pro-survival signal and enhances T cell therapy. J Exp Med. 2020, Dec. 7; 217(12), or a chimeric co-stimulatory molecule such as those described in Oda S K et al. A CD200R-CD28 fusion protein appropriates an inhibitory signal to enhance T-cell function and therapy of murine leukemia. Blood. 2017; 130(22):2410-2419.

As used herein, “altered phenotype,” also referred to herein as “phenotype-altered” means that the phenotype of at least a subpopulation of the population of T cells is altered after the culture period and/or the phenotype of at least a subpopulation of the population of T cells is altered after transfer of the T cells into the subject as compared to the phenotype of control T cells, wherein the control T cells are identical to the T cells cultured in the presence of the composition except that the control T cells are cultured without the presence of the composition.

In some embodiments, the altered phenotype is a phenotype displayed after the transfer of the T cells, obtained as described herein, into the subject, as compared to identical cells that were cultured without the presence of the composition. Non-limiting examples of such phenotypes include greater persistence, prolonged survival, greater antitumor activity, and combinations thereof as compared to control T cells, wherein the control T cells are identical to the T cells cultured in the presence of the composition except that the control T cells are not cultured in the presence of the composition.

In some embodiments, the phenotype-altered T cells have, before transfer into the subject, increased expression of one or more of CD62L, TCF1/TCF7, CCR7, and CD127, and/or decreased expression of one or more of CD69, CD39, CTLA-4, and PD-1, as compared to control T cells, wherein the control T cells are identical to the T cells cultured in the presence of the composition except that the control T cells are cultured without the presence of the composition. In some embodiments, the expression of one or more of CD62L, TCF1/TCF7, CCR7, and CD127 is increased by at least 10%, at least 20%, at least 30%, or at least 40%. In some embodiments, the expression of one or more of CD69, CD39, CTLA-4, and PD-1 is decreased by at least 10%, at least 20%, at least 30%, or at least 40%.

In some embodiments, the phenotype-altered T cells have, upon activation in a restimulation culture, increased expression of IL-2 as compared to control T cells, wherein the control T cells are identical to the T cells cultured in the presence of the composition except that the control T cells are cultured without the presence of the composition. In some embodiments, the expression of IL-2 is increased by at least 10%, at least 20%, at least 30%, or at least 40%.

In some embodiments, the T cells can be removed from the composition comprising one or more phenotype-altering agents and transferred into a restimulation culture that does not contain a phenotype-altering agent of the disclosure. In some embodiments, the restimulation culture does not contain the composition comprising one or more phenotype-altering agents but contains an anti-CD3 antibody or a combination of an anti-CD3 antibody and an anti-CD28 antibody.

In some embodiments, wherein the phenotype-altered T cells express a T Cell Receptor (TCR), the restimulation culture does not contain the composition comprising one or more phenotype-altering agents but contains tumor antigens that stimulate the T cell receptor (TCR) or cells expressing one or more tumor antigens that stimulate the T cell receptor (TCR).

In some embodiments, wherein the phenotype-altered T cells express a Chimeric Antigen Receptor (CAR), the restimulation culture does not contain the composition comprising one or more phenotype-altering agents but contains cells expressing one or more tumor antigens that stimulate the chimeric antigen receptor (CAR).

The T cells can be modified to express one or more engineered TCRs or CARs; for example, the T cells can be modified by transducing the T cells with a viral vector comprising an engineered TCR or CAR. In some embodiments, T cells can be modified prior to stimulation and activation in the presence of the phenotype altering compositions disclosed herein. In some embodiments, T cells are modified after stimulation and activation in the presence of the phenotype altering compositions disclosed herein. In some embodiments, T cells are modified within 12 hours, 24 hours, 36 hours, or 48 hours of stimulation and activation in the presence of the phenotype altering compositions disclosed herein.

In the methods of the disclosure, the population of T cells are cultured in the presence of a composition comprising one or more phenotype-altering agents described herein for a period of time sufficient to result in a change of at least one phenotype such as those phenotypes described above. In some embodiments, the population of T cells can be cultured in the presence of the compositions disclosed herein for at least about 2 days, at least about 3 days, at least about 4 days, for at least about 5 days, for at least about 6 days, for at least about 7 days, for at least about 8 days, for at least about 9 days, for at least about 10 days, for at least about 11 days, for at least about 12 days, for at least about 13 days, for at least about 14 days, for at least about 15 days, for at least about 16 days, for at least about 17 days, for at least about 18 days, for at least about 19 days, for at least about 20 days, for at least about 25 days, for at least about 30 days, or for at least about 40 days. In some embodiments, the population of T cells can be cultured in the presence of the compositions disclosed herein for up to about 2 days, up to about 3 days, up to about 4 days, up to about 5 days, for up to about 6 days, for up to about 7 days, for up to about 8 days, for up to about 9 days, for up to about 10 days, for up to about 11 days, for up to about 12 days, for up to about 13 days, for up to about 14 days, for up to about 15 days, for up to about 16 days, for up to about 17 days, for up to about 18 days, for up to about 19 days, for up to about 20 days, for up to about 25 days, for up to about 30 days, or for up to about 40 days.

In some embodiments, the number of T cells is expanded prior to administration to a patient in need thereof. Expansion of the numbers of T cells can be accomplished by any number of methods known in the art as described in, for example, U.S. Pat. Nos. 8,034,334; 8,383,099; and U.S. Patent Application Publication No. 2012/0244133. In some embodiments, the numbers of T cells are expanded by physically contacting the T cells with one or more non-specific T cell stimuli and one or more cytokines. For example, expansion of the numbers of T cells may be carried out by culturing the T cells with OKT3 antibody, IL-2, and/or feeder PBMC (e.g., irradiated allogeneic PBMC).

The phenotype-altering agents that can be used in the methods and compositions of the disclosure are further described below.

A2A Adenosine Receptor Inhibitors

In some embodiments, the phenotype altering agent is an A2A adenosine receptor inhibitor. In some embodiments, the phenotype altering agent is a combination of agents that comprises an A2A adenosine receptor inhibitor.

The A2A adenosine receptor inhibitor can be a polypeptide, an antibody, a non-peptide compound, an expression inhibitor (e.g., A2A adenosine receptor inhibitor antisense nucleic acid molecules such as antisense RNA, antisense DNA, antisense synthetic oligonucleotide analogs, ribozymes, or other RNA-interfering molecules) that inhibits A2A adenosine receptor expression, or a small molecule (e.g., a small organic or organometallic molecule). Examples of such inhibitors are known in the art; for example, those disclosed in Masoumi, E. et al. Genetic and pharmacological targeting of A2a receptor improves function of anti-mesothelin CAR T cells. J Exp Clin Cancer Res 39, 49 (2020).

In some embodiments, the A2A adenosine receptor inhibitor is a small molecule. Exemplary A2A adenosine receptor inhibitors include ZM 241385 (CAS 139180-30-6), istradefylline (CAS 155270-99-8), xanthine amine congener (CAS 96865-92-8), XCC (CAS 96865-83-7), ANR 94 (CAS 634924-89-3), PSB 1115 (CAS 409344-71-4), 3,7-dimethyl-1-propargylxanthine (CAS 14114-46-6), SCH 58261 (CAS 160098-96-4), SCH 442416 (CAS 316173-57-6), 8-(3-chlorostyryl)caffeine (CAS 147700-11-6), CGS 15943 (CAS 104615-18-1), ST4206 (CAS 246018-36-9), KF21213 (CAS 155271-17-3), regadenoson (CAS 313348-27-5), preladenant (CAS 377727-87-2), CGS 21680 (CAS 120225-54-9), tozadenant (CAS 870070-55-6), Sch412348 (CAS 377727-26-9), ST3932 (CAS 1246018-21-2), A2A receptor antagonist 1 (CPI-444 analog; CAS 443103-97-7), istradefylline (CAS 155270-99-8), AZD4635 (CAS 1321514-06-0), CGS 15943 (CAS 104615-18-1), vipadenant (CAS 442908-10-3), CPI-444 (CAS 1202402-40-1), TC-G 1004 (CAS 1061747-72-5), 4-desmethyl istradefylline (CAS 160434-48-0), PSB 0777 (CAS 2122196-16-9), and combinations thereof.

PKA Inhibitors

In some embodiments, the phenotype altering agent is a protein kinase A (PKA) inhibitor. In some embodiments, the PKA inhibitor is a PKA-RI inhibitor, a PKA-RII inhibitor, a competitive antagonist of cAMP binding to PKA-RI, a competitive antagonist of cAMP binding to PKA-RII, or a competitive antagonist of cAMP binding to both PKA-RI and PKA-RII. In some embodiments, the phenotype altering agent is a combination of agents that comprises a protein kinase A (PKA) inhibitor. The PKA inhibitor can be a polypeptide, an antibody, a non-peptide compound or small molecule (e.g., a small organic or organometallic molecule), or an expression inhibitor (e.g., PKA-Cα or PKA-Cβ antisense nucleic acid molecules such as antisense RNA, antisense DNA, antisense synthetic oligonucleotide analogs, ribozymes, or other RNA-interfering molecules) that inhibits PKA-Cα or PKA-Cβ kinase activity or expression. Examples of such inhibitors of PKA function or PKA-C expression are known in the art, for instance, those described in Liu C, Ke P, Zhang J, Zhang X, Chen X. Protein Kinase Inhibitor Peptide as a Tool to Specifically Inhibit Protein Kinase A. Front Physiol. 2020 Nov. 25; 11:574030, or Sugiyama H, Chen P, Hunter M G, Sitkovsky M V. Perturbation of the expression of the catalytic subunit C alpha of cyclic AMP-dependent protein kinase inhibits TCR-triggered secretion of IL-2 by T helper hybridoma cells. J Immunol. 1997 Jan. 1; 158(1):171-9.

In some embodiments, the PKA inhibitors useful in the methods and compositions of the disclosure are small molecules. Both orally available PKA inhibitors and PKA inhibitors with low oral availability can be used herein. In some embodiments, the protein kinase A (PKA) inhibitor is selected from the group consisting of HA-100 dihydrochloride, Rp-cAMPS, H-89 dihydrochloride, PKI (5-24), Staurosporine, Calphostin C, KT-5720, Rp-8-Br-cAMPS, 5-Iodotubercidin, Piceatannol, Fasudil (monohydrochloride salt), ML-7 hydrochloride, CGP-74514A hydrochloride, ML-9, Daphnetin, Myricetin, PKC-412, A-674563, K-252a, H-7 dihydrochloride, bisindolylmaleimide IV, cGK1alpha inhibitor-cell permeable DT-3, TX-1123, Rp-8-PIP-cAMPS, 8-bromo2′-monobutyrladenosine-3′,5′-cyclic monophosphorothioate Rp-isomer, Bisindolylmaleimide III hydrochloride, Rp-adenosine 3′,5′-cyclic monophosphorothioate sodium salt, A-3 hydrochloride, H-7, H-8-2HCl, K252c, HA-1004 dihydrochloride, K-252b, HA-1077 dihydrochloride, MDL-27,032, H-9 hydrochloride, Rp-8-CPT-cAMPS, bisindolylmaleimide III, -lacetamido-4-cyano-3-methyllisoquinoline, Ilmofosine, Rp-8-hexylaminoadenosine 3′,5′-monophosphorothioate, HA-1004 hydrochloride, PKA Inhibitor IV, Adenosine 3′,5′-cyclic monophosphorothioate 8-chloro Rp-isomer sodium salt, adenosine 3′,5′cyclic monophosphorothioate 2′-O-monobutyryl Rp-isomer sodium salt, 4-cyano-3-methylisoquinoline, 8-hydroxyadenosine-3′,5′-monophosphorothioate Rp-isomer, PKI (6-22) amide, SB 218078, Rp-8-pCPT-cyclic GMPS sodium, Sp-8-pCPT-cAMPS, N[2-(p-Cinnamylamino)shyethyl]-5-isoquinolone sulfonamide, AT7867, GSK 690693, PKI (14-22) amide (myristoylated), Rp-8-bromo-cAMPS, or combinations thereof. In some embodiments, the PKA inhibitor is identified by one of the following CAS numbers: 84468-24-6, 151837-09-1, 130964-39-5, 99534-03-9, 62996-74-1, 121263-19-2, 108068-98-0, 129735-00-8, 24386-93-4, 10083-24-6, 105628-07-7, 110448-33-4, 1173021-98-1, 105637-50-1, 486-35-1, 529-44-2, 120685-11-2, 552325-73-2, 99533-80-9, 108930-17-2, 119139-23-0, 157397-06-3, 156816-36-3, 788807-32-9, 73208-40-9, 78957-85-4, 84477-87-2, 113276-94-1, 85753-43-1, 91742-10-8, 99570-78-2, 203911-27-7, 110124-55-5, 116970-50-4, 129735-01-9, 137592-43-9, 179985-52-5, 83519-04-4, 92564-34-6, 99534-03-9, 142754-27-6, 152218-23-0, 161468-32-2, 121932-06-7, 135897-06-2, 153660-04-9, 129693-13-6, 130964-40-8, 857531-00-1, 937174-76-0, or 201422-03-9.

In some embodiments, the PKA inhibitor is a cAMP analog, such as (Rp)-8-Br-cAMPS or (Rp)-8-Cl-cAMPS as disclosed, for example, in Gjertsen B T et al. Novel (Rp)-cAMPS analogs as tools for inhibition of cAMP-kinase in cell culture. Basal cAMP-kinase activity modulates interleukin-1 beta action. J Biol Chem. 1995 Sep. 1; 270(35): 20599-607.

In some embodiments, the PKA inhibitor is a small molecule described in US Patent Application No. 20060100166 and Schwede F. et al Rp-cAMPS Prodrugs Reveal the cAMP Dependence of First-Phase Glucose-Stimulated Insulin Secretion. Mol Endocrinol. 2015 July; 29(7):988-1005.

In some embodiments, the PKA inhibitor is a compound of the following structure:

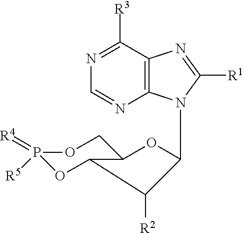

or a deaza-analog thereof, wherein:
R¹ can be independently H, halogen, azido, alkyl, aryl, amido-alkyl, amido-aryl, OH, O-alkyl, O-aryl, SH, S-alkyl, S-aryl, SeH, Se-alkyl, Se-aryl, amino, NH-alkyl, NH-aryl, N-bisalkyl, N-bisaryl, or cycloalkylamino;
R² can be independently H, halogen, azido, O-alkyl, S-alkyl, Se-alkyl, NH-alkyl, N-bisalkyl, alkyl-carbamoyl, cycloalkylamino, or silyl;
R³ can be independently H, halogen, OH, azido, amido-alkyl, amido-aryl, O-alkyl, O-aryl, SH, S-alkyl, S-aryl, amino, NH-alkyl, NH-aryl, N-bisalkyl, N-bisaryl, NH-alkyl-carbamoyl, or cycloalkylamino; and wherein
R⁴ is O(H) or S(H) and R⁵ is O(H), S(H), amino, H, alkyl, O-alkyl, O-aryl, S-alkyl, S-aryl, NH-alkyl, NH-aryl, N-bisalkyl, or N-bisaryl;
or R⁴ is O(H), S(H), amino, H, alkyl, O-alkyl, O-aryl, S-alkyl, S-aryl, NH-alkyl, NH-aryl, N-bisalkyl, N-bisaryl; and R⁵ is O(H) or S(H); and pharmaceutically acceptable salts, esters, and/or solvates thereof.

In some embodiments, the PKA inhibitor is 8-bromo-2′-deoxyadenosine-3′,5′-cyclic monophosphate; 8-(4-chloro-phenylthio)-2′-deoxyadenosine-3′,5′-cyclic monophosphate; 8-(4-chloro-phenylthio)-N.sup.6-phenyl-2′-deoxyadenosine-3′,5′-cyclic monophosphate; 8-bromo-2′-O-methyladenosine-3′,5′-cyclic monophosphate; 8-(4-chloro-phenylthio)-2′-O-methyladenosine-3′,5′-cyclic monophosphate; 8-methylamino-2′-O-methyladenosine-3′,5′-cyclic monophosphate; 8-methylthio-2′-O-methyladenosine-3′,5′-cyclic monophosphate; 8-(4-fluoro-phenylthio)-2′-O-methyladenosine-3′,5′-cyclic monophosphate; 8-(4-methyl-cumarinyl-7-thio)-2′-O-methyladenosine-3′,5′-cyclic monophosphate; 8-(naphtyl-2-thio)-2′-O-methyladenosine-3′,5′-cyclic monophosphate; 8-phenylthio-2′-O-methyladenosine-3′,5′-cyclic monophosphate; 8-(4-nitro-phenylthio)-2′-O-methyladenosine-3′,5′-cyclic monophosphate; 8-(2-amino-phenylthio)-2′-O-methyladenosine-3′,5′-cyclic monophosphate; 8-benzylthio-2′-O-methyladenosine-3′,5′-cyclic monophosphate; 8-n-hexylthio-2′-O-methyladenosine-3′,5′-cyclic monophosphate; 8-phenylethylamino-2′-O-methyladenosine-3′,5′-cyclic monophosphate; 8-(4-methoxy-phenylthio)-2′-O-methyladenosine-3′,5′-cyclic monophosphate; 8-isopropylthio-2′-O-methyladenosine-3′,5′-cyclic monophosphate; 8-(benzimidazolyl-2-thio)-2′-O-methyladenosine-3′,5′-cyclic monophosphate; 8-(2-hydroxy-ethylthio)-2′-O-methyladenosine-3′,5′-cyclic monophosphate; 8-ethylthio-2′-O-methyladenosine-3′,5′-cyclic monophosphate; 8-(2-amino-ethylthio)-2′-O-methyladenosine-3′,5′-cyclic monophosphate; 8-(pyridinyl-2-thio)-2′-O-methyladenosine-3′,5′-cyclic monophosphate; 8-(benzothiazolyl-2-thio)-2′-O-methyladenosine-3′,5′-cyclic monophosphate; 8-(4-methyl-phenylthio)-2′-O-methyladenosine-3′,5′-cyclic monophosphate; 8-(3-methoxy-phenylthio)-2′-O-methyladenosine-3′,5′-cyclic monophosphate; 8-(4-isopropyl-phenylthio)-2′-O-methyladenosine-3′,5′-cyclic monophosphate; 8-(2,3,5,6-tetrafluoro-phenylthio)-2′-O-methyladenosine-3′,5′-cyclic monophosphate; 8-(4-hydroxy-phenylthio)-2′-O-methyladenosine-3′,5′-cyclic monophosphate; 8-(2,4-dichloro-phenylthio)-2′-O-methyladenosine-3′,5′-cyclic monophosphate; 8-(4-chloro-phenylthio)-2′-(N,N-dimethyl)-carbamoyl-adenosine-3′,5′-cycli-c monophosphate; 8-methoxy-2′-O-methyladenosine-3′,5′-cyclic monophosphate; 8-benzyloxy-2′-O-methyladenosine-3′,5′-cyclic monophosphate; 8-bromo-2′-O-methyladenosine-3′,5′-cyclic monophosphorothioate, Sp-isomer; 8-bromo-2′-O-methyladenosine-3′-5′-cyclic monophophorothioate, Rp-isomer, 8-(4-chloro-phenylthio)-2′-O-methyladenosine-3′,5′-cyclic monophosphorothioate, Sp-isomer; 8-(4-chloro-phenylthio)-2′-O-methyladenosine-3′,5′-cyclic monophosphorothioate, Rp-isomer; 8-bromo-2′-deoxyadenosine-3′,5′-cyclic monophosphorothioate, Rp-isomer; 8-bromo-2′-deoxyadenosine-3′,5′-cyclic monophosphorothioate, Sp-isomer; 8-(4-chloro-phenylthio)-2′-deoxyadenosine-3′,5′-cyclic monophosphorothioate, Rp-isomer; 8-(4-chloro-phenylthio)-2′-deoxyadenosine-3′,5′-cyclic monophosphorothioate, Sp-isomer; and 8-cyclohexylamino-2′-deoxyadenosine-3′,5′-cyclic monophosphate; 8-chloro-2′-O-methyladenosine-3′,5′-cyclic monophosphate, or N⁶-tert-butyl-8-(4-chloro-phenylthio)-2′-deoxyadenosine-3′,5′-cyclic monophosphate.

In some embodiments, the PKA inhibitor is a compound disclosed in US Patent Application No. 20060100166, the disclosure of which is incorporated herein by reference in its entirety.

In some embodiments, the PKA inhibitor is a cell-permeable prodrug of a cAMP analog.

In some embodiments, PKA inhibitor is a compound of the following structure:

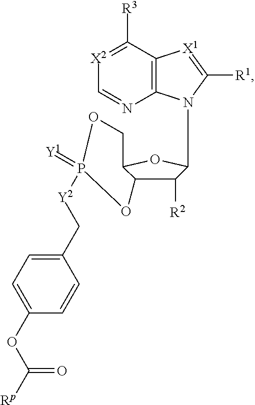

wherein:
R¹ is H, halogen, azido, alkyl, aryl, amido-alkyl, amido-aryl, OH, O-alkyl, O-aryl, SH, S-alkyl, S-aryl, SeH, Se-alkyl, Se-aryl, amino, NH-alkyl, NH-aryl, N-bisalkyl, N-bisaryl, or cycloalkylamino;
R² is H, halogen, azido, OH, O-alkyl, S-alkyl, Se-alkyl, NH-alkyl, N-bisalkyl, alkyl-carbamoyl, cycloalkylamino, or silyl;
R³ is H, halogen, OH, azido, amido-alkyl, amido-aryl, O-alkyl, O-aryl, SH, S-alkyl, S-aryl, amino, NH-alkyl, NH-aryl, N-bisalkyl, N-bisaryl, NH-alkyl-carbamoyl, or cycloalkylamino; and wherein
Y¹ and Y² are independently is O or S;
X¹ and X² are independently is CH or N; and
R^{p} is alkyl.

In some embodiments, the PKA inhibitor is 8-Br-cAMPS, Rp-isomer (Rp-8-Br-cAMPS; CAS Number 925456-59-3), or its 4-acetoxybenzyl ester (Rp-8-Br-cAMPS-pAB).

p³⁸ Inhibitors

In some embodiments, in addition to the agents described above, the compositions can further comprise a p38 inhibitor. Any suitable p38 inhibitors can be used in the method and compositions of the disclosure.

The p38 inhibitor can be any agent that inhibits the biological activity of p38 MAPK. In some embodiments, the p38 inhibitor may be an allosteric inhibitor or a non-allosteric inhibitor of p38 MAPK. In some embodiments, the p38 inhibitor can be p38 MAPK isoform-specific or p38 MAPK isoform non-specific. P38 MAPK (also referred to as mitogen-activated protein kinase 14 or MAPK14) has four isoforms: p38 MAPK-alpha (α), p38 MAPK-beta (β), p38 MAPK-gamma (γ), and p38 MAPK-delta (δ). In some embodiments, the p38 inhibitor can inhibit any one or more of p38 MAPK-α, p38 MAPK-β, p38 MAPK-γ, and p38 MAPK-δ. The isoforms p38 MAPK-α and p38 MAPK-β are expressed by T-cells. Accordingly, in some preferred embodiments, the p38 inhibitor in an agent that inhibits one or both of p38 MAPK-α and p38 MAPK-β. The PKA inhibitor can be a polypeptide, an antibody, a non-peptide compound or small molecule (e.g., a small organic or organometallic molecule), or an expression inhibitor (e.g., PKA-Cα or PKA-Cβ antisense nucleic acid molecules such as antisense RNA, antisense DNA, antisense synthetic oligonucleotide analogs, ribozymes, or other RNA-interfering molecules) that inhibits PKA-Cα or PKA-Cβ kinase activity or expression. Examples of such inhibitors are known in the art; for instance, those described in PCT Application WO2000059919A1; Duan W. et al., Am J Respir Crit Care Med Vol 171. pp 571-578, 2005; and Aoshiba K. et al., J Immunol February 1, 162 (3) 1692-1700, 1999.

In some embodiments, the p38 inhibitors useful in the methods and compositions of the disclosure are small molecules. Both orally available and p38 inhibitors with low oral availability can be used herein. In some embodiments, the p38 inhibitor is selected from the group consisting of doramapimod (CAS 285983-48-4), losmapimod (CAS 585543-15-3), SX 011 (CAS 309913-42-6), SB202190 (CAS 350228-36-3), VX 702 (CAS 745833-23-2), JX-401 (CAS 349087-34-9), p38 MAP Kinase Inhibitor VIII (CAS 321351-00-2). SCIO 469 (CAS 309913-83-5), p38 MAP Kinase Inhibitor V (CAS 271576-77-3), p38 MAP Kinase Inhibitor IX (N-(isoazol-3-yl)-4-methyl-3-(1-phenyl-1H-pyrazolo[3,4-d]pyrimidin-4-ylamino)-benzamide), PD 169316 (CAS 152121-53-4), p38 MAP Kinase Inhibitor III (CAS 581098-48-8), PH-797804 (CAS 586379-66-0), RWJ 67657 (CAS 215303-72-3), VX 745 (CAS 209410-46-8), LY 364947 (CAS 396129-53-6), p38 MAP Kinase Inhibitor (CAS 219138-24-6), SB 239063 (CAS 193551-21-2), SB 202190 (CAS 152121-30-7), SB 203580 (CAS 152121-47-6), p38 MAP Kinase Inhibitor IV (CAS 1638-41-1), SD-169 (CAS 1670-87-7), N-(5-Chloro-2-methylphenyl)-7-nitrobenzo[c][1,2,5]oxadiazol-4-amine (FGA-19), or a combination thereof.

In some embodiments, the phenotype altering agent is doramapimod (CAS 285983-48-4). In some embodiments, the phenotype altering agent comprises doramapimod in combination with Rp-8-Br-cAMPS or its 4-acetoxybenzyl ester (Rp-8-Br-cAMPS-pAB).

In some embodiments, the p38 inhibitors can be selected from the group consisting of Ralimetinib (LY2228820) Dilmapimod (SB-681323 or GW681323) Losmapimod (GW856553X), 5-(2,6-Dichlorophenyl)-2-[2,4-difluorophenyl)thio]-6H-pyrimido[1,6-b]pyridazin-6-one (Neflamapimod or VX-745), 6-(N-carbamoyl-2,6-difluoroanilino)-2-(2,4-difluorophenyl)-3-pyridinecarboxamide (VX-702), Pamapimod (RO-4402257), Talmapimod (SCIO-469), doramapimod (BIRB-796), 5-p-chlorophenyl-3-[N-(2-hydroxyacetyl)piperidin-4-yl]-4-pyrimidin-4-yl-1H-pyrazole (SD-0006), 3-[3-bromo-4-[(2,4-difluorophenyl)-methoxy]-6-methyl-2-oxo-1(2H)-pyridinyl]-N,4-dimethyl-benzamide (PH-797804), 2-(2S)-2-amino-3-phenylpropylamino-3-methyl-5-(2-naphthalenyl)-6-(4-pyridinyl)-4(3H)-pyrimidi-none (AMG-548), and combinations thereof.

PI3K Inhibitors

In some embodiments, the compositions of the disclosure can comprise a PI3K inhibitor, such as a PI3Kδ inhibitor. As used herein, the term “PI3K inhibitor” refers to a nucleic acid, peptide, compound, or small organic molecule that inhibits at least one activity of PI3K. In some embodiments, the PI3K inhibitor binds to and inhibits at least one activity of PI3K, e.g., PI3K δ. The PI3K proteins can be divided into three classes, class 1 PI3Ks, class 2 PI3Ks, and class 3 PI3Ks. Class 1 PI3Ks exist as heterodimers consisting of one of four p110 catalytic subunits (p110α, p110β, p110δ, and p110γ) and one of two families of regulatory subunits. A PI3K inhibitor of the present disclosure preferably targets the class 1 PI3K inhibitors. In some embodiments, a PI3K inhibitor is selective for one or more isoforms of the class 1 PI3K (i.e., selective for p110α, p110β, p110δ, and/or p110γ). In some embodiments, a PI3K inhibitor will not display isoform selectivity and be considered a “pan-PI3K inhibitor.” In some embodiments, a PI3K inhibitor competes for binding with ATP to the PI3K catalytic domain. Preferably, in some embodiments, the PI3K inhibitor is a PI3Kδ inhibitor.

In some embodiments, a PI3K inhibitor, such as a PI3Kδ inhibitor, can target PI3K as well as additional proteins in the PI3K-AKT-mTOR pathway. In some embodiments, a PI3K inhibitor that targets both mTOR and PI3K can be referred to as either a mTOR inhibitor or a PI3K inhibitor. A PI3K inhibitor that only targets PI3K can be referred to as a selective PI3K inhibitor. In one embodiment, a selective PI3K inhibitor can be understood to refer to an agent that exhibits a 50% inhibitory concentration with respect to PI3K that is at least 10-fold, at least 20-fold, at least 30-fold, at least 50-fold, at least 100-fold, at least 1000-fold, or more, lower than the inhibitor's IC₅₀ with respect to mTOR and/or other proteins in the pathway.

Illustrative non-limiting examples of PI3K inhibitors suitable for use in the methods of the disclosure include, but are not limited to, BKM120 (class 1 PI3K inhibitor, Novartis), XL147 (class 1 PI3K inhibitor, Exelixis), (pan-PI3K inhibitor, GlaxoSmithKline), and PX-866 (class 1 PI3K p110α, p110β, p110δ, and p110γ isoforms, Oncothyreon).

In some embodiments, the PI3K inhibitor is a selective PI3Kδ inhibitor. Non-limiting examples of selective PI3Kδ inhibitors are Acalisib (GS-9820, CAL-120), Dezapelisib (INCB040093), Idelalisib (CAL-101, GS-1101), Leniolisib (CDZ173), Inperlisib (YY-20394, PI3K(delta)-IN-2), Nemiralisib (GSK2269557), Parsaclisib (INCB050465, IBI-376), Puquitinib (XC-302), Seletalisib (UCB-5857), Zandelisib (ME-401, PWT143), ACP-319 (AMG 319), BGB-10188, GS-9901, GSK2292767, HMPL-689, IOA-244 (MSC236084), RV1729, and SHC014748M.

In some embodiments, the selective PI3Kδ inhibitor is idelalisib (CAL-101). In some embodiments, the phenotype altering agent comprises doramapimod in combination with Rp-8-Br-cAMPS (or its 4-acetoxybenzyl ester (Rp-8-Br-cAMPS-pAB)) and idelalisib (CAL-101).

GPR174 Inhibitors

In some embodiments, the phenotype altering agent is a GPR174 inhibitor. In some embodiments, the phenotype altering composition comprises a combination of agents that comprises a GPR174 inhibitor. In some embodiments, the GPR174 inhibitor is a small molecule.

In some embodiments, the GPR174 inhibitor is not an endogenous ligand of GPR174 (e.g., is a surrogate ligand). In various embodiments, the GPR174 inhibitor is a functional inhibitor of a GPR174-mediated signaling pathway (e.g., an antagonist, partial agonist, inverse agonist, partial inverse agonist, or negative allosteric modulator). The GPR174 inhibitor can be a polypeptide, an antibody, a non-peptide compound, an expression inhibitor (e.g., GPR174 antisense nucleic acid molecules such as antisense RNA, antisense DNA or antisense oligonucleotides, GPR174 ribozymes or GPR174 RNAi molecules) that inhibits GPR174 expression, or a small molecule (e.g., a small organic or organometallic molecule). Examples of such inhibitors are known in the art; for instance, SIRGT46986WQ-2OMe, a small interfering RNA (siRNA) that targets GPR174 gene, available from Creative Biolabs (London, UK).

In some embodiments, the methods and compositions of any of the aspects of the disclosure can employ any GPR174 inhibitor such as a compound having a structure according to the formulas described below, such as the exemplary compounds in Table 1, or a pharmaceutically acceptable salt thereof.

In some embodiments, the GPR174 inhibitor has a structure according to the following formula (I):

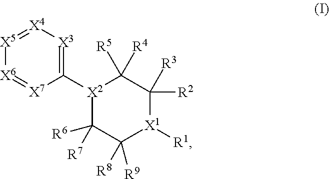

or a stereoisomer thereof, or a pharmaceutically acceptable salt thereof, wherein,
   X¹ is N or CR¹⁰;
   X² is N or CR¹¹;
   X³ is N or CR¹²;
   X⁴ is N or CR¹³;
   X⁵ is N or CR¹⁴;
   X⁶ is N or CR¹⁵;
   X⁷ is N or CR¹⁶;
each of R¹, R², R³, R⁴, R⁵, R⁶, R⁷, R⁸, and R⁹ is, independently, H, hydroxy, thiol, optionally substituted amino, optionally substituted amido, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted ester, optionally substituted carboxamide, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ alkylsulfinyl, optionally substituted C₁-C₆ alkylsulfonyl, optionally substituted C₆-C₁₀ arylsulfinyl, optionally substituted C₆-C₁₀ arylsulfonyl, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₅-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl; or
   R² and R³ combine to form ═O, ═S, or ═NR¹⁷; or
   R⁴ and R⁵ combine to form ═O, ═S, or ═NR¹⁷; or
   R⁶ and R⁷ combine to form ═O, ═S, or ═NR¹⁷; or
   R⁸ and R⁹ combine to form ═O, ═S, or ═NR¹⁷;
each of R¹⁰, R¹¹, R¹², R¹³, R¹⁴, R¹⁵, and R¹⁶ is, independently, H, hydroxy, halogen, thiol, optionally substituted amino, optionally substituted amido, cyano, nitro, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted ester, optionally substituted carboxamide, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ alkylsulfinyl, optionally substituted C₁-C₆ alkylsulfonyl, optionally substituted C₆-C₁₀ arylsulfinyl, optionally substituted C₆-C₁₀ arylsulfonyl, optionally substituted C₁-C₉ heteroarylsulfinyl, optionally substituted C₁-C₉ heteroarylsulfonyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl; or
one of:
(i) R¹² and R¹³, together with the atoms to which each is attached, combine to form an optionally substituted 5-, 6-, or 7-member ring;
(ii) R¹³ and R¹⁴, together with the atoms to which each is attached, combine to form an optionally substituted 5-, 6-, or 7-member ring;
(iii) R¹⁴ and R¹⁵, together with the atoms to which each is attached, combine to form an optionally substituted 5-, 6-, or 7-member ring; and
(iv) R¹⁵ and R¹⁶, together with the atoms to which each is attached, combine to form an optionally substituted 5-, 6-, or 7-member ring;
and
R¹⁷ is H, hydroxyl, cyano, optionally substituted amino, optionally substituted amido, optionally substituted carboxamide, optionally substituted C₁-C₆ alkyl, optionally substituted C₂-C₆alkenyl, optionally substituted C₂-C₆ alkynyl, optionally substituted C₁-C₆ alkanoyl, optionally substituted C₇-C₁₁ aryloyl, optionally substituted C₂-C₁₀ heterocyclyloyl, optionally substituted C₂-C₁₀ heteroaryloyl, optionally substituted C₁-C₆ alkylsulfinyl, optionally substituted C₁-C₆ alkylsulfonyl, optionally substituted C₆-C₁₀ arylsulfinyl, optionally substituted C₆-C₁₀ arylsulfonyl, optionally substituted C₁-C₉ heteroarylsulfinyl, optionally substituted C₁-C₉ heteroarylsulfonyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
wherein three or fewer of X³, X⁴, X⁵, X⁶, and X⁷ are N; and
at least one of X¹ and X² is N.

In some embodiments of formula (I), X¹ is N. In certain embodiments of formula (I), X² is N. In particular embodiments of formula (I), X³ is CR¹². In other embodiments of formula (I), X⁴ is CR¹³. n yet other embodiments of formula (I), X⁵ is CR¹⁴. In still other embodiments of formula (I), X⁶ is CR¹⁵. In certain other embodiments of formula (I), X⁷ is CR¹⁶.

In some embodiments of formula (I), the isolated compound has the structure according to formula (IA):

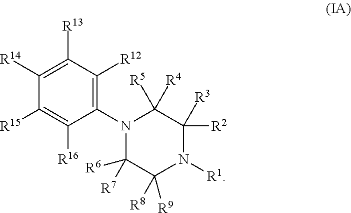

In certain embodiments of formula (I) or (IA), R² is H, optionally substituted C₁-C₆ alkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In some embodiments of formula (I) or (IA), R² is H or optionally substituted C₁-C₆ alkyl. In other embodiments of formula (I) or (IA), R² is H.

In some embodiments of formula (I) or (IA), R³ is H, optionally substituted C₁-C₆ alkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In still other embodiments of formula (I) or (IA), R³ is H or optionally substituted C₁-C₆ alkyl. In particular embodiments of formula (I) or (IA), R³ is H.

In some embodiments of formula (I) or (IA), R⁴ is H, optionally substituted C₁-C₆ alkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In certain embodiments of formula (I) or (IA), R⁴ is H or optionally substituted C₁-C₆ alkyl. In particular embodiments of formula (I) or (IA), R⁴ is H.

In some embodiments of formula (I) or (IA), R⁵ is H, optionally substituted C₁-C₆ alkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In yet other embodiments of formula (I) or (IA), R⁵ is H or optionally substituted C₁-C₆ alkyl. In still other embodiments of formula (I) or (IA), R⁵ is H.

In some embodiments of formula (I) or (IA), R⁶ is H, optionally substituted C₁-C₆ alkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In certain embodiments of formula (I) or (IA), R⁶ is H or optionally substituted C₁-C₆ alkyl. In some embodiments of formula (I) or (IA), R⁶ is H.

In some embodiments of formula (I) or (IA), R⁷ is H, optionally substituted C₁-C₆ alkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In other embodiments of formula (I) or (IA), R⁷ is H or optionally substituted C₁-C₆ alkyl. In still other embodiments of formula (I) or (IA), R⁷ is H.

In some embodiments of formula (I) or (IA), R⁸ is H, optionally substituted C₁-C₆ alkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In certain embodiments of formula (I) or (IA), R¹ is H or optionally substituted C₁-C₆ alkyl. In particular embodiments of formula (I) or (IA), R⁸ is H.

In some embodiments of formula (I) or (IA), R⁹ is H, optionally substituted C₁-C₆ alkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In yet other embodiments of formula (I) or (IA), R⁹ is H or optionally substituted C₁-C₆ alkyl. In still other embodiments of formula (I) or (IA), R⁹ is H.

In some embodiments of formula (I) or (IA), R¹³ is H, hydroxy, optionally substituted amino, optionally substituted C₁-C₆ alkyl, optionally substituted C₁-C₆ heteroalkyl, optionally substituted C₃-C₁₀ cycloalkyl, optionally substituted C₁-C₆ alkoxy, optionally substituted C₆-C₁₀ aryloxy, optionally substituted C₁-C₉ heteroaryloxy, optionally substituted C₆-C₁₀ aryl, optionally substituted C₁-C₉ heteroaryl, or optionally substituted C₁-C₉ heterocyclyl. In certain embodiments of formula (I) or (IA), R¹³ is H or optionally substituted C₁-C₆ alkyl. In other embodiments of formula (I) or (IA), R¹³ is H.

In some embodiments of formula (I) or (IA), R¹⁶ is H, halogen, cyano, nitro, optionally substituted C₁-C₆ alkyl, optionally substituted C₃-C₁₀ cycloalkyl, optionally substituted C₆-C₁₀ aryl, optionally substituted C₁-C₉ heteroaryl, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted ester, or optionally substituted C₁-C₉ heterocyclyl. In other embodiments of formula (I) or (IA), R¹⁶ is H or optionally substituted C₁-C₆ alkyl. In yet other embodiments of formula (I) or (IA), R¹⁶ is H.

In some embodiments of formula (I), the compound has the structure according to formula (IB):

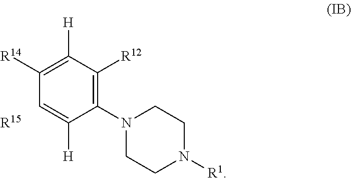

In some embodiments of formula (I), (IA), or (IB), R¹² is H, halogen, cyano, nitro, optionally substituted C₁-C₆ alkyl, optionally substituted C₁-C₆ alkoxy, optionally substituted C₁-C₆ alkanoyl, optionally substituted C₇-C₁₁ aryloyl, optionally substituted C₂-C₁₀ heterocyclyloyl, optionally substituted C₂-C₁₀ heteroaryloyl, optionally substituted C₁-C₆ alkylsulfonyl, optionally substituted C₆-C₁₀ arylsulfonyl, optionally substituted C₁-C₉ heteroarylsulfonyl, hydroxycarbonyl, optionally substituted ester, optionally substituted carboxamide, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₉ heteroaryl, or optionally substituted C₁-C₉ heterocyclyl. In particular embodiments of formula (I), (IA), or (IB), R¹² is H, halogen, cyano, nitro, optionally substituted C₁-C₆ alkyl, optionally substituted C₁-C₆ alkoxy, optionally substituted C₁-C₆ alkanoyl, optionally substituted C₁-C₆ alkylsulfonyl, hydroxycarbonyl, optionally substituted ester, optionally substituted carboxamide, optionally substituted C₁-C₆ alkanoyloxy, optionally substituted C₁-C₉ heteroaryl, or optionally substituted C₁-C₉ heterocyclyl. In other embodiments of formula (I), (IA), or (IB), R¹² is H, halogen, cyano, nitro, optionally substituted C₁-C₆ alkyl, optionally substituted ester, optionally substituted carboxamide, optionally substituted C₁-C₆ alkanoyloxy, or optionally substituted C₁-C₉ heteroaryl. In yet other embodiments of formula (I), (IA), or (IB), R¹² is H, halogen, nitro, optionally substituted ester, or optionally substituted C₁-C₆ alkanoyloxy. In still other embodiments of formula (I), (IA), or (IB), R¹² is halogen (e.g., R¹² is fluorine). In certain embodiments of formula (I), (IA), or (IB), R¹² is nitro.

In some embodiments of formula (I), (IA), or (IB), R¹⁴ is H, halogen, cyano, nitro, optionally substituted C₁-C₆ alkyl, hydroxycarbonyl, optionally substituted ester, optionally substituted carboxamide, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ alkanoyl, optionally substituted C₇-C₁₁ aryloyl, optionally substituted C₂-C₁₀ heterocyclyloyl, optionally substituted C₂-C₁₀ heteroaryloyl, optionally substituted C₁-C₆ alkylsulfonyl, optionally substituted C₆-C₁₀ arylsulfonyl, optionally substituted C₁-C₉ heteroarylsulfinyl, optionally substituted C₆-C₁₀ aryl, optionally substituted C₁-C₉ heteroaryl, or optionally substituted C₁-C₉ heterocyclyl. In some embodiments of formula (I), (IA), or (IB), R¹⁴ is H, halogen, cyano, optionally substituted C₁-C₆ alkyl, optionally substituted ester, optionally substituted carboxamide, optionally substituted C₁-C₆ alkanoyloxy, optionally substituted C₁-C₆ alkanoyl, optionally substituted C₇-C₁₁ aryloyl, optionally substituted C₂-C₁₀ heterocyclyloyl, optionally substituted C₂-C₁₀ heteroaryloyl, optionally substituted C₁-C₆ alkylsulfonyl, optionally substituted C₆-C₁₀ arylsulfonyl, or optionally substituted C₁-C₉ heteroaryl. In other embodiments of formula (I), (IA), or (IB), R¹⁴ is H, halogen, optionally substituted C₁-C₆ alkyl, optionally substituted ester, optionally substituted carboxamide, optionally substituted C₁-C₆ alkanoyloxy, optionally substituted C₁-C₆ alkanoyl. In yet other embodiments of formula (I), (IA), or (IB), R¹⁴ is halogen, optionally substituted C₁-C₆ alkyl, or optionally substituted C₁-C₆ alkanoyl. In still other embodiments of formula (I), (IA), or (IB), R¹⁴ is halogen (e.g., R¹⁴ is fluorine). In some embodiments of formula (I), (IA), or (IB), R¹⁴ is optionally substituted C₁-C₆ alkanoyl. In particular embodiments of formula (I), (IA), or (IB), R¹⁴ is optionally substituted C₂-C₄ alkanoyl. In certain embodiments of formula (I), (IA), or (IB), R¹⁴ is unsubstituted C₂-C₄ alkanoyl.

In some embodiments of formula (I), (IA), or (IB), R¹⁵ is H, optionally substituted amino, optionally substituted amido, optionally substituted C₁-C₆ alkyl, optionally substituted C₁-C₆ alkoxy, optionally substituted C₆-C₁₀ aryloxy, optionally substituted C₁-C₉ heteroaryloxy, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In certain embodiments of formula (I), (IA), or (IB), R¹⁵ is H, optionally substituted C₁-C₆ alkyl, optionally substituted C₁-C₆ alkoxy, optionally substituted C₆-C₁₀ aryloxy, optionally substituted C₁-C₉ heteroaryloxy, optionally substituted C₁-C₆ heteroalkyl, optionally substituted C₃-C₁₀ cycloalkyl, optionally substituted C₆-C₁₀ aryl, optionally substituted C₁-C₉ heteroaryl, or optionally substituted C₁-C₉ heterocyclyl. In particular embodiments of formula (I), (IA), or (IB), R¹⁵ is H or optionally substituted C₁-C₉ heterocyclyl. In other embodiments of formula (I), (IA), or (IB), R¹⁵ is H. In yet other embodiments of formula (I), (IA), or (IB), R¹⁵ is optionally substituted C₁-C₉ heterocyclyl (e.g., R¹⁵ is piperidinyl, methyl-substituted piperidinyl or benzpiperidinyl).

In some embodiments of Formula (IB), R¹ is selected from the group consisting of C₁-C₆ alkanoyl, C₆-C₁₀ aryl, C₇-C₁₁ aryloyl, C₂-C₁₀ heteroaryloyl, C₂-C₇ alkoxycarbonyl, and C₆-C₁₀ arylsulfonyl, wherein R¹ is optionally substituted;
R¹² is H, nitro, or halogen;
R¹⁴ is C₁-C₆ alkanoyl or halogen; and
R¹⁵ is H or optionally substituted C₁-C₉ heterocyclyl.

In some embodiments of Formula (IB), R¹ is selected from an optionally substituted group consisting of C₁-C₆-alkanoyl, C₇-C₁₁ aryloyl, C₂-C₁₀ heteroaryloyl, C₂-C₇ alkoxycarbonyl, and C₆-C₁₀ arylsulfonyl.

In some embodiments of formula (IB), R¹² is nitro, and R¹⁴ is fluoro.

In some embodiments of formula (IB), R¹⁵ is optionally substituted piperidin-1-yl or optionally substituted azepan-1-yl. In some embodiments of formula (I), (IA), or (IB), the compound has the structure according to formula (IC):

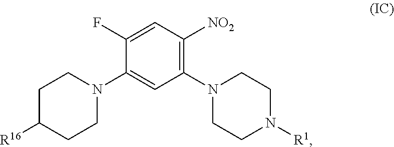

wherein R¹⁶ is H or C₁-C₆ alkyl.

In some embodiments of formula (IC), R¹⁶ is H or methyl.

In some embodiments of formula (I), (IA), (IB), or (IC), R¹ is H, optionally substituted C₁-C₆alkyl, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, optionally substituted ester, optionally substituted carboxamide, optionally substituted C₁-C₆ alkylsulfonyl, substituted C₆-C₁₀ arylsulfonyl, optionally substituted C₁-C₉ heteroarylsulfonyl, optionally substituted C₁-C₉ heterocyclylsulfonyl, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆alkyl. In certain embodiments of formula (I), (IA), (IB), or (IC), R¹ is optionally substituted C₁-C₆ alkyl, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, optionally substituted ester, optionally substituted carboxamide, optionally substituted C₁-C₆ alkylsulfonyl, substituted C₆-C₁₀ arylsulfonyl, optionally substituted C₁-C₉ heteroarylsulfonyl, optionally substituted C₁-C₉ heterocyclylsulfonyl, optionally substituted C₆-C₁₀ aryl C₁-C₆ alkyl, optionally substituted C₁-C₉ heteroaryl C₁-C₆ alkyl, or optionally substituted C₁-C₉ heterocyclyl C₁-C₆ alkyl. In particular embodiments of formula (I), (IA), (IB), or (IC), R¹ is optionally substituted C₁-C₆ alkyl, optionally substituted C₂-C₆ alkanoyl, optionally substituted C₇-C₁₁ aryloyl, optionally substituted C₂-C₁₀ heteroaryloyl, optionally substituted ester, optionally substituted carboxamide, optionally substituted C₁-C₆ alkylsulfonyl, substituted C₆-C₁₀ arylsulfonyl, optionally substituted C₁-C₉ heteroarylsulfonyl, optionally substituted C₆-C₁₀ aryl C₁-C₆ alkyl, or optionally substituted C₁-C₉ heteroaryl C₁-C₆ alkyl. In some embodiments of formula (I), (IA), (IB), or (IC), R¹ is optionally substituted C₁-C₆ alkyl, optionally substituted C₂-C₆alkanoyl, optionally substituted C₇-C₁₁ aryloyl, optionally substituted ester, optionally substituted C₆-C₁₀ arylsulfonyl, or optionally substituted C₆-C₁₀ aryl C₁-C₆ alkyl. In other embodiments of formula (I), (IA), (IB), or (IC), R¹ is optionally substituted C₁-C₆ alkyl, optionally substituted C₂-C₆ alkanoyl, optionally substituted C₇-C₁₁ aryloyl, optionally substituted C₂-C₁₀ heteroaryloyl, optionally substituted C₆-C₁₀ arylsulfonyl, or optionally substituted ester. In yet other embodiments of formula (I), (IA), (IB), or (IC), R¹ is optionally substituted C₂-C₇ alkoxycarbonyl (e.g., methyloxycarbonyl or ethyloxycarbonyl). In still other embodiments of formula (I), (IA), (IB), or (IC), R¹ is optionally substituted C₂-C₆ alkanoyl (e.g., R¹ is acetyl, propanoyl, n-butanoyl, isobutanoyl, or t-pentanoyl). In some embodiments of formula (I), (IA), (IB), or (IC), R¹ is optionally substituted C₇-C₁₁ aryloyl (e.g., R¹ is 4-fluorobenzoyl or benzoyl). In other embodiments of formula (I), (IA), (IB), or (IC), R¹ is optionally substituted C₂-C₁₀ heteroaryloyl (e.g., R¹ is 2-thiophenecarbonyl). In certain embodiments of formula (I), (IA), (IB), or (IC), R¹ is optionally substituted C₆-C₁₀ arylsulfonyl (e.g., R¹ is p-tolylsulfonyl or phenylsulfonyl). In other embodiments of formula (I), (IA), (IB), or (IC), R¹ is optionally substituted C₁-C₆ alkyl, (e.g., R¹ is ethyl or methyl).

In some embodiments of formula (I), (IA), (IB), or (1C), the isolated compound is compound 1, 2, 3, 4, 5, 6, 7, 8, 9, 10, 11, 12, 13, 14, 15, 16, 17, 18, or 53:

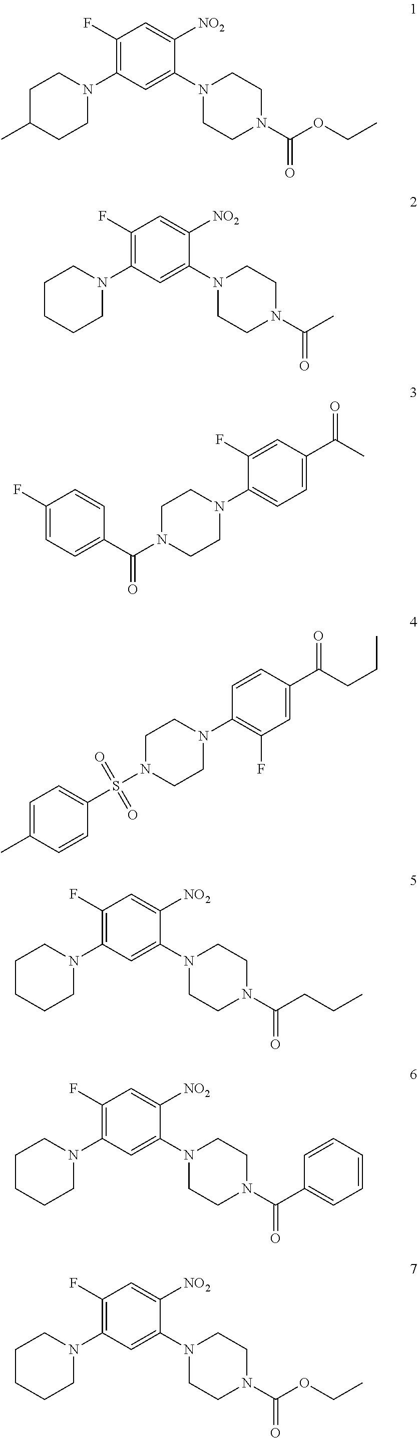

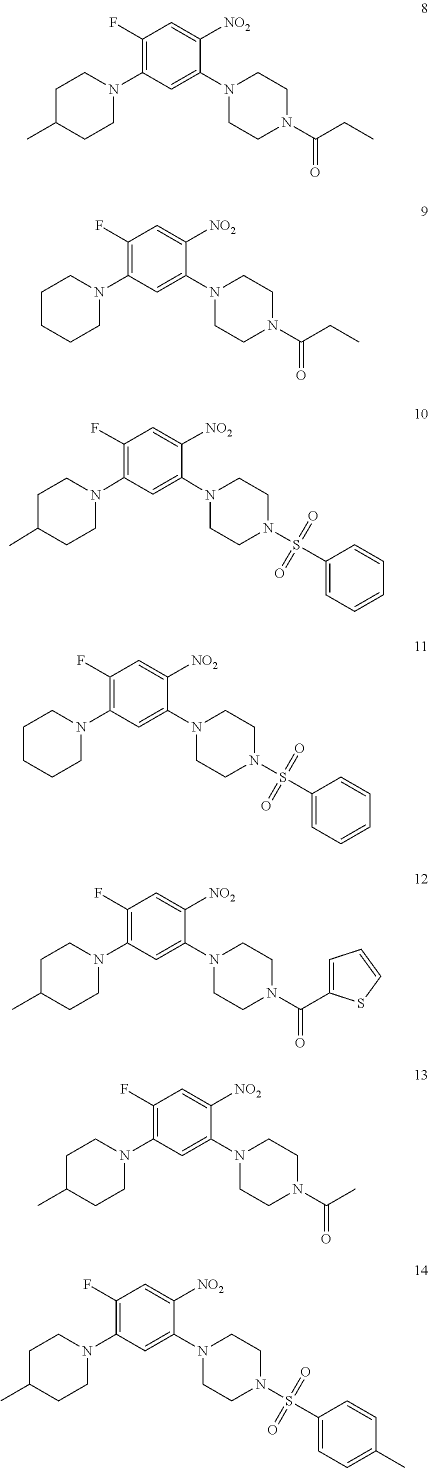

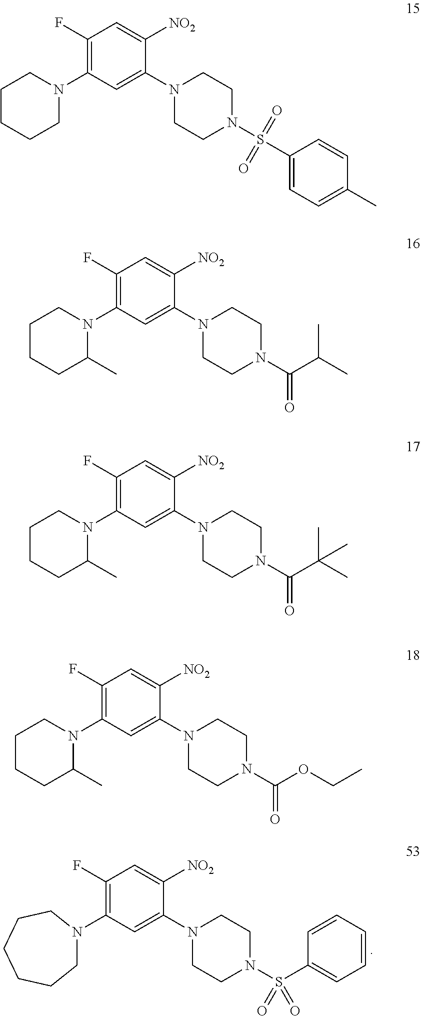 [SMILES: Cc1ccc(S(=O)(=O)N2CCN(c3cc(N4CCCCC4)c(F)cc3[N+](=O)[O-])CC2)cc1 ; CC(C)C(=O)N1CCN(c2cc(N3CCCCC3C)c(F)cc2[N+](=O)[O-])CC1 ; CC1CCCCN1c1cc(N2CCN(C(=O)C(C)(C)C)CC2)c([N+](=O)[O-])cc1F ; CCOC(=O)N1CCN(c2cc(N3CCCCC3C)c(F)cc2[N+](=O)[O-])CC1 ; O=[N+]([O-])c1cc(F)c(N2CCCCCC2)cc1N1CCN(S(=O)(=O)c2ccccc2)CC1]

In one embodiment of formula (IB), the isolated compound is compound 53:

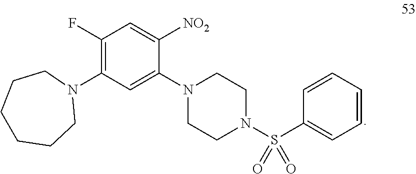 [SMILES: O=[N+]([O-])c1cc(F)c(N2CCCCCC2)cc1N1CCN(S(=O)(=O)c2ccccc2)CC1]

In some embodiments, the GPR174 inhibitor has a structure according to formula (II):

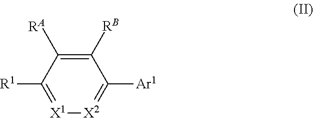

or a stereoisomer thereof, or a pharmaceutically acceptable salt thereof, wherein,
X¹ is N or CR²;
X² is N or CR³;
R^{A} and R^{B}, together with the atoms to which is attached combine to form an optionally substituted 5-membered ring, optionally substituted 6-membered ring, or optionally substituted 7-membered ring;
R¹ is H, halo, hydroxy, optionally substituted amino, optionally substituted amido, thiol, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl, optionally substituted carboxamide, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ thioalkyl, optionally substituted C₁-C₆ alkylsulfinyl, optionally substituted C₁-C₆ alkylsulfonyl, optionally substituted C₆-C₁₀ arylthio, optionally substituted C₆-C₁₀ arylsulfinyl, optionally substituted C₆-C₁₀ arylsulfonyl, optionally substituted C₁-C₉ heteroarylthio, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted sulfamoyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl; and
Ar¹ is optionally substituted C₆-C₁₀ aryl, optionally substituted C₁-C₉ heteroaryl, or optionally substituted C₁-C₉ heterocyclyl.

In some embodiments of formula (II), R^{A} and R^{B}, together with the atoms to which each is attached, combine to form an optionally substituted carbocyclic ring. In certain embodiments of formula (II), R^{A} and R^{B}, together with the atoms to which each is attached, combine to form an optionally substituted heterocyclic ring. In particular embodiments of formula (II), R^{A} and R^{B}, together with the atoms to which each is attached, combine to form an optionally substituted 6-membered ring. In other embodiments of formula (II), R^{A} and R^{B}, together with the atoms to which each is attached, combine to form an optionally substituted non-aromatic ring. In yet other embodiments of formula (II), R^{A} and R^{B}, together with the atoms to which each is attached, combine to form an optionally substituted aromatic ring.

In some embodiments of formula (II), the isolated compound has a structure according to formula (IIA):

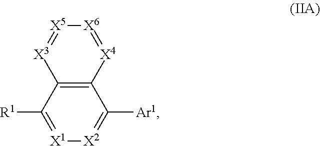

wherein
X³ is N, CR⁴;
X⁴ is N, CR⁵;
X⁵ is N, CR⁶;
X⁶ is N, CR⁷, or absent; and
each of R⁴, R⁵, R⁶, and R⁷ is, independently, H, halo, hydroxy, optionally substituted amino, optionally substituted amido, thiol, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl, optionally substituted carboxamide, optionally substituted C₁-C₆alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ thioalkyl, optionally substituted C₁-C₆ alkylsulfinyl, optionally substituted C₁-C₆ alkylsulfonyl, optionally substituted C₆-C₁₀ arylthio, optionally substituted C₆-C₁₀ arylsulfinyl, optionally substituted C₆-C₁₀ arylsulfonyl, optionally substituted C₁-C₉ heteroarylthio, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted sulfamoyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
wherein
three or fewer of X¹, X², X³, X⁴, X⁵, and X⁶ are N.

In some embodiments of formula (IIA), X³ is CR⁴.

In some embodiments of formula (IIA), X⁴ is CR⁵.

In some embodiments of formula (IIA), X⁵ is CR⁶. In yet other embodiments of formula (IIA), X⁶ is CR⁷.

In some embodiments of formula (II) or (IIA), X¹ is N. In certain embodiments of formula (II) or (IIA), X² is N.

In some embodiments of formula (II), the isolated compound has a structure of formula (IIB):

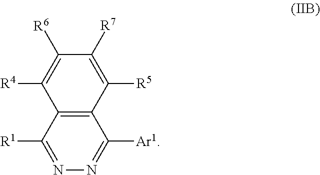

In some embodiments of formula (IIA) or (IIB), R⁴ is H, optionally substituted amino, halo, optionally substituted amido, cyano, optionally substituted C₁-C₆ alkyl, optionally substituted C₁-C₆alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl, optionally substituted carboxamide, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ alkylsulfonyl, optionally substituted C₆-C₁₀ arylsulfonyl, optionally substituted C₁-C₉ heteroarylsulfonyl, optionally substituted C₁-C₉ heterocyclylsulfonyl, optionally substituted sulfamoyl, optionally substituted C₁-C₆ thioalkyl, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In certain embodiments of formula (IIA) or (IIB), R⁴ is H, optionally substituted amino, halo, optionally substituted amido, cyano, optionally substituted C₁-C₆ alkyl, optionally substituted C₂-C₇ alkoxycarbonyl, optionally substituted C₂-C₇ alkoxycarbonyl, optionally substituted carboxamide, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ alkylsulfonyl, optionally substituted C₆-C₁₀ arylsulfonyl, optionally substituted sulfamoyl, optionally substituted C₁-C₆ heteroalkyl, optionally substituted C₆-C₁₀ aryl, optionally substituted C₁-C₉ heteroaryl, or optionally substituted C₁-C₉ heterocyclyl. In particular embodiments of formula (IIA) or (JIB), R⁴ is H, halo, optionally substituted C₁-C₆ alkyl, optionally substituted C₁-C₆ alkylsulfonyl, optionally substituted carboxamide, or optionally substituted sulfamoyl. In other embodiments of formula (IIA) or (JIB), R⁴ is H.

In yet other embodiments of formula (IIA) or (IIB), R⁵ is H, optionally substituted amino, halo, optionally substituted amido, optionally substituted carboxamide, cyano, optionally substituted C₁-C₆ alkyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl, optionally substituted C₁-C₆ alkylsulfonyl, optionally substituted C₆-C₁₀ arylsulfonyl, optionally substituted C₁-C₉ heteroarylsulfonyl, optionally substituted C₁-C₉ heterocyclylsulfonyl, optionally substituted C₁-C₆ thioalkyl, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In still other embodiments of formula (IIA) or (IIB), R⁵ is H, optionally substituted amino, halo, cyano, optionally substituted C₁-C₆ alkyl, optionally substituted C₁-C₆ alkoxy, optionally substituted C₆-C₁o aryloxy, optionally substituted C₁-C₉ heteroaryloxy, optionally substituted C₁-C₆ heteroalkyl, optionally substituted C₆-C₁₀ aryl, optionally substituted C₁-C₉ heteroaryl, or optionally substituted C₁-C₉ heterocyclyl. In some embodiments of formula (IIA) or (IIB), R⁵ is H, optionally substituted amino, halo, optionally substituted C₁-C₆ alkyl, optionally substituted C₆-C₁₀ aryloxy, optionally substituted C₁-C₉ heteroaryloxy, or optionally substituted C₆-C₁₀ aryl. In particular embodiments of formula (IIA) or (IIB), R⁵ is H.

In some embodiments of formula (IIA) or (IIB), R⁶ is H, optionally substituted amino, halo, optionally substituted amido, optionally substituted carboxamide, cyano, optionally substituted C₁-C₆ alkyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁o heterocyclyloyl, hydroxycarbonyl, optionally substituted C₂-C₇ alkoxycarbonyl, optionally substituted C₁-C₆ alkylsulfonyl, optionally substituted C₆-C₁₀ arylsulfonyl, optionally substituted C₁-C₉ heteroarylsulfonyl, optionally substituted C₁-C₉ heterocyclylsulfonyl, optionally substituted C₁-C₆ thioalkyl, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In other embodiments of formula (IIA) or (IIB), R⁶ is H, optionally substituted amino, halo, optionally substituted amido, optionally substituted carboxamide, optionally substituted C₁-C₆ alkyl, optionally substituted C₂-C₇ alkoxycarbonyl, optionally substituted C₁-C₆ alkoxy, optionally substituted C₆-C₁₀ aryloxy, optionally substituted C₁-C₉ heteroaryloxy, optionally substituted C₁-C₆ heteroalkyl, optionally substituted C₆-C₁₀ aryl, optionally substituted C₁-C₉ heteroaryl, or optionally substituted C₁-C₉ heterocyclyl. In yet other embodiments of formula (IIA) or (IIB), R⁶ is H, optionally substituted amino, optionally substituted amido, halo, or optionally substituted C₁-C₆ alkyl. In still other embodiments of formula (IIA) or (IIB), R⁶ is H.

In some embodiments of formula (IIA) or (IIB), R⁷ is H, optionally substituted amino, halo, optionally substituted amido, cyano, optionally substituted C₁-C₆ alkyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl, optionally substituted carboxamide, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ alkylsulfonyl, optionally substituted C₆-C₁₀ arylsulfonyl, optionally substituted C₁-C₉ heteroarylsulfonyl, optionally substituted C₁-C₉ heterocyclylsulfonyl, optionally substituted sulfamoyl, optionally substituted C₁-C₆ thioalkyl, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In certain embodiments of formula (IIA) or (IIB), R⁷ is H, optionally substituted amino, halo, optionally substituted amido, cyano, optionally substituted C₁-C₆ alkyl, optionally substituted C₂-C₇ alkoxycarbonyl, optionally substituted carboxamide, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ alkylsulfonyl, optionally substituted C₆-C₁₀ arylsulfonyl, optionally substituted sulfamoyl, optionally substituted C₁-C₆ heteroalkyl, optionally substituted C₆-C₁₀ aryl, optionally substituted C₁-C₉ heteroaryl, or optionally substituted C₁-C₉ heterocyclyl. In particular embodiments of formula (IIA) or (IIB), R⁷ is H, halo, optionally substituted C₁-C₆ alkyl, optionally substituted C₁-C₆ alkylsulfonyl, optionally substituted carboxamide, or optionally substituted sulfamoyl. In other embodiments of formula (IIA) or (JIB), R⁷ is H.

In some embodiments of formula (II), the isolated compound has the structure according to formula (IIC):

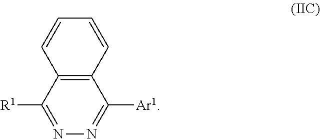

In some embodiments of formula (II), (IIA), (IIB), or (IIC), Ari is optionally substituted C₆-C₁₀ aryl. In other embodiments of formula (II), (IIA), (IIB), or (IIC), Ar¹ is optionally substituted C₆ aryl.

In some embodiments of formula (II), the isolated compound has the structure according to formula (IID):

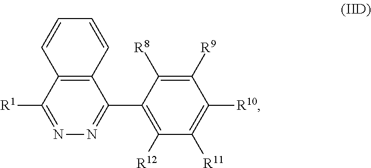

wherein
each of R¹, R⁹, R¹⁰, R¹¹, and R¹² is, independently, H, halo, hydroxy, optionally substituted amino, optionally substituted amido, thiol, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl, optionally substituted carboxamide, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ thioalkyl, optionally substituted C₁-C₆ alkylsulfinyl, optionally substituted C₁-C₆ alkylsulfonyl, optionally substituted C₆-C₁₀ arylthio, optionally substituted C₆-C₁₀ arylsulfinyl, optionally substituted C₆-C₁₀ arylsulfonyl, optionally substituted C₁-C₉ heteroarylthio, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted sulfamoyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₅-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
or any two of adjacent R⁸, R⁹, R¹⁰, R¹¹, and R¹², together with the two adjacent carbon atoms to which they are attached, form a 5, 6, or 7-membered optionally substituted carbocyclic or heterocyclic ring.

In some embodiments of formula (IID), R⁸ is H, halo, or optionally substituted C₁-C₆ alkyl. In some embodiments of formula (IID), R⁸ is H.

In some embodiments of formula (IID), R¹¹ is H.

In some embodiments of formula (IID), R¹² is H, halo, or optionally substituted C₁-C₆ alkyl. In other embodiments of formula (IID), R¹² is H.

In some embodiments of formula (IID), R⁹ is H, optionally substituted amino, halo, optionally substituted amido, optionally substituted carboxamide, cyano, nitro, optionally substituted C₁-C₆ alkyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl, optionally substituted C₁-C₆ alkylsulfinyl, optionally substituted C₁-C₆ alkylsulfonyl, optionally substituted C₆-C₁₀ arylsulfinyl, optionally substituted C₆-C₁₀ arylsulfonyl, optionally substituted C₁-C₉ heteroarylsulfinyl, optionally substituted C₁-C₉ heteroarylsulfonyl, optionally substituted C₁-C₉ heterocyclylsulfonyl, optionally substituted sulfamoyl, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In other embodiments of formula (IID), R⁹ is H, optionally substituted amido, halo, cyano, optionally substituted C₁-C₆ alkyl, optionally substituted C₂-C₇ alkoxycarbonyl, optionally substituted C₁-C₆ alkylsulfonyl, optionally substituted C₆-C₁₀ arylsulfonyl, optionally substituted C₁-C₉ heteroarylsulfonyl, optionally substituted C₁-C₉ heterocyclylsulfonyl, optionally substituted sulfamoyl, optionally substituted C₁-C₆ heteroalkyl, optionally substituted C₆-C₁₀ aryl, optionally substituted C₁-C₉ heteroaryl, or optionally substituted C₁-C₉ heterocyclyl. In yet other embodiments of formula (IID), R⁹ is H, optionally substituted carboxamide, halo, optionally substituted C₁-C₆ alkylsulfonyl, optionally substituted C₁-C₉ heterocyclylsulfonyl, or optionally substituted sulfamoyl. In still other embodiments of formula of formula (IID), R⁹ is optionally substituted sulfamoyl (e.g., unsubstituted sulfamoyl).

In some embodiments of formula (IID), R¹⁰ is H, halo, cyano, optionally substituted C₁-C₆ alkyl, optionally substituted C₂-C₆ alkenyl, optionally substituted C₂-C₆ alkynyl, optionally substituted C₂-C₆ alkanoyl, hydroxycarbonyl, optionally substituted C₂-C₇ alkoxycarbonyl, optionally substituted carboxamide, optionally substituted C₁-C₆ thioalkyl, optionally substituted C₁-C₆ heteroalkyl, optionally substituted C₃-C₁₀ cycloalkyl, optionally substituted C₆-C₁₀ aryl C₁-C₆alkyl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In yet other embodiments of formula (IID), R¹⁰ is H, halo, optionally substituted C₁-C₆ alkyl, optionally substituted C₁-C₆ heteroalkyl, optionally substituted C₃-C₁₀ cycloalkyl, optionally substituted C₆-C₁₀ aryl C₁-C₆ alkyl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In still other embodiments of formula (IID), R¹⁰ is optionally substituted C₁-C₆ alkyl, optionally substituted C₁-C₆ heteroalkyl, or optionally substituted C₃-C₁₀ cycloalkyl. In some embodiments of formula of formula (IID), R¹⁰ is optionally substituted C¹-C⁶ alkyl (e.g., methyl).

In some embodiments of formula (II), (IIA), (IIB), (IIC), or (IID), R¹ is H, hydroxy, optionally substituted amino, halo, thiol, optionally substituted amido, optionally substituted carboxamide, cyano, optionally substituted C₁-C₆ alkyl, optionally substituted C₁-C₆ alkoxy, optionally substituted C₆-C₁₀ aryloxy, optionally substituted C₁-C₉ heteroaryloxy, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆thioalkyl, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In certain embodiments of formula (IID), R¹⁰ is H, optionally substituted amino, optionally substituted amido, optionally substituted C₁-C₆ alkyl, optionally substituted C₁-C₆ alkoxy, optionally substituted C₆-C₁₀ aryloxy, optionally substituted C₁-C₉ heteroaryloxy, optionally substituted C₁-C₆heteroalkyl, optionally substituted C₆-C₁₀ aryl C₁-C₆ alkyl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In some embodiments of formula (II), (IIA), (IIB), (IIC), or (IID), R¹ is H, optionally substituted amino, optionally substituted C₆-C₁₀ aryloxy, optionally substituted C₁-C₉ heteroaryloxy, optionally substituted C₆-C₁₀ aryl, optionally substituted C₁-C₉ heteroaryl, or optionally substituted C₁-C₉ heterocyclyl. In other embodiments of formula (II), (IIA), (IIB), (IIC), or (IID), R¹ is optionally substituted amino. In yet other embodiments of formula (IID), R¹⁰ is substituted amino, wherein at least one substituent is phenyl. In still other embodiments of formula (II), (IIA), (IIB), (IIC), or (IID), R¹ is substituted amino, wherein at least one substituent is o-tolyl.

In some embodiments of formula (II), the compound of formula (II) has a structure of formula (IIE):

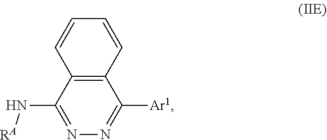

wherein
R^{A} is an optionally substituted phenyl, and
Ar¹ is an optionally substituted phenyl.

In some embodiments of formula (IIE), R^{A} is phenyl or 2-methylphenyl.

In some embodiments of formula (IIE), Ar¹ is 3-aminosulfonyl-4-methylphenyl.

In some embodiments of formula (II), (IIA), (IIB), or (IIC), the isolated compound is compound 19 or 20:

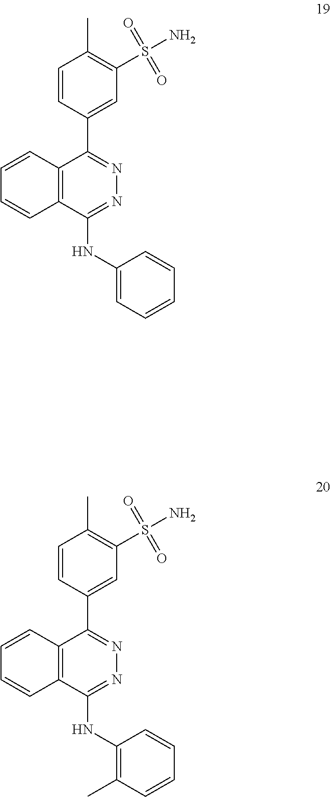 [SMILES: Cc1ccc(-c2nnc(Nc3ccccc3)c3ccccc23)cc1S(N)(=O)=O ; Cc1ccccc1Nc1nnc(-c2ccc(C)c(S(N)(=O)=O)c2)c2ccccc12]

In some embodiments, the GPR174 inhibitor has a structure according to formula (III):

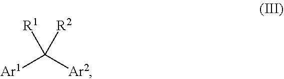

or a stereoisomer thereof, or a tautomer, or a pharmaceutically acceptable salt thereof, wherein
each of R¹ and R² is, independently, H, halo, cyano, optionally substituted C₁-C₆ alkyl, or optionally substituted C₁-C₆ heteroalkyl; and
each of Ar¹ and Ar² is, independently, optionally substituted C₆-C₁₀ aryl or optionally substituted C₁-C₉ heteroaryl.

In some embodiments of formula (III), Ar¹ is optionally substituted C₆-C₁₀ aryl. In other embodiments of formula (III), Ar¹ is optionally substituted C₆ aryl, e.g., optionally substituted phenyl.

In some embodiments of formula (III), Ar² is optionally substituted C₆-C₁₀ aryl. In other embodiments of formula (III), Ar² is optionally substituted C₆ aryl, e.g., optionally substituted phenyl.

In some embodiments of formula (III), the isolated compound has the structure according to formula (IIIA):

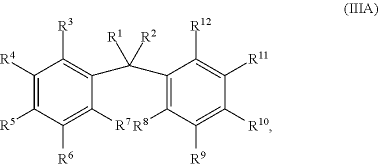

wherein
each of R³, R⁴, R⁵, R⁶, R⁷, R⁸, R⁹, R¹⁰, R¹¹, and R¹² is, independently, H, halo, hydroxy, optionally substituted amino, optionally substituted amido, thiol, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl, optionally substituted carboxamide, optionally substituted C₂-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₂-C₆ alkanoylamino, optionally substituted C₇-C₁₁ aryloylamino, optionally substituted C₂-C₁₀ heteroaryloylamino, optionally substituted C₂-C₁₀ heterocyclyloylamino, optionally substituted C₁-C₆ thioalkyl, optionally substituted C₁-C₆ alkylsulfinyl, optionally substituted C₁-C₆ alkylsulfonyl, optionally substituted C₆-C₁₀ arylthio, optionally substituted C₆-C₁₀ arylsulfinyl, optionally substituted C₆-C₁₀ arylsulfonyl, optionally substituted C₁-C₉ heteroarylthio, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted sulfamoyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₅-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl.

In some embodiments of formula (III) or (IIIA), R¹ is H, halo, or optionally substituted C₁-C₆ alkyl. In other embodiments of formula (III) or (IIIA), R¹ is H, halo, or methyl. In yet other embodiments of formula (III) or (IIIA), R¹ is H.

In some embodiments of formula (III) or (IIIA), R² is H, halo, or optionally substituted C₁-C₆alkyl. In other embodiments of formula (III) or (IIIA), R² is H, halo, or methyl. In yet other embodiments of formula (III) or (IIIA), R² is H.

In some embodiments of formula (III) or (IIIA), the isolated compound has the structure according to formula (IIIB):

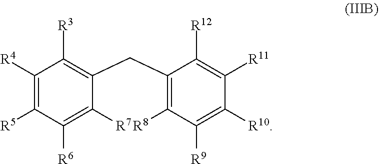

In some embodiments of formula (III), (IIIA), or (IIIB), R³ is H, halo, or optionally substituted C₁-C₆ alkyl. In other embodiments of formula (III), (IIIA), or (IIIB), R³ is H.

In some embodiments of formula (III), (IIIA), or (IIIB), R⁴ is H, halo, or optionally substituted C₁-C₆ alkyl. In other embodiments of formula (III), (IIIA), or (IIIB), R⁴ is H.

In some embodiments of formula (III), (IIIA), or (IIIB), R⁷ is H, halo, or optionally substituted C₁-C₆ alkyl. In other embodiments of formula (III), (IIIA), or (IIIB), R⁷ is H.

In some embodiments of formula (III), (IIIA), or (IIIB), the isolated compound has the structure according to formula (IIIC):

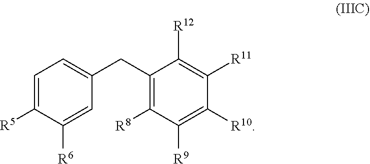

In some embodiments of formula (III), (IIIA), (IIIB), or (IIIC), R¹¹ is H, halo, or optionally substituted C₁-C₆ alkyl. In other embodiments of formula (III), (IIIA), (IIIB), or (IIIC), R¹¹ is H.

In some embodiments of formula (III), (IIIA), (IIIB), or (IIIC), R¹² is H, halo, or optionally substituted C₁-C₆ alkyl. In other embodiments of formula (III), (IIIA), (IIIB), or (IIIC), R¹² is H.

In some embodiments of formula (III), (IIIA), (IIIB), or (IIIC), R⁸ is H, halo, or optionally substituted C₁-C₆ alkyl. In other embodiments of formula (III), (IIIA), (IIIB), or (IIIC), R⁸ is H.

In some embodiments of formula (III), (IIIA), (IIIB), or (IIIC), the isolated compound has the structure according to formula (IIID):

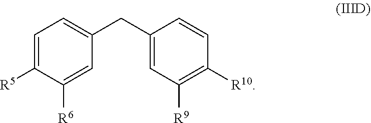

In some embodiments of formula (III), (IIIA), (IIIB), (IIIC), or (IIID), R⁵ is H, halo, cyano, optionally substituted amino, optionally substituted C₁-C₆ alkyl, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, optionally substituted C₂-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₂-C₆ alkanoylamino, optionally substituted C₇-C₁₁ aryloylamino, optionally substituted C₂-C₁₀ heteroaryloylamino, optionally substituted C₂-C₁₀ heterocyclyloylamino, hydroxycarbonyl, optionally substituted carboxamide, optionally substituted C₁-C₆ alkylsulfonyl, substituted C₆-C₁₀ arylsulfonyl, optionally substituted C₁-C₉ heteroarylsulfonyl, optionally substituted C₁-C₉ heterocyclylsulfonyl, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In other embodiments of formula (III), (IIIA), (IIIB), (IIIC), or (IIID), R⁵ is optionally substituted amino, optionally substituted C₂-C₆ alkanoyloxy, optionally substituted C₇-C₁₁ aryloyloxy, optionally substituted C₂-C₁o heteroaryloyloxy, optionally substituted C₂-C₁₀ heterocyclyloyloxy, optionally substituted C₂-C₆alkanoylamino, optionally substituted C₇-C₁₁ aryloylamino, optionally substituted C₂-C₁₀ heteroaryloylamino, optionally substituted C₂-C₁₀ heterocyclyloylamino, hydroxycarbonyl, or optionally substituted carboxamide. In yet other embodiments of formula (III), (IIIA), (IIIB), (IIIC), or (IIID), R⁵ is optionally substituted amino, optionally substituted C₂-C₆ alkanoylamino, optionally substituted C₇-C₁₁ aryloylamino, optionally substituted C₂-C₁₀ heteroaryloylamino, or optionally substituted C₂-C₁₀ heterocyclyloylamino.

In some embodiments of formula (III), (IIIA), (IIIB), (IIIC), or (IIID), R¹⁰ is H, halo, cyano, optionally substituted amino, optionally substituted C₁-C₆ alkyl, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, optionally substituted C₂-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₂-C₆ alkanoylamino, optionally substituted C₇-C₁₁ aryloylamino, optionally substituted C₂-C₁₀ heteroaryloylamino, optionally substituted C₂-C₁₀ heterocyclyloylamino, hydroxycarbonyl, optionally substituted carboxamide, optionally substituted C₁-C₆ alkylsulfonyl, substituted C₆-C₁₀ arylsulfonyl, optionally substituted C₁-C₉ heteroarylsulfonyl, optionally substituted C₁-C₉ heterocyclylsulfonyl, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In other embodiments of formula (III), (IIIA), (IIIB), (IIIC), or (IIID), R¹⁰ is optionally substituted amino, optionally substituted C₂-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₂-C₆alkanoylamino, optionally substituted C₇-C₁₁ aryloylamino, optionally substituted C₂-C₁₀ heteroaryloylamino, optionally substituted C₂-C₁₀ heterocyclyloylamino, hydroxycarbonyl, or optionally substituted carboxamide. In yet other embodiments of formula (III), (IIIA), (IIIB), (IIIC), or (IIID), R¹⁰ is optionally substituted amino, optionally substituted C₂-C₆ alkanoylamino, optionally substituted C₇-C₁₁ aryloylamino, optionally substituted C₂-C₁₀ heteroaryloylamino, or optionally substituted C₂-C₁₀ heterocyclyloylamino.

In some embodiments of formula (III), (IIIA), (IIIB), (IIIC), or (IIID), the isolated compound has the structure according to formula (IIIE):

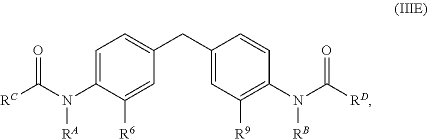

wherein
each of R^{A} and R^{B} is, independently, H or optionally substituted C₁-C₆ alkyl; and
each of R^{C} and R^{D} is, independently, H, optionally substituted C₁-C₆ alkyl, optionally substituted C₃-C₁₀ cycloalkyl, optionally substituted C₆-C₁₀ aryl, optionally substituted C₁-C₉ heteroaryl, or optionally substituted C₁-C₉ heterocyclyl.

In some embodiments of formula (III), (IIIA), (IIIB), (IIIC), (IIID), or (IIIE), R^{A} is H. In other embodiments of formula (III), (IIIA), (IIIB), (IIIC), (IIID), or (IIIE), R^{B} is H.

In some embodiments of formula (III), (IIIA), (IIIB), (IIIC), (IIID), or (IIIE), R^{C} is optionally substituted C₆-C₁₀ aryl, optionally substituted C₁-C₉ heteroaryl. In other embodiments of formula (III), (IIIA), (IIIB), (IIIC), (IIID), or (IIIE), R^{C} is optionally substituted C₄ heteroaryl, e.g., thiophen-2-yl.

In some embodiments of formula (III), (IIIA), (IIIB), (IIIC), (IIID), or (IIIE), RD is optionally substituted C₆-C₁₀ aryl, optionally substituted C₁-C₉ heteroaryl. In still other embodiments of formula (III), (IIIA), (IIIB), (IIIC), (IIID), or (IIIE), RD is optionally substituted C₄ heteroaryl, e.g., thiophen-2-yl.

In some embodiments of formula (III), (IIIA), (IIIB), (IIIC), (IIID), or (IIIE), R⁶ is H, halo, optionally substituted C₁-C₆ alkyl, optionally substituted C₂-C₆ alkenyl, optionally substituted C₂-C₆alkynyl, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In other embodiments of formula (III), (IIIA), (IIIB), (IIIC), (IIID), or (IIIE), R⁶ is H, halo, optionally substituted C₁-C₆ alkyl, optionally substituted C₂-C₆ alkenyl, optionally substituted C₂-C₆ alkynyl, optionally substituted C₁-C₆ heteroalkyl, or optionally substituted C₃-C₁₀ cycloalkyl. In other embodiments of formula (III), (IIIA), (IIIB), (IIIC), (IIID), or (IIIE), R⁶ is H, halo, optionally substituted C₁-C₆ alkyl, optionally substituted C₂-C₆ alkenyl, optionally substituted C₂-C₆ alkynyl, optionally substituted C₁-C₆ heteroalkyl, or optionally substituted C₃-C₁₀ cycloalkyl. In still other embodiments of formula (III), (IIIA), (IIIB), (IIIC), (IIID), or (IIIE), R⁶ is H or optionally substituted C₁-C₆ alkyl. In some embodiments of formula (III), (IIIA), (IIIB), (IIIC), or (IIID), R⁶ is H. In other embodiments of formula (III), (IIIA), (IIIB), (IIIC), (IIID), or (IIIE), R⁶ is C₁-C₆ alkyl, e.g., methyl.

In some embodiments of formula (III), (IIIA), (IIIB), (IIIC), (IIID), or (IIIE), R⁹ is H, halo, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₃-C₁₀ cycloalkyl, optionally substituted C₆-C₁₀ aryl, optionally substituted C₆-C₁₀ aryl C₁-C₆ alkyl, optionally substituted C₁-C₉ heteroaryl, optionally substituted C₁-C₉ heteroaryl C₁-C₆ alkyl, optionally substituted C₁-C₉ heterocyclyl, or optionally substituted C₁-C₉ heterocyclyl C₁-C₆ alkyl. In other embodiments of formula (III), (IIIA), (IIIB), (IIIC), (IIID), or (IIIE), R⁹ is H, halo, optionally substituted C₁-C₆ alkyl, optionally substituted C₂-C₆ alkenyl, optionally substituted C₂-C₆ alkynyl, optionally substituted C₁-C₆ heteroalkyl, or optionally substituted C₃-C₁₀ cycloalkyl. In other embodiments of formula (III), (IIIA), (IIIB), (IIIC), (IIID), or (IIIE), R⁹ is H, halo, optionally substituted C₁-C₆ alkyl, optionally substituted C₂-C₆ alkenyl, optionally substituted C₂-C₆ alkynyl, optionally substituted C₁-C₆ heteroalkyl, or optionally substituted C₃-C₁₀ cycloalkyl. In still other embodiments of formula (III), (IIIA), (IIIB), (IIIC), (IIID), or (IIIE), R⁹ is H or optionally substituted C₁-C₆ alkyl. In some embodiments of formula (III), (IIIA), (IIIB), (IIIC), (IIID), or (IIIE), R⁹ is H. In other embodiments of formula (III), (IIIA), (IIIB), (IIIC), (IIID), or (IIIE), R⁹ is C₁-C₆ alkyl, e.g., methyl.

In some embodiments, the compound of formula (III) has a structure of formula (IIIF):

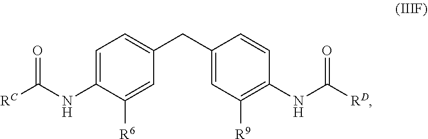

wherein
each of R^{C} and R^{D} is independently optionally substituted C₁-C₉ heteroaryl; and
each of R⁶ and R⁹ is independently optionally substituted C₁-C₆ alkyl.

In some embodiments of formula (IIIF), each of R^{C} and R^{D} is independently unsubstituted C₁-C₉ heteroaryl; and each of R⁶ and R⁹ is independently unsubstituted C₁-C₆ alkyl.

In some embodiments of formula (IIIF), each of R^{C} and R^{D} is thien-2-yl.

In some embodiments of formula (IIIF), each of R⁶ and R⁹ is methyl.

In some embodiments of formula (III), (IIIA), (IIIB), (IIIC), (IIID), or (IIIE), the isolated compound is compound 21:

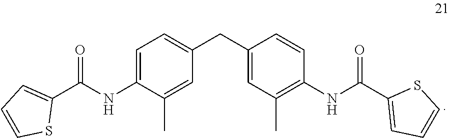 [SMILES: Cc1cc(Cc2ccc(NC(=O)c3cccs3)c(C)c2)ccc1NC(=O)c1cccs1]

In some embodiments, the GPR174 inhibitor has a structure according to formula (IV):

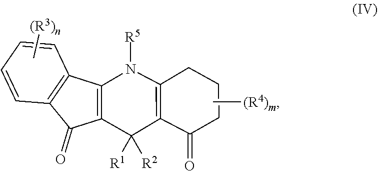

or a stereoisomer thereof, or a tautomer thereof, or a pharmaceutically acceptable salt thereof, wherein
each of R¹ and R² is, independently, H, hydroxy, halo, optionally substituted amino, optionally substituted amido, thiol, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl optionally substituted carboxamide, optionally substituted C₁-C₆alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ thioalkyl, optionally substituted C₁-C₆ alkylsulfinyl, optionally substituted C₁-C₆ alkylsulfonyl, optionally substituted C₆-C₁₀ arylthio, optionally substituted C₆-C₁₀ arylsulfinyl, optionally substituted C₆-C₁₀ arylsulfonyl, optionally substituted C₁-C₉ heteroarylthio, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₅-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
each of R³ and R⁴ is, independently, H, hydroxy, halo, optionally substituted amino, optionally substituted amido, thiol, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl, optionally substituted carboxamide, optionally substituted C₁-C₆alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ thioalkyl, optionally substituted C₁-C₆ alkylsulfonyl, optionally substituted C₆-C₁₀ arylthio, optionally substituted C₆-C₁₀ arylsulfonyl, optionally substituted C₁-C₉ heteroarylthio, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
R⁵ is H, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, optionally substituted C₁-C₆ alkyloxycarbonyl, optionally substituted C₁-C₆ alkylsulfinyl, optionally substituted C₁-C₆ alkylsulfonyl, optionally substituted C₆-C₁₀ arylsulfinyl, optionally substituted C₆-C₁₀ arylsulfonyl, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₅-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl.
n is 0, 1, 2, 3, or 4; and
m is 0, 1, 2, 3, 4, 5, or 6.

In some embodiments of formula (IV), m is 0.

In some embodiments of formula (IV), the isolated compound has a structure according to formula (IVA):

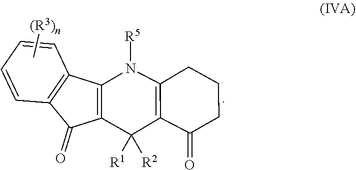

In some embodiments of formula (IV) or (IVA), R¹ is H, halo, optionally substituted amino, optionally substituted amido, thiol, cyano, or optionally substituted C₁-C₆ alkyl. In other embodiments of formula (IV) or (IVA), R¹ is H, halo, or optionally substituted C₁-C₆ alkyl. In yet other embodiments of formula (IV) or (IVA), R¹ is H.

In some embodiments of formula (IV) or (IVA), the isolated compound has a structure according to formula (IVB):

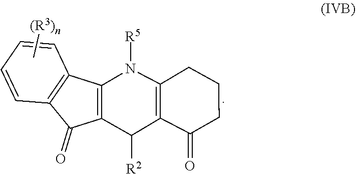

In some embodiments of formula (IV), (IVA), or (IVB), R⁵ is H, optionally substituted C₁-C₆alkyl, optionally substituted C₂-C₆ alkenyl, optionally substituted C₂-C₆ alkynyl, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, optionally substituted C₁-C₆ alkyloxycarbonyl, optionally substituted C₁-C₆ alkylsulfonyl, or optionally substituted C₆-C₁₀ arylsulfonyl. In other embodiments of formula (IV), (IVA), or (IVB), R⁵ is H, optionally substituted C₁-C₆ alkyl, optionally substituted C₂-C₆ alkanoyl, optionally substituted C₇-C₁₁ aryloyl, or optionally substituted C₁-C₆ alkyloxycarbonyl. In yet other embodiments of formula (IV), (IVA), or (IVB), R⁵ is H, optionally substituted C₁-C₆ alkyl, or optionally substituted C₂-C₆ alkanoyl. In still other embodiments of formula (IV), (IVA), or (IVB), R⁵ is H.

In some embodiments of formula (IV), (IVA), or (IVB), the isolated compound has a structure according to formula (IVC):

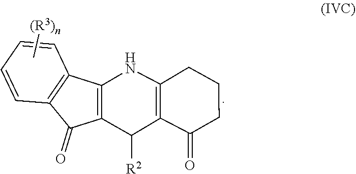

In some embodiments of formula (IV), (IVA), (IVB), or (IVC), R² is H, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl. In other embodiments of formula (IV), (IVA), (IVB), or (IVC), R² is optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₁-C₉ heteroaryl, or optionally substituted C₁-C₉ heterocyclyl. In yet other embodiments of formula (IV), (IVA), (IVB), or (IVC), R² is optionally substituted C₆-C₁₀ aryl, optionally substituted C₁-C₉ heteroaryl, or optionally substituted C₁-C₉ heterocyclyl. In still other embodiments of formula (IV), (IVA), (IVB), or (IVC), R² is optionally substituted C₆-C₁₀ aryl or optionally substituted C₁-C₉ heteroaryl. In other embodiments of formula (IV), (IVA), (IVB), or (IVC), R² is optionally substituted pyridyl (e.g., 2-pyridyl, 3-pyridyl, or 4-pyridyl). In certain embodiments of formula (IV), (IVA), (IVB), or (IVC), R² is optionally substituted phenyl.

In some embodiments of formula (IV), (IVA), (IVB), or (IVC), the isolated compound has a structure according to formula (IVD):

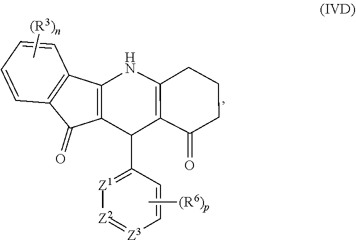

wherein R⁶ at each occurrence is independently, halo, optionally substituted C₁-C₆ alkyl, optionally substituted C₂-C₆ alkenyl, optionally substituted C₆-C₁₀ aryl, optionally substituted C₂-C₆ heteroaryl, optionally substituted C₂-C₆ heterocyclyl, optionally substituted C₂-C₆ alkynyl, optionally substituted C₁-C₆ alkyloxy, optionally substituted amino, optionally substituted amido, thiol, cyano, nitro, C₁-C₆ alkylsulfonyl, hydroxycarbonyl, optionally substituted C₂-C₇ alkoxycarbonyl, optionally substituted C₆-C₁₀ aryloxy, or optionally substituted C₂-C₆ heteroaryloxy;
Z¹ is C or N;
Z² is C or N;
Z³ is N or C; and
p is 0, 1, 2, 3, 4, or 5.

In some embodiments of formula (IVD), Z¹ is C, Z² is C, and Z³ is N. In other embodiments of formula (IVD), Z¹ is C, Z² is N, and Z³ is C. In certain embodiments of formula (IVD), Z¹ is N, Z² is C, and Z³ is C. In certain other embodiments of formula (IVD), Z¹ is C, Z² is C, and Z³ is C.

In some embodiments of formula (IV), (IVA), (IVB), or (IVC), the isolated compound has a structure according to formula IVD):

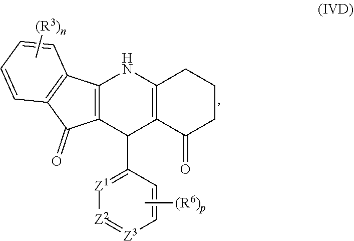

wherein R⁶ at each occurrence is independently, halo, optionally substituted C₁-C₆ alkyl, optionally substituted C₂-C₆ alkenyl, optionally substituted C₆-C₁₀ aryl, optionally substituted C₂-C₆heteroaryl, optionally substituted C₂-C₆ heterocyclyl, optionally substituted C₂-C₆ alkynyl, optionally substituted C₁-C₆ alkyloxy, optionally substituted amino, optionally substituted amido, thiol, cyano, nitro, C₁-C₆ alkylsulfonyl, hydroxycarbonyl, optionally substituted C₂-C₇ alkoxycarbonyl, optionally substituted C₂-C₆ alkoxy, optionally substituted C₂-C₆ alkenoxy, optionally substituted C₆-C₁₀ aryloxy, or optionally substituted C₂-C₆ heteroaryloxy;
Z¹ is CH or N;
Z² is CH or N;
Z³ is N or CH; and
p is 0, 1, 2, 3, 4, or 5.

In some embodiments of formula (IVD), Z¹ is C, Z² is C, and Z³ is N. In other embodiments of formula (IVD), Z¹ is C, Z² is N, and Z³ is CH. In certain embodiments of formula (IVD), Z¹ is N, Z² is CH, and Z³ is CH. In certain other embodiments of formula (IVD), Z¹ is CH, Z² is CH, and Z³ is CH.

In some embodiments of formula (IVD), p is 0. In other embodiments of formula (IVD), p is 1. In certain embodiments of formula (IVD), p is 2. In some embodiments of formula (IVD), p is 1, and R⁶ is in the p- or m-position.

In some embodiments of formula (IVD), R⁶ is methoxy, methyl, hydroxyl, ethoxy, ethyl, optionally substituted phenoxy, optionally substituted cyclopentyloxy, t-butoxy, allyoxy, isopropyloxy, n-pentyloxy, trifluoromethyloxy, difluoromethyloxy, fluoro, chloro, nitro, 2-hydroxyethyloxy, optionally substituted 1,3,4-oxadiazolyl, or optionally substituted pyrrolidyl.

In some embodiments of formula (IV), (IVA), (IVB), (IVC), or (IVD), R³ is H, halo, optionally substituted amino, optionally substituted amido, thiol, cyano, hydroxycarbonyl, optionally substituted C₂-C₇ alkoxycarbonyl, or optionally substituted C₁-C₆ alkyl.

In some embodiments of formula (IV), (IVA), (IVB), (IVC), or (IVD), n is 0.

In some embodiments of formula (IV), the compound has a structure of formula (IVE):

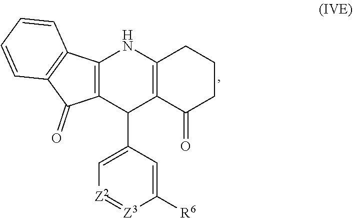

wherein
each of Z² and Z³ is independently CR⁶ or N; and
each of R⁶ is independently H, halogen, hydroxy, nitro, optionally substituted C₁-C₆ alkylsulfonyl, optionally substituted C₁-C₆ alkyl, optionally substituted C₁-C₆ alkoxy, optionally substituted C₄-C₁₁ cycloalkoxy, optionally substituted C₁-C₆ haloalkoxy, optionally substituted C₂-C₆-alkenoxy, optionally substituted C₆-C₁₀ aryloxy, optionally substituted C₁-C₉ heterocyclyl, optionally substituted C₆-C₁₀ aryl, or optionally substituted C₁-C₉ heteroaryl;
or two adjacent R⁶ groups, taken together with the carbon atoms to which they are attached, form a C₁-C₉ heterocyclyl.

In some embodiments of formula IV, (IVA), (IVB), or (IVC), the isolated compound is compound 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54 or 55:

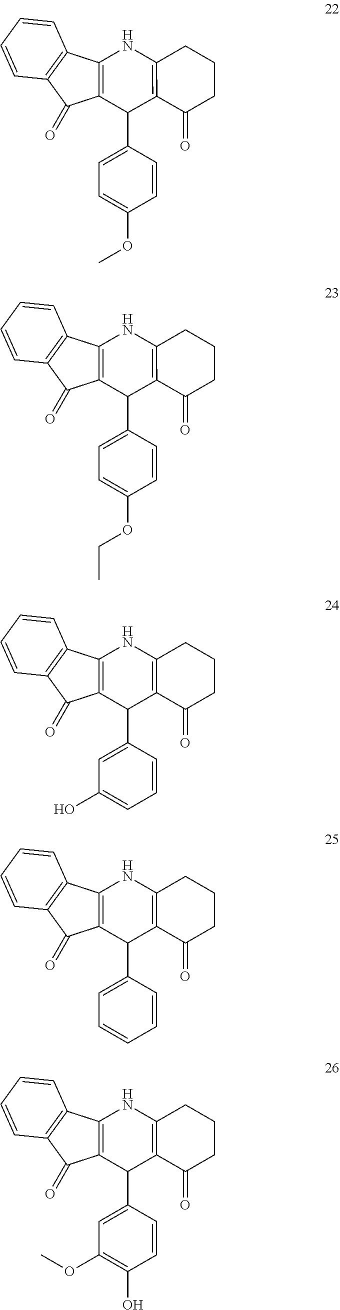

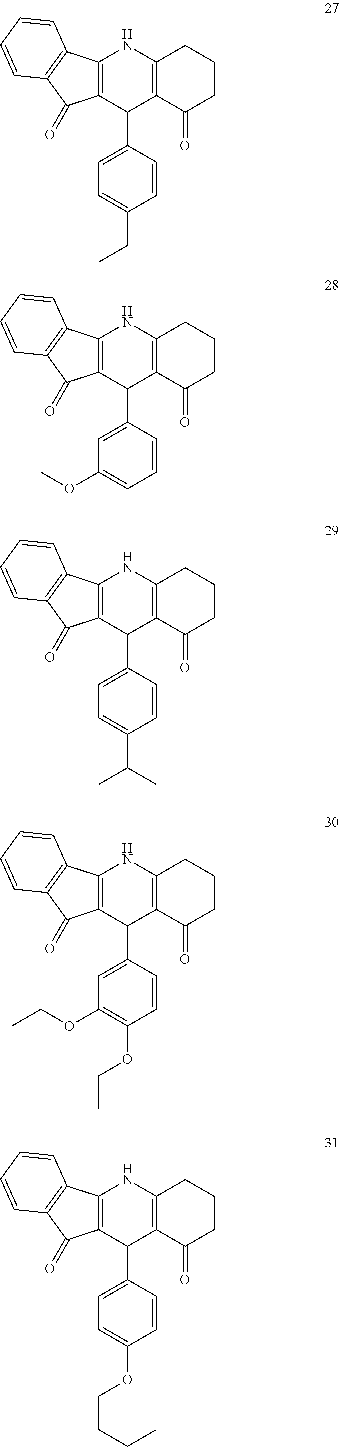 [SMILES: CCc1ccc(C2C3=C(CCCC3=O)NC3=C2C(=O)c2ccccc23)cc1 ; COc1cccc(C2C3=C(CCCC3=O)NC3=C2C(=O)c2ccccc23)c1 ; CC(C)c1ccc(C2C3=C(CCCC3=O)NC3=C2C(=O)c2ccccc23)cc1 ; CCOc1ccc(C2C3=C(CCCC3=O)NC3=C2C(=O)c2ccccc23)cc1OCC ; CCCCOc1ccc(C2C3=C(CCCC3=O)NC3=C2C(=O)c2ccccc23)cc1]

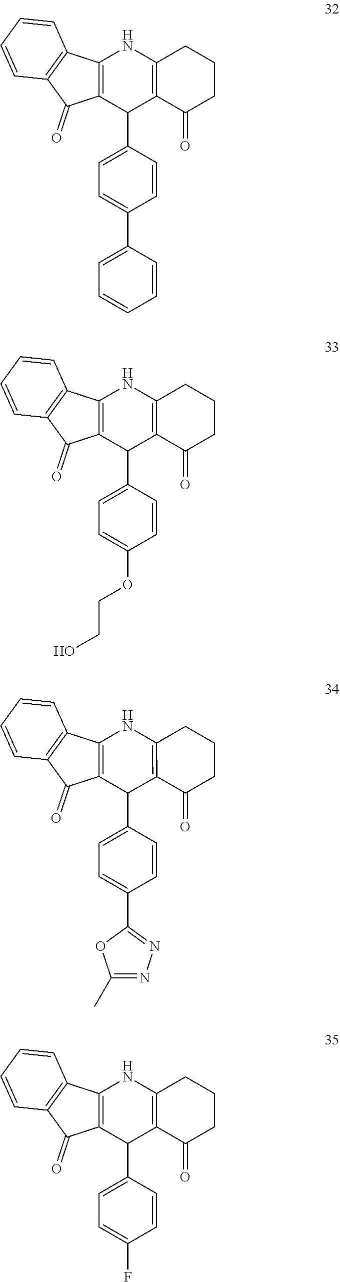 [SMILES: O=C1CCCC2=C1C(c1ccc(-c3ccccc3)cc1)C1=C(N2)c2ccccc2C1=O ; O=C1CCCC2=C1C(c1ccc(OCCO)cc1)C1=C(N2)c2ccccc2C1=O ; Cc1nnc(-c2ccc(C3C4=C(CCCC4=O)NC4=C3C(=O)c3ccccc34)cc2)o1 ; O=C1CCCC2=C1C(c1ccc(F)cc1)C1=C(N2)c2ccccc2C1=O]

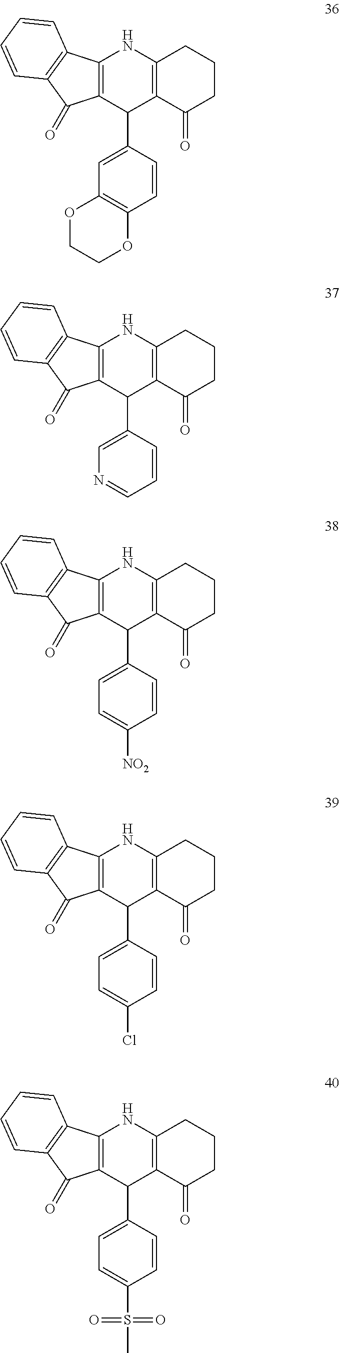 [SMILES: O=C1CCCC2=C1C(c1ccc3c(c1)OCCO3)C1=C(N2)c2ccccc2C1=O ; O=C1CCCC2=C1C(c1cccnc1)C1=C(N2)c2ccccc2C1=O ; O=C1CCCC2=C1C(c1ccc([N+](=O)[O-])cc1)C1=C(N2)c2ccccc2C1=O ; O=C1CCCC2=C1C(c1ccc(Cl)cc1)C1=C(N2)c2ccccc2C1=O ; CS(=O)(=O)c1ccc(C2C3=C(CCCC3=O)NC3=C2C(=O)c2ccccc23)cc1]

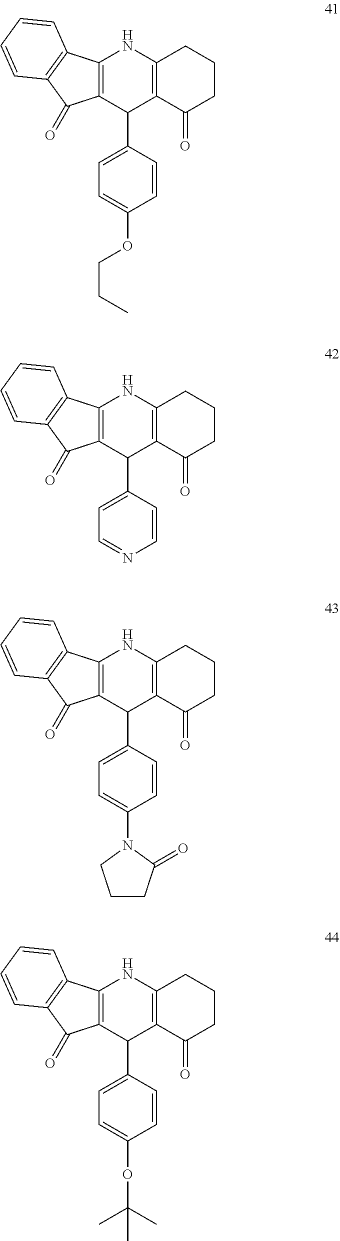 [SMILES: CCCOc1ccc(C2C3=C(CCCC3=O)NC3=C2C(=O)c2ccccc23)cc1 ; O=C1CCCC2=C1C(c1ccncc1)C1=C(N2)c2ccccc2C1=O ; O=C1CCCC2=C1C(c1ccc(N3CCCC3=O)cc1)C1=C(N2)c2ccccc2C1=O ; CC(C)(C)Oc1ccc(C2C3=C(CCCC3=O)NC3=C2C(=O)c2ccccc23)cc1]

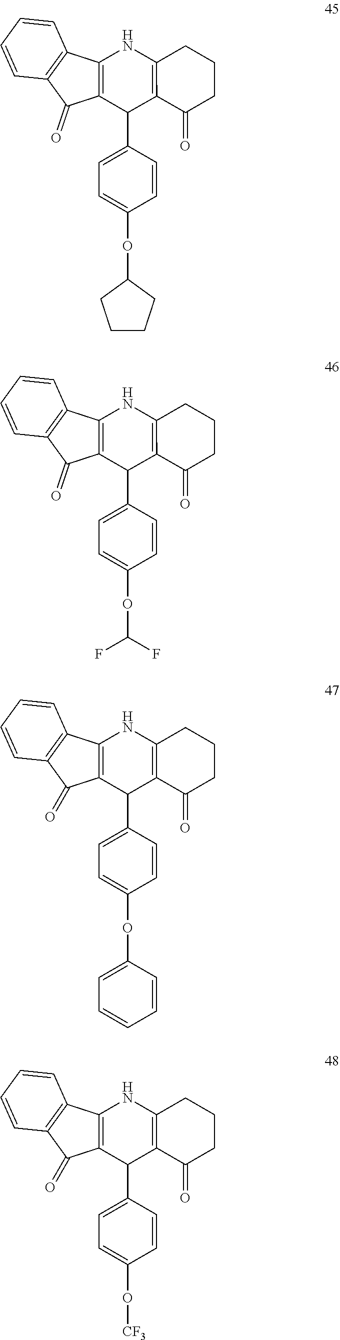 [SMILES: O=C1CCCC2=C1C(c1ccc(OC3CCCC3)cc1)C1=C(N2)c2ccccc2C1=O ; O=C1CCCC2=C1C(c1ccc(OC(F)F)cc1)C1=C(N2)c2ccccc2C1=O ; O=C1CCCC2=C1C(c1ccc(Oc3ccccc3)cc1)C1=C(N2)c2ccccc2C1=O ; O=C1CCCC2=C1C(c1ccc(OC(F)(F)F)cc1)C1=C(N2)c2ccccc2C1=O]

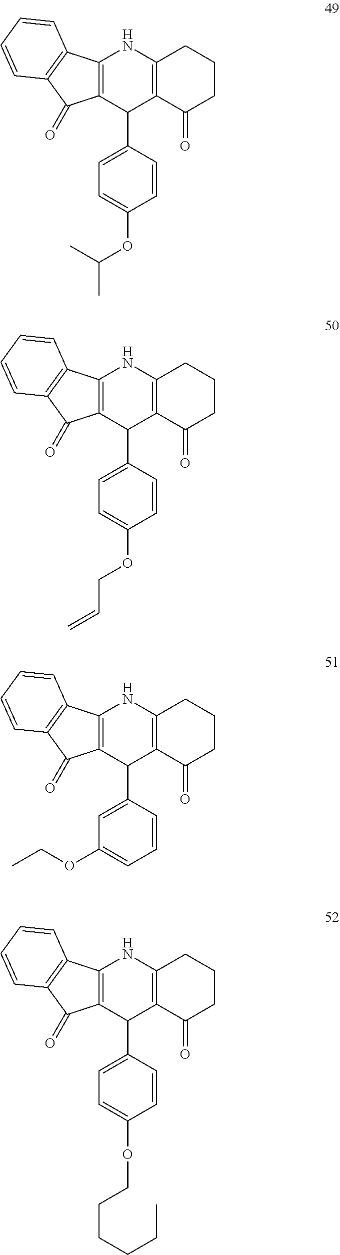 [SMILES: CC(C)Oc1ccc(C2C3=C(CCCC3=O)NC3=C2C(=O)c2ccccc23)cc1 ; C=CCOc1ccc(C2C3=C(CCCC3=O)NC3=C2C(=O)c2ccccc23)cc1 ; CCOc1cccc(C2C3=C(CCCC3=O)NC3=C2C(=O)c2ccccc23)c1 ; CCCCCCOc1ccc(C2C3=C(CCCC3=O)NC3=C2C(=O)c2ccccc23)cc1]

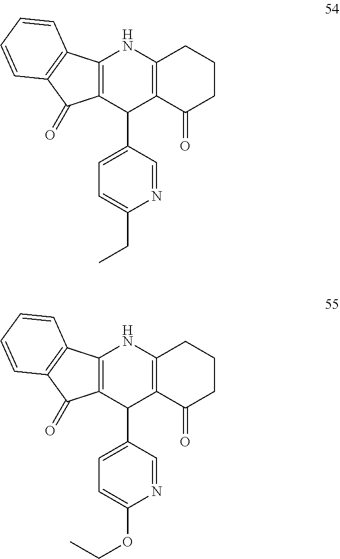 [SMILES: CCc1ccc(C2C3=C(CCCC3=O)NC3=C2C(=O)c2ccccc23)cn1 ; CCOc1ccc(C2C3=C(CCCC3=O)NC3=C2C(=O)c2ccccc23)cn1]

In some embodiments, the compound of formula (IVE) is compound 54 or 55.

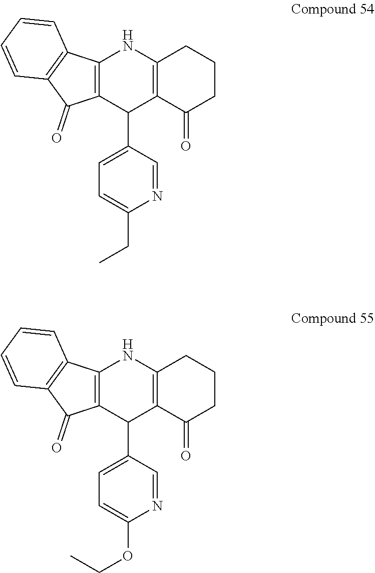 [SMILES: CCc1ccc(C2C3=C(CCCC3=O)NC3=C2C(=O)c2ccccc23)cn1 ; CCOc1ccc(C2C3=C(CCCC3=O)NC3=C2C(=O)c2ccccc23)cn1]

In some embodiments, the GPR174 inhibitor has a structure according to the following formula (V):

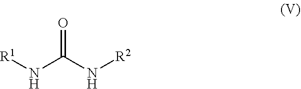

or a stereoisomer thereof, or a tautomer thereof, or a pharmaceutically acceptable salt thereof, wherein
R¹ is phenyl, and
R² is optionally substituted C₆-C₁₀ aryl or optionally substituted C₁-C₉ heteroaryl.

In some embodiments of formula (V), R² is optionally substituted phenyl.

In some embodiments of formula (V), R² is phenyl substituted with para-C₂-C₆ alkenoxy.

In some embodiments of formula (V), R² is phenyl substituted with para-(2-methylallyl)oxy.

In some embodiments of formula (V), the compound of formula (V) is compound 56:

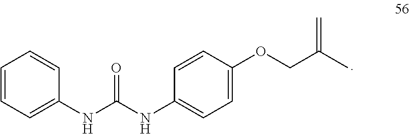 [SMILES: C=C(C)COc1ccc(NC(=O)Nc2ccccc2)cc1]

In some embodiments, the GPR174 inhibitor has a structure according to the following formula (Va):

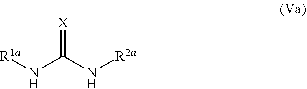

or a stereoisomer thereof, or a tautomer thereof, or a pharmaceutically acceptable salt thereof, wherein
X is O or S;
R^{1a} is an optionally substituted phenyl; and
R^{2a} is an optionally substituted C₆-C₁₀ aryl, an optionally substituted C₃-C₉ heteroaryl or an optionally substituted C₃-C₁₀ heteroarylalkyl.

In some embodiments, R^{1a} is a substituted phenyl. In some more specific embodiments, R^{1a} is optionally substituted with halo (e.g., F, Br, Cl, or I). In some embodiments, R^{1a} has the following structure:

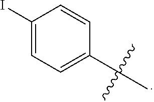 [SMILES: *c1ccc(I)cc1]

In some embodiments, R^{2a} is an optionally substituted C₃-C₁₀ heteroarylalkyl. In some embodiments, R^{2a} is unsubstituted. In some more specific embodiments, R^{2a} has the following structure:

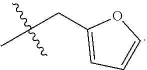 [SMILES: *Cc1ccco1]

In some embodiments, X is O. In certain embodiments, X is S.

In some embodiments, provided herein is a GPR174 inhibitor according to formula (VI):

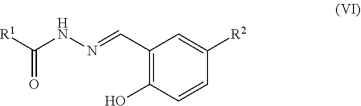

or a stereoisomer thereof, or a tautomer thereof, or a pharmaceutically acceptable salt thereof, wherein
R¹ is an optionally substituted C₁-C₉ heteroaryl, and
R² is halogen.

In some embodiments of formula (VI), the N═C bond has the (E) configuration.

In some embodiments of formula (VI), the N═C bond has the (Z) configuration.

In some embodiments of formula (VI), R¹ is an optionally substituted pyridinyl or an optionally substituted furanyl.

In some embodiments of formula (VI), R¹ is pyridin-4-yl.

In some embodiments of formula (VI), R¹ is 2,5-dimethyl-fur-3-yl.

In some embodiments of formula (VI), R² is halo.

In some embodiments of formula (VI), R² is chloro or bromo.

In some embodiments of formula (VI), the compound of formula (VI) is compound 57 or 58:

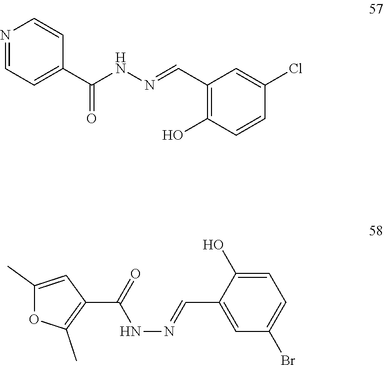 [SMILES: O=C(N/N=C/c1cc(Cl)ccc1O)c1ccncc1 ; Cc1cc(C(=O)N/N=C/c2cc(Br)ccc2O)c(C)o1]

or a stereoisomer thereof, or a tautomer thereof, or a pharmaceutically acceptable salt thereof.

In some embodiments, the GPR174 inhibitor has a structure according to formula (VII):

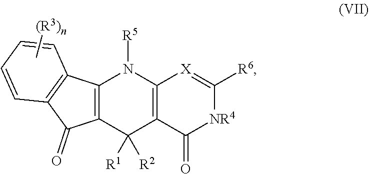

or a stereoisomer thereof, or a tautomer thereof, or a pharmaceutically acceptable salt thereof, wherein
each of R¹ and R² is, independently, H, hydroxy, halo, optionally substituted amino, optionally substituted amido, thiol, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl optionally substituted carboxamide, optionally substituted C₁-C₆alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ thioalkyl, optionally substituted C₁-C₆ alkylsulfinyl, optionally substituted C₁-C₆ alkylsulfonyl, optionally substituted C₆-C₁₀ arylthio, optionally substituted C₆-C₁₀ arylsulfinyl, optionally substituted C₆-C₁₀ arylsulfonyl, optionally substituted C₁-C₉ heteroarylthio, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₅-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
each of R³ and R⁶ is, independently, H, hydroxy, halo, optionally substituted amino, optionally substituted amido, thiol, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl, optionally substituted carboxamide, optionally substituted C₁-C₆alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ alkylthio, optionally substituted C₁-C₆ alkylsulfonyl, optionally substituted C₆-C₁₀ arylthio, optionally substituted C₆-C₁₀ arylsulfonyl, optionally substituted C₁-C₉ heteroarylthio, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
each of R⁴ and R⁵ is independently H, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, optionally substituted C₁-C₆ alkyloxycarbonyl, optionally substituted C₁-C₆ alkylsulfinyl, optionally substituted C₁-C₆ alkylsulfonyl, optionally substituted C₆-C₁₀ arylsulfinyl, optionally substituted C₆-C₁₀ arylsulfonyl, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₅-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
X is N or CR⁷, wherein R⁷ is H, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, optionally substituted C₁-C₆ alkyloxycarbonyl, optionally substituted C₁-C₆ alkylsulfinyl, optionally substituted C₁-C₆ alkylsulfonyl, optionally substituted C₆-C₁₀ arylsulfinyl, optionally substituted C₆-C₁₀ arylsulfonyl, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl; and
n is 0, 1, 2, 3, or 4.

In some embodiments of formula (VII), the isolated compound has a structure according to formula (VIIA):

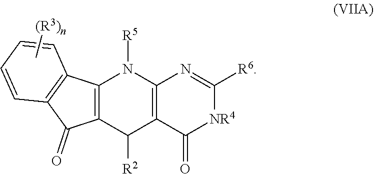

In some embodiments of formula (VII) or (VIIA), R⁵ is H, optionally substituted C₁-C₆ alkyl, optionally substituted C₂-C₆ alkenyl, optionally substituted C₂-C₆ alkynyl, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, optionally substituted C₁-C₆ alkyloxycarbonyl, optionally substituted C₁-C₆ alkylsulfonyl, or optionally substituted C₆-C₁₀ arylsulfonyl. In other embodiments of formula (VII) or (VIIA), R⁵ is H, optionally substituted C₁-C₆alkyl, optionally substituted C₂-C₆ alkanoyl, optionally substituted C₇-C₁₁ aryloyl, or optionally substituted C₁-C₆ alkyloxycarbonyl. In yet other embodiments of formula (VII) or (VIIA), R⁵ is H, optionally substituted C₁-C₆ alkyl, or optionally substituted C₂-C₆ alkanoyl. In some embodiments of formula (VII) or (VIIA), R⁵ is H.

In some embodiments of formula (VII) or (VIIA), the isolated compound has a structure according to formula (VIIB):

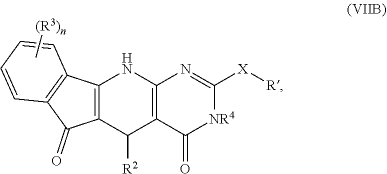

wherein X is O, S, or NR″, and R′ and R″ are independently selected from H, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl.

In some embodiments of formula (VII), (VIIA), or (VIIB), R² is H, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl. In some embodiments of formula (VII), (VIIA), or (VIIB), R² is H, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl. In other embodiments of formula (VII), (VIIA), or (VIIB), R² is optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₁-C₉ heteroaryl, or optionally substituted C₁-C₉ heterocyclyl. In some embodiments of formula (VII), (VIIA), or (VIIB), R² is optionally substituted C₆-C₁₀ aryl, optionally substituted C₁-C₉ heteroaryl, or optionally substituted C₁-C₉ heterocyclyl. In some embodiments of formula (VII), (VIIA), or (VIIB), R² is optionally substituted C₆-C₁₀ aryl or optionally substituted C₁-C₉ heteroaryl. In some embodiments of formula (VII), (VIIA), or (VIIB), R² is optionally substituted pyridyl (e.g., 2-pyridyl, 3-pyridyl, or 4-pyridyl). In some embodiments of formula (VII), (VIIA), or (VIIB), R² is optionally substituted phenyl.

In some embodiments of formula (VII), (VIIA), or (VIIB), the isolated compound has a structure according to formula (VIIC):

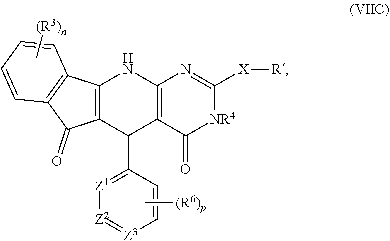

wherein R⁶ at each occurrence is independently, H, halo, optionally substituted C₁-C₆ alkyl, optionally substituted C₂-C₆ alkenyl, optionally substituted C₆-C₁₀ aryl, optionally substituted C₂-C₆ heteroaryl, optionally substituted C₂-C₆ heterocyclyl, optionally substituted C₂-C₆ alkynyl, optionally substituted amino, optionally substituted amido, thiol, cyano, nitro, C₁-C₆ alkylsulfonyl, hydroxycarbonyl, optionally substituted C₂-C₇ alkoxycarbonyl, optionally substituted C₆-C₁₀ aryloxy, or optionally substituted C₂-C₆ heteroaryloxy;
Z¹ is CR⁶ or N;
Z² is CR⁶ or N;
Z³ is N or CR⁶; and
p is 0, 1, 2, or 3.

In some embodiments of formula (VIIC), Z¹ is CR⁶, Z² is CR⁶, and Z³ is N. In other embodiments of formula (VIIC), Z¹ is CR⁶, Z² is N, and Z³ is CR⁶. In certain embodiments of formula (VIIC), Z¹ is N, Z² is CR⁶, and Z³ is CR⁶. In certain other embodiments of formula (VIIC), Z¹ is CR⁶, Z² is CR⁶, and Z³ is CR⁶.

In some embodiments of formula (VII), (VIIA), (VIIB), or (VIIC), X is S or O. In some embodiments of formula (VII), (VIIA), (VIIB), or (VIIC), R⁴ is H or optionally substituted C₁-C₆ alkyl. In some embodiments of formula (VII), R′ is H or optionally substituted C₁-C₆ alkyl.

In some embodiments, the GPR174 inhibitor has a structure according to formula (VIII):

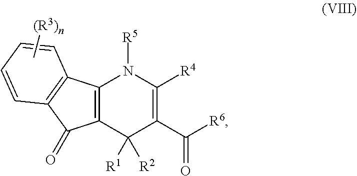

or a stereoisomer thereof, or a tautomer thereof, or a pharmaceutically acceptable salt thereof, wherein
each of R¹ and R² is, independently, H, hydroxy, halo, optionally substituted amino, optionally substituted amido, thiol, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl optionally substituted carboxamide, optionally substituted C₁-C₆alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ thioalkyl, optionally substituted C₁-C₆ alkylsulfinyl, optionally substituted C₁-C₆ alkylsulfonyl, optionally substituted C₆-C₁₀ arylthio, optionally substituted C₆-C₁₀ arylsulfinyl, optionally substituted C₆-C₁₀ arylsulfonyl, optionally substituted C₁-C₉ heteroarylthio, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₅-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
each of R³ and R⁶ is, independently, H, hydroxy, halo, optionally substituted amino, optionally substituted amido, thiol, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted C₂-C₇ alkoxycarbonyl, optionally substituted carboxamide, optionally substituted C₁-C₆ alkanoyloxy, optionally substituted C₇-C₁₁ aryloyloxy, optionally substituted C₂-C₁o heteroaryloyloxy, optionally substituted C₂-C₁₀ heterocyclyloyloxy, optionally substituted C₁-C₆ thioalkyl, optionally substituted C₁-C₆ alkylsulfonyl, optionally substituted C₆-C₁₀ arylthio, optionally substituted C₆-C₁₀ arylsulfonyl, optionally substituted C₁-C₉ heteroarylthio, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁o cycloalkenyl, optionally substituted C₅-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
each of R⁵ and R⁶ is independently H, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, optionally substituted C₁-C₆ alkyloxycarbonyl, optionally substituted C₁-C₆ alkylsulfinyl, optionally substituted C₁-C₆ alkylsulfonyl, optionally substituted C₆-C₁₀ arylsulfinyl, optionally substituted C₆-C₁₀ arylsulfonyl, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₅-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
X is N or CR⁷, wherein R⁷ is H, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, optionally substituted C₁-C₆ alkyloxycarbonyl, optionally substituted C₁-C₆ alkylsulfinyl, optionally substituted C₁-C₆ alkylsulfonyl, optionally substituted C₆-C₁₀ arylsulfinyl, optionally substituted C₆-C₁₀ arylsulfonyl, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₈-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, or optionally substituted C₁-C₉ heterocyclyl C₂-C₆ alkynyl;
R⁶ is H, hydroxy, optionally substituted amino, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, or optionally substituted C₁-C₉ heteroaryloxy; and
n is 0, 1, 2, 3, or 4.

In some embodiments of formula (VIII), R⁶ is hydroxy or optionally substituted C₁-C₆ alkyl. In some embodiments of formula (VIII), R⁵ is H. In some embodiments of formula (VIII), R¹ is H.

In any of the embodiments described herein, the compound may be a compound described in Table 1 (e.g., any one of compounds 1-59).

In some embodiments, the inhibitor is one of the following compounds as set forth below in Table 1.

TABLE 1
Representative compounds

| | | | Human GPR174 |
|---|---|---|---|
| | | | CRE |
| | | | Activity, EC₅₀; |
| Compound | | CRA | Fold decrease in |
| No./Formula | | EC₅₀ | Gs signaling |
| Group | Structure | (μM) | activity |
| 1 (Group I) | 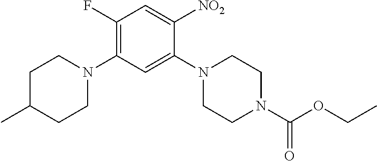 [SMILES: CCOC(=O)N1CCN(c2cc(N3CCC(C)CC3)c(F)cc2[N+](=O)[O-])CC1] | 2.1, 2.1, 1.5, 0.9 | IA; 0.4, 0.8; 2.9 fold decrease |
| 2 (Group I) | 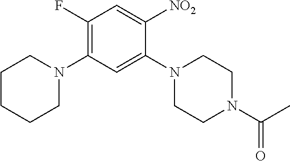 [SMILES: CC(=O)N1CCN(c2cc(N3CCCCC3)c(F)cc2[N+](=O)[O-])CC1] | 3.3, 3.0, 5.1, 0.4 | IA; 0.4, 0.3; 1.7 fold decrease |
| 3 (Group I) | 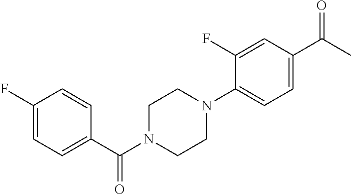 [SMILES: CC(=O)c1ccc(N2CCN(C(=O)c3ccc(F)cc3)CC2)c(F)c1] | 4.1. 2.0, >10 | Antagonist or allosteric modulator |
| 4 (Group I) | 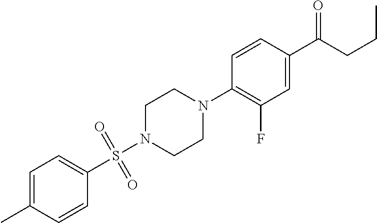 [SMILES: CCCC(=O)c1ccc(N2CCN(S(=O)(=O)c3ccc(C)cc3)CC2)c(F)c1] | 0.2, 2.0, >1 | Antagonist |
| 5 (Group I) | 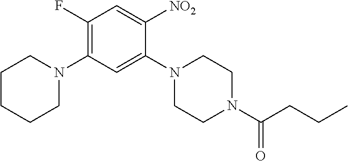 [SMILES: CCCC(=O)N1CCN(c2cc(N3CCCCC3)c(F)cc2[N+](=O)[O-])CC1] | >1.2 | IA; 0.9, 2.4 fold decrease |
| 6 (Group I) | 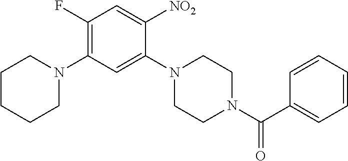 [SMILES: O=C(c1ccccc1)N1CCN(c2cc(N3CCCCC3)c(F)cc2[N+](=O)[O-])CC1] | 1.4, >0.4 | IA; 0.4, 0.3; 3.8 fold decrease |
| 7 (Group I) | 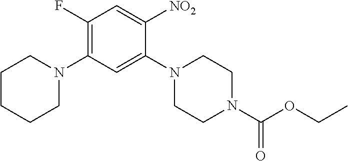 [SMILES: CCOC(=O)N1CCN(c2cc(N3CCCCC3)c(F)cc2[N+](=O)[O-])CC1] | >0.6, >0.7 | IA; 0.3, 0.5; 2.4 fold decrease |
| 8 (Group I) | 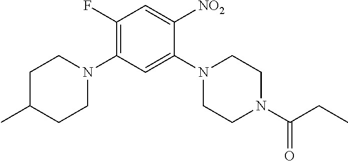 [SMILES: CCC(=O)N1CCN(c2cc(N3CCC(C)CC3)c(F)cc2[N+](=O)[O-])CC1] | >0.7, >1.6 | IA; 0.7, 0.4; 3.3 fold decrease |
| 9 (Group I) | 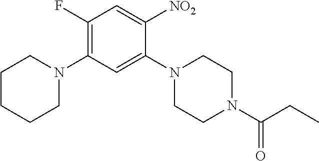 [SMILES: CCC(=O)N1CCN(c2cc(N3CCCCC3)c(F)cc2[N+](=O)[O-])CC1] | >2 | IA; 0.7, 0.5; 2.1 fold decrease |
| 10 (Group I) | 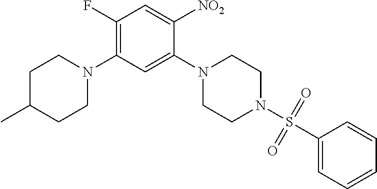 [SMILES: CC1CCN(c2cc(N3CCN(S(=O)(=O)c4ccccc4)CC3)c([N+](=O)[O-])cc2F)CC1] | 0.6, 0.5 | IA, 0.2, 0.5; 5.1 fold decrease |
| 11 (Group I) | 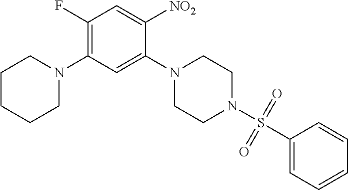 [SMILES: O=[N+]([O-])c1cc(F)c(N2CCCCC2)cc1N1CCN(S(=O)(=O)c2ccccc2)CC1] | 1.5, >1.1 | IA, 0.5, 1.3, 0.6; 3.1 fold decrease |
| 12 (Group I) | 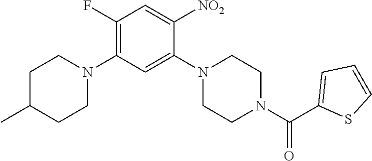 [SMILES: CC1CCN(c2cc(N3CCN(C(=O)c4cccs4)CC3)c([N+](=O)[O-])cc2F)CC1] | 0.3 | IA, 0.4, 0.7, 3.9 fold decrease |
| 13 (Group I) | 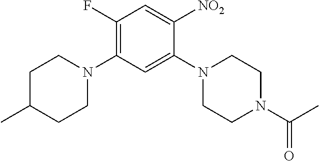 [SMILES: CC(=O)N1CCN(c2cc(N3CCC(C)CC3)c(F)cc2[N+](=O)[O-])CC1] | >2 | IA, 0.8, 1.2; 2.2 fold decrease |
| 14 (Group I) | 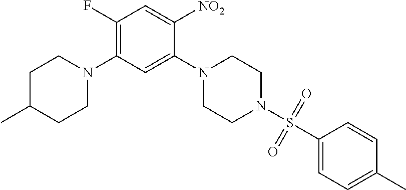 | ND | IA, 1.1; 3 fold decrease |
| 15 (Group I) | 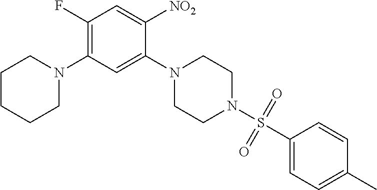 [SMILES: Cc1ccc(S(=O)(=O)N2CCN(c3cc(N4CCCCC4)c(F)cc3[N+](=O)[O-])CC2)cc1] | ND | IA, 1.7, 2.6 fold decrease |
| 16 (Group I) | 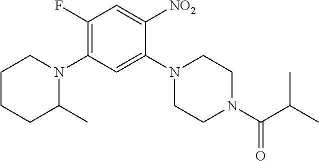 [SMILES: CC(C)C(=O)N1CCN(c2cc(N3CCCCC3C)c(F)cc2[N+](=O)[O-])CC1] | ND | IA, 0.8, 2.1 fold decrease |
| 17 (Group I) | 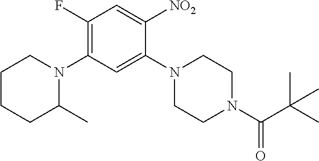 [SMILES: CC1CCCCN1c1cc(N2CCN(C(=O)C(C)(C)C)CC2)c([N+](=O)[O-])cc1F] | ND | IA, 3.0, 2.0 fold decrease |
| 18 (Group I) | 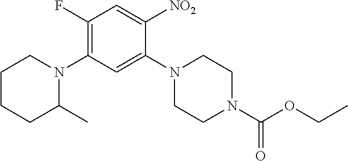 [SMILES: CCOC(=O)N1CCN(c2cc(N3CCCCC3C)c(F)cc2[N+](=O)[O-])CC1] | ND | IA, 1.0; 1.9 fold decrease |
| 53 (Group I) | 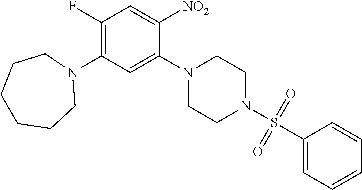 [SMILES: O=[N+]([O-])c1cc(F)c(N2CCCCCC2)cc1N1CCN(S(=O)(=O)c2ccccc2)CC1] | ND | IA, 1.8; 3.4 decrease |
| 19 (Group II) | 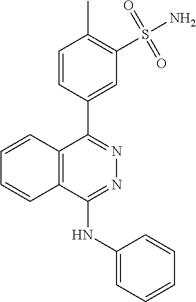 [SMILES: Cc1ccc(-c2nnc(Nc3ccccc3)c3ccccc23)cc1S(N)(=O)=O] | 1.0, 2.0, 1.6 | Antagonist or allosteric modulator |
| 20 (Group II) | 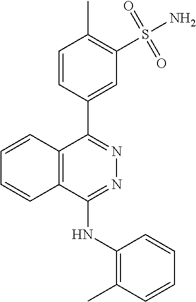 [SMILES: Cc1ccccc1Nc1nnc(-c2ccc(C)c(S(N)(=O)=O)c2)c2ccccc12] | 0.5. 1.0, 5.0 | Antagonist or allosteric modulator |
| 21 (Group III) | 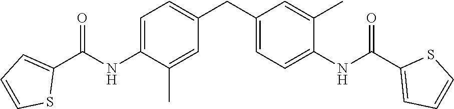 [SMILES: Cc1cc(Cc2ccc(NC(=O)c3cccs3)c(C)c2)ccc1NC(=O)c1cccs1] | 2.7, 0.5, >0.5 | Antagonist or allosteric modulator |
| 22 (Group IV) | 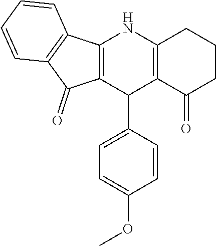 [SMILES: COc1ccc(C2C3=C(CCCC3=O)NC3=C2C(=O)c2ccccc23)cc1] | 6.7, 5.3 | IA, 1.0, 1.5; 10.2 fold decrease |
| 23 (Group IV) | 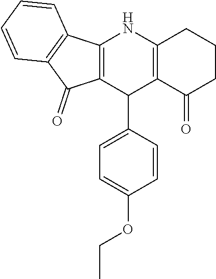 [SMILES: CCOc1ccc(C2C3=C(CCCC3=O)NC3=C2C(=O)c2ccccc23)cc1] | >10, 2.9 | IA, 0.4, 0.9, 1.2, 1.0; 14.1 fold decrease |
| 24 (Group IV) | 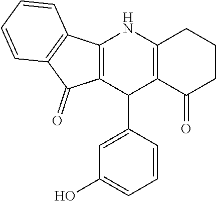 [SMILES: O=C1CCCC2=C1C(c1cccc(O)c1)C1=C(N2)c2ccccc2C1=O] | ND | IA, 4.4; 2.8 fold decrease |
| 25 (Group IV) | 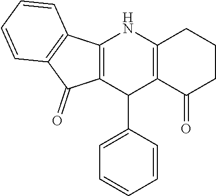 [SMILES: O=C1CCCC2=C1C(c1ccccc1)C1=C(N2)c2ccccc2C1=O] | ND | IA, 4.6, 4.2 fold decrease |
| 26 (Group IV) | 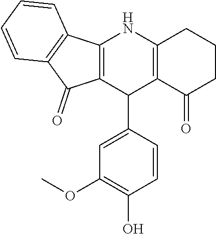 [SMILES: COc1cc(C2C3=C(CCCC3=O)NC3=C2C(=O)c2ccccc23)ccc1O] | ND | IA, 3.6, 1.7 fold decrease |
| 27 (Group IV) | 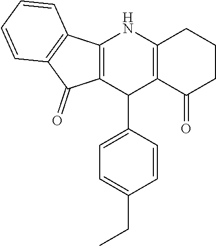 [SMILES: CCc1ccc(C2C3=C(CCCC3=O)NC3=C2C(=O)c2ccccc23)cc1] | ND | IA, 0.8, 0.5, 6.2 fold decrease |
| 28 (Group IV) | 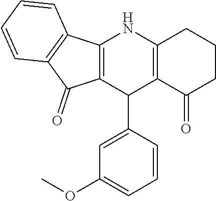 [SMILES: COc1cccc(C2C3=C(CCCC3=O)NC3=C2C(=O)c2ccccc23)c1] | ND | IA, 3.6, 1.9 fold decrease |
| 29 (Group IV) | 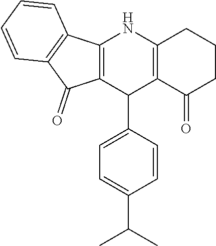 [SMILES: CC(C)c1ccc(C2C3=C(CCCC3=O)NC3=C2C(=O)c2ccccc23)cc1] | ND | IA, 1.2, 5.3 fold decrease |
| 30 (Group IV) | 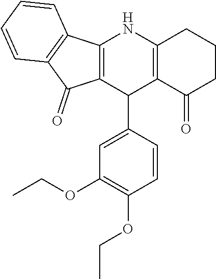 [SMILES: CCOc1ccc(C2C3=C(CCCC3=O)NC3=C2C(=O)c2ccccc23)cc1OCC] | ND | IA, 3.8, 2.2 fold decrease |
| 31 (Group IV) | 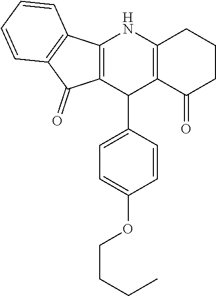 [SMILES: CCCCOc1ccc(C2C3=C(CCCC3=O)NC3=C2C(=O)c2ccccc23)cc1] | ND | IA, 1.2, 8.9 fold decrease |
| 32 (Group IV) | 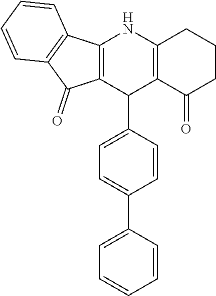 [SMILES: O=C1CCCC2=C1C(c1ccc(-c3ccccc3)cc1)C1=C(N2)c2ccccc2C1=O] | ND | IA, 2.5, 3.9 fold decrease |
| 33 (Group IV) | 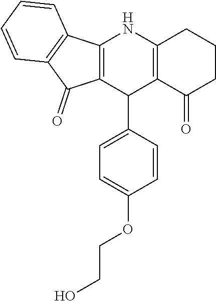 [SMILES: O=C1CCCC2=C1C(c1ccc(OCCO)cc1)C1=C(N2)c2ccccc2C1=O] | ND | IA, 4.4, 9.2 fold decrease |
| 34 (Group IV) | 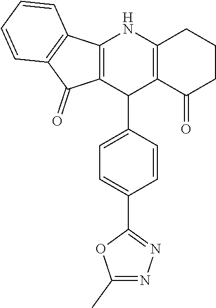 [SMILES: Cc1nnc(-c2ccc(C3C4=C(CCCC4=O)NC4=C3C(=O)c3ccccc34)cc2)o1] | ND | IA, 2.8, 8.4 fold decrease |
| 35 (Group IV) | 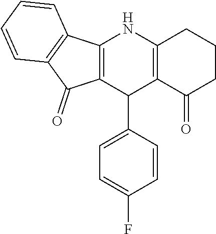 [SMILES: O=C1CCCC2=C1C(c1ccc(F)cc1)C1=C(N2)c2ccccc2C1=O] | ND | IA, 4.3, 2.8 fold decrease |
| 36 (Group IV) | 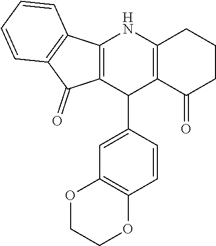 [SMILES: O=C1CCCC2=C1C(c1ccc3c(c1)OCCO3)C1=C(N2)c2ccccc2C1=O] | ND | IA, 2.6, 8.3 fold decrease |
| 37 (Group IV) | 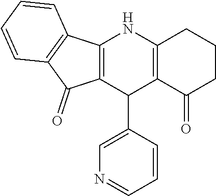 [SMILES: O=C1CCCC2=C1C(c1cccnc1)C1=C(N2)c2ccccc2C1=O] | ND | IA, 5.4, 2.8 fold decrease |
| 38 (Group IV) | 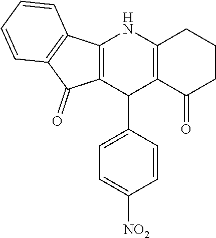 [SMILES: O=C1CCCC2=C1C(c1ccc([N+](=O)[O-])cc1)C1=C(N2)c2ccccc2C1=O] | ND | IA, 2.7, 8.6 fold decrease |
| 39 (Group IV) | 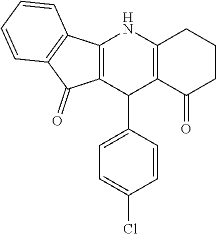 [SMILES: O=C1CCCC2=C1C(c1ccc(Cl)cc1)C1=C(N2)c2ccccc2C1=O] | ND | IA, 3, 7.8 fold decrease |
| 40 (Group IV) | 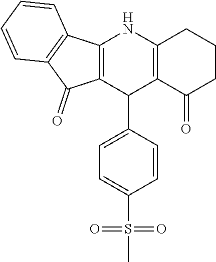 [SMILES: CS(=O)(=O)c1ccc(C2C3=C(CCCC3=O)NC3=C2C(=O)c2ccccc23)cc1] | ND | IA, 3.3, 8.8 fold decrease |
| 41 (Group IV) | 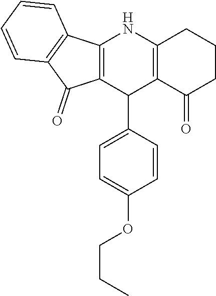 [SMILES: CCCOc1ccc(C2C3=C(CCCC3=O)NC3=C2C(=O)c2ccccc23)cc1] | ND | IA, 1.0; 13.7 fold decrease |
| 42 (Group IV) | 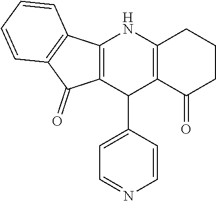 [SMILES: O=C1CCCC2=C1C(c1ccncc1)C1=C(N2)c2ccccc2C1=O] | ND | IA, 6.6, 4.3 fold decrease |
| 43 (Group IV) | 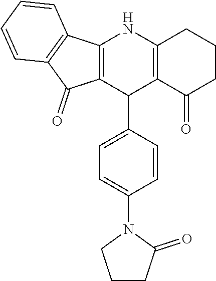 [SMILES: O=C1CCCC2=C1C(c1ccc(N3CCCC3=O)cc1)C1=C(N2)c2ccccc2C1=O] | ND | IA, 5.2, 5.1 fold decrease |
| 44 (Group IV) | 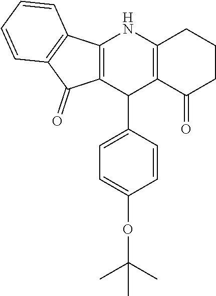 [SMILES: CC(C)(C)Oc1ccc(C2C3=C(CCCC3=O)NC3=C2C(=O)c2ccccc23)cc1] | ND | IA, 1.3, 4.7 fold decrease |
| 45 (Group IV) | 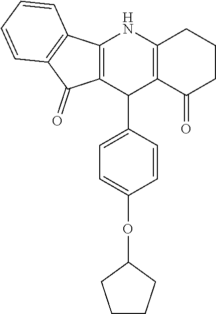 [SMILES: O=C1CCCC2=C1C(c1ccc(OC3CCCC3)cc1)C1=C(N2)c2ccccc2C1=O] | ND | IA, 1.0; 7.6 fold decrease |
| 46 (Group IV) | 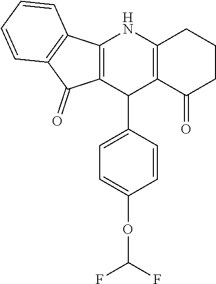 [SMILES: O=C1CCCC2=C1C(c1ccc(OC(F)F)cc1)C1=C(N2)c2ccccc2C1=O] | ND | IA, 1.2, 8.1 fold decrease |
| 47 (Group IV) | 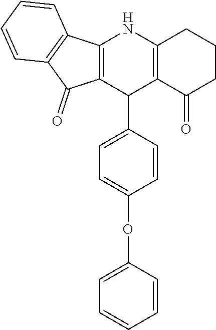 [SMILES: O=C1CCCC2=C1C(c1ccc(Oc3ccccc3)cc1)C1=C(N2)c2ccccc2C1=O] | ND | IA, 1.0, 7.1 fold decrease |
| 48 (Group IV) | 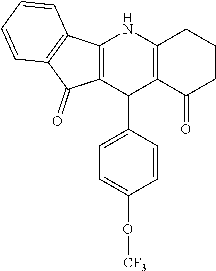 [SMILES: O=C1CCCC2=C1C(c1ccc(OC(F)(F)F)cc1)C1=C(N2)c2ccccc2C1=O] | ND | IA, 1.2, 5 fold decrease |
| 49 (Group IV) | 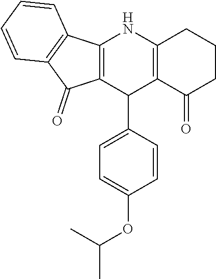 [SMILES: CC(C)Oc1ccc(C2C3=C(CCCC3=O)NC3=C2C(=O)c2ccccc23)cc1] | ND | IA, 0.6; 14.3 fold decrease |
| 50 (Group IV) | 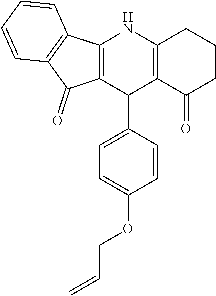 [SMILES: C=CCOc1ccc(C2C3=C(CCCC3=O)NC3=C2C(=O)c2ccccc23)cc1] | ND | IA, 1.2, 10.1 fold decrease |
| 51 (Group IV) | 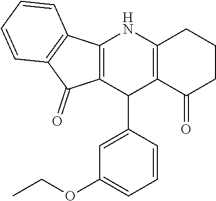 [SMILES: CCOc1cccc(C2C3=C(CCCC3=O)NC3=C2C(=O)c2ccccc23)c1] | ND | IA, 1.6, 1.7 fold decrease |
| 52 (Group IV) | 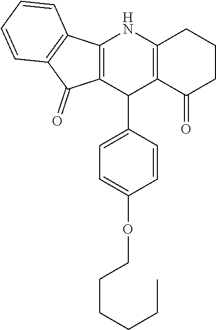 [SMILES: CCCCCCOc1ccc(C2C3=C(CCCC3=O)NC3=C2C(=O)c2ccccc23)cc1] | ND | IA, 0.5, 3.1 fold decrease |
| 54 (Group IV) | 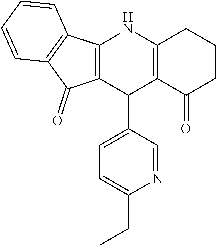 [SMILES: CCc1ccc(C2C3=C(CCCC3=O)NC3=C2C(=O)c2ccccc23)cn1] | ND | IA, 2.0, 7 fold decrease |
| 55 (Group IV) | 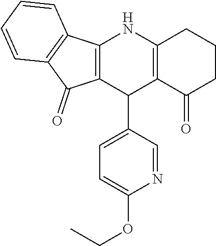 [SMILES: CCOc1ccc(C2C3=C(CCCC3=O)NC3=C2C(=O)c2ccccc23)cn1] | ND | IA, 0.6, 7 fold decrease |
| 56 (Group V) | 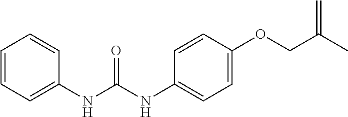 [SMILES: C=C(C)COc1ccc(NC(=O)Nc2ccccc2)cc1] | 0.5 | IA, 0.5, 3 fold decrease |
| 57 (Group VI) | 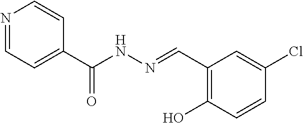 [SMILES: O=C(N/N=C/c1cc(Cl)ccc1O)c1ccncc1] | 5.6 | Antagonist or allosteric modulator |
| 58 (Group VI) | 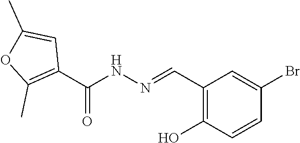 [SMILES: Cc1cc(C(=O)N/N=C/c2cc(Br)ccc2O)c(C)o1] | 3.7 | Antagonist or allosteric modulator |
| 59 (Group Va) | 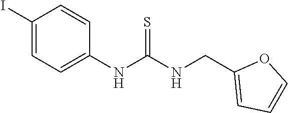 [SMILES: S=C(NCc1ccco1)Nc1ccc(I)cc1] | ND | NM |

For the EC₅₀ values, it is noted that all the compounds included in Table 1 showed at least a minimum activity level of greater than 3 times the average background of the assay when tested at 40 μM. Where present, multiple EC₅₀ values correspond to values obtained in separate experiments. “IA” refers to “inverse agonist.” “NM” refers to “non-modulator.” Compound 4 is labeled as an antagonist as it was found to compete with the GPR174 agonist LysoPS. The activity of the compounds was tested as described in US 20200276190, the disclosure of which is incorporated herein by reference.

In some embodiments, the GPR174 inhibitor is a compound of the formula VIII:

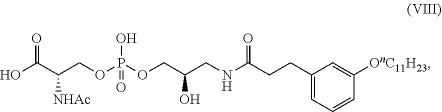

or an isomer or a salt thereof. In some embodiments, the GPR174 inhibitor is a compound disclosed in Sayama M. et al., Switching Lysophosphatidylserine G Protein-Coupled Receptor Agonists to Antagonists by Acylation of the Hydrophilic Serine Amine, Journal of Medicinal Chemistry, 2021 64 (14), 10059-10101.

As used herein, the term “G-protein coupled receptor” or “GPCR,” or “GPR” refers to a transmembrane receptor that is capable of transmitting a signal from the outside of a cell to the inside of a cell through a G-protein pathway and/or an arrestin pathway. Hundreds of such receptors are known in the art; see, e.g., Fredriksson et al., Mol. Pharmacol. 63:1256-1272, 2003, and Vassilatis, D. K., Proc Natl Acad Sci USA 100: 4903-4908 (2003), each of which are hereby incorporated by reference. These references have characterized the human and mouse GPCRs based on sequence homology and function. Human GPCRs can be broken down into five classes: secretin, rhodopsin, glutamate, frizzled/Tas2, and adhesion. Alternatively, receptors may be classified by their ligands, e.g., peptide hormones or small molecules (e.g., biogenic amines). Other classification schemes include the A-F classification, where class A represents receptors related to rhodopsin and the adrenergic receptors, class B, receptors related to the calcitonin and parathyroid hormone receptors, class C, receptors related to the metabotropic receptors, and classes D-F represent receptors found in fungi and archaebacteria.

The terms “G-protein coupled receptor 174,” “GPR174,” “FKSG79,” or “GPCR17” refer to any naturally occurring forms of the GPR174 protein, e.g., SEQ ID NO:1, or naturally occurring variants thereof, such as variants having at least 90% identity (such as at least 91%, 92%, 93%, 94%, 95%, 96%, 97%, 98% or 99% identity) to SEQ ID NO:1. Preferable forms of GPR174 have the ability to signal through at least one G-protein coupled receptor pathway such as Gs.

The term “G-protein” refers to a heterotrimeric protein complex that transmits a signal from an activated GPCR to effector molecule(s) inside the cell such as enzymes and ion channels. G-proteins are made up of Gα, Gβ, and Gγ subunits. Families of Ga subunits include Gq, Gi, Gs, and Gα12/13. G-protein signaling pathways are named for the activated Ga subunit, i.e., Gαs, Gαi, Gαq, and Gα12/13. A heterotrimeric G-protein binds to an activated GPCR protein, that is, a GPCR protein that is bound to a ligand or surrogate ligand. When bound to a GPCR protein, the Ga subunit exchanges bound guanosine diphosphate (GDP) for guanosine-5′-triphosphate (GTP) and dissociates from the Gβ and Gγ subunits, which are typically associated in a heterodimeric complex. Once dissociated, both the Gα-GTP-bound protein and the Gβγ complex can activate signaling pathways. The Gq family includes Gαq, Gα11, Gα14, and Gα15/16. The Gi family includes Gαi1-3, Gαo, Gαt, Gαgust, and Gαz. The Gs family includes Gαs and Gαolf The G12/13 includes Gα12 and Gα13.

The term “contacting” is used herein interchangeably with the following: combined with, added to, mixed with, introducing to, passed over, incubated with, flowed over, etc. For purposes of clarity, the phrase “contacting a cell” includes introducing a compound into a mammal (e.g., orally, into the plasma, or intramuscularly) such that the compound contacts the cells of the mammal in vivo.

An “inhibitor” is a compound that decreases signaling in an indicated pathway. Inhibitors are compounds that functionally interact with a substrate and partially or totally block activity, decrease, prevent, delay activation, inactivate, antagonize, desensitize, drive the conformation of the substrate to the inactive conformation, block the ability of another compound (e.g., an endogenous agonist ligand) to interact with the substrate, or otherwise down-regulate the activity of the substrate. Inhibitors can reduce basal activity of the substrate (e.g., an inverse agonist) or can block or reduce activity of another compound (e.g., a partial agonist or antagonist). Inhibitors include antagonists, inverse agonists, partial agonists, partial inverse agonists, and negative allosteric modulators. Inhibitors do not include compounds that act solely by decreasing expression of the receptor nucleic acid or protein.

A “ligand” is a compound that binds to a receptor or substrate and modulates the activity of the receptor.

The term “compound” or grammatical equivalents as used herein refers to molecules, either naturally occurring or synthetic, e.g., protein; antibody, oligopeptide (e.g., from about 5 to about 25 amino acids in length, such as from about 10 to 20 or 12 to 18 amino acids in length, for example, 12, 15, or 18 amino acids in length); nucleotides (e.g., inhibitory RNA) that inhibits expression, small molecule chemical compounds, e.g., small organic, organometallic, or inorganic molecule; polysaccharide; oligonucleotides; lipid; and fatty acid. The compound can be included in a library of compounds, such as a combinatorial, synthetic, natural, heterocyclic, drug-like, lead-like, organic, inorganic, unrandomized, or randomized library that provides a sufficient range of diversity or it may be a focused or targeted collection of the above compounds. Compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a compound (called a “lead compound”) having some desirable property or activity, e.g., inhibitory activity, creating variants of the lead compound, and evaluating the property and activity of those variant compounds. Often, high-throughput screening (“HTS”) methods are employed for such an analysis.

The terms “small molecule,” “small organic molecule,” and “small inorganic molecule” refer to molecules (either organic, organometallic, or inorganic), organic molecules, and inorganic molecules, respectively, which are either naturally occurring or synthetic and that have a molecular weight of more than about 50 Da and less than about 2500 Da. Small organic (for example) molecules may be less than about 2000 Da, between about 100 Da to about 1000 Da, or between about 100 to about 600 Da, or between about 200 to 500 Da.

By “therapeutically effective amount” or “effective amount” is meant an amount that produces a desired effect for which it is administered, e.g., improvement or delay of at least one symptom associated with the disease or condition being treated. The exact dose will depend on the purpose of the treatment and can be ascertained by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)). In some embodiments, the “effective amount” refers to the amount and/or concentration of the one or more phenotype altering agent in the composition for culturing T cells as disclosed herein that results in the change of the phenotype of at least a sub-population of the T cells.

By “substantially pure” or “isolated” is meant a compound (e.g., a polypeptide or conjugate) that has been separated from other chemical components. Typically, the compound is substantially pure when it is at least 30%, by weight, free from other components. In certain embodiments, the preparation is at least 50%, 60%, 75%, 85%, 90%, 95%, 96%, 97%, 98%, or 99% by weight, free from other components. A purified polypeptide may be obtained, for example, by expression of a recombinant polynucleotide encoding such a polypeptide or by chemically synthesizing the polypeptide. Purity can be measured by any appropriate method, for example, column chromatography, polyacrylamide gel electrophoresis, or by HPLC analysis.

In the context of a naturally occurring compound, the term “isolated” is one which is altered or removed from the natural state (e.g., through human intervention).

The term “alkanoyl,” as used herein, refers to a group having the structure —C(O)—R, in which R is alkyl. Alkanoyl may be unsubstituted or substituted (e.g., optionally substituted alkanoyl) as described for alkyl. The suffix “oyl” may be used to define other groups having the structure —C(O)—R. For example, in alkenoyl group, R is alkenyl; in alknynoyl group, R is alkynyl; in cycloalkanoyl group, R is cycloalkyl; in cycloalkenoyl group, R is cycloalkenyl; and in cycloalkynoyl group, R is cycloalkynyl (all groups are as defined herein). Further, the groups defined with a suffix “oyl” may be further used to define groups having the structure —O—C(O)—R′ by adding the suffix “oxy,” e.g., when R′ is alkyl, this group is “alkanoyloxy.” For example, in alkenoyloxy group, R′ is alkenyl; in alknynoyloxy group, R′ is alkynyl; in cycloalkanoyloxy group, R′ is cycloalkanyl; in cycloalkenoyloxy group, R′ is cycloalkenyl; and in cycloalkynoyloxy group, R′ is cycloalkynyl (all groups are as defined herein). Each of these groups may be unsubstituted or substituted (e.g., optionally substituted) as described for each respective group.

The term “alkenyl,” as used herein, refers to a straight-chain or branched-chain monovalent substituent including one or two carbon-carbon double bonds and containing only C and H when unsubstituted. Alkenyl group may contain, unless otherwise specified, 2, 3, 4, 5, or 6 carbon atoms, excluding the carbon atoms of any substituents, if present. Non-limiting examples of alkenyl groups include ethenyl, prop-i-enyl, prop-2-enyl, 1-methylethenyl, but-i-enyl, but-2-enyl, but-3-enyl, 1-methylprop-i-enyl, 2-methylprop-i-enyl, and 1-methylprop-2-enyl. Alkenyl may be unsubstituted or substituted (e.g., optionally substituted alkenyl) as described for alkyl.

The term “alkenylene,” as used herein, refers to a straight-chain or branched-chain divalent substituent including one or two carbon-carbon double bonds and containing only C and H when unsubstituted. Alkenylene group may contain, unless otherwise specified, 2, 3, 4, 5, or 6 carbon atoms, excluding the carbon atoms of any substituents, if present. Non-limiting examples of alkenylene groups include ethen-1,1-diyl; ethen-1,2-diyl; prop-1-en-1,1-diyl, prop-2-en-1,1-diyl; prop-1-en-1,2-diyl, prop-1-en-1,3-diyl; prop-2-en-1,1-diyl; prop-2-en-1,2-diyl; but-1-en-1,1-diyl; but-1-en-1,2-diyl; but-1-en-1,3-diyl; but-1-en-1,4-diyl; but-2-en-1,1-diyl; but-2-en-1,2-diyl; but-2-en-1,3-diyl; but-2-en-1,4-diyl; but-2-en-2,3-diyl; but-3-en-1,1-diyl; but-3-en-1,2-diyl; but-3-en-1,3-diyl; but-3-en-2,3-diyl; buta-1,2-dien-1,1-diyl; buta-1,2-dien-1,3-diyl; buta-1,2-dien-1,4-diyl; buta-1,3-dien-1,1-diyl; buta-1,3-dien-1,2-diyl; buta-1,3-dien-1,3-diyl; buta-1,3-dien-1,4-diyl; buta-1,3-dien-2,3-diyl; buta-2,3-dien-1,1-diyl; and buta-2,3-dien-1,2-diyl. Alkenylene may be unsubstituted or substituted (e.g., optionally substituted alkenylene) as described for alkylene.

The term “alkoxy” represents a chemical substituent of formula —OR, where R is an optionally substituted alkyl group (e.g., optionally substituted C₁-C₆ alkyl group). The substituted alkoxy group can have 1, 2, 3, 4, 5, or 6 substituent groups as defined herein. Similarly, the term “arylalkoxy” represents a chemical substituent of formula —OR, where R is an optionally substituted arylalkyl group. The term “cycloalkoxy” represents a substituent of formula —OR′, where R′ is an optionally substituted cycloalkyl group as described herein. Similarly, the term “alkenoxy” represents a chemical substituent of formula —OR″, where R″ is an optionally substituted alkenyl group as described herein.

The term “alkyl,” as used herein, refers to a saturated straight-chain or branched-chain monovalent substituent, containing only C and H when unsubstituted. Alkyl group may contain, unless otherwise specified, 1, 2, 3, 4, 5, or 6 carbon atoms, excluding the carbon atoms of any substituents, if present. Non-limiting examples of alkyl group include methyl, ethyl, isobutyl, tert-butyl, and the like. Alkyl group may be unsubstituted or substituted (e.g., optionally substituted alkyl) with 1, 2, 3, 4, 5, or 6 substituents independently selected from the group consisting of: halo (e.g., F, Cl, Br, or I), CN, NO₂, CF₃, OCF₃, COOR′, CONR′₂, OR′, SR′, SOR′, SO₂R′, NR′₂, NR′(CO)R′, NR′C(O)OR′, NR′C(O)NR′₂, NR′SO₂NR′₂, NR′SO₂R′, oxo (═O), or oximido (═NOR″), where each R′ is, independently, H or an optionally substituted group selected from alkyl, alkenyl, alkynyl, cycloalkyl, cycloalkenyl, cycloalkynyl, heteroalkyl, heteroalkenyl, heteroalkynyl, heteroaryl, and aryl (all as defined herein); and R″ is H or an optionally substituted group selected from alkyl, alkenyl, alkynyl, heteroalkyl, heteroalkenyl, heteralkynyl, heteroaryl, and aryl (all as defined herein). Alternatively, a substituted alkyl group may be a perfluoroalkyl group. In certain embodiments, when at least one of the substituents on alkyl group is oxo, the oxo group is not bonded to the carbon atom bonded to the parent molecular group.

The term “alkylene,” as used herein, refers to a saturated straight-chain or branched-chain divalent substituent, containing only C and H when unsubstituted. Alkylene group may contain, unless otherwise specified, 1, 2, 3, 4, 5, or 6 carbon atoms, excluding the carbon atoms of any substituents, if present. Non-limiting examples of alkylene group include methylene, ethane-1,2-diyl, ethane-1,1-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, and butane-2,2-diyl, butane-2,3-diyl. Alkylene group may be unsubstituted or substituted (e.g., optionally substituted alkylene) with 1, 2, 3, 4, 5, or 6 substituents independently selected from the group consisting of: halo (e.g., F, Cl, Br, or I), CN, NO₂, CF₃, OCF₃, COOR′, CONR′₂, OR′, SR′, SOR′, SO₂R′, NR′₂, NR′(CO)R′, NR′C(O)OR′, NR′C(O)NR′₂, NR′SO₂NR′₂, NR′SO₂R′, oxo (═O), or oximido (═NOR″), where each R′ is, independently, H or an optionally substituted group selected from alkyl, alkenyl, alkynyl, cycloalkyl, cycloalkenyl, cycloalkynyl, heteroalkyl, heteroalkenyl, heteroalkynyl, heteroaryl, and aryl (all as defined herein); and R″ is H or an optionally substituted group selected from alkyl, alkenyl, alkynyl, heteroalkyl, heteroalkenyl, heteralkynyl, heteroaryl, and aryl (all as defined herein). Alternatively, a substituted alkylene group may be a perfluoroalkylene group.

The term “alkylsulfinyl” refers to a group having the structure alkyl-S(O)—, in which alkyl is as described herein. Alkylsulfinyl may be unsubstituted or substituted (e.g., optionally substituted alkylsulfinyl) as described for alkyl.

The term “alkylsulfonyl” refers to a group having the structure alkyl-S(O)₂—, in which alkyl is as described herein. Alkylsulfonyl may be unsubstituted or substituted (e.g., optionally substituted alkylsulfonyl) as described for alkyl.

The term “alkynyl,” as used herein, refers to a straight-chain or branched-chain monovalent substituent including one or two carbon-carbon triple bonds and containing only C and H when unsubstituted. Alkynyl group may contain, unless otherwise specified, 2, 3, 4, 5, or 6 carbon atoms, excluding the carbon atoms of any substituents, if present. Non-limiting examples of alkynyl groups include ethynyl, prop-1-ynyl, prop-2-ynyl, buty-1-nyl, but-2-ynyl, but-3-ynyl, 1-methylprop-2-ynyl, and the like. Alkynyl may be unsubstituted or substituted (e.g., optionally substituted alkynyl) as described for alkyl.

The term “alkynylene,” as used herein, refers to a straight-chain or branched-chain divalent substituent including one or two carbon-carbon triple bonds and containing only C and H when unsubstituted. Alkynylene group may contain, unless otherwise specified, 2, 3, 4, 5, or 6 carbon atoms, excluding the carbon atoms of any substituents, if present. Non-limiting examples of alkenylene groups include ethyn-1,2-diyl; prop-1-yn-1,3-diyl; prop-2-yn-1,1-diyl; but-1-yn-1,3-diyl; but-1-yn-1,4-diyl; but-2-yn-1,1-diyl; but-2-yn-1,4-diyl; but-3-yn-1,1-diyl; but-3-yn-1,2-diyl; but-3-yn-2,2-diyl; and buta-1,3-diyn-1,4-diyl. Alkenylene may be unsubstituted or substituted (e.g., optionally substituted alkenylene) as described for alkylene.

The term “amido,” as used herein, refers to a group having a structure —N(R^{N1})R^{N2}, in which R^{N1} is —H, —OH, —N(R^{N3})₂, —C(O)R^{N4}, —SO₂OR^{N4}, —SO₂R^{N4}, —SOR^{N4}, alkyl, alkenyl, alkynyl, alkoxy, aryl, arylalkyl, cycloalkyl, cycloalkylalkyl, heterocyclyl (e.g., heteroaryl), or heterocyclylalkyl (e.g., heteroarylalkyl); R^{N2} is —C(O)R^{N5}, SO₂OR^{N5}, SO₂R^{N5}, or SOR^{N5}; or R^{N1} and R^{N5} combine to form a 5-, 6-, 7-, or 8-membered ring. R^{N3} is H, alkyl, aryl, arylalkyl, cycloalkyl, cycloalkylalkyl, heterocyclyl (e.g., heteroaryl), or heterocyclylalkyl (e.g., heteroarylalkyl); each of R^{N4} and R^{N5} is, independently, alkyl, alkenyl, alkynyl, alkoxy, aryl, arylalkyl, cycloalkyl, cycloalkylalkyl, heterocyclyl (e.g., heteroaryl), or heterocyclylalkyl (e.g., heteroarylalkyl). In a preferred embodiment, R^{N1} is H. Amido may be unsubstituted, when R^{N1} is H and the group in R^{N2} is unsubstituted (e.g., R^{N3} is H, unsubstituted alkyl, unsubstituted aryl, unsubstituted arylalkyl, unsubstituted cycloalkyl, unsubstituted cycloalkylalkyl, unsubstituted heterocyclyl (e.g., unsubstituted heteroaryl), or unsubstituted heterocyclylalkyl (e.g., unsubstituted heteroarylalkyl); or each of R^{N4} and R^{N5} is unsubstituted alkyl, unsubstituted alkenyl, unsubstituted alkynyl, unsubstituted alkoxy, unsubstituted aryl, unsubstituted arylalkyl, unsubstituted cycloalkyl, unsubstituted cycloalkylalkyl, unsubstituted heterocyclyl (e.g., unsubstituted heteroaryl), or unsubstituted heterocyclylalkyl (e.g., unsubstituted heteroarylalkyl)). Alternatively, amido may be substituted, when at least one of the groups listed under R^{N3}, R^{N4}, or R^{N5} is substituted, and/or when R^{N1} is not H.

The term “amino,” as used herein, represents —N(R^{N1})₂, wherein each R^{N1} is, independently, H, OH, NO₂, N(R^{N2})₂, an N-protecting group, alkyl, alkenyl, alkynyl, alkoxy, aryl, arylalkyl, cycloalkyl, cycloalkylalkyl, heterocyclyl (e.g., heteroaryl), heterocyclylalkyl (e.g., heteroarylalkyl), or two R^{N1} combine to form a heterocyclyl or an N-protecting group, and wherein each R^{N2} is, independently, H, alkyl, or aryl. Amino may be unsubstituted, when each R^{N1} is H, or substituted, when at least one R^{N1} is not H (e.g., optionally substituted amino). In a preferred embodiment, amino is —NH₂ or —NHR^{N1}, wherein R^{N1} is, independently, OH, NO₂, NH₂, NR^{N2}₂, SO₂OR^{N2}, SO₂R^{N2}, SOR^{N2}, alkyl, or aryl, and each R^{N2} can be H, alkyl, or aryl.

The terms “aromatic moiety” and “aryl,” as used herein, refer to a carbocyclic monovalent group (monocyclic or fused ring bicyclic), in which the carbocycle satisfies Hückel's rule (4n+2 electrons in a single π system) and has the characteristics of aromatic stabilization relative to a hypothetical molecule not having aromatic stabilization (e.g., benzene as compared to cyclohexatriene). Aryl may contain 6-10 carbons, excluding the carbon atoms of any substituents, if present. Non-limiting examples of monocyclic and fused bicyclic aromatic moieties include phenyl and naphthyl, respectively. Aryl may be unsubstituted or substituted as defined herein. The term “arylene” refers to an aryl group, as described herein, except in that arylene is a divalent substituent. Arylene may be unsubstituted or substituted as defined herein.

The term “arylalkyl,” as used herein, represents a chemical substituent (aryl)-(alkylene)-, in which each of aryl and alkylene group is as described herein. Arylalkyl group may be unsubstituted or substituted (e.g., optionally substituted C₆-C₁₀ aryl C₁-C₆ alkyl). A non-limiting example of arylalkyl is phenylmethyl, commonly referred to as benzyl. Arylalkenyl (e.g., C₆-C₁₀ aryl C₂-C₆ alkenyl) and arylalkynyl (e.g., C₆-C₁₀ aryl C₂-C₆ alkynyl) are similarly defined as having the general structure of (aryl)-(alkenylene)- and (aryl)-(alkynylene)-, respectively. Arylheteroalkyl, arylheteroalkenyl, and arylheteroalkynyl are similarly defined as having the structure (aryl)-(heteroalkylene)-, (aryl)-(heteroalkenylene)-, and (aryl)-(heteroalkynylene)-, respectively. Similarly, other groups can be defined through the combination of the term defining a group with “alkyl.” For example, “heteroarylalkyl” is a chemical substituent having the general structure (heteroaryl)-(alkylene)-, which may be unsubstituted or substituted (e.g., optionally substituted C₁-C₉ heteroaryl C₁-C₆ alkyl) according to the respective definitions of each portion of heteroarylalkyl group. Each of the groups may be unsubstituted or substituted (e.g., optionally substituted). The substituents for aryl or heteroaryl portion are those described for aromatic groups. The substituents for alkyl, heteroalkyl, alkenyl, heteroalkenyl, alkynyl, or heteroalkynyl portion are those described in the respective definitions of these groups.

The term “aryloyl,” as used herein, refers to a group having the structure (C₆-C₁₀ aryl)-C(O)—. Aryloyl may be unsubstituted or substituted according to the definition of an aryl group (e.g., optionally substituted aryloyl). A typical example of aryloyl group is benzoyl group. Similarly, the term “heteroaryloyl,” as used herein, refers to a group having the structure (C₁-C₉ heteroaryl)-C(O)—. Heteroaryloyl may be unsubstituted or substituted (e.g., optionally substituted heteroaryloyl) as described for heteroaryl.

The term “aryloxy,” as used herein, refers to a carbocyclic aromatic system linked to another residue through an oxygen atom, e.g., (C₆-C₁₀ aryl)-O—. Aryloxy group may be unsubstituted or substituted (e.g., optionally substituted aryl) as described for the aromatic groups. A typical example of an aryloxy is phenoxy (e.g., optionally substituted phenoxy).

The term “aryloyloxy,” as used herein, refers to a group having the structure (C₆-C₁₀ aryl)-C(O)—O—. Aryloyloxy may be unsubstituted or substituted according to the definition of an aryl group (e.g., optionally substituted aryloyloxy). A typical example of the aryloyloxy group is benzoate. Similarly, the term “heteroaryloyloxy,” as used herein, refers to a group having the structure (C₁-C₉ hetereoaryl)-C(O)—O—. Heteroaryloyloxy may be unsubstituted or substituted (e.g., optionally substituted heteroaryloyloxy) as described for heteroaryl.

The term “arylsulfinyl” refers to a group having the structure (C₆-C₁₀ aryl)-S(O)—. Arylsulfinyl group may be unsubstituted or substituted as described herein (e.g., optionally substituted arylsulfinyl). A non-limiting example of arylsulfinyl is phenylsulfinyl.

The term “arylsulfonyl” refers to a group having the structure (C₆-C₁₀ aryl)-S(O)₂—. Arylsulfonyl group may be unsubstituted or substituted as described herein (e.g., optionally substituted arylsulfonyl). A non-limiting example of arylsulfonyl is phenylsulfonyl.

The term “arylthio” refers to a group having the structure (C₆-C₁₀ aryl)-S—. Arylthio group may be unsubstituted or substituted as described herein (e.g., optionally substituted arylthio). A non-limiting example of arylthio is phenylthio.

The term “carbocyclic,” as used herein, represents an optionally substituted C₃₋₁₂ monocyclic, bicyclic, or tricyclic structure in which the rings, which may be aromatic or non-aromatic, are formed by carbon atoms. Carbocyclic structures include cycloalkyl, cycloalkenyl, cycloalkynyl, and aryl groups.

The term “carbonyl,” as used herein, refers to a divalent group consisting of a C═O, in which the two valences are on the carbon atom. This term can be used to define other groups having the general structure R—C(O)—. Thus, in alkoxycarbonyl group, R is alkoxy; in aryloxycarbonyl group, R is aryloxy, in aminocarbonyl group, R is amino; in heteroaryloxycarbonyl group, R is heteroaryloxy; in heterocyclyloxycarbonyl group, R is heterocyclyloxy; or in hydroxycarbonyl group, R is hydroxy. Each of the groups may be unsubstituted or substituted in accordance with the definition provided herein. For example, an alkoxycarbonyl group may be unsubstituted or substituted as defined for alkoxy group.

The terms “carboxamide” and “carboxylic acid amide,” as used herein, refer to a group having the structure —CONR′R″, where each R′ and R″ is selected, independently, from H, optionally substituted C₁₋₆ alkyl, optionally substituted C₃-C₁₀ cycloalkyl, optionally substituted C₁-C₉ heterocyclyl, optionally substituted C₆-C₁₀ aryl, optionally substituted C₁-C₉ heteroaryl, or R′ and R″ combine to form an optionally substituted heterocyclyl. Carboxamide may be unsubstituted, when the R′ group and the R″ group are unsubstituted, or substituted, when at least one of R′ and R″ is a substituted group as defined herein. Accordingly, optionally substituted carboxamide is a carboxamide that may be unsubstituted or substituted.

The terms “carboxylic acid ester” and “ester,” as used herein, refer to a group having the structure —CO₂R′, where R′ is selected from optionally substituted alkyl, optionally substituted cycloalkyl, optionally substituted heterocyclyl, optionally substituted aryl, or optionally substituted heteroaryl. Ester may be unsubstituted, when the R′ group is an unsubstituted group, or substituted, when R′ group is a substituted group as defined herein. Accordingly, optionally substituted ester is an ester that may be unsubstituted or substituted.

By “cyano” is meant a group having the structure —CN.

The term “cycloalkenyl,” as used herein, refers to a non-aromatic carbocyclic group having one, two, or three carbon-carbon double bonds and having from three to ten carbons (e.g., a C₃-C₁₀ cycloalkylene), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenylene,” as used herein, refers to a divalent non-aromatic carbocyclic group having one, two, or three carbon-carbon double bonds and having from three to ten carbons (e.g., C₃-C₁₀ cycloalkenylene), unless otherwise specified. Non-limiting examples of the cycloalkenylene include cycloprop-1-en-1,2-diyl; cycloprop-2-en-1,1-diyl; cycloprop-2-en-1,2-diyl; cyclobut-1-en-1,2-diyl; cyclobut-1-en-1,3-diyl; cyclobut-1-en-1,4-diyl; cyclobut-2-en-1,1-diyl; cyclobut-2-en-1,4-diyl; cyclopent-1-en-1,2-diyl; cyclopent-1-en-1,3-diyl; cyclopent-1-en-1,4-diyl; cyclopent-1-en-1,5-diyl; cyclopent-2-en-1,1-diyl; cyclopent-2-en-1,4-diyl; cyclopent-2-en-1,5-diyl; cyclopent-3-en-1,1-diyl; cyclopent-1,3-dien-1,2-diyl; cyclopent-1,3-dien-1,3-diyl; cyclopent-1,3-dien-1,4-diyl; cyclopent-1,3-dien-1,5-diyl; cyclopent-1,3-dien-5,5-diyl; norbornadien-1,2-diyl; norbornadien-1,3-diyl; norbornadien-1,4-diyl; norbornadien-1,7-diyl; norbornadien-2,3-diyl; norbornadien-2,5-diyl; norbornadien-2,6-diyl; norbornadien-2,7-diyl; and norbornadien-7,7-diyl. The cycloalkenylene may be unsubstituted or substituted (e.g., optionally substituted cycloalkenylene) as described for cycloalkyl.

The term “cycloalkyl,” as used herein, refers to a monovalent carbocyclic group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1]heptyl, 2-bicyclo[2.2.1]heptyl, 5-bicyclo[2.2.1]heptyl, 7-bicyclo[2.2.1]heptyl, and decalinyl. The cycloalkyl group may be unsubstituted or substituted (e.g., optionally substituted cycloalkyl) with 1, 2, 3, 4, 5, or 6 substituents independently selected from the group consisting of: alkyl, alkenyl, alkynyl, heteroalkyl, heteroalkenyl, heteroalkynyl, aryl, arylalkyl, heteroaryl, halo (e.g., F, Cl, Br, or I), CN, NO₂, CF₃, OCF₃, COOR′, CONR′₂, OR′, SR′, SOR′, SO₂R′, NR′₂, NR′(CO)R′,NR′C(O)OR′, NR′C(O)NR′₂, NR′SO₂NR′₂, NR′SO₂R′, oxo (═O), or oximido (═NOR″), where each R′ is, independently, H or an optionally substituted group selected from alkyl, alkenyl, alkynyl, cycloalkyl, cycloalkenyl, cycloalkynyl, heteroalkyl, heteroalkenyl, heteroalkynyl, heteroaryl, and aryl (all as defined herein); and R″ is H or an optionally substituted group selected from alkyl, alkenyl, alkynyl, heteroalkyl, heteroalkenyl, heteralkynyl, heteroaryl, and aryl (all as defined herein). Alternatively, a substituted cycloalkyl group may be a perfluorocycloalkyl group.

The term “cycloalkylene,” as used herein, refers to a divalent carbocyclic group having from three to ten carbons (e.g., C₃-C₁₀ cycloalkyl), unless otherwise specified. Non-limiting examples of cycloalkylene include cyclopropane-1,1-diyl; cyclopropane-1,2-diyl; cyclobutane-1,1-diyl; cyclobutane-1,2-diyl; cyclobutane-1,3-diyl; bicyclo[2.2.1]hepta-1,2-diyl; bicyclo[2.2.1]hepta-1,3-diyl; bicyclo[2.2.1]hepta-1,4-diyl; bicyclo[2.2.1]hepta-1,7-diyl; bicyclo[2.2.1]hepta-2,2-diyl; bicyclo[2.2.1]hepta-2,3-diyl; bicyclo[2.2.1]hepta-2,7-diyl; decalin-1,2-diyl; decalin-1,3-diyl; decalin-1,4-diyl; decalin-1,5-diyl; decalin-1,6-diyl; decalin-2,2-diyl; decalin-2,3-diyl; decalin-2,4-diyl; and decalin-2,5-diyl. The cycloalkylene group may be unsubstituted or substituted (e.g., optionally substituted cycloalkylene) as described for cycloalkyl.

The term “cycloalkynyl,” as used herein, refers to a monovalent carbocyclic group having one or two non-contiguous carbon-carbon triple bonds and having from eight to ten carbons (e.g., a C₈-C₁₀ cycloalkyl), unless otherwise specified. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as described for cycloalkyl.

Halo may be any halogen atom, especially F, Cl, Br, or I, and more particularly it is fluoro or chloro.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens. Haloalkyl groups include perfluoroalkyls. In some embodiments, the haloalkyl group can be further substituted with 1, 2, 3, or 4 substituent groups as described herein for alkyl groups.

The term “heteroalkenyl,” as used herein refers to an alkenyl group in which alkenyl chain is interrupted once by one, two, or three heteroatoms; twice, each time, independently, by one, two, or three heteroatoms; three times, each time, independently, by one, two, or three heteroatoms; or four times, each time, independently, by one, two, or three heteroatoms. Each heteroatom is, independently, O, N, or S. None of the heteroalkenyl groups includes more than two contiguous oxygen atoms. The heteroalkenyl group may be unsubstituted or substituted (e.g., optionally substituted heteroalkenyl). When the heteroalkenyl group is substituted and the substituent is bonded to the heteroatom, the substituent is selected accordingly. The substituent bonded to the heteroatom, valency permitting, is selected from the group consisting of: alkyl, alkanoyl, alkenyl, alkenoyl, alkynyl, alkynoyl, cycloalkyl, cycloalkanoyl, cycloalkenyl, cycloalkenoyl, cycloalkynyl, cycloalkynoyl, aryl, aryloyl, heteroaryl, heteroaryloyl, heterocyclyl, heterocycloyl, amino, aminocarbonyl, alkoxycarbonyl, aryloxycarbonyl, heteroaryloxycarbonyl, and heterocyclyloxycarbonyl. When the heteroalkenyl group is substituted and the substituent is bonded to carbon, the substituent is selected from those described for alkyl, provided that the substituent on the carbon atom bonded to the heteroatom is not halo. In some embodiments, the heteroalkenyl group has C at the terminus that attaches to other groups. In some embodiments, the heteroatom is O or N.

The term “heteroalkenylene,” as used herein refers to an alkenylene group in which alkenylene chain is interrupted once by one, two, or three heteroatoms; twice, each time, independently, by one, two, or three heteroatoms; three times, each time, independently, by one, two, or three heteroatoms; or four times, each time, independently, by one, two, or three heteroatoms. Each heteroatom is, independently, O, N, or S. None of the heteroalkenylene groups includes more than two contiguous oxygen atoms. The heteroalkenylene group may be unsubstituted or substituted (e.g., optionally substituted heteroalkenylene). When the heteroalkenylene group is substituted and the substituent is bonded to the heteroatom, the substituent is selected accordingly. The substituent bonded to the heteroatom, valency permitting, is selected from the group consisting of: alkyl, alkanoyl, alkenyl, alkenoyl, alkynyl, alkynoyl, cycloalkyl, cycloalkanoyl, cycloalkenyl, cycloalkenoyl, cycloalkynyl, cycloalkynoyl, aryl, aryloyl, heteroaryl, heteroaryloyl, heterocyclyl, heterocycloyl, amino, aminocarbonyl, alkoxycarbonyl, aryloxycarbonyl, heteroaryloxycarbonyl, and heterocyclyloxycarbonyl. When the heteroalkenylene group is substituted and the substituent is bonded to carbon, the substituent is selected from those described for alkyl, provided that the substituent on the carbon atom bonded to the heteroatom is not halo. In some embodiments, the heteroalkenylene group has C at each terminus that attaches to other groups. In some embodiments, the heteroatom is O or N.

The term “heteroalkyl,” as used herein refers to an alkyl group in which alkyl chain is interrupted once by one, two, or three heteroatoms; twice, each time, independently, by one, two, or three heteroatoms; three times, each time, independently, by one, two, or three heteroatoms; or four times, each time, independently, by one, two, or three heteroatoms. Each heteroatom is, independently, O, N, or S. None of the heteroalkyl groups includes more than two contiguous oxygen atoms. The heteroalkyl group may be unsubstituted or substituted (e.g., optionally substituted heteroalkyl). When the heteroalkyl group is substituted and the substituent is bonded to the heteroatom, the substituent is selected accordingly. The substituent bonded to the heteroatom, valency permitting, is selected from the group consisting of: alkyl, alkanoyl, alkenyl, alkenoyl, alkynyl, alkynoyl, cycloalkyl, cycloalkanoyl, cycloalkenyl, cycloalkenoyl, cycloalkynyl, cycloalkynoyl, aryl, aryloyl, heteroaryl, heteroaryloyl, heterocyclyl, heterocycloyl, amino, aminocarbonyl, alkoxycarbonyl, aryloxycarbonyl, heteroaryloxycarbonyl, and heterocyclyloxycarbonyl. When the heteroalkyl group is substituted and the substituent is bonded to carbon, the substituent is selected from those described for alkyl, provided that the substituent on the carbon atom bonded to the heteroatom is not halo. In some embodiments, the heteroalkyl group has C at the terminus that attaches to another group. In some embodiments, the heteroatom is O or N.

The term “heteroalkylene,” as used herein refers to an alkylene group in which alkylene chain is interrupted once by one, two, or three heteroatoms; twice, each time, independently, by one, two, or three heteroatoms; three times, each time, independently, by one, two, or three heteroatoms; or four times, each time, independently, by one, two, or three heteroatoms. Each heteroatom is, independently, O, N, or S. None of the heteroalkylene groups includes more than two contiguous oxygen atoms. The heteroalkylene group may be unsubstituted or substituted (e.g., optionally substituted heteroalkylene). When the heteroalkylene group is substituted and the substituent is bonded to the heteroatom, the substituent is selected accordingly. The substituent bonded to the heteroatom, valency permitting, is selected from the group consisting of: alkyl, alkanoyl, alkenyl, alkenoyl, alkynyl, alkynoyl, cycloalkyl, cycloalkanoyl, cycloalkenyl, cycloalkenoyl, cycloalkynyl, cycloalkynoyl, aryl, aryloyl, heteroaryl, heteroaryloyl, heterocyclyl, heterocycloyl, amino, aminocarbonyl, alkoxycarbonyl, aryloxycarbonyl, heteroaryloxycarbonyl, and heterocyclyloxycarbonyl. When the heteroalkylene group is substituted and the substituent is bonded to carbon, the substituent is selected from those described for alkylene, provided that the substituent on the carbon atom bonded to the heteroatom is not halo. In some embodiments, the heteroalkylene group has C at each terminus that attaches to other groups. In some embodiments, the heteroatom is O or N.

The term “heteroalkynyl,” as used herein, refers to an alkynyl group in which alkynyl chain is interrupted once by one, two, or three heteroatoms; twice, each time, independently, by one, two, or three heteroatoms; three times, each time, independently, by one, two, or three heteroatoms; or four times, each time, independently, by one, two, or three heteroatoms. Each heteroatom is, independently, O, N, or S. None of the heteroalkynyl groups includes more than two contiguous oxygen atoms. The heteroalkynyl group may be unsubstituted or substituted (e.g., optionally substituted heteroalkynyl) as described for heteroalkenyl.

The term “heteroalkynylene,” as used herein refers to an alkynylene group in which alkynylene chain is interrupted once by one, two, or three heteroatoms; twice, each time, independently, by one, two, or three heteroatoms; three times, each time, independently, by one, two, or three heteroatoms; or four times, each time, independently, by one, two, or three heteroatoms. Each heteroatom is, independently, O, N, or S. None of the heteroalkynylene groups includes more than two contiguous oxygen atoms. The heteroalkynylene group may be unsubstituted or substituted (e.g., optionally substituted heteroalkynylene). The heteroalkynylene group may be unsubstituted or substituted (e.g., optionally substituted heteroalkynylene) as described for heteroalkenylene.

The terms “heteroaromatic moiety” and “heteroaryl,” as used herein, refer to heterocyclic structure (monocyclic or fused bicyclic) satisfying Hückel's rule (4n+2 electrons in a single π system) and thus having the characteristics of aromatic stabilization. Excluding the heteroatoms of any substituents, if present, heteroaryl group contains one, two, three, or four heteroatoms selected from the group consisting of O, S, and N. Heteroaryl group contains 1, 2, 3, 4, 5, 6, 7, 8, or 9 carbon atoms, excluding the carbon atoms of any substituents, if present. The inclusion of a heteroatom permits inclusion of 5-membered rings to be considered aromatic as well as 6-membered rings. Thus, non-limiting examples of heteroaromatic moieties include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, isoxazolyl, benzoxazolyl, benzoisoxazolyl, and imidazolyl. Because tautomers are theoretically possible, phthalimido is also considered aromatic. Typically, heteroaryl ring systems contain 5-12 ring member atoms. For example, heteroaryl group can be a five- to twelve-membered ring system. In some embodiments, the heteroaromatic moiety is a 6-membered aromatic ring system containing 1-2 nitrogen atoms. In some embodiments, heteroaryl group is an optionally substituted pyridyl, indolyl, pyrimidyl, pyridazinyl, benzothiazolyl, benzimidazolyl, pyrazolyl, imidazolyl, isoxazolyl, thiazolyl, benzothiazolyl, or indolyl. In certain embodiments, the heteroaromatic moiety is pyridyl or pyrimidyl. The term “heteroarylene” refers to a heteroaryl group, as described herein, except in that heteroarylene is a divalent substituent.

The term “heteroarylalkylthio,” as used herein, represents a chemical substituent of formula —SR, where R is a heteroarylalkyl group. In some embodiments, heteroarylalkyl group can be further substituted with 1, 2, 3, or 4 substituent groups as described herein.

The term “heteroarylsulfinyl” refers to a group having the structure heteroaryl-S(O)—, in which heteroaryl is as described herein. Heteroarylsulfinyl group may be unsubstituted or substituted as described herein.

The term “heteroarylsulfonyl” refers to a group having the structure heteroaryl-S(O)₂—, in which heteroaryl is as described herein. Heteroarylsulfonyl group may be unsubstituted or substituted as described herein.

The term “heteroarylthio” refers to a group having the structure heteroaryl-S—, in which heteroaryl is as described herein. Heteroarylthio group may be unsubstituted or substituted as described herein.

The term “heterocyclyl,” as used herein represents cyclic heteroalkyl or heteroalkenyl that is, e.g., a 3-, 4-, 5-, 6-, or 7-membered ring, unless otherwise specified. Excluding the heteroatoms of any substituents, if present, heterocyclyl group contains one, two, three, or four heteroatoms selected from the group consisting of O, S, and N. The heterocyclyl group contains, unless otherwise specified, 1, 2, 3, 4, 5, 6, 7, 8, or 9 carbon atoms (e.g., C₁-C₉ heterocyclyl), excluding the carbon atoms of any substituents, if present. Sulfur may be included as divalent sulfur (—S—), tetravalent sulfur (—S(═O)—), or hexavalent sulfur (—S(═O)₂—). The 5-membered ring has zero to two double bonds, and the 6- and 7-membered rings have zero to three double bonds. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring, such as indolyl, quinolyl, isoquinolyl, tetrahydroquinolyl, benzofuryl, benzothienyl and the like. Exemplary heterocycles include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, purinyl, thiadiazolyl (e.g., 1,3,4-thiadiazole), tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, benzofuranyl, benzothienyl and the like. Still other exemplary heterocyclyls include: 2,3,4,5-tetrahydro-2-oxo-oxazolyl; 2,3-dihydro-2-oxo-1H-imidazolyl; 2,3,4,5-tetrahydro-5-oxo-1H-pyrazolyl (e.g., 2,3,4,5-tetrahydro-2-phenyl-5-oxo-1H-pyrazolyl); 2,3,4,5-tetrahydro-2,4-dioxo-1H-imidazolyl (e.g., 2,3,4,5-tetrahydro-2,4-dioxo-5-methyl-5-phenyl-1H-imidazolyl); 2,3-dihydro-2-thioxo-1,3,4-oxadiazolyl (e.g., 2,3-dihydro-2-thioxo-5-phenyl-1,3,4-oxadiazolyl); 4,5-dihydro-5-oxo-1H-triazolyl (e.g., 4,5-dihydro-3-methyl-4-amino 5-oxo-1H-triazolyl); 1,2,3,4-tetrahydro-2,4-dioxopyridinyl (e.g., 1,2,3,4-tetrahydro-2,4-dioxo-3,3-diethylpyridinyl); 2,6-dioxo-piperidinyl (e.g., 2,6-dioxo-3-ethyl-3-phenylpiperidinyl); 1,6-dihydro-6-oxopyridiminyl; 1,6-dihydro-4-oxopyrimidinyl (e.g., 2-(methylthio)-1,6-dihydro-4-oxo-5-methylpyrimidin-1-yl); 1,2,3,4-tetrahydro-2,4-dioxopyrimidinyl (e.g., 1,2,3,4-tetrahydro-2,4-dioxo-3-ethylpyrimidinyl); 1,6-dihydro-6-oxo-pyridazinyl (e.g., 1,6-dihydro-6-oxo-3-ethylpyridazinyl); 1,6-dihydro-6-oxo-1,2,4-triazinyl (e.g., 1,6-dihydro-5-isopropyl-6-oxo-1,2,4-triazinyl); 2,3-dihydro-2-oxo-1H-indolyl (e.g., 3,3-dimethyl-2,3-dihydro-2-oxo-1H-indolyl and 2,3-dihydro-2-oxo-3,3′-spiropropane-1H-indol-1-yl); 1,3-dihydro-1-oxo-2H-iso-indolyl; 1,3-dihydro-1,3-dioxo-2H-iso-indolyl; 1H-benzopyrazolyl (e.g., 1-(ethoxycarbonyl)-1H-benzopyrazolyl); 2,3-dihydro-2-oxo-1H-benzimidazolyl (e.g., 3-ethyl-2,3-dihydro-2-oxo-1H-benzimidazolyl); 2,3-dihydro-2-oxo-benzoxazolyl (e.g., 5-chloro-2,3-dihydro-2-oxo-benzoxazolyl); 2,3-dihydro-2-oxo-benzoxazolyl; 2-oxo-2H-benzopyranyl; 1,4-benzodioxanyl; 1,3-benzodioxanyl; 2,3-dihydro-3-oxo,4H-1,3-benzothiazinyl; 3,4-dihydro-4-oxo-3H-quinazolinyl (e.g., 2-methyl-3,4-dihydro-4-oxo-3H-quinazolinyl); 1,2,3,4-tetrahydro-2,4-dioxo-3H-quinazolyl (e.g., 1-ethyl-1,2,3,4-tetrahydro-2,4-dioxo-3H-quinazolyl); 1,2,3,6-tetrahydro-2,6-dioxo-7H-purinyl (e.g., 1,2,3,6-tetrahydro-1,3-dimethyl-2,6-dioxo-7H-purinyl); 1,2,3,6-tetrahydro-2,6-dioxo-1H-purinyl (e.g., 1,2,3,6-tetrahydro-3,7-dimethyl-2,6-dioxo-1H-purinyl); 2-oxobenz[c,d]indolyl; 1,1-dioxo-2H-naphth[1,8-c,d]isothiazolyl; and 1,8-naphthylenedicarboxamido. Heterocyclyl group may be unsubstituted or substituted (e.g., optionally substituted heterocyclyl). The term “heterocyclylene” refers to a heterocyclyl group, as described herein, except in that heterocyclylene is a divalent substituent.

The term “heterocyclyloxy,” as used herein, refers to a group having the structure (C₁-C₉ heterocyclyl)-O—. Heterocyclyloxy may be unsubstituted or substituted (e.g., optionally substituted heterocyclyloxy) according to the definition of heterocyclyl.

The term “heterocyclyloyl,” as used herein, refers to a group having the structure (C₁-C₉ heterocyclyl)-C(O). Heterocyclyloyl may be unsubstituted or substituted (e.g., optionally substituted heterocyclyloyl) according to the definition of heterocyclyl.

The term “heterocyclyloyloxy,” as used herein, refers to a group having the structure (C₁-C₉ heterocyclyl)-C(O)—O—. Heterocyclyloyloxy may be unsubstituted or substituted (e.g., optionally substituted heterocyclyloyloxy) according to the definition of hetercyclyl.

The term “heterocyclylsulfinyl” refers to a group having the structure heterocyclyl-S(O)—, in which heterocyclyl is as described herein. Heterocyclylsulfinyl group may be unsubstituted or substituted as described herein.

The term “heterocyclylsulfonyl” refers to a group having the structure heterocyclyl-S(O)₂—, in which heterocyclyl is as described herein. Heterocyclylsulfonyl group may be unsubstituted or substituted as described herein.

The term “heterocyclylthio” refers to a group having the structure heterocyclyl-S—, in which heterocyclyl is as described herein. Heterocyclylthio group may be unsubstituted or substituted as described herein.

The term “hydroxy,” as used herein, represents an —OH group.

The term “hydroxyalkyl,” as used herein, represents an alkyl group, as defined herein, substituted by one to three hydroxy groups, with the proviso that no more than one hydroxy group may be attached to a single carbon atom of alkyl group, and is exemplified by hydroxymethyl, dihydroxypropyl, and the like.

The term “nitro,” as used herein refers to —NO₂ group.

The term “n-membered ring,” in which n is 5, 6, 7, or 8, as used herein, refers to a carbocyclic or heterocyclic structure that may be aromatic or non-aromatic. When the n-membered ring is carbocyclic aromatic, it is subject to the definition for aromatic moiety. When the n-membered ring is carbocyclic non-aromatic, it is subject to the definition for cycloalkylene. When the n-membered ring is heterocyclic aromatic, it is subject to the definition for heteroarylene. When the n-membered ring is heterocyclic non-aromatic, it is subject to the definition for heterocyclylene. The n-membered ring may be unsubstituted or substituted (e.g., optionally substituted n-membered ring) according to the respective definition provided herein, unless otherwise specified. In some embodiments, the n-membered ring can be substituted with 1, 2, 3, 4, or 5 substituents, each substituent being independently selected from the group consisting of H, halo, hydroxy, optionally substituted amino, optionally substituted amido, thiol, cyano, optionally substituted C₁-C₆ alkyl, optionally substituted C₂-C₆ alkenyl, optionally substituted C₂-C₆ alkynyl, optionally substituted C₁-C₆ alkoxy, optionally substituted C₆-C₁₀ aryloxy, optionally substituted C₁-C₉ heteroaryloxy, optionally substituted C₂-C₆ alkanoyl, optionally substituted C₇-C₁₁ aryloyl, optionally substituted C₂-C₁₀ heteroaryloyl, optionally substituted C₂-C₁₀ heterocyclyloyl, hydroxycarbonyl, optionally substituted ester, optionally substituted carboxamide, optionally substituted C₁-C₆ alkanoyloxy, optionally substituted C₇-C₁₁ aryloyloxy, optionally substituted C₂-C₁₀ heteroaryloyloxy, optionally substituted C₂-C₁₀ heterocyclyloyloxy, optionally substituted C₁-C₆ thioalkyl, optionally substituted C₁-C₆ alkylsulfinyl, optionally substituted C₁-C₆ alkylsulfonyl, optionally substituted C₆-C₁₀ arylthio, optionally substituted C₆-C₁₀ arylsulfinyl, optionally substituted C₆-C₁₀ arylsulfonyl, optionally substituted C₁-C₉ heteroarylthio, optionally substituted C₁-C₉ heteroarylsulfinyl, optionally substituted C₁-C₉ heteroarylsulfonyl, optionally substituted C₁-C₉ heterocyclylsulfinyl, optionally substituted C₁-C₉ heterocyclylsulfonyl, optionally substituted sulfamoyl, optionally substituted C₁-C₆ heteroalkyl, optionally substituted C₂-C₆ heteroalkenyl, optionally substituted C₂-C₆ heteroalkynyl, optionally substituted C₃-C₁₀ cycloalkyl, optionally substituted C₄-C₁₀ cycloalkenyl, optionally substituted C₅-C₁₀ cycloalkynyl, optionally substituted C₆-C₁₀ aryl, optionally substituted C₆-C₁₀ aryl C₁-C₆ alkyl, optionally substituted C₆-C₁₀ aryl C₂-C₆ alkenyl, optionally substituted C₆-C₁₀ aryl C₂-C₆ alkynyl, optionally substituted C₁-C₉ heteroaryl, optionally substituted C₁-C₉ heteroaryl C₁-C₆ alkyl, optionally substituted C₁-C₉ heteroaryl C₂-C₆ alkenyl, optionally substituted C₁-C₉ heteroaryl C₂-C₆ alkynyl, optionally substituted C₁-C₉ heterocyclyl, optionally substituted C₁-C₉ heterocyclyl C₁-C₆ alkyl, optionally substituted C₁-C₉ heterocyclyl C₂-C₆ alkenyl, and optionally substituted C₁-C₉ heterocyclyl C₂-C₆ alkynyl.

An “oxo” group is a divalent substituent consisting of oxygen atom, e.g., ═O.

The term “pharmaceutically acceptable salt,” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, S. M. Berge et al. describe pharmaceutically acceptable salts in detail in J. Pharm. Sci. 66:1-19, 1977. The salts can be prepared in situ during the final isolation and purification of the compounds of the disclosure or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylammonium, dimethylammonium, trimethylammonium, triethylammonium, ethylammonium, and the like.

The term “protecting group,” as used herein, represents a group intended to protect a functional group (e.g., a hydroxy, an amino, or a carbonyl) from participating in one or more undesirable reactions during chemical synthesis (e.g., polynucleotide synthesis). The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkylene ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkylene groups such as benzyl, triphenylmethyl, benzyloxymethyl, and the like and silyl groups such as trimethylsilyl, and the like. Useful N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “sulfamoyl,” as used herein, refers to a group having the structure —SO₂—N(R^{N1})₂, wherein each R^{N1} is, independently, H, alkyl, alkenyl, alkynyl, aryl, arylalkyl, cycloalkyl, cycloalkylalkyl, heterocyclyl (e.g., heteroaryl), heterocyclylalkyl (e.g., heteroarylalkyl), or two R^{N1} combine to form a heterocyclyl. The sulfamoyl group may be unsubstituted, when each R^{N1} is H, or substituted, when at least one R^{N1} is not H (e.g., optionally substituted sulfamoyl). In a preferred embodiment, sulfamoyl is —SO₂NH₂ or —SO₂NHR^{N1}, wherein R^{N1} is, independently, alkyl, aryl, arylalkyl, cycloalkyl, cycloalkylalkyl, heterocyclyl (e.g., heteroaryl), heterocyclylalkyl (e.g., heteroarylalkyl).

The term “alkylthio” or “thioalkyl,” as used herein, represents a chemical substituent of formula —SR, where R is an alkyl group. In some embodiments, alkyl group can be further substituted with 1, 2, 3, or 4 substituent groups as described herein.

The term “thiol” represents an —SH group.

Each of the above-described groups may be optionally substituted, when chemically appropriate. As used herein, the term “optionally substituted” means that one or more hydrogens may be replaced by a non-hydrogen substituent and includes fully substituted, partially substituted, and unsubstituted groups. Typical optional substituents on aromatic or heteroaromatic groups include independently halo (e.g., F, Cl, Br, or I), optionally substituted alkyl, optionally substituted alkenyl, optionally substituted alkynyl, optionally substituted cycloalkyl, optionally substituted cycloalkenyl, optionally substituted cycloalkynyl, CN, NO₂, CF₃, OCF₃, COOR′, CONR′₂, OR′, SR′, SOR′, SO₂R′, NR′₂, NR′(CO)R′,NR′C(O)OR′, NR′C(O)NR′₂, NR′SO₂NR′₂, or NR′SO₂R′, wherein each R′ is independently H or an optionally substituted group selected from alkyl, alkenyl, alkynyl, cycloalkyl, cycloalkenyl, cycloalkynyl, heteroalkyl, heteroalkenyl, heteroalkynyl, heteroaryl, and aryl (all as defined above); or the substituent may be an optionally substituted group selected from alkyl, alkenyl, alkynyl, cycloalkyl, cycloalkenyl, cycloalkynyl, heteroalkyl, heteroalkenyl, heteroalkynyl, aryl, heteroaryl, O-aryl, O-heteroaryl, and arylalkyl.

Unless otherwise specified, typical optional substituents on non-aromatic groups include independently halo (e.g., F, Cl, Br, or I), CN, NO₂, CF₃, OCF₃, COOR′, CONR′₂, OR′, SR′, SOR′, SO₂R′, NR′₂, NR′(CO)R′, NR′C(O)OR′, NR′C(O)NR′₂, NR′SO₂NR′₂, or NR′SO₂R′, wherein each R′ is independently H or an optionally substituted group selected from alkyl, alkenyl, alkynyl, cycloalkyl, cycloalkenyl, cycloalkynyl, heteroalkyl, heteroalkenyl, heteroalkynyl, heteroaryl, and aryl (all as defined above); or the substituent may be an optionally substituted group selected from alkyl, alkenyl, alkynyl, cycloalkyl, cycloalkenyl, cycloalkynyl, heteroalkyl, heteroalkenyl, heteroalkynyl, aryl, heteroaryl, O-aryl, O-heteroaryl, and arylalkyl. A non-aromatic group may also include a substituent selected from ═O and =NOR′ where R′ is H or an optionally substituted group selected from alkyl, alkenyl, alkynyl, heteroalkyl, heteroalkenyl, heteralkynyl, heteroaryl, and aryl (all as defined above).

In general, a substituent group (e.g., alkyl, alkenyl, alkynyl, or aryl (including all heteroforms defined above) may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included. For example, where a group is substituted, the group may be substituted with 1, 2, 3, 4, 5, or 6 substituents. Optional substituents include, but are not limited to: C1-C6 alkyl or heteroalkyl, C2-C6 alkenyl or heteroalkenyl, C2-C6 alkynyl or heteroalkynyl, halogen; aryl, heteroaryl, azido(-N₃), nitro (—NO₂), cyano (—CN), acyloxy(-OC(═O)R′), acyl (—C(═O)R′), alkoxy (—OR′), amido (—NR′C(═O)R″), carboxamide (e.g., —C(═O)NRR′), amino (—NRR′), carboxylic acid (—CO₂H), carboxylic ester (—CO₂R′), carbamoyl (—OC(═O)NR′R″ or —NRC(═O)OR′), hydroxy (—OH), isocyano (—NC), sulfonate (—S(═O)₂OR), sulfonamide (—S(═O)₂NRR′ or —NRS(═O)₂R′), or sulfonyl (—S(═O)₂R), where each R or R′ is selected, independently, from H, C1-C6 alkyl or heteroalkyl, C2-C6 alkenyl or heteroalkenyl, C2-C6 alkynyl or heteroalkynyl, aryl, or heteroaryl. A substituted group may have, for example, 1, 2, 3, 4, 5, 6, 7, 8, or 9 substituents.

In some embodiments, the GPR174 inhibitor is a compound of Formula (I), Formula (IV), or Formula (VIII). In some embodiments, the GPR174 inhibitor is Compound 10. Any combination of a PKA inhibitor, A2A inhibitor, and GPR174 inhibitor with or without p38 inhibitor or a PI3Kδ inhibitor, or a combination thereof can be used in the methods of generating phenotype-altered T cells of the disclosure. Thus, in some embodiments, a combination of RP-8-Br-cAMPS, doramapimod, and idelalisib can be used in the compositions and methods of the disclosure.

The agents described above can be dissolved in a suitable solvent (e.g., water, DMSO) prior to contacting the resulting solution with a population of T cells in vitro in the methods of the disclosure. In some embodiments, the agents (e.g., PKA inhibitors, A2A inhibitors, GPR174 inhibitors, and p38 inhibitors and/or a PI3Kb inhibitor, or a combination thereof.) can be formulated so that the transfer of said inhibitors into cells is improved. For example, such formulations can utilize lipids, lipid particles or vesicles, polymers, proteins, or other materials that carry the inhibitor across the cell membrane or otherwise assist in transferring the inhibitor into T cells. Exemplary methods and formulations that facilitate transfer of inhibitors such as those disclosed herein into cells are known in the art, for example, those described in Yang N J, Hinner M J. Getting across the cell membrane: an overview for small molecules, peptides, and proteins. Methods Mol Biol. 2015; 1266:29-53 and Zhang R, Qin X, Kong F, Chen P, Pan G. Improving cellular uptake of therapeutic entities through interaction with components of cell membrane. Drug Deliv. 2019; 26(1): 328-342.

Accordingly, in another aspect, the disclosure provides a composition for improving therapeutic potential of T cells suitable for adoptive cell-based therapies, the composition comprising a p38 inhibitor, a PI3Kδ inhibitor, or a combination thereof and at least one agent selected from the group consisting of a protein kinase A (PKA) inhibitor, an A2A adenosine receptor inhibitor, and a GPR174 inhibitor, wherein the composition alters a phenotype of at least a subpopulation of immune cells cultured in vitro in the presence of the composition. Typically, the agents are included in a medium suitable for culturing T cells. In some embodiments, the compositions of the disclosure further comprise a cell culture medium suitable for culturing T cells. In some embodiments, provided herein are kits comprising one or more of the phenotype-altering agents (such as a protein kinase A (PKA) inhibitor, an A2A adenosine receptor inhibitor, a GPR174 inhibitor, or a combination thereof optionally in combination with at least one of p38 inhibitor and a PI3Kδ inhibitor) and a cell culture medium suitable for culturing T cells, wherein the one or more agents are included in the amounts sufficient to alter at least one phenotype of a subpopulation of T cells when added to the cell culture medium. In some embodiments of the kits, the one or more agents are contained in separate containers or pre-mixed in a single container in the amount suitable for addition to the culture medium and the culture medium is included in yet another separate container.

The compositions and kits of the disclosure can be used to generate therapeutic T cells. Thus, in another aspect, disclosed herein is a method of producing an isolated population of T cells comprising phenotype-altered T cells, the method comprising culturing a population of T cells in vitro in the presence of a phenotype-altering composition comprising at least one phenotype-altering agent selected from the group consisting of a protein kinase A (PKA) inhibitor, an A2A adenosine receptor inhibitor, a GPR174 inhibitor, and combinations thereof, wherein the phenotype-altering composition alters a phenotype of at least a subpopulation of T cells. In some embodiments, the composition further comprises a p38 inhibitor and/or a PI3Kb inhibitor. In some embodiments, the population of T cells comprises T cells isolated from a subject suffering from a disease, T cells isolated from a universal donor, or universal donor T cells derived from stem cells. In some embodiments, the population of T cells comprises naïve T cells, stem cell memory T cells, central memory T cells, or combinations thereof.

In some embodiments, the method further comprises transferring the phenotype-altered T cells to a re-stimulation environment, e.g., an environment such as cell culture medium comprising one or more tumor antigens. In some embodiments, the re-stimulation environment is in vivo.

Accordingly, in another aspect, the disclosure provides an isolated population of T cells produced by the methods described above.

Therapeutic Applications

The T cell compositions and methods of their generation disclosed herein are useful in treatment of diseases treatable by administration of an effective amount of therapeutic T cells, such as by adoptive cell-based therapy. In some embodiments, the disease is cancer, such as a solid tumor or blood cancer.

It is contemplated that phenotype-altered populations of T cells produced according to the methods of the disclosure can be used in methods of treating or preventing cancer in a patient. In this regard, the invention provides a method of treating or preventing cancer in a patient, comprising administering to the mammal any of the pharmaceutical compositions or populations of T cells described herein in an amount effective to treat or prevent cancer in the mammal. In some embodiments, the methods of treatment disclosed herein further comprise lymphodepleting the patient prior to administering the isolated population of T cells. Examples of lymphodepletion include, but may not be limited to, nonmyeloablative lymphodepleting chemotherapy, myeloablative lymphodepleting chemotherapy, total body irradiation, etc.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods of the disclosure can provide any amount of any level of treatment or prevention of cancer in a mammal. Furthermore, the treatment or prevention provided by the method can include treatment or prevention of one or more conditions or symptoms of the disease, e.g., cancer, being treated or prevented. Also, as used herein, “prevention” can encompass delaying the onset or recurrence of the disease, or a symptom or condition thereof.

The cancer can be any cancer, including any of leukemia (e.g., B cell leukemia), sarcomas (e.g., synovial sarcoma, osteogenic sarcoma, leiomyosarcoma uteri, and alveolar rhabdomyosarcoma), lymphomas (e.g., Hodgkin lymphoma and non-Hodgkin lymphoma), hepatocellular carcinoma, glioma, head-neck cancer, acute lymphocytic cancer, acute myeloid leukemia, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer (e.g., colon carcinoma), esophageal cancer, cervical cancer, gastrointestinal carcinoid tumor, hypopharynx cancer, larynx cancer, liver cancer, lung cancer, malignant mesothelioma, melanoma, multiple myeloma, nasopharynx cancer, ovarian cancer, pancreatic cancer, peritoneum, omentum, and mesentery cancer, pharynx cancer, prostate cancer, rectal cancer, renal cancer, small intestine cancer, soft tissue cancer, stomach cancer, testicular cancer, thyroid cancer, ureter cancer, and urinary bladder cancer.

In some embodiments, the patient is suffering from, or is harboring, a malignant neoplasm (i.e., cancer), selected from the group consisting of: acoustic neuroma, anal cancer (including carcinoma in situ), squamous cell carcinoma, adrenal tumor (including adenoma, hyperaldosteronism, adrenalcortical cancer), Cushing's syndrome, benign paraganglioma, appendix cancer (including pseudomyxoma peritonei, carcinoid tumors, non-carcinoid appendix tumors), bile duct cancer (including intrahepatic bile duct cancer, extrahepatic bile duct cancer, perihilar bile duct cancer, distal bile duct cancer), gallbladder cancer, bone cancer (including chondrosarcoma, osteosarcoma, malignant fibrous histiocytoma, fibrosarcoma, chordoma), brain tumor (including craniopharyngioma, dermoid cysts, epidermoid tumors, glioma, astrocytoma, low-grade astrocytoma, anaplastic astrocytoma, ependymoma, glioblastoma, oligodendrogliomas, hemangioblastoma, pineal gland tumors, pituitary tumors, sarcoma, chordoma), breast cancer (including lobular carcinoma, triple negative breast cancer, recurrent breast cancer, brain metastases), bladder cancer (including transitional cell bladder cancer, squamous cell carcinoma, adenocarcinoma), cancers of unknown primary (CUP), (including adenocarcinoma, poorly differentiated carcinoma, squamous cell carcinoma, poorly differentiated malignant neoplasm, neuroendocrine carcinoma), cervical cancer (including squamous cell carcinoma, adenocarcinoma, mixed carcinoma), carcinoid tumors, childhood germ cell tumors (including yolk sac tumors, teratoma, embryonal carcinoma, polyembryoma, germinoma), childhood brain tumors, (including ependymoma, craniopharyngioma, chordoma, pleomorphic xanthoastrocytoma, meningioma, primitive neuroectodermal tumors, ganglioglioma, pineoblastoma, germ cell tumors, mixed glial and neuronal tumors, astrocytoma, choroid plexus tumors), childhood leukemias (including lymphoblastic leukemia, myeloid leukemia), childhood hematology disorders (including Fanconi anemia, Diamond-Blackfan anemia, aplastic anemia, Shwachman-Diamond syndrome, Kostmann's syndrome, Neutropenia, Thrombocytopenia, Hemoglobinopathies, erythrocytosis, histiocytic disorders, iron overload, clotting and bleeding disorders), childhood liver cancers (including hepatoblastoma, hepatocellular carcinoma), childhood lymphomas (including Hodgkin's lymphoma, Non-Hodgkin's lymphoma, Burkitt's lymphoma, lymphoblastic lymphoma, large cell lymphoma), childhood osteosarcomas; childhood melanomas; childhood soft tissue sarcomas, colon cancer (including adenocarcinoma, hereditary nonpolyposis colorectal cancer syndrome, familial adenomatous polyposis), desmoplastic Small Round Cell Tumors (DSRCT); esophageal cancers (including adenocarcinoma, squamous cell carcinoma), Ewing's sarcomas (including Ewing's Sarcoma of the bone, extraosseous Ewing tumor, peripheral primitive neuroectodermal tumors), Eye cancers (including uveal melanoma, basal cell carcinoma, squamous cell carcinoma, melanoma of the eyelid, melanoma of the conjunctiva, sebaceous carcinoma, merkel cell carcinoma, mucosa-associated lymphoid tissue lymphoma, orbital lymphoma, orbital sarcoma, orbital and optic nerve meningioma, metastic orbital tumors, lacrimal gland lymphoma, adenoid cystic carcinoma, pleomorphic adenoma, transitional cell carcinoma, lacrimal sac lymphoma); Fallopian tube cancers (including endometrioid adenocarcinoma, serous adenocarcinoma, leiomyosarcoma, transitional cell fallopian tube cancer); Hodgkin's lymphomas (including classical Hodgkin's lymphoma, nodular sclerosing Hodgkin's lymphoma, lymphocyte-rich classical Hodgkin's lymphoma, mixed cellularity Hodgkin's lymphoma, lymphocyte depletion Hodgkin's lymphoma, lymphocyte-predominant Hodgkin's lymphoma), Implant-Associated Anaplastic Large Cell Lymphomas (ALCL); Inflammatory Breast Cancers (IBC); Kidney cancers (including renal cell carcinoma, urothelial cancer of the kidney, pelvis and ureter); Leukemias, (including acute lymphocyte leukemia, acute myeloid leukemia, chronic lymphoblastic leukemia, chronic myeloid leukemia), Liver cancers (including hepatocellular carcinoma, fibrolamellar hepatocellular carcinoma, angiosarcoma, hepatoblastoma, hemangiosarcoma), Lung cancers (including non-small cell lung cancer, adenocarcinoma, squamous cell carcinoma, large cell carcinoma, small cell lung cancer, carcinoid tumor, salivary gland carcinoma, lung metastases, sarcoma); Medulloblastomas; Melanomas (including cutaneous melanoma, superficial spreading melanoma, nodular melanoma, lentigo maligna melanoma, acral lentiginous melanoma, ocular melanoma, mucosal melanoma); Mesotheliomas (including sarcomatoid mesothelioma, biphasic mesothelioma), Multiple Endocrine Neoplasias (MEN), (including multiple endocrine neoplasia type 1, multiple endocrine neoplasia type 2); Multiple Myelomas; Myelodysplastic syndromes (MDS) (including refractory anemia, refractory cytopenia with multilineage dysplasia, refractory anemia with ringed sideroblasts, refractory anemia with excess blasts, refractory cytopenia with multilineage dysplasia and ringed sideroblasts); Myeloproliferative disorders (MPD), (including polycythemia vera, primary myelofibrosis, essential thrombocythemia, systemic mastocytosis, hypereosinophilic syndrome); Neuroblastomas; Neurofibromatosis (including neurofibromatosis type 1, neurofibromatosis type 2, schwannomatosis); Non-Hodgkin's Lymphomas (including b-cell lymphoma, t-cell lymphoma, NK-cell lymphoma, mucosa-associated lymphoid tissue lymphoma, follicular lymphoma, mantle cell lymphoma, diffuse large cell lymphoma, primary mediastinal large cell lymphoma, anaplastic large cell lymphoma, burkitt's lymphoma, lymphoblastic lymphoma, marginal zone lymphoma); Oral cancers (including squamous cell carcinoma); Ovarian cancers (including epithelial ovarian cancer, germ cell ovarian cancer, stromal ovarian cancer, primary peritoneal ovarian cancer); Pancreatic cancers (including islet cell carcinoma, sarcoma, lymphoma, pseudopapillary neoplasms, ampullary cancer, pancreatoblastoma, adenocarcinoma); Parathyroid diseases (including hyperparathyroidism, hypoparathyroidism, parathyroid cancer), Penile cancers (including squamous cell carcinoma, kaposi sarcoma, adenocarcinoma, melanoma, basal cell carcinoma); Pituitary tumors (including non-functioning tumors, functioning tumors, pituitary cancer), Prostate cancers (including adenocarcinoma, prostatic intraepithelial neoplasia), Rectal cancers (including adenocarcinoma), Retinoblastomas (including unilateral retinoblastoma, bilateral retinoblastoma, PNET retinoblastoma), Skin cancers (including basal cell carcinoma, squamous cell carcinoma, actinic (solar) keratosis); Skull base tumors (including meningioma, pituitary adenoma, acoustic neuroma, glomus tumors, squamous cell carcinoma, basal cell carcinoma, adenoid cystic carcinoma, adenocarcinoma, chondrosarcoma, rhabdomyosarcoma, osteosarcoma, esthesioblastoma, neuroendocrine carcinoma, mucosal melanoma), Soft tissue sarcomas; Spinal tumors (including intramedullary spinal tumors, intradural extramedullary spinal tumors, extradural spinal tumors, osteoblastoma, enchondroma, aneurysmal bone cysts, giant cell tumors, hangioma, eosinophilic granuloma, osteosarcoma, chordoma, chondrosarcoma, plasmacytoma); Stomach cancers (including lymphoma, gastrointestinal stromal tumors, carcinoid tumors); Testicular cancers (including germ cell tumors, nonseminoma, seminoma, embryonal carcinoma, yolk sac tumors, teratoma, sertoli cell tumors, choriocarcinoma, stromal tumors, leydig cell tumors); Throat cancers (including squamous cell carcinoma); Thyroid cancers (including papillary thyroid cancer, follicular thyroid cancer, hurthle cell carcinoma, medullary thyroid cancer, anaplastic thyroid cancer); Uterine cancers (including endometrioid adenocarcinoma, uterine carcinosarcoma, uterine sarcoma); Vaginal cancers (including squamous cell carcinoma, adenocarcinoma, melanoma, sarcoma); Vulvar cancers (including squamous cell carcinoma, adenocarcinoma, melanoma, sarcoma); von Hippel Lindau Diseases; Waldenström's Macroglobulinemias; and Wilms' Tumors.

As used herein, the term “neoplasm” refers to any new and abnormal growth of cells, specifically one in which cell multiplication is uncontrolled and progressive. Neoplasms may be non-malignant (i.e., benign) or malignant. As used herein, the term “tumors” means neoplasms, including solid and liquid (i.e., blood) neoplasms, and benign and malignant neoplasms, including primary and/or metastatic neoplasms.

The population of T cells produced according to the methods described herein can be included in a composition, such as a pharmaceutical composition. Accordingly, the disclosure provides a pharmaceutical composition comprising isolated or purified population of the phenotype-altered T cells described herein and a pharmaceutically acceptable carrier.

Any carrier suitable for formulating T cells can be used in the compositions of the disclosure. Preferably, the carrier is a pharmaceutically acceptable carrier, such as any carrier conventionally used for the administration of cells. Such pharmaceutically acceptable carriers are well-known to those skilled in the art and are readily available to the public. It is preferred that the pharmaceutically acceptable carrier has no detrimental side effects or toxicity under the conditions of use.

The choice of carrier can be determined in part by the particular method used to administer the population of T cells of the disclosure. A variety of suitable formulations of the pharmaceutical compositions disclosed herein exists in the art. Suitable formulations include any of those for parenteral, subcutaneous, intravenous, intramuscular, intraarterial, intrathecal, intratumoral, or intraperitoneal administration. More than one route can be used to administer the phenotype-altered T cells, and in certain instances, a particular route can provide a more immediate and more effective response than another route.

The T cells of the disclosure can be administered by any suitable route. Preferably, the T cells are administered by injection, e.g., intravenously. A suitable pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In some embodiments, the pharmaceutically acceptable carrier is supplemented with human serum albumin.

As used herein, the effective dose, e.g., a number of T cells, is a dose sufficient to generate a therapeutic or prophylactic response in the subject over a reasonable time period. In some embodiments, the dose is a number of T cells administered sufficient to bind to a cancer antigen or treat or prevent cancer in a period of from about 2 hours or longer, e.g., about 12 to 24 or more hours, from the time of administration. In certain embodiments, the time period could be even longer. The number of T cells administered can be determined by methods known in the art, such as taking into account the efficacy of the particular population of T cells to be administered, the condition of the subject (e.g., human), the body weight of the subject (e.g., human) to be treated, etc. Typically, the treating oncologist will determine the number of T cells used to treat each individual subject, taking into consideration a variety of factors, such as age, body weight, general health, diet, sex, route of administration, and the severity of the condition being treated. In some non-limiting examples, the number of the T cells of the disclosure to be administered can be about 1×10⁶ to about 1×10¹² cells per infusion, about 1×10⁹ to about 1×10¹² cells per infusion, or about 1×10⁸ to about 1×10¹⁰ cells per infusion. Assays useful for determining a suitable number of T cells are known in the art.

In some embodiments, the patient is a mammalian patient. In some specific embodiments, the patient is a human. In more specific embodiments, the patient is suffering from cancer. The term “mammal” includes all mammals, including without limitation humans, non-human primates, dogs, cats, horses, sheep, goats, cows, rabbits, pigs, and rodents. As used herein, the terms “subject” or “patient” are used interchangeably and refer to any organism to which phenotype-altered T cells in accordance with the disclosure may be administered, e.g., for experimental, diagnostic, prophylactic, and/or therapeutic purposes. A subject to be treated with phenotype-altered T cells described herein may be one who has been diagnosed by a medical practitioner as having a disease, disorder, or condition, described herein, or one at risk for developing the disease, disorder, or condition described herein. Diagnosis may be performed by any technique or method known in the art. One skilled in the art will understand that a subject may have been diagnosed as having the disease, disorder, or condition, using a standard test or examination or may have been identified, without examination, as one at high risk due to the presence of one or more risk factors. Typical subjects include animals (e.g., mammals such as mice, rats, rabbits, non-human primates, and humans).

In some embodiments, the methods of treatment disclosed herein further comprise transfer of the phenotype-altered cultured T cells to a re-stimulation environment. In certain embodiments, the re-stimulation environment comprises one or more tumor antigens. In some embodiments, the re-stimulation environment is in vivo. In certain embodiments, the re-stimulation environment is in a human.

In one embodiment, the disclosure provides a method of preparing phenotype-altered T cells for adoptive immunotherapy comprising a step of contacting T cells isolated from a patient with a composition comprising at least one inhibitor selected from the group consisting of a protein kinase A (PKA) inhibitor, an A2A inhibitor, a GPR174 inhibitor, and combinations thereof, optionally in combination with a p38 inhibitor and/or PI3Kδ inhibitor, thereby altering a phenotype of at least a subpopulation of the T cells. The methods of the disclosure can comprise additional steps, for example, harvesting a source of T cells from a subject, stimulating and activating the T cells in the presence of the phenotype-altering composition of the present disclosure, modifying the T cells to express an engineered TCR or CAR, and expanding the T cells in culture. In some embodiments, the steps of modifying and/or expanding are also performed in the presence of a phenotype-altering composition. In some embodiments, the steps of modifying and/or expanding are performed without a phenotype-altering composition.

In some embodiments, the methods of treatment of the disclosure further include administering one or more additional therapeutic agents to the patient undergoing the treatment.

In some embodiments, the patient suffering from cancer has one or more tumors infiltrated with regulatory T cells, such as, for example, breast, lung (such as small-cell lung cancer or non-small cell lung cancer), colorectal, cervical, renal, ovarian, melanoma, pancreatic, hepatocellular, gastric, glioblastoma, glioma, bladder, myeloma (such as multiple myeloma), prostate, thyroid, testicular, and esophageal cancer.

In some embodiments, the patient is suffering from cancer resistant to checkpoint inhibitors, such as anti-PD-1 (e.g., Keytruda® and Opdivo®) and anti-CTLA-4 (e.g., Yervoy®).

In some embodiments, the patient has been treated or is undergoing treatment with one or more therapeutic agents, such as one or more known chemotherapeutic agent. In some embodiments, the patient has been treated or is undergoing treatment with one or more checkpoint inhibitors, such as anti-PD-1 (e.g., Keytruda® and Opdivo®) and anti-CTLA-4 (e.g., Yervoy®).

A therapeutic amount of phenotype-altered T cells disclosed herein means an amount or number of T cells effective to yield the desired therapeutic response, for example, an amount effective to delay the growth of a cancer or to cause a cancer to shrink or not metastasize. Within such methods described herein, pharmaceutical compositions are typically administered to a patient. The compositions, i.e., phenotype-altered T cells of the disclosure, can be administered either prior to or following surgical removal of primary tumors and/or treatment such as administration of radiotherapy or conventional chemotherapeutic drugs, or bone marrow transplantation (autologous, allogeneic, or syngeneic).

The phenotype-altered T cells provided herein may be used alone or in combination with one or more additional therapeutic agents suitable for treatment of a particular indication. For example, phenotype-altered T cells of the disclosure may be co-administered to a subject who has, or is at risk for developing, cancer with conventional anti-cancer therapeutic regimens such as surgery, irradiation, chemotherapy and/or bone marrow transplantation (autologous, syngeneic, allogeneic, or unrelated).

In general, for use in treatment, the phenotype-altered T cells described herein can be used in combination with other agents, compounds, and/or pharmaceuticals. Examples of such other agents include agents that are known to be used for the treatment of inflammatory conditions, autoimmune disorders, or cancers. Each component of a combination therapy can be formulated in a variety of ways that are known in the art and/or suitably administered to the patient at one time or over a series of treatments.

As described herein, in some embodiments, the phenotype-altered T cells of the disclosure can provide “synergy” and prove to be “synergistic” with additional therapeutic agents, i.e., the effect achieved when the agents are used together is greater than the sum of the effects that results from using the agents separately. A synergistic effect can be attained when the agents are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds, agents, and/or treatments are administered or delivered sequentially, e.g., by different injections in separate syringes. In general, during alternation therapy, an effective dosage of each agent is administered sequentially, i.e., serially, whereas in combination therapy, effective dosages of two or more active ingredients are administered together. Suitable dosages for any of the above co-administered agents are those presently used and may be lowered due to the combined action (synergy) of phenotype-altered T cells of the present disclosure and other co-administered agents or treatments. Each component of a combination therapy, as described herein, can be formulated in a variety of ways that are known in the art.

### CERTAIN NONLIMITING EXEMPLARY EMBODIMENTS

Embodiment 1. A method for treating a disease, comprising administering to a subject in need thereof a therapeutically effective amount of phenotype-altered T cells, wherein the phenotype-altered T cells are prepared by a method comprising culturing a population of T cells in vitro in the presence of a phenotype-altering composition comprising a phenotype-altering agent selected from the group consisting of a protein kinase A (PKA) inhibitor, an A2A adenosine receptor inhibitor, a GPR174 inhibitor, and combinations thereof for a time sufficient to alter a phenotype of at least a subpopulation of the population of T cells.

Embodiment 2. The method of c Embodiment 1, wherein the PKA inhibitor is a PKA-RI or RII inhibitor or a competitive antagonist of cAMP binding to PKA-RI or RII.

Embodiment 3. The method of Embodiment 1 or Embodiment 2, wherein the composition further comprises a p38 inhibitor, a PI3Kδ inhibitor, or a combination thereof.

Embodiment 4. The method of any one of Embodiments 1-3, wherein the at least one phenotype-altering agent has been removed from the cell culture prior to administration to the subject.

Embodiment 5. The method of any one of Embodiments 1-4, wherein the at least one phenotype-altering agent is an exogenous agent.

Embodiment 6. The method of any one of Embodiments 1-5, wherein the population of T cells comprises genetically modified T cells.

Embodiment 7. The method of Embodiment 6, wherein the genetically modified T cells comprise an exogenous nucleic acid.

Embodiment 8. The method of Embodiment 7, wherein the exogenous nucleic acid encodes a T Cell Receptor (TCR), an exogenous nucleic acid encoding a Chimeric Antigen Receptor (CAR), or a combination thereof.

Embodiment 9. The method of Embodiment 6, wherein the genetically modified T cells comprise a deletion of a gene or a portion of a gene.

Embodiment 10. The method of any of Embodiments 1-9, wherein the population of T cells comprises autologous T cells or allogenic T cells, including T cells isolated from a cancer patient that naturally express TCRs specific for antigens expressed by the patient's tumor.

Embodiment 11. The method of any one of Embodiments 1-10, wherein the disease is a disease treatable by adoptive cell-based therapy.

Embodiment 12. The method of any one of Embodiments 1-11, wherein the disease is cancer.

Embodiment 13. The method of Embodiment 12, wherein the cancer is a solid tumor or blood cancer.

Embodiment 14. The method of any one of Embodiments 1-13, wherein the phenotype altering agent is a GPR174 inhibitor.

Embodiment 15. The method of Embodiment 14, wherein the GPR174 inhibitor is a small molecule GPR174 inhibitor or an antibody that specifically binds to GPR174.

Embodiment 16. The method of Embodiment 15, wherein the GPR174 inhibitor is a small molecule represented by any one of Formulae I, II, III, IV, V, or VIII.

Embodiment 17. The method of any one of Embodiments 1-13, wherein the phenotype altering agent is a protein kinase A (PKA) inhibitor.

Embodiment 18. The method of Embodiment 17, wherein the PKA inhibitor is a small molecule or a peptide inhibitor of PKA-C, or an antisense oligonucleotide targeting PKA-Cα and/or PKA-Cβ.

Embodiment 19. The method of Embodiment 17, wherein the protein kinase A (PKA) inhibitor is selected from the group consisting of HA-100 dihydrochloride, Rp-cAMPS, H-89 dihydrochloride, PKI (5-24), Staurosporine, Calphostin C, KT 5720, Rp-8-Br-cAMPS, 5-Iodotubercidin, Piceatannol, Fasudil (monohydrochloride salt), ML-7 hydrochloride, CGP-74514A hydrochloride, ML-9, Daphnetin, Myricetin, PKC-412, A-674563, K-252a, H-7 dihydrochloride, bisindolylmaleimide IV, cGK1alpha inhibitor-cell permeable DT-3, TX-1123, Rp-8-PIP-cAMPS, 8-bromo2′-monobutyrladenosine-3′,5′-cyclic monophosphorothioate Rp-isomer, Bisindolylmaleimide III hydrochloride, Rp-adenosine 3′,5′-cyclic monophosphorothioate sodium salt, A-3 hydrochloride, H-7, H-8-2HCl, K252c, HA-1004 dihydrochloride, K-252b, HA-1077 dihydrochloride, MDL-27,032, H-9 hydrochloride, Rp-8-CPT-cAMPS, bisindolylmaleimide III, -lacetamido-4-cyano-3-methyllisoquinoline, Ilmofosine, Rp-8-hexylaminoadenosine 3′,5′-monophosphorothioate, HA-1004 hydrochloride, PKA Inhibitor IV, Adenosine 3′,5′-cyclic monophosphorothioate 8-chloro Rp-isomer sodium salt, adenosine 3′,5′cyclic monophosphorothioate 2′-O-monobutyryl Rp-isomer sodium salt, 4-cyano-3-methylisoquinoline, 8-hydroxyadenosine-3′,5′-monophosphorothioate Rp-isomer, PKI (6-22) amide, SB 218078, Rp-8-pCPT-cyclic GMPS sodium, Sp-8-pCPT-cAMPS, N[2-(p-Cinnamylamino)shyethyl]-5-isoquinolone sulfonamide, AT7867, GSK 690693, PKI (14-22) amide (myristoylated), Rp-8-bromo-cAMPS, or combinations thereof.

Embodiment 20. The method of any one of Embodiments 1-13, wherein the phenotype altering agent is an A2A adenosine receptor inhibitor.

Embodiment 21. The method of Embodiment 20, wherein the A2A adenosine receptor inhibitor is selected from the group consisting of ZM 241385 (CAS 139180-30-6), istradefylline (CAS 155270-99-8), xanthine amine congener (CAS 96865-92-8), XCC (CAS 96865-83-7), ANR 94 (CAS 634924-89-3), PSB 1115 (CAS 409344-71-4), 3,7-dimethyl-1-propargylxanthine (CAS 14114-46-6), SCH 58261 (CAS 160098-96-4), SCH 442416 (CAS 316173-57-6), 8-(3-chlorostyryl)caffeine (CAS 147700-11-6), CGS 15943 (CAS 104615-18-1), ST4206 (CAS 246018-36-9), KF21213 (CAS 155271-17-3), regadenoson (CAS 313348-27-5), preladenant (CAS 377727-87-2), CGS 21680 (CAS 120225-54-9), tozadenant (CAS 870070-55-6), Sch412348 (CAS 377727-26-9), ST3932 (CAS 1246018-21-2), A2A receptor antagonist 1 (CPI-444 analog; CAS 443103-97-7), istradefylline (CAS 155270-99-8), AZD4635 (CAS 1321514-06-0), CGS 15943 (CAS 104615-18-1), vipadenant (CAS 442908-10-3), CPI-444 (CAS 1202402-40-1), TC-G 1004 (CAS 1061747-72-5), 4-desmethyl istradefylline (CAS 160434-48-0), PSB 0777 (CAS 2122196-16-9), or a combination thereof.

Embodiment 22. The method of any one of Embodiments 2-21, wherein the p38 inhibitor is selected from the group consisting of doramapimod (CAS 285983-48-4), losmapimod (CAS 585543-15-3), SX 011 (CAS 309913-42-6), SB202190 (CAS 350228-36-3), VX 702 (CAS 745833-23-2), JX-401 (CAS 349087-34-9), p38 MAP Kinase Inhibitor VIII (CAS 321351-00-2). SCIO 469 (CAS 309913-83-5), p38 MAP Kinase Inhibitor V (CAS 271576-77-3), p38 MAP Kinase Inhibitor IX (N-(isoazol-3-yl)-4-methyl-3-(1-phenyl-1H-pyrazolo[3,4-d]pyrimidin-4-ylamino)benzamide), PD 169316 (CAS 152121-53-4), p38 MAP Kinase Inhibitor III (CAS 581098-48-8), PH-797804 (CAS 586379-66-0), RWJ 67657 (CAS 215303-72-3), VX 745 (CAS 209410-46-8), LY 364947 (CAS 396129-53-6), p38 MAP Kinase Inhibitor (CAS 219138-24-6), SB 239063 (CAS 193551-21-2), SB 202190 (CAS 152121-30-7), SB 203580 (CAS 152121-47-6), p38 MAP Kinase Inhibitor IV (CAS 1638-41-1), SD-169 (CAS 1670-87-7), N-(5-Chloro-2-methylphenyl)-7-nitrobenzo[c][1,2,5]oxadiazol-4-amine (FGA-19), or a combination thereof.

Embodiment 23. The method of any one of Embodiments 2-21, wherein the PI3Kδ inhibitor is Acalisib (GS-9820, CAL-120), Dezapelisib (INCB040093), Idelalisib (CAL-101, GS-1101), Leniolisib (CDZ173), Inperlisib (YY-20394, PI3K(delta)-IN-2), Nemiralisib (GSK2269557), Parsaclisib (INCB050465, IBI-376), Puquitinib (XC-302), Seletalisib (UCB-5857), Zandelisib (ME-401, PWT143), ACP-319 (AMG 319), BGB-10188, GS-9901, GSK2292767, HMPL-689, IOA-244 (MSC236084), RV1729, or SHC014748M.

Embodiment 24. The method of any one of Embodiments 2-21, wherein the phenotype-altering composition comprises a PKA inhibitor and a p38 inhibitor.

Embodiment 25. The method of any one of Embodiments 2-21, wherein the phenotype-altering composition comprises a PKA inhibitor, a p38 inhibitor, and a PI3Kδ inhibitor.

Embodiment 26. The method of Embodiment 25, wherein the PKA inhibitor is Rp-8-Br-cAMPS, the p38 inhibitor is doramapimod, and the PI3Kδ inhibitor is idelalisib.

Embodiment 27. The method of any one of Embodiments 1-26, wherein the phenotype of at least a subpopulation of the population of T cells is altered after the culture period and/or the phenotype of at least a subpopulation of the population of T cells is altered after transfer of the T cells into the subject.

Embodiment 28. The method of Embodiment 27, wherein the phenotype altered after transfer into the subject is selected from the group consisting of greater persistence, prolonged survival, greater antitumor activity, and combinations thereof as compared to control T cells, wherein the control T cells are identical to the T cells cultured in the presence of the composition except that the control T cells are not cultured in the presence of the composition.

Embodiment 29. The method of any one of Embodiments 1-28, wherein phenotype-altered T cells have, before transfer into the subject, increased expression of one or more of CD62L, TCF1/TCF7, CCR7, and CD127, and/or decreased expression of one or more of CD69, CD39, CTLA-4, and PD-1, as compared to control T cells, wherein the control T cells are identical to the T cells cultured in the presence of the composition except that the control T cells are not cultured in the presence of the composition.

Embodiment 30. The method of Embodiment 29, wherein the expression of one or more of CD62L, TCF1/TCF7, CCR7, and CD127 is increased by at least 10%, at least 20%, at least 30%, or at least 40%.

Embodiment 31. The method of Embodiment 29, wherein the expression of one or more of CD69, CD39, CTLA-4, and PD-1 is decreased by at least 10%, at least 20%, at least 30%, or at least 40%.

Embodiment 32. The method of any one of Embodiments 1-29, wherein the phenotype-altered T cells have, upon activation in a restimulation culture, increased expression of IL-2 as compared to control T cells, wherein the control T cells are identical to the T cells cultured in the presence of the composition except that the control T cells are not cultured in the presence of the composition.

Embodiment 33. The method of any one of Embodiments 1-30, wherein the phenotype-altered T cells have, upon activation in a restimulation culture, increased expression of one or more of IL-2, INF-γ, TNF-α, or GM-CSF, as compared to control T cells, wherein the control T cells are identical to the T cells cultured in the presence of the composition except that the control T cells are not cultured in the presence of the composition.

Embodiment 34. The method of Embodiment 32 or Embodiment 33, wherein the restimulation culture does not contain the composition, but contains an anti-CD3 antibody or a combination of an anti-CD3 antibody and an anti-CD28 antibody.

Embodiment 35. The method of Embodiment 32 or Embodiment 33, wherein the phenotype-altered T cells express a T Cell Receptor (TCR), and wherein the restimulation culture does not contain the composition but contains cells expressing one or more tumor antigens that stimulates the T cell receptor (TCR).

Embodiment 36. The method of Embodiment 32 or Embodiment 33, wherein the phenotype-altered T cells express a Chimeric Antigen Receptor (CAR) and wherein the restimulation culture does not contain the composition but contains cells expressing one or more tumor antigens that stimulate the chimeric antigen receptor (CAR).

Embodiment 37. The method of Embodiment 32, wherein the expression of IL-2 is increased by at least 10%, at least 20%, at least 30%, or at least 40%.

Embodiment 38. The method of Embodiment 32, wherein the expression of one or more of IL-2, INF-γ, TNF-α, or GM-CSF is increased by at least 10%, at least 20%, at least 30%, or at least 40%.

Embodiment 39. The method of any one of Embodiments 1-31, wherein the population of T cells are cultured in the presence of the composition for at least 2 days, at least 3 days, at least 4 days, for at least 5 days, for at least 6 days, for at least 7 days, for at least 8 days, for at least 9 days, for at least 10 days, for at least 11 days, for at least 12 days, for at least 13 days, for at least 14 days, for at least 15 days, for at least 15 days, for at least 17 days, for at least 18 days, for at least 19 days, for at least 20 days, for at least 25 days, for at least 30 days, or for at least 40 days.

Embodiment 40. The method of any one of Embodiments 1-31, wherein the population of T cells are cultured in the presence of the composition for up to 2 days, up to 3 days, up to 4 days, up to 5 days, for up to 6 days, for up to 7 days, for up to 8 days, for up to days, for up to 10 days, for up to 11 days, for up to 12 days, for up to 13 days, for up to 14 days, for up to 15 days, for up to 15 days, for up to 17 days, for up to 18 days, for up to 19 days, for up to 20 days, for up to 25 days, for up to 30 days, or for up to 40 days.

Embodiment 41. A composition for improving therapeutic potential of T cells suitable for adoptive cell-based therapies, comprising: (1) a p38 inhibitor, a PI3Kδ inhibitor, or a combination thereof and (2) at least one agent selected from the group consisting of a protein kinase A (PKA) inhibitor, an A2A adenosine receptor inhibitor, and a GPR174 inhibitor, wherein the composition alters a phenotype of at least a subpopulation of immune cells cultured in vitro in the presence of the composition.

Embodiment 42. The composition of Embodiment 41, wherein the composition further comprises a cell culture medium suitable for culturing T cells.

Embodiment 43. The composition of Embodiment 41 or Embodiment 42, wherein the at least one agent is a GPR174 inhibitor.

Embodiment 44. The composition of Embodiment 43, wherein the GPR174 inhibitor is a small molecule GPR174 inhibitor or an antibody that specifically binds to GPR174.

Embodiment 45. The composition of Embodiment 43, wherein the GPR174 inhibitor is a small molecule represented by any one of Formulae I, II, II, IV, or V.

Embodiment 46. The composition of Embodiment 41 or Embodiment 42, wherein the at least one agent is a protein kinase A (PKA) inhibitor.

Embodiment 47. The composition of Embodiment 46, wherein the protein kinase A (PKA) inhibitor is selected from the group consisting of HA-100 dihydrochloride, Rp-cAMPS, H-89 dihydrochloride, PKI (5-24), Staurosporine, Calphostin C, KT 5720, Rp-8-Br-cAMPS, 5-Iodotubercidin, Piceatannol, Fasudil (monohydrochloride salt), ML-7 hydrochloride, CGP-74514A hydrochloride, ML-9, Daphnetin, Myricetin, PKC-412, A-674563, K-252a, H-7 dihydrochloride, bisindolylmaleimide IV, cGK1alpha inhibitor-cell permeable DT-3, TX-1123, Rp-8-PIP-cAMPS, 8-bromo2′-monobutyrladenosine-3′,5′-cyclic monophosphorothioate Rp-isomer, Bisindolylmaleimide III hydrochloride, Rp-adenosine 3′,5′-cyclic monophosphorothioate sodium salt, A-3 hydrochloride, H-7, H-8-2HCl, K252c, HA-1004 dihydrochloride, K-252b, HA-1077 dihydrochloride, MDL-27,032, H-9 hydrochloride, Rp-8-CPT-cAMPS, bisindolylmaleimide III, -lacetamido-4-cyano-3-methyllisoquinoline, Ilmofosine, Rp-8-hexylaminoadenosine 3′,5′-monophosphorothioate, HA-1004 hydrochloride, PKA Inhibitor IV, Adenosine 3′,5′-cyclic monophosphorothioate 8-chloro Rp-isomer sodium salt, adenosine 3′,5′cyclic monophosphorothioate 2′-O-monobutyryl Rp-isomer sodium salt, 4-cyano-3-methylisoquinoline, 8-hydroxyadenosine-3′,5′-monophosphorothioate Rp-isomer, PKI (6-22) amide, SB 218078, Rp-8-pCPT-cyclic GMPS sodium, Sp-8-pCPT-cAMPS, N[2-(p-Cinnamylamino)shyethyl]-5-isoquinolone sulfonamide, AT7867, GSK 690693, PKI (14-22) amide (myristoylated), Rp-8-bromo-cAMPS, or combinations thereof.

Embodiment 48. The composition of Embodiment 41 or Embodiment 42, wherein the at least one agent is an A2A adenosine receptor inhibitor.

Embodiment 49. The composition of Embodiment 48, wherein, wherein the A2A adenosine receptor inhibitor is selected from the group consisting of ZM 241385 (CAS 139180-30-6), istradefylline (CAS 155270-99-8), xanthine amine congener (CAS 96865-92-8), XCC (CAS 96865-83-7), ANR 94 (CAS 634924-89-3), PSB 1115 (CAS 409344-71-4), 3,7-dimethyl-1-propargylxanthine (CAS 14114-46-6), SCH 58261 (CAS 160098-96-4), SCH 442416 (CAS 316173-57-6), 8-(3-chlorostyryl)caffeine (CAS 147700-11-6), CGS 15943 (CAS 104615-18-1), ST4206 (CAS 246018-36-9), KF21213 (CAS 155271-17-3), regadenoson (CAS 313348-27-5), preladenant (CAS 377727-87-2), CGS 21680 (CAS 120225-54-9), tozadenant (CAS 870070-55-6), Sch412348 (CAS 377727-26-9), ST3932 (CAS 1246018-21-2), A2A receptor antagonist 1 (CPI-444 analog; CAS 443103-97-7), istradefylline (CAS 155270-99-8), AZD4635 (CAS 1321514-06-0), CGS 15943 (CAS 104615-18-1), vipadenant (CAS 442908-10-3), CPI-444 (CAS 1202402-40-1), TC-G 1004 (CAS 1061747-72-5), 4-desmethyl istradefylline (CAS 160434-48-0), PSB 0777 (CAS 2122196-16-9), or a combination thereof.

Embodiment 50. The composition of any one of Embodiments 41-49, wherein the p38 inhibitor is selected from the group consisting of doramapimod (CAS 285983-48-4), losmapimod (CAS 585543-15-3), SX 011 (CAS 309913-42-6), SB202190 (CAS 350228-36-3), VX 702 (CAS 745833-23-2), JX-401 (CAS 349087-34-9), p38 MAP Kinase Inhibitor VIII (CAS 321351-00-2). SCIO 469 (CAS 309913-83-5), p38 MAP Kinase Inhibitor V (CAS 271576-77-3), p38 MAP Kinase Inhibitor IX (N-(isoazol-3-yl)-4-methyl-3-(1-phenyl-1H-pyrazolo[3,4-d]pyrimidin-4-ylamino)benzamide), PD 169316 (CAS 152121-53-4), p38 MAP Kinase Inhibitor III (CAS 581098-48-8), PH-797804 (CAS 586379-66-0), RWJ 67657 (CAS 215303-72-3), VX 745 (CAS 209410-46-8), LY 364947 (CAS 396129-53-6), p38 MAP Kinase Inhibitor (CAS 219138-24-6), SB 239063 (CAS 193551-21-2), SB 202190 (CAS 152121-30-7), SB 203580 (CAS 152121-47-6), or a combination thereof.

Embodiment 51. The composition of any one of Embodiments 41-50, wherein the PI3Kδ inhibitor is Acalisib (GS-9820, CAL-120), Dezapelisib (INCB040093), Idelalisib (CAL-101, GS-1101), Leniolisib (CDZ173), Inperlisib (YY-20394, PI3K(delta)-IN-2), Nemiralisib (GSK2269557), Parsaclisib (INCB050465, IBI-376), Puquitinib (XC-302), Seletalisib (UCB-5857), Zandelisib (ME-401, PWT143), ACP-319 (AMG 319), BGB-10188, GS-9901, GSK2292767, HMPL-689, IOA-244 (MSC236084), RV1729, SHC014748M, or a combination thereof.

Embodiment 52. The composition of any one of Embodiments 41-50, wherein the composition comprises a PKA inhibitor and a p38 inhibitor.

Embodiment 53. The composition of any one of Embodiments 41-50, wherein the composition comprises a PKA inhibitor, a p38 inhibitor, and a PI3Kδ inhibitor.

Embodiment 54. The composition of Embodiment 53, wherein the PKA inhibitor is Rp-8-Br-cAMPS, the p38 inhibitor is doramapimod, and the PI3Kδ inhibitor is idelalisib.

Embodiment 55. A method of producing a population of phenotype-altered T cells, the method comprising culturing a population of T cells in vitro in the presence of a phenotype-altering composition comprising at least one agent selected from the group consisting of a protein kinase A (PKA) inhibitor, an A2A adenosine receptor inhibitor, a GPR174 inhibitor, and combinations thereof, wherein the phenotype-altering composition alters at least one phenotype of at least a subpopulation of the T cells.

Embodiment 56. The method of Embodiment 55, wherein the composition further comprises a p38 inhibitor, a PI3Kδ inhibitor, or a combination thereof.

Embodiment 57. The method of Embodiment 55 or Embodiment 56, wherein the at least one phenotype-altering agent is an exogenous agent.

Embodiment 58. The method of any one of Embodiments 55-57, wherein the population of T cells comprises genetically modified T cells comprising an exogenous nucleic acid encoding a T Cell Receptor (TCR), an exogenous nucleic acid encoding a Chimeric Antigen Receptor (CAR), or a combination thereof.

Embodiment 59. The method of any of Embodiments 55-58, wherein the population of T cells comprises T cells isolated from a subject suffering from a disease, T cells isolated from a universal donor, or universal donor T cells derived from stem cells.

Embodiment 60. The method of any of Embodiments 55-58, wherein the population of T cells comprises naïve T cells, stem cell memory T cells, central memory T cells, or combinations thereof.

Embodiment 61. The method of Embodiment 59, wherein the disease is a disease treatable by adoptive cell-based therapy.

Embodiment 62. The method of Embodiment 61, wherein the disease is cancer.

Embodiment 63. The method of Embodiment 62, wherein the cancer is a solid tumor or blood cancer.

Embodiment 64. The method of any one of Embodiments 55-63, wherein the phenotype altering agent is a GPR174 inhibitor.

Embodiment 65. The method of Embodiment 64, wherein the GPR174 inhibitor is a small molecule GPR174 inhibitor or an antibody that specifically binds to GPR174.

Embodiment 66. The method of Embodiment 64 or Embodiment 65, wherein the GPR174 inhibitor is a small molecule represented by any one of Formulae (I)-(VIII) or Table 1.

Embodiment 67. The method of any one of Embodiments 55-63, wherein the phenotype altering agent is a protein kinase A (PKA) inhibitor.

Embodiment 68. The method of Embodiment 67, wherein the protein kinase A (PKA) inhibitor is selected from the group consisting of HA-100 dihydrochloride, Rp-cAMPS, H-89 dihydrochloride, PKI (5-24), Staurosporine, Calphostin C, KT 5720, Rp-8-Br-cAMPS, 5-Iodotubercidin, Piceatannol, Fasudil (monohydrochloride salt), ML-7 hydrochloride, CGP-74514A hydrochloride, ML-9, Daphnetin, Myricetin, PKC-412, A-674563, K-252a, H-7 dihydrochloride, bisindolylmaleimide IV, cGK1alpha inhibitor-cell permeable DT-3, TX-1123, Rp-8-PIP-cAMPS, 8-bromo2′-monobutyrladenosine-3′,5′-cyclic monophosphorothioate Rp-isomer, Bisindolylmaleimide III hydrochloride, Rp-adenosine 3′,5′-cyclic monophosphorothioate sodium salt, A-3 hydrochloride, H-7, H-8-2HCl, K252c, HA-1004 dihydrochloride, K-252b, HA-1077 dihydrochloride, MDL-27,032, H-9 hydrochloride, Rp-8-CPT-cAMPS, bisindolylmaleimide III, -lacetamido-4-cyano-3-methyllisoquinoline, Ilmofosine, Rp-8-hexylaminoadenosine 3′,5′-monophosphorothioate, HA-1004 hydrochloride, PKA Inhibitor IV, Adenosine 3′,5′-cyclic monophosphorothioate 8-chloro Rp-isomer sodium salt, adenosine 3′,5′cyclic monophosphorothioate 2′-O-monobutyryl Rp-isomer sodium salt, 4-cyano-3-methylisoquinoline, 8-hydroxyadenosine-3′,5′-monophosphorothioate Rp-isomer, PKI (6-22) amide, SB 218078, Rp-8-pCPT-cyclic GMPS sodium, Sp-8-pCPT-cAMPS, N[2-(p-Cinnamylamino)shyethyl]-5-isoquinolone sulfonamide, AT7867, GSK 690693, PKI (14-22) amide (myristoylated), Rp-8-bromo-cAMPS, or combinations thereof.

Embodiment 69. The method of any one of Embodiments 55-63, wherein the phenotype altering agent is an A2A adenosine receptor inhibitor.

Embodiment 70. The method of Embodiment 69, wherein the A2A adenosine receptor inhibitor is selected from the group consisting of ZM 241385 (CAS 139180-30-6), istradefylline (CAS 155270-99-8), xanthine amine congener (CAS 96865-92-8), XCC (CAS 96865-83-7), ANR 94 (CAS 634924-89-3), PSB 1115 (CAS 409344-71-4), 3,7-dimethyl-1-propargylxanthine (CAS 14114-46-6), SCH 58261 (CAS 160098-96-4), SCH 442416 (CAS 316173-57-6), 8-(3-chlorostyryl)caffeine (CAS 147700-11-6), CGS 15943 (CAS 104615-18-1), ST4206 (CAS 246018-36-9), KF21213 (CAS 155271-17-3), regadenoson (CAS 313348-27-5), preladenant (CAS 377727-87-2), CGS 21680 (CAS 120225-54-9), tozadenant (CAS 870070-55-6), Sch412348 (CAS 377727-26-9), ST3932 (CAS 1246018-21-2), A2A receptor antagonist 1 (CPI-444 analog; CAS 443103-97-7), istradefylline (CAS 155270-99-8), AZD4635 (CAS 1321514-06-0), CGS 15943 (CAS 104615-18-1), vipadenant (CAS 442908-10-3), CPI-444 (CAS 1202402-40-1), TC-G 1004 (CAS 1061747-72-5), 4-desmethyl istradefylline (CAS 160434-48-0), PSB 0777 (CAS 2122196-16-9), or a combination thereof.

Embodiment 71. The method of any one of Embodiments 56-70, wherein the p38 inhibitor is selected from the group consisting of doramapimod (CAS 285983-48-4), losmapimod (CAS 585543-15-3), SX 011 (CAS 309913-42-6), SB202190 (CAS 350228-36-3), VX 702 (CAS 745833-23-2), JX-401 (CAS 349087-34-9), p38 MAP Kinase Inhibitor VIII (CAS 321351-00-2). SCIO 469 (CAS 309913-83-5), p38 MAP Kinase Inhibitor V (CAS 271576-77-3), p38 MAP Kinase Inhibitor IX (N-(isoazol-3-yl)-4-methyl-3-(1-phenyl-1H-pyrazolo[3,4-d]pyrimidin-4-ylamino)benzamide), PD 169316 (CAS 152121-53-4), p38 MAP Kinase Inhibitor III (CAS 581098-48-8), PH-797804 (CAS 586379-66-0), RWJ 67657 (CAS 215303-72-3), VX 745 (CAS 209410-46-8), LY 364947 (CAS 396129-53-6), p38 MAP Kinase Inhibitor (CAS 219138-24-6), SB 239063 (CAS 193551-21-2), SB 202190 (CAS 152121-30-7), SB 203580 (CAS 152121-47-6), or a combination thereof.

Embodiment 72. The method of any one of Embodiments 56-70, wherein the PI3Kδ inhibitor is Acalisib (GS-9820, CAL-120), Dezapelisib (INCB040093), Idelalisib (CAL-101, GS-1101), Leniolisib (CDZ173), Inperlisib (YY-20394, PI3K(delta)-IN-2), Nemiralisib (GSK2269557), Parsaclisib (INCB050465, IBI-376), Puquitinib (XC-302), Seletalisib (UCB-5857), Zandelisib (ME-401, PWT143), ACP-319 (AMG 319), BGB-10188, GS-9901, GSK2292767, HMPL-689, IOA-244 (MSC236084), RV1729, SHC014748M, or a combination thereof.

Embodiment 73. The method of any one of Embodiments 56-70, wherein the phenotype-altering composition comprises a PKA inhibitor and a p38 inhibitor.

Embodiment 74. The method of any one of Embodiments 56-70, wherein the phenotype-altering composition comprises a PKA inhibitor, a p38 inhibitor, and a PI3Kδ inhibitor.

Embodiment 75. The method of Embodiment 74, wherein the PKA inhibitor is Rp-8-Br-cAMPS, the p38 inhibitor is doramapimod, and the PI3Kδ inhibitor is idelalisib.

Embodiment 76. The method of any one of Embodiments 55-75, wherein the at least one phenotype is selected from the group consisting of greater persistence, greater antitumor activity, and combinations thereof as compared to control T cells, wherein the control T cells are identical to the T cells cultured in the presence of the composition except that the control T cells are not cultured in the presence of the composition.

Embodiment 77. The method of any one of Embodiments 55-76, wherein the population of phenotype-altered T cells have increased expression of one or more of CD62L, IL-2, INF-γ, TNF-α, GM-CSF, CCR7, and IL-7R as compared to control T cells, wherein the control T cells are identical to the T cells cultured in the presence of the composition except that the control T cells are not cultured in the presence of the composition.

Embodiment 78. The method of Embodiment 77, wherein the expression of one or more of IL-2, INF-γ, TNF-α, GM-CSF, CCR7, and IL-7R is increased by at least 10%, at least 20%, at least 30%, or at least 40%.

Embodiment 79. The method of any one of Embodiments 55-77, wherein the population of phenotype-altered T cells have been cultured in the presence of the composition for at least 4 days, for at least 5 days, for at least 6 days, for at least 7 days, for at least 8 days, for at least 9 days, for at least 10 days, for at least 11 days, for at least 12 days, for at least 13 days, for at least 14 days, or for at least 20 days.

Embodiment 80. The method of any one of Embodiments 55-79, wherein the method further comprises transfer of the phenotype-altered T cells to a re-stimulation environment.

Embodiment 81. The of method of Embodiment 80, wherein the re-stimulation environment comprises one or more tumor antigens.

Embodiment 82. The of method of Embodiment 80, wherein the re-stimulation environment is in vivo.

Embodiment 83. An isolated population of T cells comprising a subpopulation of phenotype-altered T cells produced by the method of any one of Embodiments 55-82.

Embodiment 84. A method of treating a disease, comprising administering to a subject in need thereof a therapeutically effective amount of T cells produced by the method of any one of Embodiments 47-70.

Embodiment 85. The method of Embodiment 84, wherein the method further comprises removal of the cultured T cells from the phenotype-altering composition prior to administering to the subject.

Embodiment 86. The method of Embodiment 84 or Embodiment 85, wherein the disease is cancer.

Embodiment 87. The methods or compositions of any one of Embodiments 1-86, wherein the T cells are T cells obtained from the subject in need thereof, T cells isolated from a universal donor, or universal donor T cells derived from stem cells.

The use of the terms “a” and “an” and “the” and “at least one” and the like in the context of describing the invention are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of examples, or exemplary language (e.g., “such as”) provided herein, is intended merely to illustrate the invention and does not pose a limitation on the scope of the invention unless otherwise claimed. All references mentioned in the disclosure are incorporated herein by reference in their entirety.

The following examples are intended to illustrate, rather than limit, the disclosure.

### EXAMPLES

### Example 1

This Example describes the effects of growing mouse CD8 T cells for 10 days with inhibitors of GPR174 and A2A on their production of IL-2 following restimulation in the absence of the inhibitors. For the experimental system, CD8 T cells were derived from mice transgenic for a T cell receptor specific for the hen ovalbumin peptide (OVA 257-264) presented by MHC class I H-2 K^{b}, termed OT-I. OT-I T cells were activated with the ovalbumin 257-264 peptide (pOVA commercially available from multiple suppliers, for example, Sigma-Aldrich, St Louis, Mo., catalog number: S7951) in the presence of mouse splenocytes and cultured with IL-7 and IL-15 to stimulate growth and survival.

Background/Rationale:

Culture conditions for ACT T cell manufacturing contain products of cellular metabolism and death, including adenosine and phosphatidylserine/lysophosphatidylserine (PS/lysoPS) which act on the Gas-coupled GPCRs A2A/A2B and GPR174, respectively. The inventors have previously shown that the combination of GPR174 and A2A inhibition results in synergistic enhancement of T cell activation and IL-2 production in culture conditions where endogenous PS/lysoPS and adenosine are present (Marc A. Gavin et al., Abstract B45: Phosphatidylserine suppresses T cells through GPR174, and co-inhibition of adenosine receptors and GPR174 synergistically enhances T cell responses. Cancer Immunol Res Mar. 1, 2020 (8) (3 Supplement)). The inventors hypothesized that extended growth of T cells in the presence of these inhibitors may imprint an elevated capacity to produce IL-2 following restimulation in the absence of inhibitors. Improved IL-2 production following T cell expansion with A2A and GPR174 inhibition should translate into better efficacy for NTR-T, CAR-T, and TCR-T cell therapy.

Methods:

Purified OT-I mouse CD8 T cells (StemCell mouse CD8 T cell purification kit) were cultured in 24-well plates with C₅₇BL/6 mouse splenocytes (pre-treated with mitomycin C to prevent growth) and pOVA according to the following condition per well:
2 mL RP10 media (RPMI, 10% fetal calf serum, 6 mM L-Glutamine, 12.5 mM HEPES, 50 μM 2-mercaptoethanol, Penicillin, Streptomycin)
0.2 million OT-I CD8 T cells
1.4 million mitomycin C treated splenocytes
100 nM pOVA

and the following individual conditions:
1. DMSO vehicle control
2. 300 nM GPR174 inhibitor Compound #10
3. 100 nM A2A inhibitor ZM-241385+300 nM GPR174 inhibitor Compound #10

On day 3, cultures were split 1:4 with new media, the inhibitors were added back to original conditions, and mouse IL-7 and mouse IL-15 were added to 10 ng/mL. On days 5 and 8, cells were washed once, counted, and replated at 0.5 million per well with IL-7 and IL-15 and the original inhibitor conditions. On day 10, cells were counted, washed twice with 12 mL RP10, and restimulated in quadruplicate with EG7 cells (H-2 K^{b} thymoma expressing OVA) in 96-well round bottom plates according to the following conditions:
0.2 mL RP10;
0.1 million OT-I T cells from each T cell expansion condition; and
0.5 million EG7 cells.

Supernatants were collected after 6 hours and assayed for IL-2 levels (MesoScale Discovery platform).

Results:

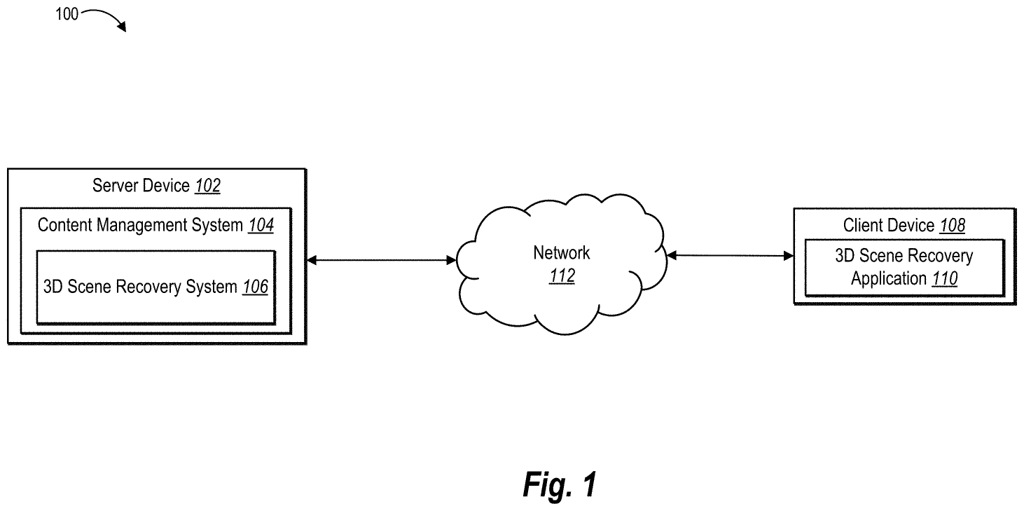

FIG. 1 graphically illustrates the impact of OT-I CD8 T cell expansion in the presence or absence of the exemplary GPR174 and A2A inhibitors on IL-2 production following restimulation. OT-I cells grown for 10 days with GPR174 inhibitor Compound #10 produced more IL-2 (1.8-fold) than cells grown with vehicle. Inclusion of the A2A inhibitor ZM-241385 with Compound #10 further improved the IL-2-production capacity (2.4-fold above Vehicle) (FIG. 1). Error bars represent standard deviations.

Discussion of Results:

This experiment demonstrated that 10-day culture of activated OT-I CD8 T cells with Compound #10 increased IL-2 production following T cell restimulation in the absence of Compound #10. Furthermore, the inclusion of the A2A inhibitor ZM-241385 with Compound #10 further enhanced IL-2 production from OT-I T cells. This suggests that the culture conditions contained PS/lysoPS and adenosine, which activated GPR174 and A2A, respectively, resulting in an attenuation of OT-I T cells to produce IL-2 upon restimulation. As the T cell culture conditions contained clumps of activated T cells and death of both the mitomycin C treated splenocytes and some OT-I T cells, it is reasonable to conclude that these processes lead to the release of PS-exposing vesicles and ATP from dying cells, which would be converted into lysoPS and adenosine by phospholipases and ectonucleotidases present in the culture.

Extrapolating to the generation of T cells for ACT, the culture conditions for their expansion also likely contain lysoPS and adenosine, which should attenuate their ability to express IL-2 following transfer into cancer patients. Thus, NTR-T cells, CAR-T cells, or TCR-T cells expanded for ACT-generated in the presence of a GPR174 inhibitor, an A2A inhibitor, or both together-should demonstrate improved anti-tumor activity, as greater IL-2 production should lead to greater growth, survival, and tumor-killing activity of the transferred cells in cancer patients.

### Example 2

This Example describes an experiment with human CD8 T cells similar to that described in Example 1. Rather than stimulating with antigen, human CD8 T cells were stimulated with anti-CD3/CD28 beads, as is typically done for CAR-T and TCR-T cell generation. Cells were expanded with IL-2 for 10 days in the presence of vehicle control, an exemplary GPR174 inhibitor, an exemplary A2A inhibitor, or both inhibitors combined.

Background/Rationale:

This experiment was performed to determine if the findings with mouse CD8 T cells could be replicated with human T cells, using a T cell stimulation and growth condition similar to those used for CAR/TCR-T cells.

Methods:

Purified human CD8 T cells were cultured in 24-well plates according to the following conditions per well:
1 mL X-VIVO™ 15 media (Lonza)
1 million human CD8 T cells
2 million Human T-Activator CD3/CD28 Dynabeads® (ThermoFisher)

and the following individual conditions:
1. DMSO vehicle control
2. A2A inhibitor ZM-241385
3. 300 nM GPR174 inhibitor Compound #10
4. 300 nM Compound #10+100 nM ZM-241385

On days 4 and 7, cultures were washed and seeded at 0.5 million cells per well with the same GPR174 and A2A inhibitor conditions, and with 100 U/mL human IL-2. On day 10, cells were washed twice (12 mL media each), and restimulated in quadruplicate in 96-well round-bottom plates with the following conditions:
0.1 mL X-VIVO™ 15 media (Lonza)
0.1 million cells from each condition
0.1 million Human T-Activator CD3/CD28 Dynabeads® (ThermoFisher)

After 7 hours of culture, supernatants were harvested and analyzed for IL-2 (MesoScale Discovery).

In addition to restimulation, expanded T cells were characterized by flow cytometry to evaluate expression of CCR7, CD39, CD69, TIGIT, CD45RA, LAG3, T-BET. The following detection reagents were used: anti-CD8 BV510, anti-CCR7 PE-Cy7, anti-CD39 BV605, anti-CD69 BV421, anti-TIGIT eFluor450, anti-CD45RA BV750, anti-LAG3 APC-Cy7, anti-T-BET PE-dazzle594 (all from Biolegend, Invitrogen, or BD Biosciences), and LIVE/DEAD™ Fixable Green Dead Cell Stain (ThermoFisher).

Data was collected with a Cytek Northern Lights flow cytometer, and was analyzed in FlowJo, in which automated clustering was performed on a concatenated file (containing all 4 culture conditions) with the FlowSOM plugin to generate 16 clusters. The abundance of each cluster in each cell sample was exported using the Cluster Explorer plugin.

Results:

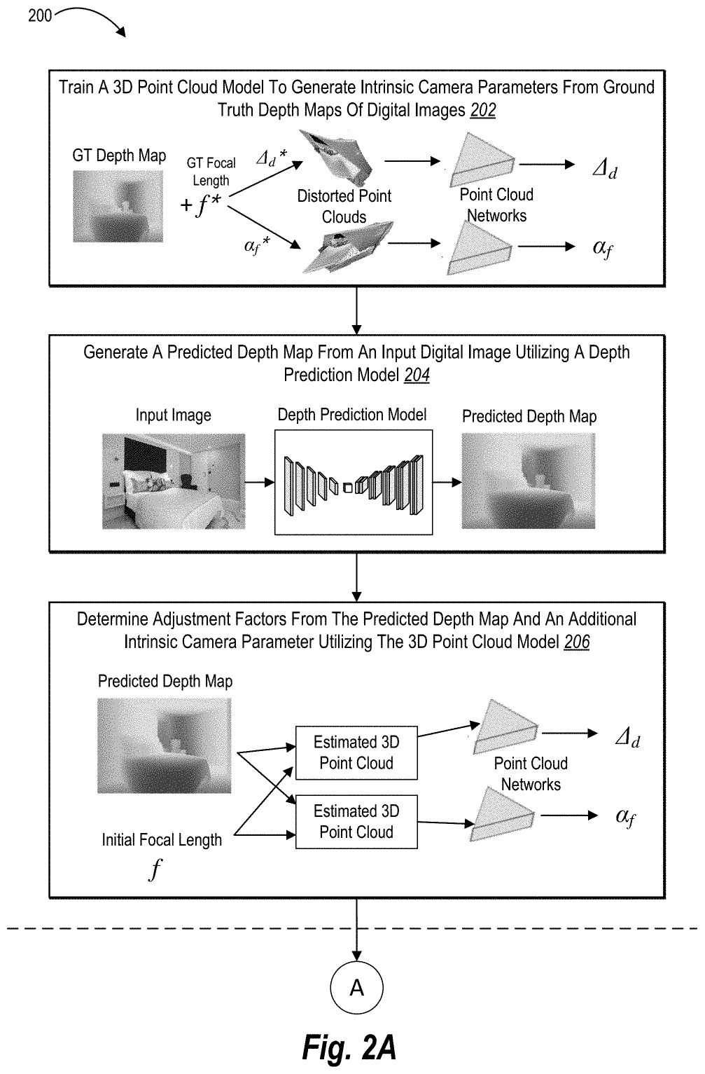

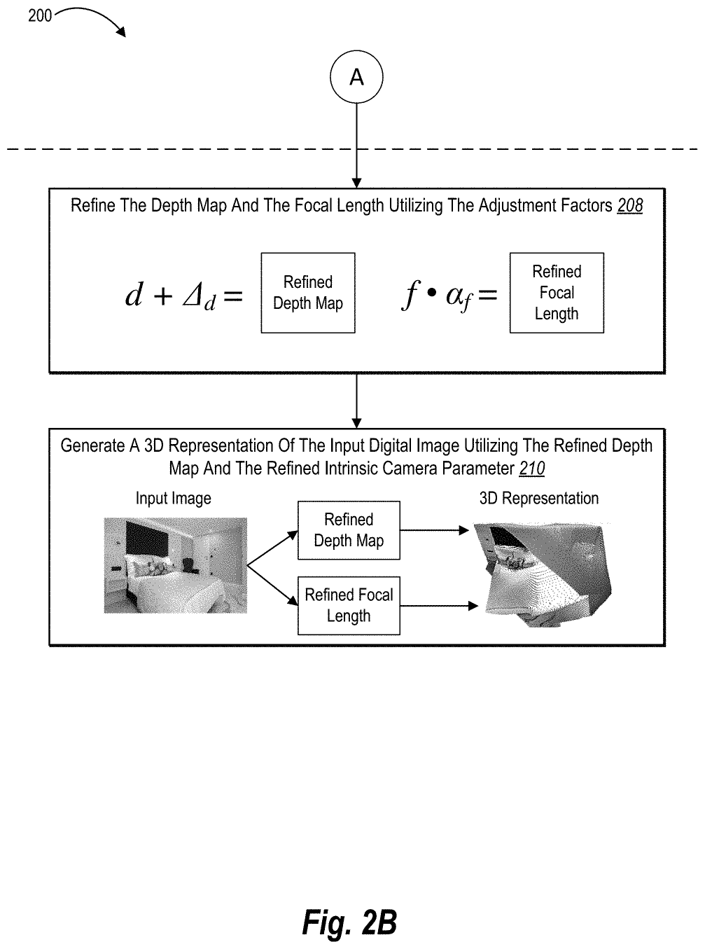

FIG. 2 graphically illustrates quantities of IL-2 in the supernatants of restimulated CD8 T cells. While preconditioning with the A2A inhibitor ZM-241385 alone did not alter IL-2 production upon restimulation, T cell expansion with the GPR174 inhibitor Compound #10 resulted in a 1.7-fold increase in IL-2 production (p<0.00001, t-test), and the combination of GPR174 and A2A inhibition resulted in a 2.8-fold increase in IL-2 (p<0.00001, t-test) (FIG. 2).

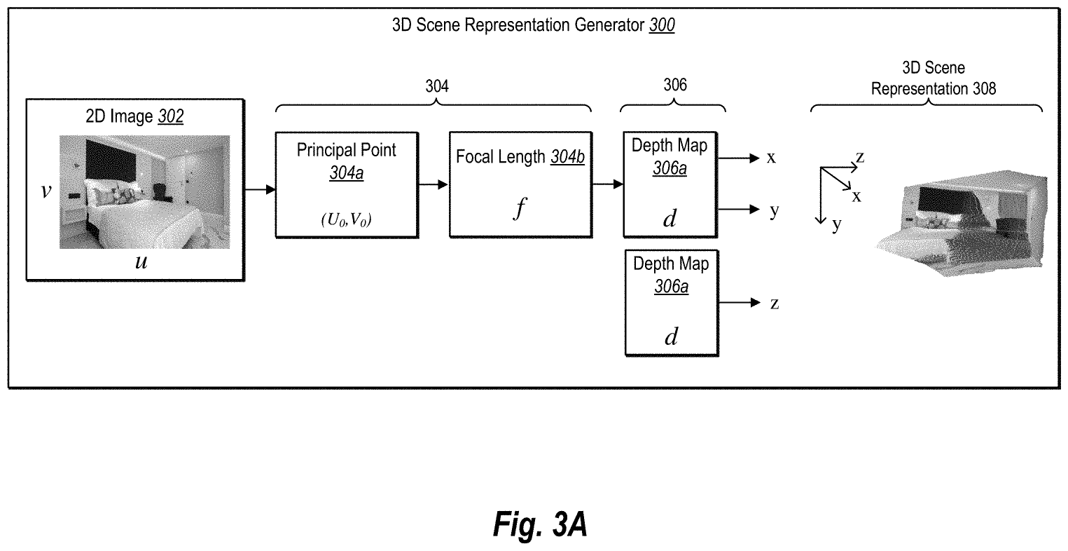

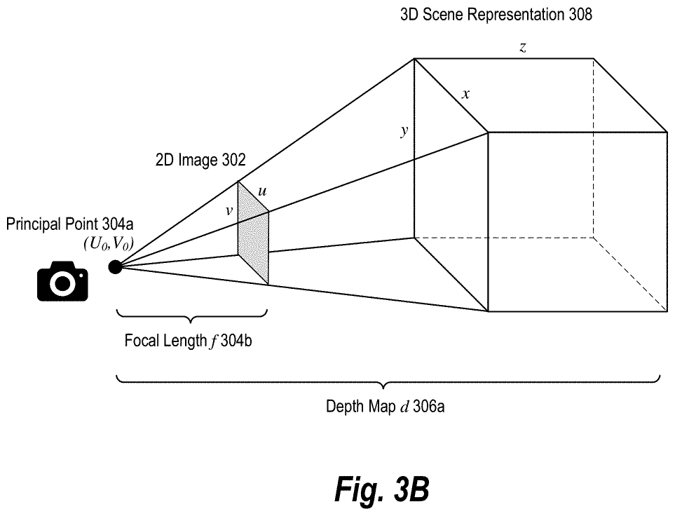

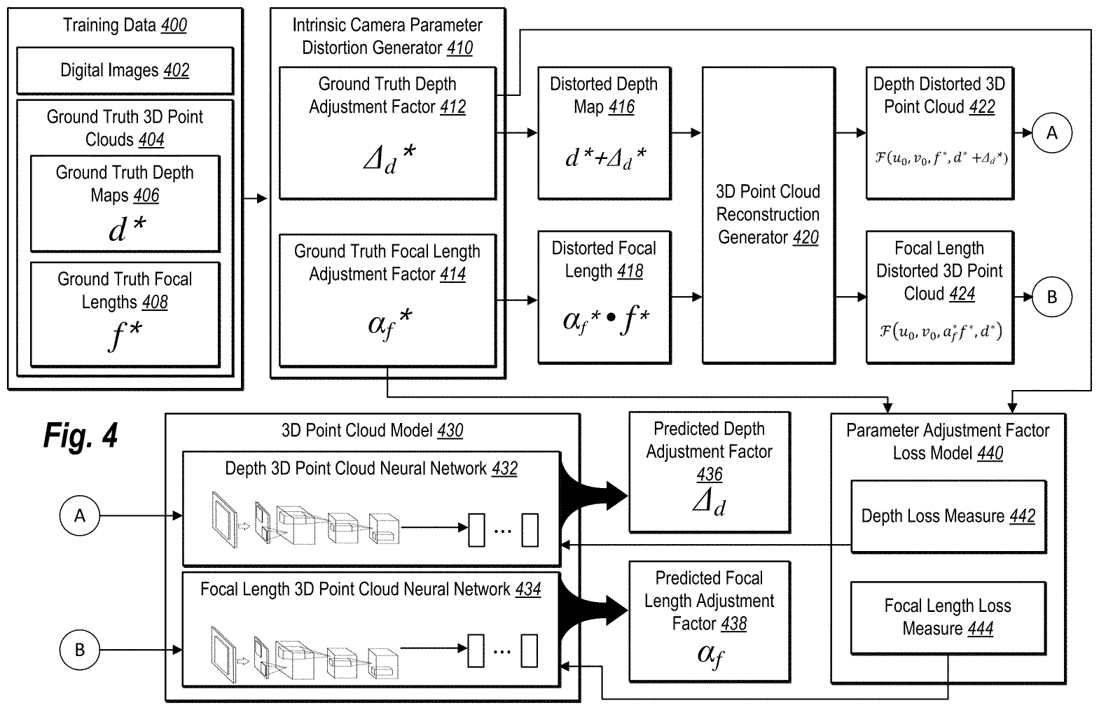

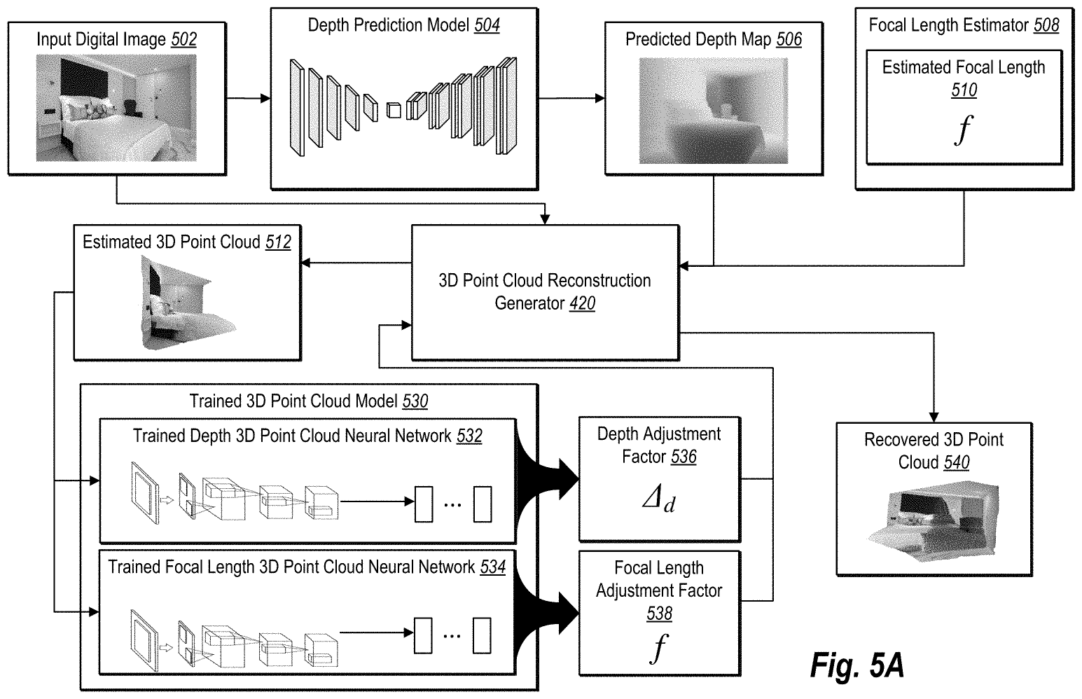

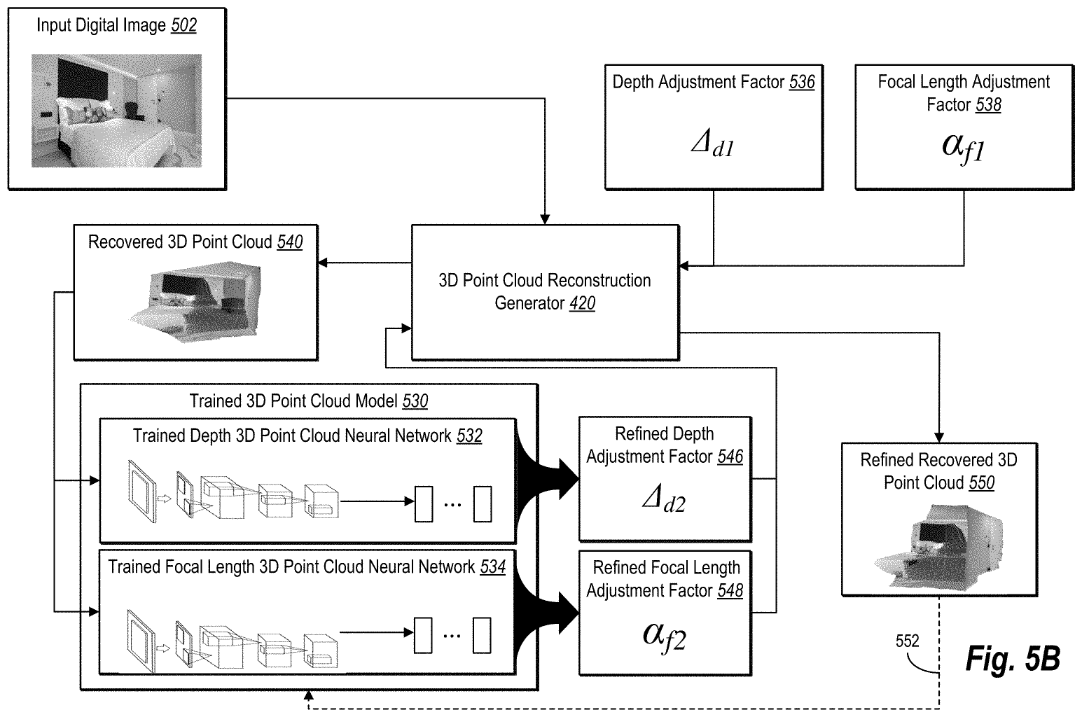

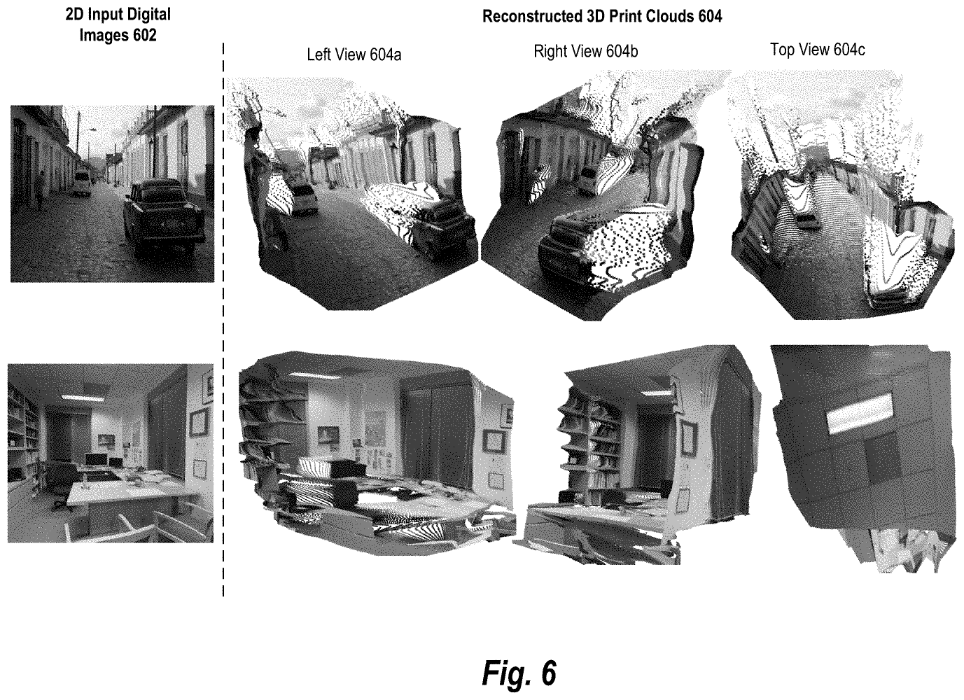

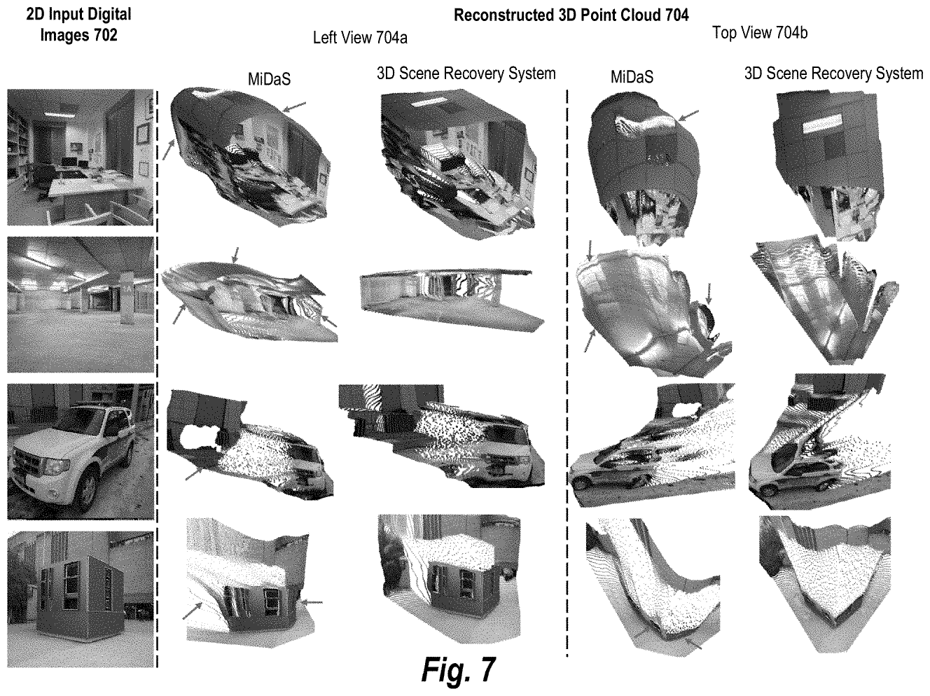

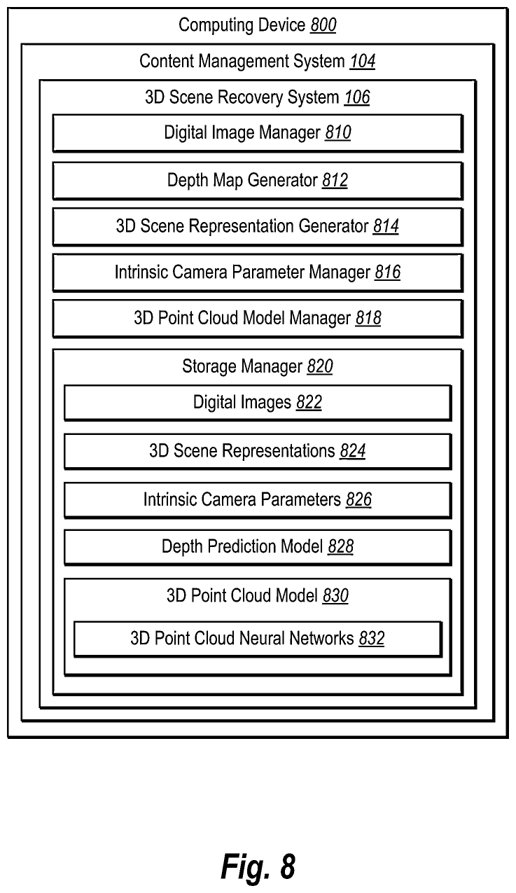

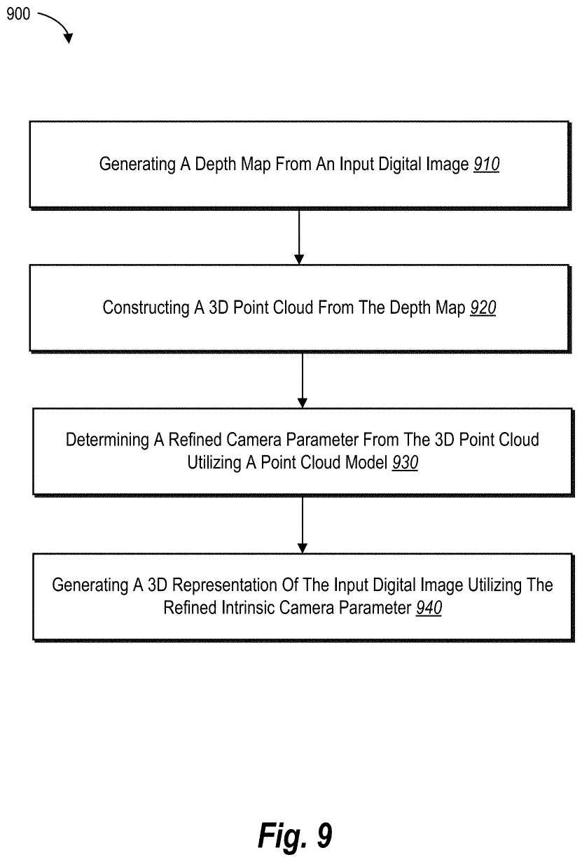

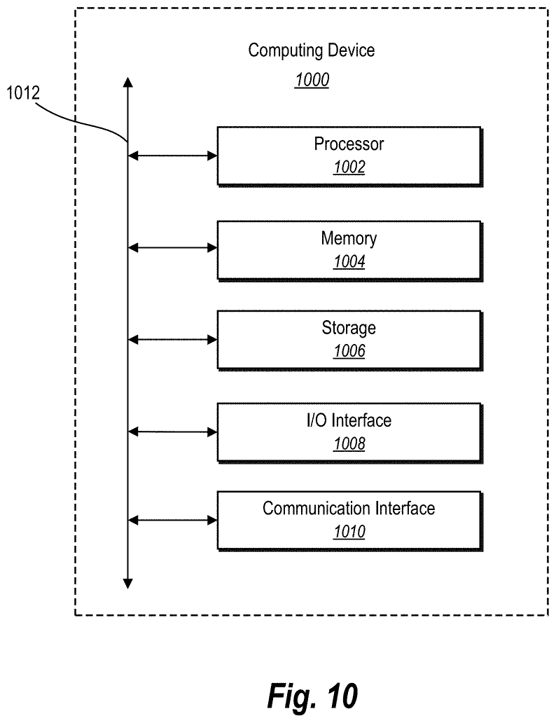

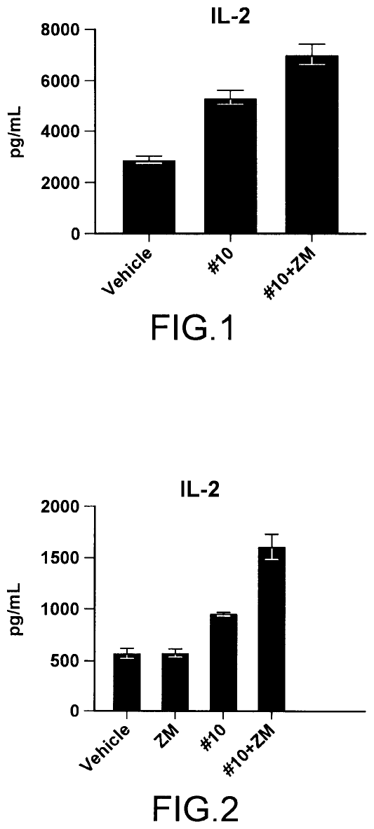

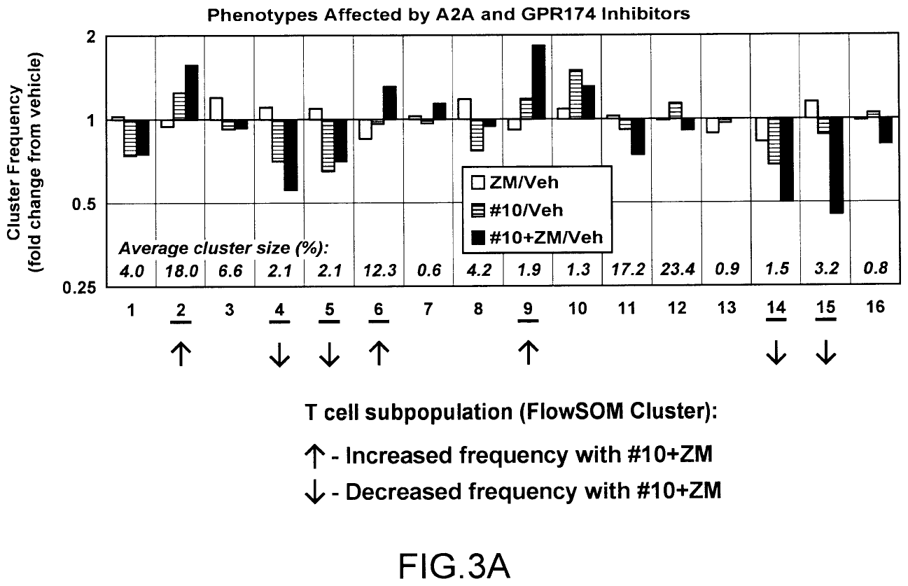

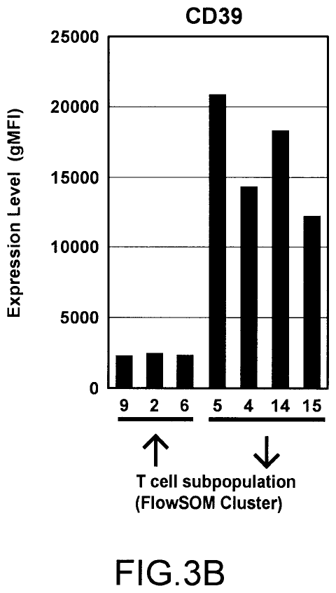

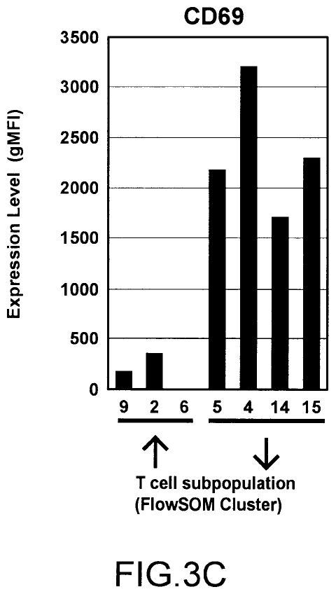

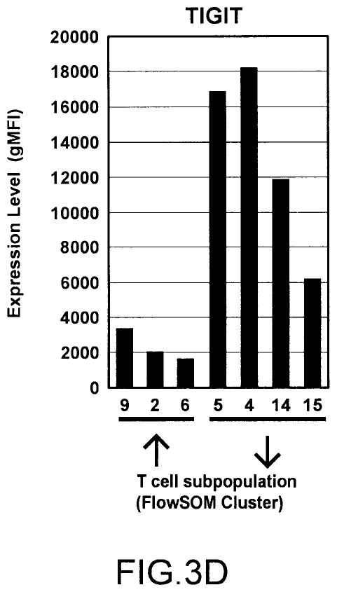

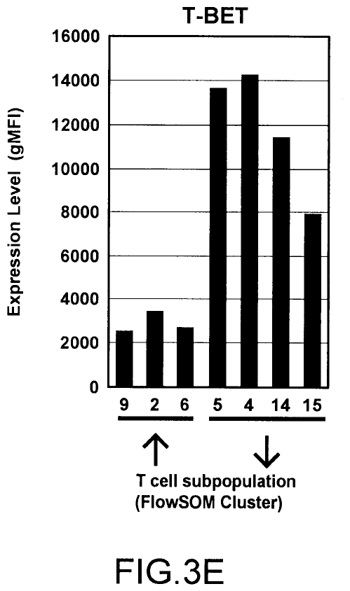

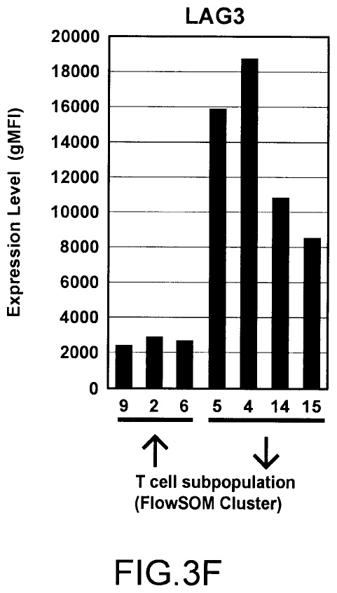

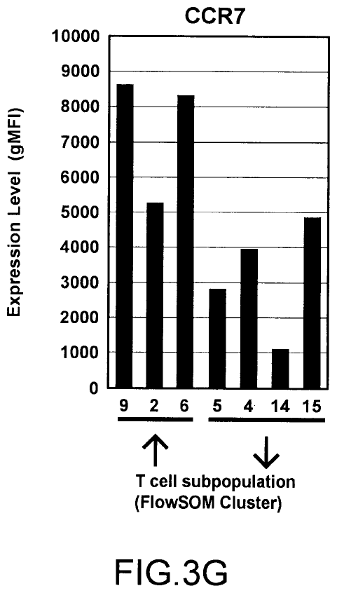

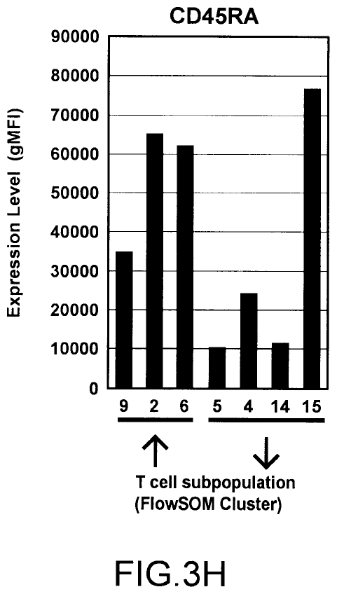

FIG. 3 graphically illustrates the T cell phenotypes that were enriched or reduced by the 10-day culture with the A2A inhibitor ZM-241385 and/or the GPR174 inhibitor Compound #10. FlowSOM automated clustering of the concatenated flow cytometry data was performed to generate 16 clusters of cellular phenotypes. The abundance of each cluster was plotted for each of the 3 experimental conditions as a fold-change relative to the vehicle control condition (FIG. 3A), and the size of each cluster (average percentage of all cells) is shown in italics (FIG. 3A). The defining characteristics of the 3 most upregulated clusters (2, 6, 9) compared to the reduced clusters (4, 5, 14, 15) were reduced expression of CD39, CD69, TIGIT, T-BET, and LAG3, and elevated expression of CCR7 and CD45RA (FIG. 3B). These clusters were relatively unchanged in cells treated with the A2A inhibitor alone, while cells treated with the GPR174 inhibitor displayed this same trend, albeit to a lesser degree (FIG. 3A), similar to what was observed for IL-2 expression for the same cells following restimulation (FIG. 2).

Discussion of Results:

Together, the functional and phenotypic analysis revealed that human CD8 T cells stimulated and grown in the presence of GPR174 and A2A inhibitors are enriched for cells with a central memory phenotype and contain fewer terminally differentiated effector T cells. Expression of the checkpoint molecules TIGIT and LAG3 and transcription factor T-BET indicate terminal differentiation or exhaustion of effector T cells, while expression of IL-2, CCR7 and CD45RA and downregulation of CD39 and CD69 are associated with central memory T cells with self-replicating potential (Matthew D. Martin, and Vladimir P. Badovinac, Defining Memory CD8 T Cell. Frontiers in Immunology. 2018, Vol. 9, p. 2692; Krishna, S. et al, Stem-like CD8 T cells mediate response of adoptive cell immunotherapy against human cancer. Science 2020: 1328-1334). The phenotypic alterations of CD8 T cells expanded in the presence of a GPR174 inhibitor or combined GPR174 and A2A inhibitors supports the inventor's expectation that CAR-T cells or ACT T cells generated with these inhibitors will persist and grow more effectively following transfer into cancer patients.

### Example 3

This Example describes an experiment similar to Example 2, in which IL-7 and IL-15 are used in place of IL-2 during the 10-day T cell expansion. An additional exemplary GPR174 inhibitor of a different structural class (Compound #49) was also tested in this Example.

Background/Rationale:

Optimized protocols for the generation of CAR-T cells have incorporated the T cell growth factors IL-7 and IL-15 rather than IL-2, because the former were found to be more effective at maintaining a central memory phenotype and enabling persistence and anti-tumor activity following transfer into mice (Zhou, J., Jin, L., Wang, F. et al. Chimeric antigen receptor T (CAR-T) cells expanded with IL-7/IL-15 mediate superior antitumor effects. Protein Cell 10, 764-769 (2019); Xu, Y. et al, Molecular Therapy, 21, S2-S21 (2013); Tessa Gargett 1, Michael P Brown Cytotherapy, 17(4):487-95 (2015)).

The inventors investigated if the GPR174 and A2A inhibitors would still enhance the IL-2 production capacity of CD8 T cells expanded with IL-7 and IL-15 or if the effects of these cytokines would override, or be redundant with, the effects of the inhibitors. A separate GPR174 inhibitor belonging to a separate chemical class (Compound #49) was tested to provide further evidence that the observed outcomes were due to specific inhibition of GPR174.

Methods:

Purified human CD8 T cells were cultured in 24-well plates with the following conditions per well:
2 mL X-VIVO™ 15 media (Lonza)
1 million human CD8 T cells
2 million Human T-Activator CD3/CD28 Dynabeads® (ThermoFisher)
10 ng/mL human IL-7 (R&D Systems)
10 ng/mL human IL-15 (R&D Systems)

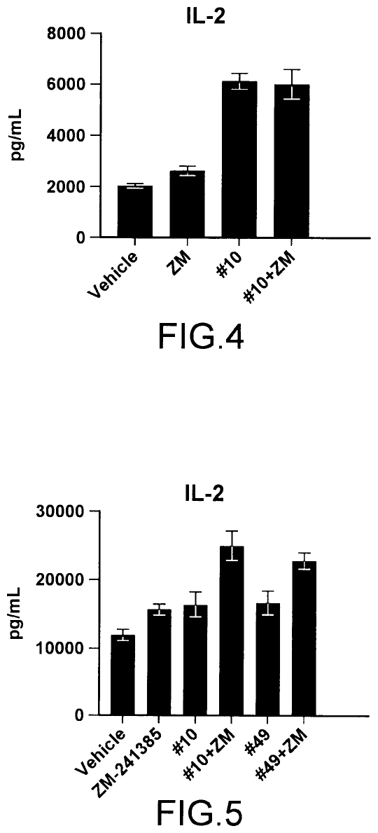

and the following individual conditions for FIG. 4:
1. DMSO vehicle control
2. 100 nM A2A inhibitor ZM-241385
3. 300 nM GPR174 inhibitor Compound #10
4. 300 nM Compound #10+100 nM ZM-241385

or the following individual conditions for FIG. 5:
1. DMSO vehicle control
2. 100 nM A2A inhibitor ZM-241385
3. 300 nM GPR174 inhibitor Compound #10
4. 300 nM Compound #10+100 nM ZM-241385
5. 500 nM GPR174 inhibitor Compound #49
6. 500 nM GPR174 inhibitor Compound #49+100 nM ZM-241385

On days 4 and 7 (FIG. 4) or days 3, 5, and 7 (FIG. 5), cultures were washed and seeded at 0.5 cells million per well with the same GPR174 and A2A inhibitor conditions, and with 10 ng/mL IL-7 and 10 ng/mL IL-15. On day 10, cells were washed twice (12 mL media each), and restimulated in quadruplicate in 96-well round-bottom plates with the following conditions:
0.1 mL X-VIVO™ 15 media (Lonza)
0.1 million cells from each condition
0.2 million Human T-Activator CD3/CD28 Dynabeads® (ThermoFisher)

After 18 hours of culture, supernatants were harvested and analyzed for IL-2 (MesoScale Discovery).

Results:

FIG. 4 graphically illustrates quantities of IL-2 in the supernatants of restimulated CD8 T cells. As observed with cells expanded with IL-2, the GPR174 inhibitor compound #10 also elevated IL-2 production when cells were expanded with IL-7+IL-15. Cells cultured in compound #10 produced 3-fold more IL-2 than vehicle control (p<0.00001) (FIG. 3). In this experiment, the effect of the A2A inhibitor ZM-241385 was negligible.

FIG. 5 graphically illustrates the same readouts as FIG. 4. In this experiment, the impact of ZM-241385 or Compound #10 alone on IL-2 production were modest (approximately 1.3-fold, p<0.01). In contrast, the combination of Compound #10 and ZM-241385 resulted in a synergistic enhancement of IL-2 production following restimulation (2.1-fold, p=0.00003) as observed in Example 2, and similar effects were seen with a separate GPR174 inhibitor Compound #49 (1.9-fold, p=0.000006).

Discussion of Results:

Example 3 demonstrates that CD8 T cells stimulated and expanded with IL-7 and IL-15 produced more IL-2 following restimulation when a GPR174 inhibitor was included in the expansion culture. Because recently improved ACT T cell culture conditions utilize IL-7 and IL-15 rather than IL-2, these results suggest GPR174 inhibition should enable the production of more effective T cell therapies with current optimized T cell expansion conditions. Similar activities with two GPR174 inhibitors representing distinct chemical classes supports the conclusion that the effect of the compounds was GPR174-specific.

In the Examples presented thus far, the inventors observe variability in the effect of the GPR174 inhibitors relative to the A2A inhibitor, and the effect of their combination. In FIGS. 2 and 5, inhibition of individual GPCRs had a modest effect (or no effect for A2A inhibition in FIG. 2), while the combination of GPR174 and A2A inhibitors resulted in a synergistic increase in IL-2 production following restimulation. In contrast, the experiment in FIG. 4 shows that T cell expansion conditions can occur where the GPR174 inhibitor alone has a large effect, and A2A inhibition has no effect either alone or in combination with a GPR174 inhibitor. This variability may have resulted from differences in PS/lysoPS and adenosine abundance among the different T cell expansion cultures. Because T cells express multiple Gas-coupled GPCRs, this observation also suggested that global inhibition of cAMP signaling may be more effective at maintaining a memory T cell phenotype.

### Example 4

This Example describes the effects of inhibition of GPR174, A2A, or proteins in the cAMP signaling pathway (PKA or exchange protein directly activated by cAMP (EPAC)) during 10-day human CD8 T cell expansion on IL-2 production following restimulation in the absence of inhibitors.

Background/Rationale:

GPR174 and A2A both signal through the Gas/cAMP signaling pathway. Because the inventors observed variable effects of their inhibitors and because the T cell expansion conditions could contain agonists of other Gas-coupled GPCRs such as low pH activating GPR65, the inventors reasoned that direct inhibition of cAMP signaling pathways should have a greater and more reproducible effect in this system. The signaling molecules PKA and EPAC are activated by cAMP and inhibited by Rp-8-Br-cAMPS and ESI-09 (CAS Number 263707-16-0), respectively. In the following experiment, the inventors tested ZM (ZM-241385), Rp-8-Br-cAMPS, and ESI-09, each alone or in combination with Compound #10, to determine if inhibition of PKA or EPAC had a greater effect on IL-2 production than Compound #10 or the combination of Compound #10 and ZM-241385, and if any effects of Compound #10 would be masked by PKA or EPAC inhibition.

Methods:

Purified human CD8 T cells were cultured in 24-well plates with the following conditions per well:
2 mL X-VIVO™ 15 media (Lonza)
0.4 million human CD8 T cells (Donor A)
2 million Human T-Activator CD3/CD28 Dynabeads® (ThermoFisher)
10 ng/mL human IL-7 (R&D Systems)
10 ng/mL human IL-15 (R&D Systems)

and the following individual conditions, ±300 nM Compound #10:
1. DMSO vehicle control
2. 100 nM ZM-241385
3. 500 μM Rp-8-Br-cAMPS
4. 5 μM ESI-09

On days 3, 5, and 7, cultures were washed and seeded at 0.5 cells million per well with the same small molecule inhibitor conditions, and with IL-7 and IL-15. On day 10, cells were washed twice (12 mL media each), and restimulated in triplicate in 96-well round-bottom plates with the following conditions:
0.1 mL X-VIVO™ 15 media (Lonza)
0.05 million cells from each condition
0.1 million Human T-Activator CD3/CD28 Dynabeads® (ThermoFisher).

After 5.5 hours of culture, supernatants were harvested and analyzed for IL-2 content (MesoScale Discovery).

Results:

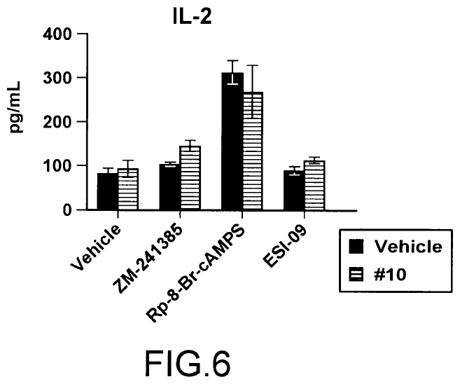

FIG. 6 graphically illustrates the production of IL-2 following restimulation of CD8 T cells that had been cultured with the various small molecule inhibitors. The largest increase in IL-2 production was observed with the PKA inhibitor Rp-8-Br-cAMPS (3.7-fold, p=0.0002). In contrast, Compound #10 and ZM-241385 had negligible effect on their own, and together elicited a 1.7-fold increase in IL-2 production (p=0.003). Compound #10 did not enhance IL-2 production in the presence of Rp-8-Br-cAMPS, and the EPAC inhibitor ESI-09 had no impact on IL-2 production.

Discussion of Results:

These findings suggest that, during the 10-day T cell expansion, PKA inhibition was more effective than the combined GPR174 and A2A inhibition at increasing IL-2 production capacity, and that EPAC did not play a role in the effects of cAMP signaling on IL-2 expression.

### Example 5

This Example describes the effects of inhibitors of GPR174 and A2A together, a PKA inhibitor (Rp-8-Br-cAMPS), and a p38 inhibitor (doramapimod), during a 10-day human CD8 T cell expansion, on IL-2 production following restimulation in the absence of inhibitors. In a separate experiment, the effects of the PKA inhibitor and two EPAC inhibitors were evaluated in the presence or absence of the p38 inhibitor.

Background/Rationale:

The goal of increasing CAR-T and ACT T cell survival and anti-tumor activity in patients by preconditioning with small molecule inhibitors is an area of active research. A recent study found that inhibition of p38 kinase with doramapimod during T cell expansion rendered CD8 T cells more effective at producing IL-2 and eradicating tumors in mice (Gurusamy D, et al. Multi-phenotype CRISPR-Cas9 Screen Identifies p38 Kinase as a Target for Adoptive Immunotherapies. Cancer Cell. 2020; 37(6):818-833). Thus, the inventors sought to determine if the effects observed with the PKA inhibitor were similar to those of doramapimod, and if the combination of the PKA and p38 inhibitors further increased IL-2 production through additive or synergistic cooperativity, or if the actions of the two compounds was redundant.

Methods:

Purified human CD8 T cells were cultured in 24-well plates with the following conditions per well:
2 mL X-VIVO™ 15 media (Lonza)
1 million human CD8 T cells (Donor 224)
2 million Human T-Activator CD3/CD28 Dynabeads® (ThermoFisher)
10 ng/mL human IL-7 (R&D Systems)
10 ng/mL human IL-15 (R&D Systems)

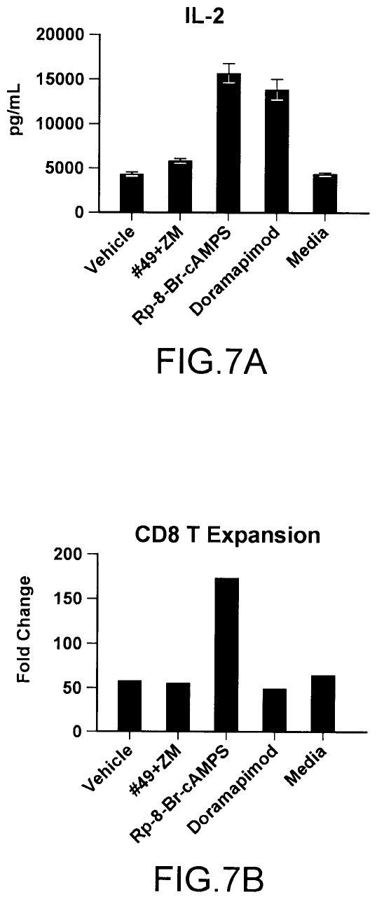

and the following individual conditions for the experiment shown in FIG. 7:
1. DMSO vehicle control
2. 300 nM Compound #10+100 nM ZM-241385
3. 500 μM Rp-8-Br-cAMPS
4. 1 μM KT-5720 (CAS Number 108068-98-0, a PKA inhibitor)
5. 0.5 μM doramapimod
6. Media control

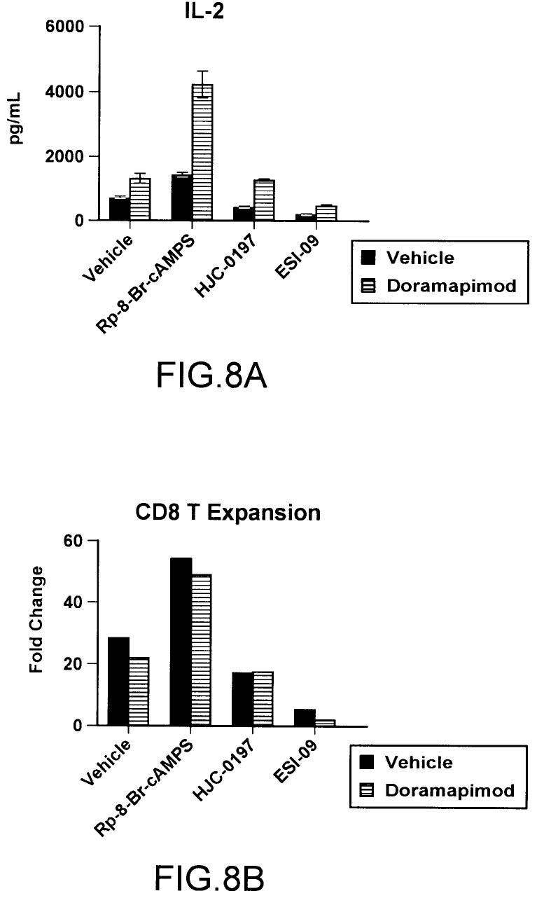

or the following individual conditions for the experiment shown in FIG. 8, each ±0.5 μM doramapimod:
1. DMSO vehicle control
2. 500 μM Rp-8-Br-cAMPS
3. 1 μM KT-5720
4. 10 μM HJC-0197 (an EPAC antagonist, CAS Number 1383539-73-8)
5. 5 μM ESI-09

On days 3, 5, and 7, cultures were washed and seeded at 0.5 cells million per well with the same small molecule inhibitor conditions, and with IL-7 and IL-15. On day 10, cells were washed twice (12 mL media each), and restimulated in quadruplicate in 96-well round-bottom plates with the following conditions:
0.1 mL X-VIVO™ 15 media (Lonza)
0.05 million cells from each condition
0.1 million Human T-Activator CD3/CD28 Dynabeads® (ThermoFisher)

After overnight culture, supernatants were harvested and analyzed for IL-2 content (MesoScale Discovery).

Results:

FIG. 7 graphically illustrates the production of IL-2 following restimulation of human CD8 T cells that had been cultured with the indicated small molecule inhibitors, and fold-increases in T cell number during the 10-day expansion with the compounds. The PKA inhibitor Rp-8-Br-cAMPS and the p38 inhibitor doramapimod increased to the same degree IL-2 production from CD8 T cells (3.6- and 3.2-fold, respectively; p<0.0001) (FIG. 7A). In contrast, in this experiment the combination of the GPR174 inhibitor Compound #49 and the A2A inhibitor ZM-241385 had a modest effect on IL-2 production following restimulation (1.3-fold; p<0.0001). During the 10-day T cell expansion, cell numbers increased approximately 50-fold for the vehicle and media control samples, as well as the Compound #49+ZM-241385 combination and the doramapimod condition (FIG. 7E). In contrast, a 170-fold increase in cell numbers was observed for the PKA inhibitor Rp-8-Br-cAMPS.

FIG. 8 graphically illustrates the same readouts shown in FIG. 7 for human CD8 T cells expanded with 3 inhibitors of cAMP signaling (PKA inhibitor Rp-8-Br-cAMPS, and EPAC inhibitors HJC-0197 and ESI-09), each with or without the p38 inhibitor doramapimod. Similar to the previous experiment, Rp-8-Br-cAMPS and doramapimod each increased IL-2 production by 2-fold and 1.8-fold, respectively (p<0.0001). In contrast, the combination of the two inhibitors synergistically enhanced IL-2 production to 5.8-fold above vehicle control (p<0.00001) (FIG. 8A). Effects of the 2 EPAC inhibitors HJC-0197 and ESI-09 were absent or negligible. Regarding T cell growth during the 10-day culture, Rp-8-Br-cAMPS-treated cells expanding nearly 2-fold more than vehicle-treated cells, (FIG. 8E). One of the 2 EPAC inhibitors (ESI-09) attenuated T cell expansion, and doramapimod had very little effect on cell growth (FIG. 8E).

Discussion of Results:

In this Example, inhibitors of cAMP signaling were compared to and combined with the p38 inhibitor doramapimod. These studies were performed because doramapimod was recently shown to promote activities similar to those promoted by GPR174, A2A, and PKA inhibitors as disclosed herein in similar T cell expansion and restimulation assays (Gurusamy D, et al. Multi-phenotype CRISPR-Cas9 Screen Identifies p38 Kinase as a Target for Adoptive Immunotherapies. Cancer Cell. 2020; 37(6):818-833). The results described in this Example demonstrated that PKA and p38 inhibition increased IL-2 production from restimulated CD8 T cells to the same degree. Importantly, the activities did not appear to be redundant. Instead, inhibition of both pathways together resulted in synergistic augmentation of IL-2 to levels greater than the product of the increases seen with each inhibitor alone (FIGS. 8A-D). Furthermore, only PKA inhibition markedly augmented T cell expansion, while p38 inhibition had no effect in this parameter. The absence of IL-2 induction following culture with two unique EPAC inhibitors indicates that PKA is the cAMP-responsive signaling molecule responsible for the improved growth and IL-2 production. Together, these results demonstrate that the combination of Rp-8-Br-cAMPS and doramapimod, or other specific PKA and p38 inhibitors, should enhance the production of T cells for ACT by improving both their growth during manufacturing and their IL-2 production following transfer into cancer patients.

### Example 6

This Example describes the effects of the PKA and p38 inhibitors, Rp-8-Br-cAMPS and doramapimod, on mouse CD8 T cell phenotypes following stimulation and growth with either inhibitor or both combined. As in Example 1, the inventors used OT-I TCR transgenic T cells, stimulating with its peptide antigen OVA 257-264 (pOVA), or with the EG7 T cell line that endogenously expresses this antigen.

Background/Rationale:

To characterize the potential for T cells grown in the presence of PKA and p38 inhibitors to eradicate tumors, it is important to evaluate this approach in mouse models of tumor ACT and CAR-T therapy. Thus, in this example, the effects of the inhibitors on mouse OT-I CD8 T cells were examined. To more comprehensively characterize the expanded T cells, in addition to measuring IL-2 production following restimulation, the inventors also phenotyped the expanded OT-I T cells for markers of memory T cells (Krishna S. et al. Stem-like CD8 T cells mediate response of adoptive cell immunotherapy against human cancer. Science, 2020: 1328-1334). Increased IL-2 production and increased capacity for self-renewal should correlate with a memory T cell phenotype characterized by higher expression of the transcription factor TCF1/TCF7 and the lymphoid tissue homing molecule CD62L Furthermore, a recent publication on the phenotype of long lived memory cells among T cells prepared for ACT found that the lack of CD39 and CD69 expression correlated with a memory T cell phenotype featuring increased TCF1/TCF7 and CD62L expression, and that ACT T cell preparations that contained a greater proportion of these cells correlated with improved progression-free survival. Thus, CD39 and CD69 were included in the analysis, as well as the T cell-inhibiting checkpoint molecules PD-1 and CTLA-4. Lastly, to determine if the length of culture with the inhibitors influenced the degree to which they augmented IL-2 production potential, the OT-I cultures were restimulated on both day 8 and day 10.

Methods:

Mouse OT-I TCR transgenic CD8 T cells were purified from splenocytes (StemCell 19853; Mouse CD8⁺ T Cell Isolation Kit), cultured in a 24-well plate with the following conditions per well:
2 mL RP10 media
4 million mitomycin C treated C57BL/6 mouse splenocytes
0.2 million OT-I T cells
10 nM pOVA

and the following individual conditions:
1. DMSO vehicle control
2. 0.2 μM doramapimod
3. 500 μM Rp-8-Br-cAMPS
4. 0.2 μM doramapimod+500 μM Rp-8-Br-cAMPS

On days 2, 4, 6, and 8, cells were counted, washed, and reseeded at 0.5 million per well in 2 mL media with the same 4 doramapimod/Rp-8-Br-cAMPS conditions and with recombinant mouse IL-2 and IL-7 (5 ng/mL each). On day 8, cells were immunophenotyped with the following antibodies: anti-CD8 BV570, anti-CD62L eFluor450, anti-TCF1/TCF7 PE, anti-CD39 PerCP-eFluor710, anti-CD69 BV605, anti-PD-1 BV711, anti-CTLA-4 APC, and LIVE/DEAD™ Fixable Green Dead Cell Stain.

On days 8 and 10, cells were washed three times (12 mL media each), and restimulated in quintuplicate in 96-well round-bottom plates with the following conditions:
0.1 mL RP10 media
0.05 million OT-I T cells from each condition
0.05 million EG7 cells

After overnight culture, supernatants were harvested and analyzed for IL-2 content (MesoScale Discovery).

Results:

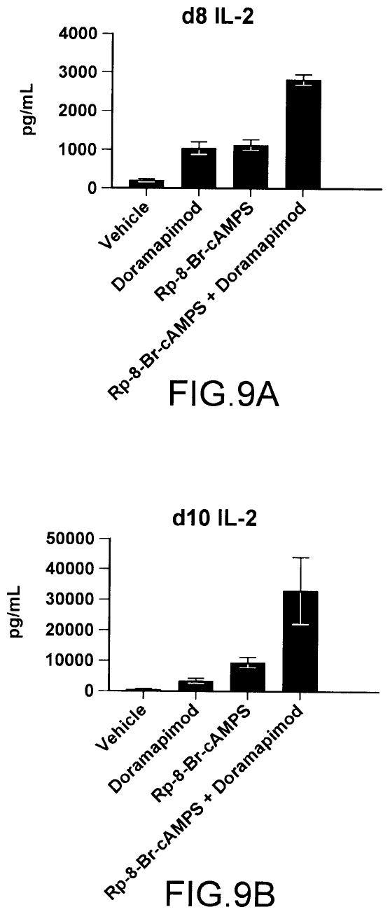

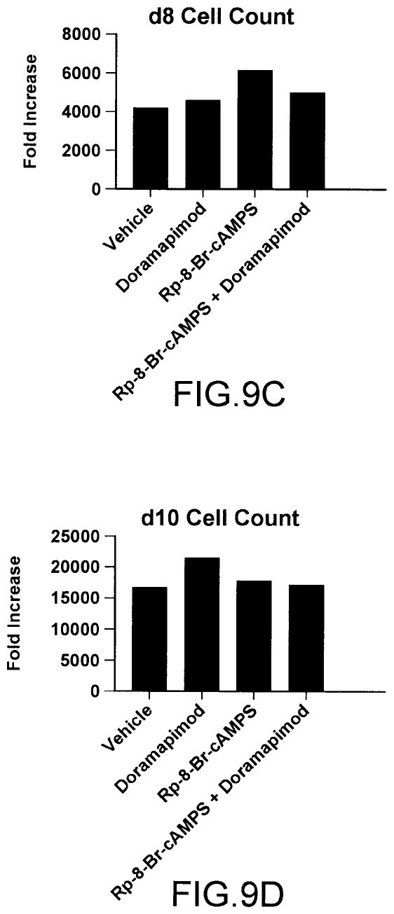

FIG. 9 graphically illustrates the concentration of IL-2 (FIGS. 9A and 9B) in supernatants from the day 8 and day 10 restimulations. The fold-increase in OT-I cells from the start of the culture (day 0) is also shown for day 8 (FIG. 9G) and day 10 (FIG. 9H).

IL-2 production from 8-day OT-I cultures was enhanced most dramatically with the combination of doramapimod and Rp-8-Br-cAMPS (13-fold), with less enhancement from culture with either inhibitor alone (doramapimod: 4.9-fold; Rp-8-Br-cAMPS: 5.3-fold). The effect of the combination treatment was further amplified following two additional days of culture, with day 10 OT-I cells producing 64-fold more IL-2 than vehicle control. This effect of two additional days of growth appears to have been driven by the PKA inhibitor, because OT-I cells cultured for 10 days in Rp-8-Br-cAMPS alone increased their IL-2 production to 18-fold over vehicle control, while the 10-day doramapimod culture (6.8-fold) was similar to the 8-day culture (p<0.0002 for all fold-change comparisons).

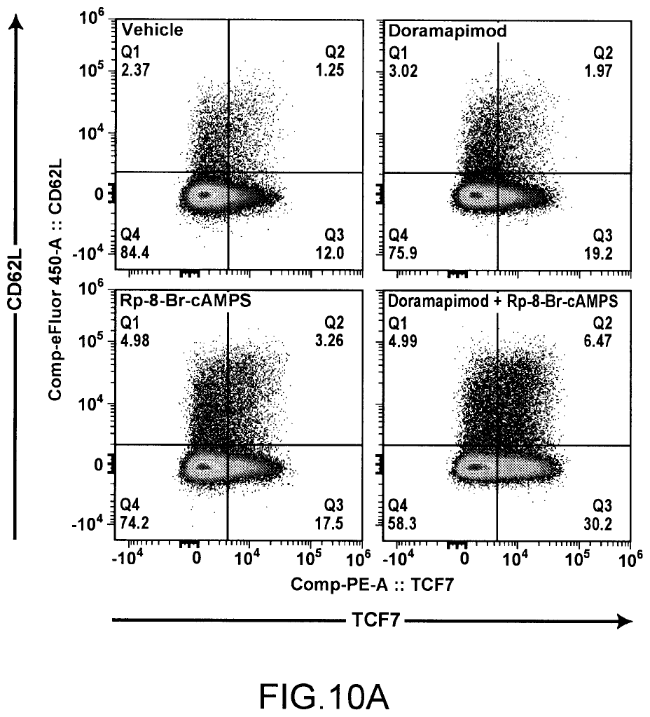

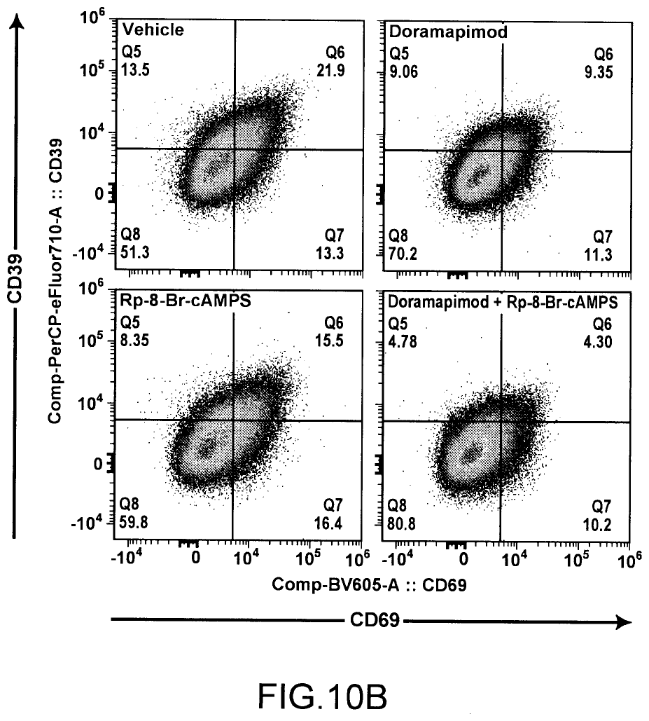

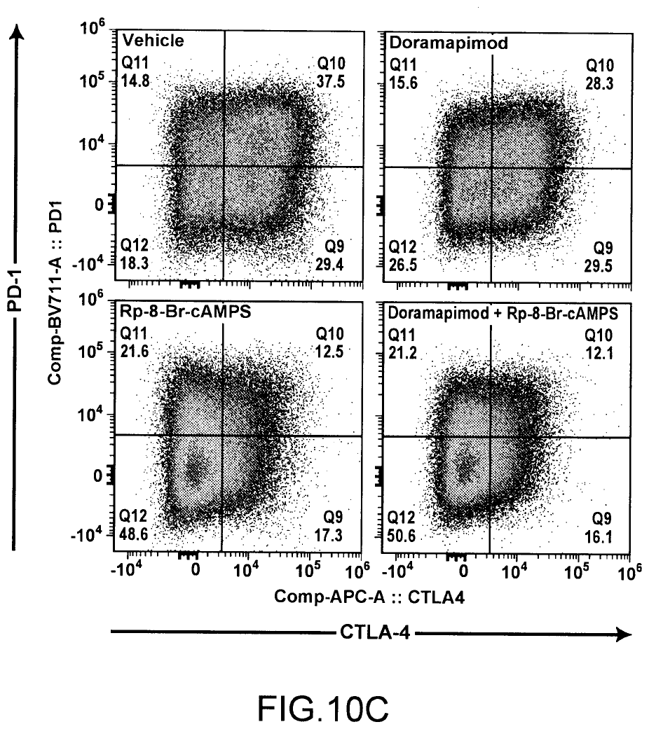

FIG. 10 depicts the expression of CD62L, TCF1/TCF7, CD39, CD69, PD-1, and CTLA-4 in OT-I cells after 8 days of culture with vehicle control, doramapimod, Rp-8-Br-cAMPS, or both compounds combined. Expression of these markers were consistent with an increased proportion of effector-memory T cells and a decrease in terminally differentiated or exhausted effector T cells (higher expression of CD62L and TCF1/TCF7, and decreased CD39, CD69, PD-1, and CTLA-4). Relative to vehicle control, cells from the doramapimod+Rp-8-Br-cAMPS culture exhibited a 3.2-fold increase in CD62L⁺ cells, a 2.8-fold increase in TCF1/TCF7+ cells, and a 5.2-fold increase in cells expressing both markers, with smaller increases observed for cells cultured with each inhibitor alone (FIG. 10A). Consistent with the report cited above (Krishna S. et al. Stem-like CD8 T cells mediate response of adoptive cell immunotherapy against human cancer. Science, 2020: 1328-1334), the doramapimod+Rp-8-Br-cAMPS combination decreased the percentage of OT-I T cells expressing both CD39 and CD69 from 22% to 4.3%, with each inhibitor alone promoting smaller reductions (FIG. 10B). Expression of the checkpoint molecules PD-1 and CTLA-4 was also measured, and it was found that their downregulation was also mediated by doramapimod and Rp-8-Br-cAMPS. As with the other markers, the greatest effects were seen when both inhibitors were combined, with cells expressing both PD-1 and CTLA-4 declining from 37.5% in the vehicle control culture to 12.1% in the doramapimod+Rp-8-Br-cAMPS culture (FIG. 10C). Notably, Rp-8-Br-cAMPS was particularly effective at downregulating CTLA-4, consistent with a previously described role for cAMP/PKA signaling in promoting CTLA-4 expression (Li J, Lin K W, Murray F, et al. Regulation of cytotoxic T lymphocyte antigen 4 by cyclic AMP. Am J Respir Cell Mol Biol. 2013; 48(1):63-70).

Discussion of Results:

In this Example, observations with PKA and p38 inhibition during human CD8 T cell growth were extended to effects on mouse CD8 T cells. As it was found with human T cells, combined inhibition of both PKA and p38 during OT-I CD8 T cell stimulation and growth with IL-2 and IL-7 markedly increased their IL-2 production. Most strikingly, 8 days of culture with doramapimod and Rp-8-Br-cAMPS increased IL-2 production by 13-fold, while an additional 2 days of growth further augmented this effect to 64-fold high IL-2 levels relative to the vehicle control cells. This demonstrates that CAR-T or ACT T cells expanded with PKA and p38 inhibitors should produce high levels of IL-2 upon tumor antigen recognition, leading to greatly improved growth and persistence in cancer patients. Furthermore, the observed enrichment of T cells with a memory phenotype (TCF1/TCF7⁺, CD62L⁺, CD39⁻, CD69⁻) and reduced PD-1 and CTLA-4 expression demonstrates that CAR-T or ACT T cells grown with combined PKA and p38 inhibitors should exhibit improved persistence and responsiveness following transfer into patients.

### Example 7

This Example demonstrates that T cells cultured with both the p38 inhibitor doramapimod and the PKA inhibitor Rp-8-Br-cAMPS exhibit dramatically enhanced tumor killing properties in mice, and that T cells cultured with the PKA inhibitor Rp-8-Br-cAMPS alone were also significantly better at reducing tumor growth in vivo, relative to control T cells treated with vehicle.

Background/Rationale:

The preceding examples show that T cells cultured with doramapimod or Rp-8-Br-cAMPS display phenotypes consistent with augmented central memory T cell phenotypes and a greater capacity to produce IL-2 following restimulation in the absence of the inhibitors, and that the combination of both inhibitors further augments these effects. To explore whether these changes translate into more potent anti-tumor activity in vivo, the inventors performed an adoptive T cell therapy experiment in which OT-1 T cells expanded for 10 days with the different inhibitor treatments were transferred into mice bearing EG7 tumors.

Methods:

Mouse OT-I TCR transgenic CD8 T cells were purified from splenocytes (StemCell 19853; Mouse CD8⁺ T Cell Isolation Kit), cultured in a 24-well plate with the following conditions per well:
2 mL RP10 media
4 million mitomycin C treated C₅₇BL/6 mouse splenocytes
0.2 million OT-I T cells
10 nM pOVA

and the following individual conditions:
1. DMSO vehicle control
2. 0.2 μM doramapimod
3. 500 μM Rp-8-Br-cAMPS
4. 0.2 μM doramapimod+500 μM Rp-8-Br-cAMPS

On days 2, 4, 6, and 8, cells were counted, washed, and reseeded at 0.5 million per well with the same 4 doramapimod/Rp-8-Br-cAMPS conditions and with recombinant mouse IL-2 and IL-7 (5 ng/mL each). On day 6 cells were seeded in 6-well plates with 10 mL per well, and on day 8 cells were seeded in T75 flasks in 30 mL media to obtain enough cells to transfer into EG7 tumor-bearing mice.

C57BL/6 female mice were implanted with 6 million EG7 tumor cells subcutaneously on day 4 of the OT-1 T cell expansion. On day 6 of tumor growth (day 10 of OT-1 T cell expansion) OT-1 T cells were washed and injected retro-orbitally into tumor bearing mice that had been sorted into 4 groups with equal tumor volume distributions (n=8). Via retroorbital injection, mice received 2 million OT1 T cells of each of the 4 culture conditions. Tumor volume (mm³) was calculated as 0.5×(length×width²), where length represents the largest tumor diameter and width represents the perpendicular tumor diameter. Mice were euthanized when the sum of the two measurements exceeded 30 mm.

Results:

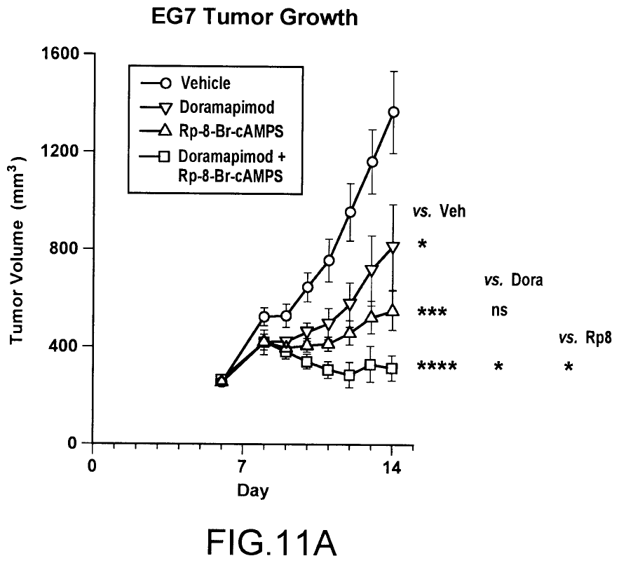

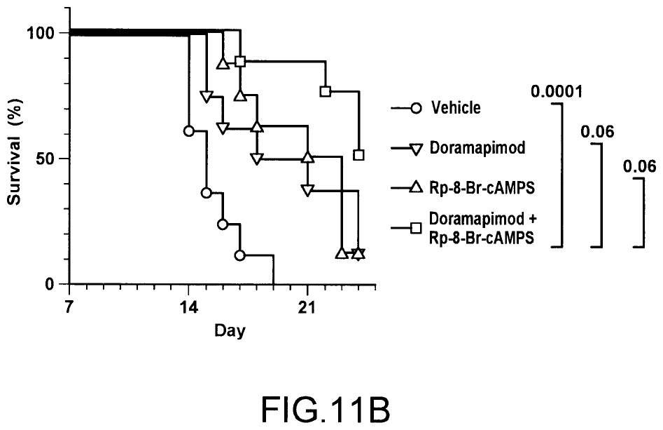

FIG. 11 graphically illustrates the tumor volume (FIG. 11A) and survival (FIG. 11B) of EG7 tumor-bearing mice following the transfer of OT-1 cells precultured with vehicle, doramapimod, Rp-8-Br-cAMPS, or both compounds combined (n=8). Error bars in (FIG. 11A) represent standard error of the mean. Student T-tests were performed for the indicated pair-wise comparisons, and the indicated p-values were observed for at least two days, including day 14 (ns, not significant; *, p<0.05; ***, p<0.0001; ****, p<0.00001). P-values from Mantel-Cox tests were derived for the indicated pair-wise comparisons in the Kaplan Meier survival curves (FIG. 11B).

Discussion of Results:

OT-1 T cells activated and expanded in the presence of both the p38 inhibitor doramapimod and the PKA inhibitor Rp-8-Br-cAMPS displayed potent and prolonged tumor-killing activity in mice harboring large pre-established EG7 tumors. While treatment with each individual inhibitor generated T cells that significantly halted and delayed tumor growth relative to vehicle control cells (doramapimod, p<0.05 on days 8-14; Rp-8-Br-cAMPS, p<0.05 on days 9-15), the combination resulted in T cells that durably reduced tumor volume for several days, resulting in significantly smaller tumors relative to doramapimod- (p<0.05 on days 10-15), Rp-8-Br-cAMPS-(p<0.05 on days 11, 12, 14), and vehicle- (p<0.05 on days 9-16, p<0.00001 on days 13, 14) exposed T cells (FIG. 11A). Accordingly, survival of tumor-bearing mice was significantly extended in the combination treatment group relative to the vehicle group (p=0.0001), and intermediate survival times were observed for cells treated with each individual inhibitor (FIG. 11B).

### Example 8

This Example describes the effects of different combinations of exemplary PKA, p38, and PI3Kδ inhibitors (Rp-8-Br-cAMPS, doramapimod, and idelalisib, respectively) on mouse CD8 T cell phenotypes during their stimulation and growth. As in Example 1, the inventors used OT-I TCR transgenic T cells, stimulating with its peptide antigen OVA 257-264 (pOVA), or with the EG7 T cell line that endogenously expresses this antigen.

Background/Rationale:

Inhibition of PI3Kδ has been reported to increase memory phenotype of cultured T cells similar to what the inventors observed with combined PKA and p38 inhibition and to increase tumor killing following adoptive transfer into tumor-bearing mice. To determine whether a PI3Kδ inhibitor would override or amplify the effects of combined PKA and p38 inhibition, the 4 conditions tested in EXAMPLE 6 were tested with and without the PI3Kδ inhibitor idelalisib.

Methods:

Mouse OT-I TCR transgenic CD8 T cells were purified from splenocytes (StemCell 19853; Mouse CD8⁺ T Cell Isolation Kit), cultured in a 24-well plate with the following conditions per well:
2 mL RP10 media
4 million mitomycin C treated C₅₇BL/6 mouse splenocytes
0.2 million OT-I T cells
10 nM pOVA
   and the following individual conditions:
1. DMSO vehicle control
2. 0.5 μM doramapimod
3. 500 μM Rp-8-Br-cAMPS
4. 0.5 μM doramapimod+500 μM Rp-8-Br-cAMPS
5. 1 μM idelalisib
6. 1 μM idelalisib+0.5 μM doramapimod
7. 1 μM idelalisib+500 μM Rp-8-Br-cAMPS
8. 1 μM idelalisib+0.5 μM doramapimod+500 μM Rp-8-Br-cAMPS

On days 2, 4, 6, and 8, cells were counted, washed and reseeded at 0.5 million per well in 2 mL media with the same 8 conditions and with recombinant mouse IL-2 and IL-7 (5 ng/mL each). On day 9, cells were immunophenotyped with the same panel described in EXAMPLE 6. Cells were also washed three times (12 mL media each), and restimulated in quintuplicate in 96-well round-bottom plates with the following conditions:
0.1 mL RP10 media
0.05 million OT-I T cells from each condition
0.05 million EG7 cells

After overnight culture, supernatants were harvested and analyzed for IL-2 content (MesoScale Discovery).

Results:

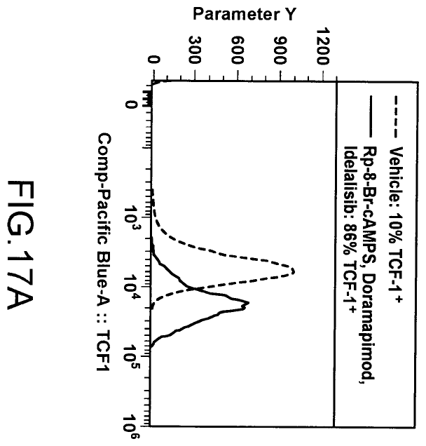

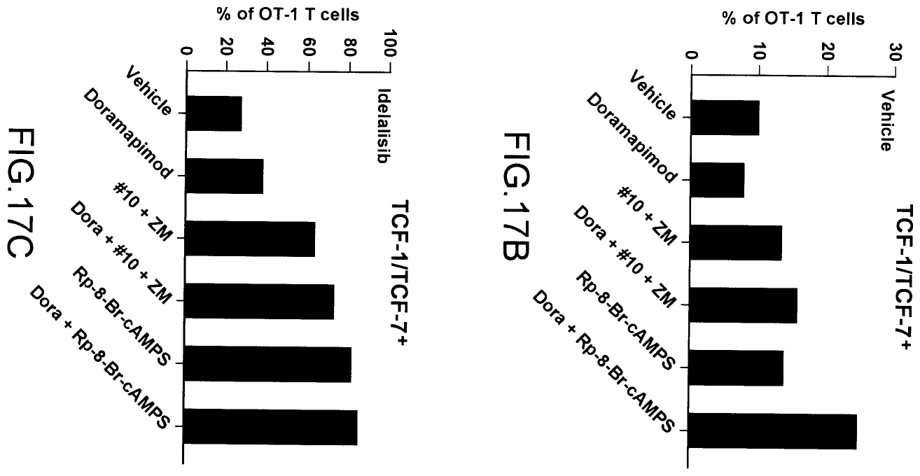

FIGS. 13A and 13B graphically illustrate the concentration of IL-2 in supernatants from the day 9 restimulation. FIG. 13B shows the same data as in FIG. 13A but with a split y-axis. Error bars represent standard deviation. IL-2 production was enhanced most dramatically with the triple combination of Rp-8-Br-cAMPS, doramapimod, and idelalisib (316-fold). Lower fold-change values were obtained with each inhibitor individually, or with the various dual combinations (Table 2). Importantly, while the PI3Kδ inhibitor idelalisib alone was markedly more effective at increasing IL-2 production capacity, it did not nullify or override the effects of doramapimod or Rp-8-Br-cAMPS, nor did it reduce the synergistic activity observed with dual p38 and PKA inhibition.

TABLE 2
Fold-change in IL-2 production relative to Vehicle control.

| | Vehicle | Idelalisib |
|---|---|---|
| Vehicle | 1.0 | 24.0 |
| Doramapimod | 2.2 | 65.0 |
| Rp-8-Br-cAMPS | 1.6 | 89.9 |
| Doramapimod + Rp-8-Br-cAMPS | 5.2 | 316.1 |

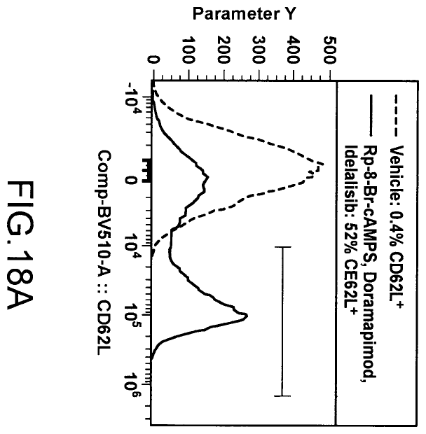

Large changes in T cell growth were not observed among the different conditions, with 4256-fold and 6433-fold increases in OT-1 cell number obtained from the Vehicle and triple-compound combination, respectively (FIG. 14).

Discussion of Results:

In this Example, the inventors evaluated a small molecule inhibitor against a 3^{rd} protein kinase, PI3Kδ, in the mouse CD8 T cell expansion system. Expansion of OT-I CD8 T cells with the PI3Kδ inhibitor idelalisib markedly increased IL-2 production following restimulation with antigen-expressing tumor cells in the absence of the inhibitor; and this effect did not override the synergistic activity of combined PKA and p38 inhibitors, making the triple compound combination at least 3-fold more effective at increasing the capacity for IL-2 production than any single inhibitor or pairwise combination. These findings indicate that T cells cultured with all 3 inhibitors should be more effective at reducing tumor burden and persist for an extended time frame following adoptive transfer into tumor bearing hosts.

### Example 9

As in the previous example, this Example describes the effects of various combinations of exemplary PKA, p38, and PI3Kδ inhibitors (Rp-8-Br-cAMPS, doramapimod, and idelalisib, respectively) on mouse CD8 T cell phenotypes during their stimulation and growth, and in addition, combined A2A and GPR174 inhibitors (ZM-241385 and Compound #10, respectively) were used in place of the PKA inhibitor Rp-8-Br-cAMPS.

Background/Rationale:

The initial work with the T cell conditioning protocols employed inhibitors of two Gas-coupled GPCRs (A2A and GPR174), and a global inhibitor of the cAMP/PKA pathway (Rp-8-Br-cAMPS). With human CD8 T cells, as shown in Example 5 above, the PKA inhibitor was found to be superior to the combined A2A and GPR174 inhibitors in its ability to create or sustain a central memory T cell phenotype during T cell expansion. The inventors then observed that this effect was further enhanced when p38 and/or PI3Kδ was also inhibited; however, whether the GPCR inhibitors behaved similarly to the PKA inhibitor when combined with the p38 and PI3Kδ inhibitors had not been addressed. Similar synergism between the ZM-241385+Compound #10 combination and p38/PI3Kδ inhibitors would demonstrate the utility of A2A+GPR174 inhibitors with p38 and PI3Kδ inhibitors in generating T cells for adoptive T cell therapy. Furthermore, because adenosine and PS/lysoPS may be the predominant ligands in long-term T cell cultures stimulating the Gαs-GPCR/cAMP/PKA pathway, it would provide further evidence that the high concentrations of Rp-8-Br-cAMPS required for biological effects were indeed acting on PKA rather than through an off-target effect.

Methods:

Mouse OT-I TCR transgenic CD8 T cells were purified from splenocytes (StemCell 19853; Mouse CD8⁺ T Cell Isolation Kit), cultured in a 24-well plate with the following conditions per well:
2 mL RP10 media
4 million mitomycin C treated C57BL/6 mouse splenocytes
0.2 million OT-I T cells
10 nM pOVA

and the following individual conditions:
1. DMSO vehicle control
2. 0.5 μM doramapimod
3. 0.1 μM ZM-241385+0.3 μM Compound #10
4. 0.5 μM doramapimod+0.1 μM ZM-241385+0.3 μM Compound #10
5. 500 μM Rp-8-Br-cAMPS
6. 0.5 μM doramapimod+500 μM Rp-8-Br-cAMPS
7. 1 μM idelalisib
8. 1 μM idelalisib+0.5 μM doramapimod
9. 1 μM idelalisib+0.1 μM ZM-241385+0.3 μM Compound #10
10. 1 μM idelalisib+0.5 μM doramapimod+0.1 μM ZM-241385+0.3 μM Compound #10
11. 1 μM idelalisib+500 μM Rp-8-Br-cAMPS
12. 1 μM idelalisib+0.5 μM doramapimod+500 μM Rp-8-Br-cAMPS

On days 2, 4, 6, and 8, cells were counted, washed and reseeded at 0.5 million per well in 2 mL media with the same 8 conditions and with recombinant mouse IL-2 and IL-7 (5 ng/mL each). On day 10, cells were immunophenotyped with the same panel described in EXAMPLE 6. Cells were also washed three times (12 mL media each), and restimulated in quintuplicate in 96-well round-bottom plates with the following conditions:
0.1 mL RP10 media
0.05 million OT-I T cells from each condition
0.05 million EG7 cells

After overnight culture, supernatants were harvested and analyzed for IL-2 content (MesoScale Discovery).

Results:

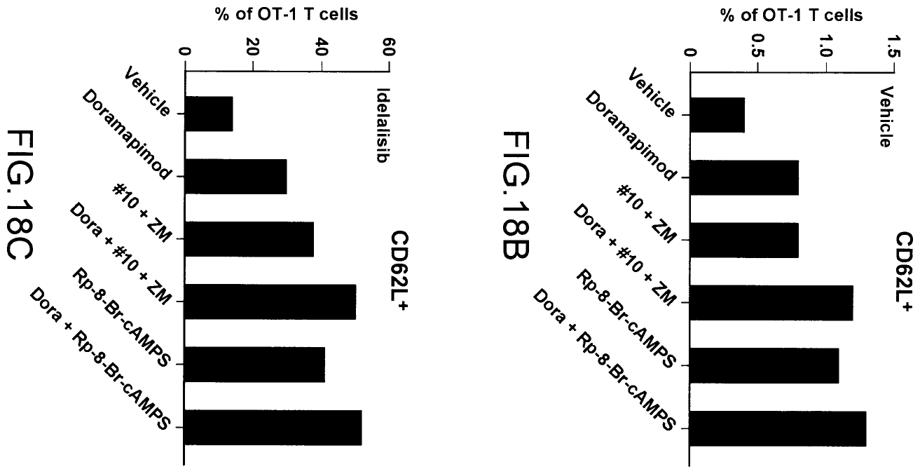

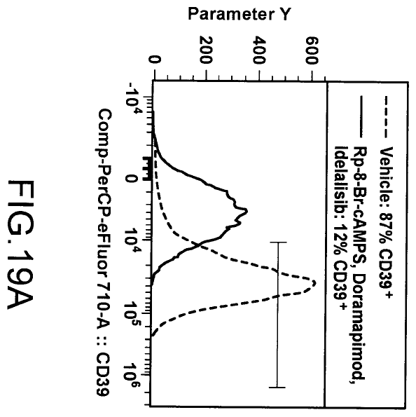

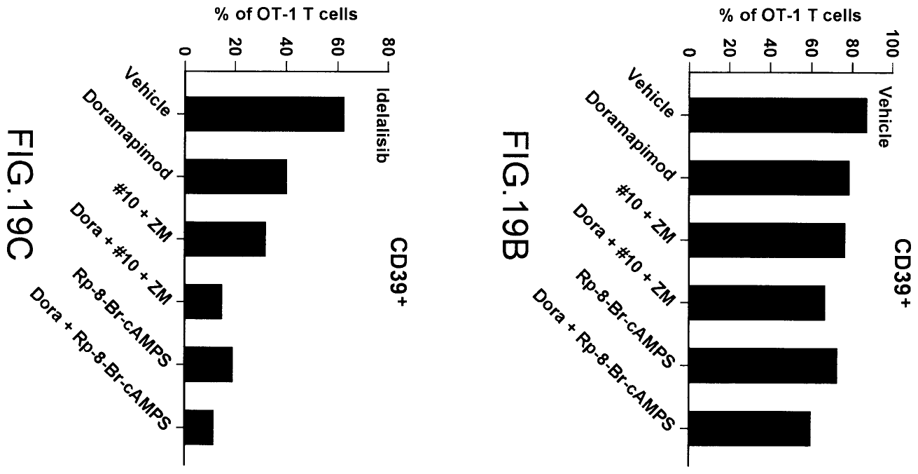

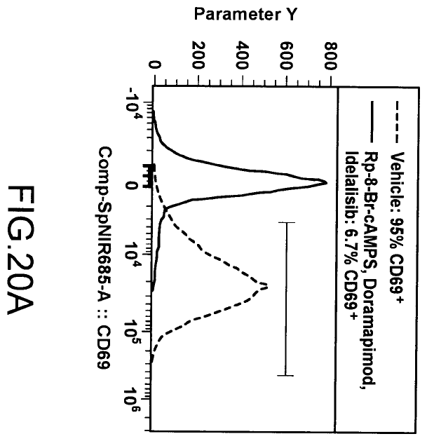

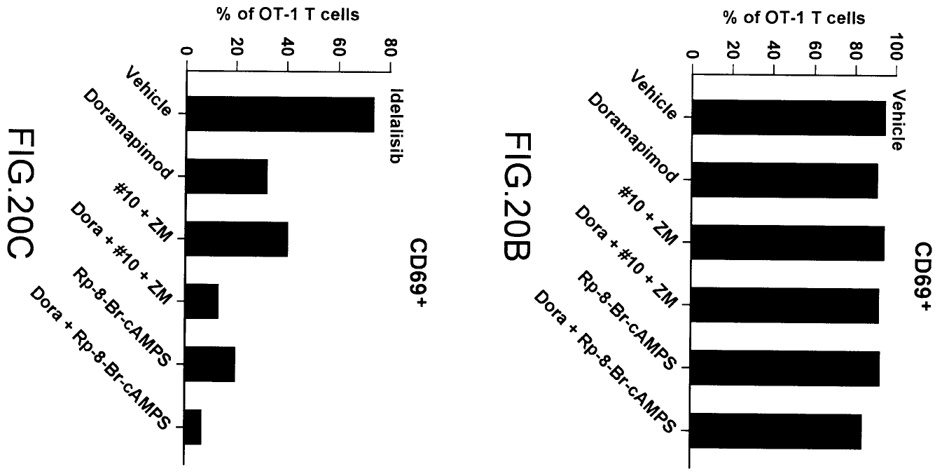

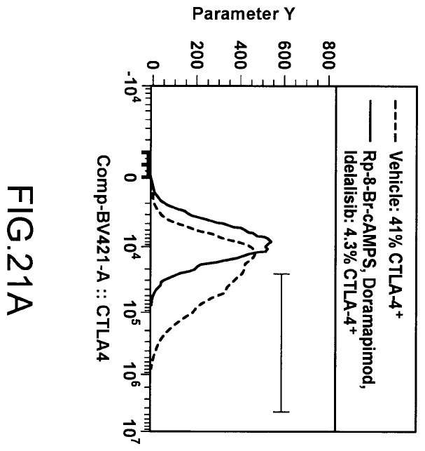

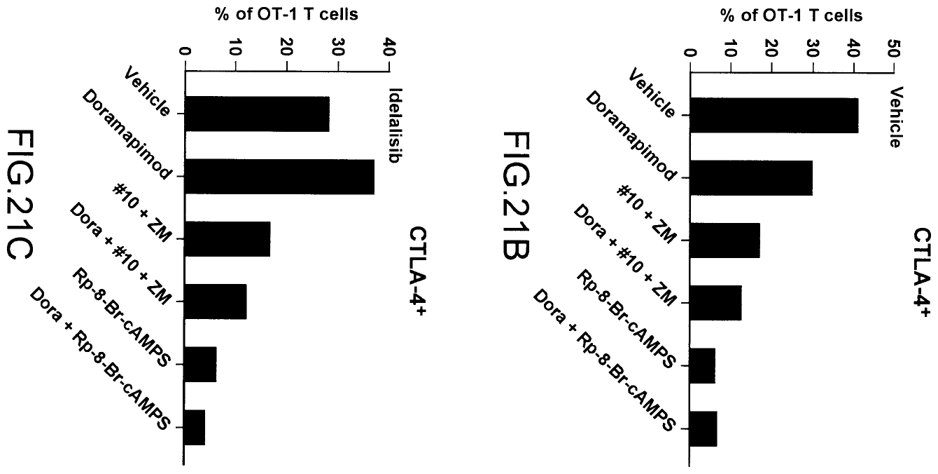

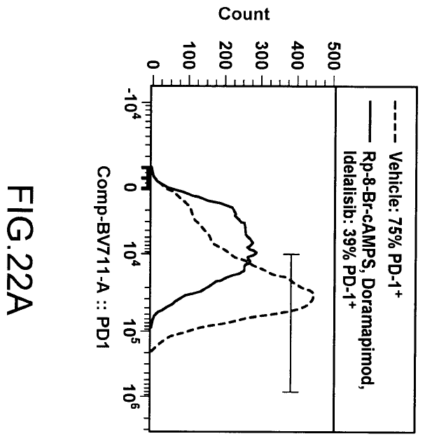

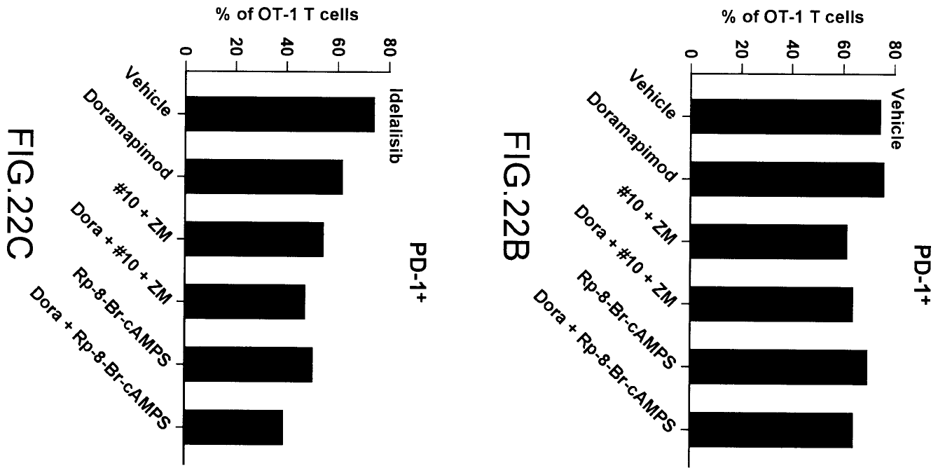

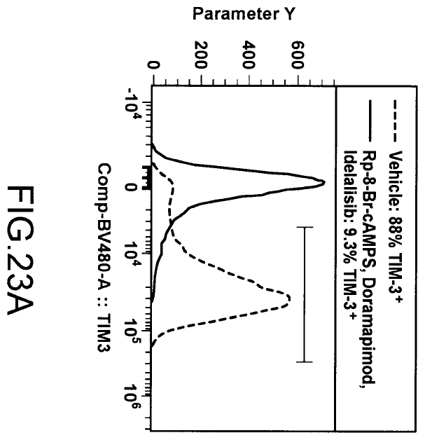

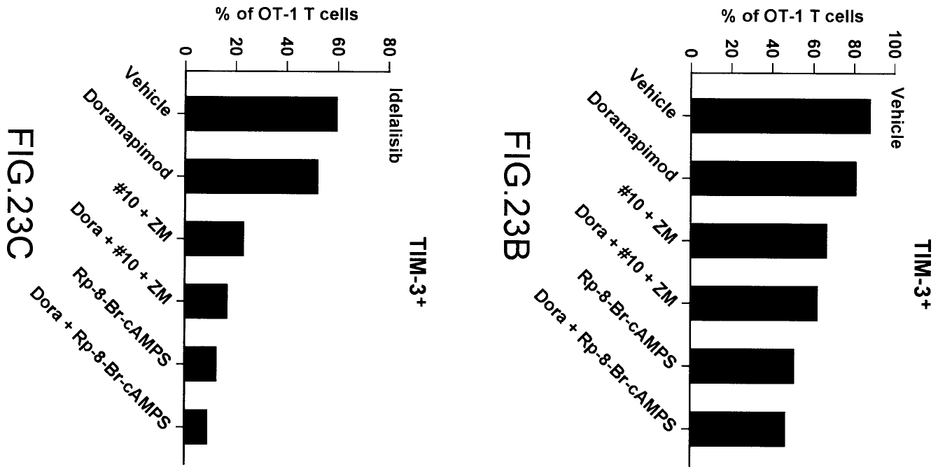

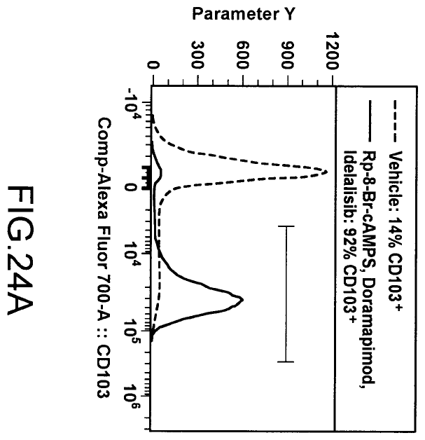

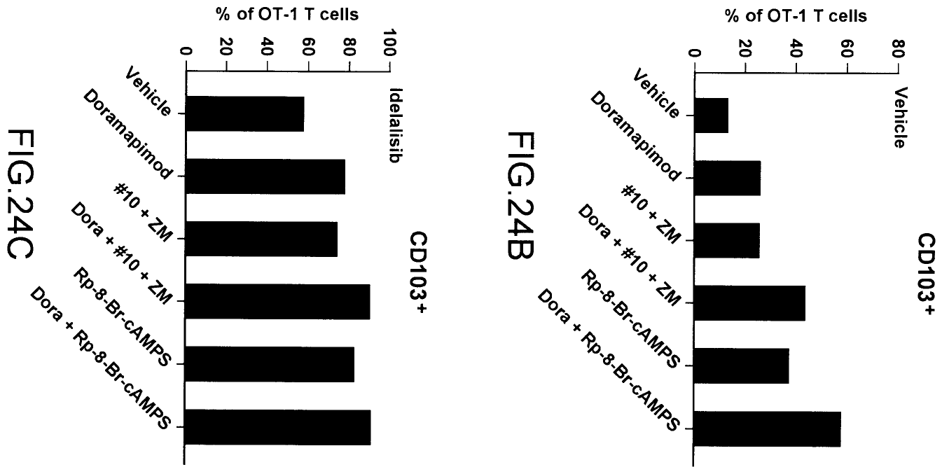

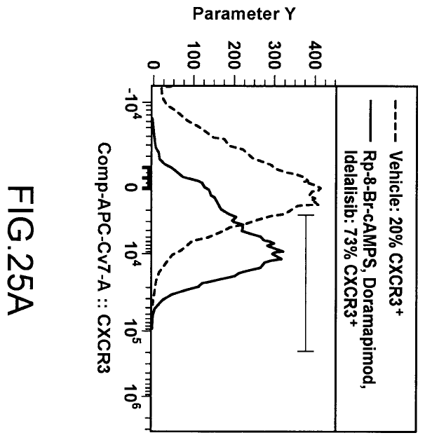

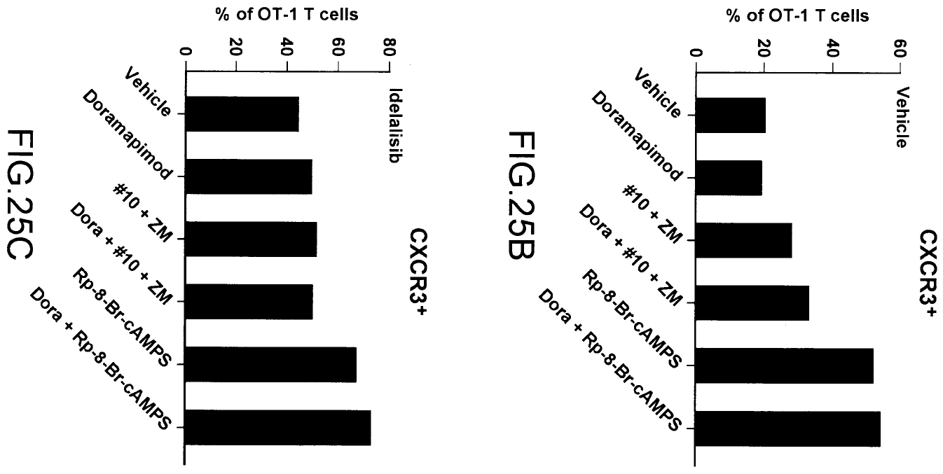

FIGS. 15A and B graphically illustrate the concentration of IL-2 in supernatants from the day 10 restimulation, and FIGS. 16A and B show the fold-change increase in OT-1 T cell numbers. In this experiment, the phenotype of the expanded OT-1 T cells was also determined by flow cytometry, and FIGS. 17-25 show representative expression levels (A) and the percentage of cells expressing each protein for the two different culture conditions (B and C) for the following phenotypic markers: TCF-1/TCF-7, CD62L, CD39, CD69, CTLA-4, PD-1, TIM-3, CD103, and CXCR3. Overall, the effects of the PKA inhibitor Rp-8-Br-cAMPS were recapitulated—although to a lesser extent—by the combined inhibition of the Gas-coupled GPCRs A2A and GPR174, and these effects were further amplified by the inclusion of doramapimod and/or idelalisib such that the greatest effects were seen in the combination of doramapimod and idelalisib with either ZM-241385+Compound #10 or with Rp-8-Br-cAMPS. These effects consisted of increased capacity for IL-2 production (FIG. 15), increased expression of central memory T cell markers TCF-1/TCF-7 and CD62L (FIGS. 17,18), decreased expression of T cell exhaustion and terminal differentiation markers CD39, CD69, CTLA-4, PD-1, TIM-3 (FIGS. 19-23), and increased expression of tissue-homing molecules CD103 and CXCR3 (FIGS. 24, 25).

Discussion of Results:

In this example the inventors provide evidence that inhibition of either PKA or of 2 Gαs-coupled GPCRs (A2A and GPR174) during mouse CD8 T cell expansion increases the representation of cells with a central memory phenotype, and that inclusion of p38 and/or PI3Kδ inhibitors enhanced the effect of either PKA or A2A/GPR174 inhibition without markedly reducing overall T cell numbers. These findings support the model that Rp-8-Br-cAMPS is acting through the cAMP/PKA pathway, as the A2A and GPR174 inhibitors are known to block cAMP production in T cells. The A2A and GPR174 ligands (adenosine and lysoPS) are produced by T cells during in vitro growth, resulting in increased cAMP/PKA signaling; however, there likely exist other Gas-coupled GPCRs in T cells responding to components in the tissue culture media, such as GPR65 responding to acidic pH. For this reason, it is not surprising that inhibition of all PKA signaling with Rp-8-Br-cAMPS was more effective than combined A2A/GPR174 inhibition in increasing central memory T cell phenotypes.

It is also noteworthy that, for several central memory T cell phenotypes, PI3Kδ inhibition had modest or no effect unless the cAMP/PKA pathway or the cAMP/PKA and p38 pathways were also inhibited. These include:
1) upregulation of TCF-1 (Vehicle: 10%; idelalisib: 27%; idelalisib+Rp-8-Br-cAMPS: 82%),
2) downregulation of CD39 (Vehicle: 87%; idelalisib: 63%; idelalisib+Rp-8-Br-cAMPS: 19%),
3) downregulation of CD69 (Vehicle: 95%; idelalisib: 74%; idelalisib+Rp-8-Br-cAMPS: 20%; idelalisib+doramapimod+Rp-8-Br-cAMPS: 7%),
4) downregulation of CTLA-4 (Vehicle: 41%; idelalisib: 28%; idelalisib+Rp-8-Br-cAMPS: 6%; idelalisib+doramapimod+Rp-8-Br-cAMPS: 4%),
5) downregulation of PD-1 (Vehicle: 75%; idelalisib: 75%; idelalisib+Rp-8-Br-cAMPS: 51%; idelalisib+doramapimod+Rp-8-Br-cAMPS: 39%), and
6) downregulation of TIM-3 (Vehicle: 88%; idelalisib: 60%; idelalisib+Rp-8-Br-cAMPS: 13%; idelalisib+doramapimod+Rp-8-Br-cAMPS: 9%).

Thus, protocols including a PI3Kδ inhibitor for the production of T cells with a central memory phenotype for adoptive T cell therapy are likely to be markedly more effective if a PKA inhibitor or combined Gas-GPCR inhibitors are also included, with or without a p38 inhibitor.

In this example, the inventors also observed upregulation of T cell homing receptors CD103 and CXCR3 (FIGS. 24B-C, 25B-C). Expression of these molecules on T cells should increase the trafficking of adoptively transferred T cells to tumors. CD103 is an integrin important for tissue retention of tissue-resident memory T cells, and CXCR3 is a chemokine receptor important for migration of cytotoxic T cells into tumors. Thus, in addition to the increased survival and expansion of T cells associated with the central memory phenotype, T cells treated with PKA+p38+PI3Kδ inhibitors should more effectively migrate to tumor tissues to promote prolonged tumor killing.

Final Discussion:

The phenotype of T cells cultured and expanded for adoptive T cell therapy for cancer is influenced by multiple factors, some of which are intentional, such as anti-CD3/CD28 and cytokines, and some of which are byproducts of T cell proliferation and death. The latter includes molecules that activate immunosuppressive Gas-coupled GPCRs such as adenosine, lysophosphatidylserine, and low pH, acting on A2A, GPR174, and GPR65 receptors, respectively. How these pathways may influence T cell phenotype and function after in vitro expansion and after transfer into cancer patients has not been explored until the present disclosure.

The inventors first addressed whether inhibiting individual Gas-coupled GPCRs during T cell stimulation and growth influenced the phenotype of the expanded T cells and found that the combination of GPR174 and A2A inhibitors was more effective than each inhibitor alone at maintaining a memory T cell phenotype and a high capacity for IL-2 production (Examples 1-3). It was subsequently found that inhibiting cAMP signaling more globally with the PKA-R (regulatory) subunit antagonist Rp-8-Br-cAMPS was even more effective at maintaining IL-2-production potential (Example 4). In contrast, inhibition of an alternative cAMP signaling effector, EPAC, did not elicit this phenotype (Examples 4, 5).

A recently published investigation into kinases that regulate the retention of memory phenotypes in cultured T cells reported that the MAP kinase p38 attenuated memory T cell differentiation and that a p38 inhibitor, doramapimod, increased memory T cell numbers, resulting in T cells that were more effective at reducing tumor growth in mice (Gurusamy D, et al. Multi-phenotype CRISPR-Cas9 Screen Identifies p38 Kinase as a Target for Adoptive Immunotherapies. Cancer Cell. 2020; 37(6):818-833). Because these findings were similar to those obtained herein with the PKA inhibitor Rp-8-Br-cAMPS, the inventors sought to determine whether the two effects were redundant or if the inhibitors would work together to further enhance memory T cell phenotype and function. It was found that the combination of Rp-8-Br-cAMPS and doramapimod promoted a synergistic (more-than-additive) increase in IL-2 production potential compared to either inhibitor alone (Examples 5, 6) and that memory T cell phenotypes were also further augmented (Example 6). Accordingly, in a mouse adoptive T cell therapy experiment, T cells cultured with both Rp-8-Br-cAMPS and doramapimod were significantly and dramatically more effective at attenuating tumor growth compared to T cells cultured with either inhibitor alone (Example 7). In addition to p38 inhibition, PI3K or AKT inhibitors have also been shown to support expansion of central memory T cells in ACT protocols (see Example 8). The inventors discovered that the benefit obtained with a PI3Kδ inhibitor alone was modest, and that inclusion of PKA and p38 inhibitors resulted in marked and synergistic enrichment of multiple central memory markers without attenuating overall T cell expansion. Relative to Vehicle control, cells cultured with all three inhibitors exhibited large increases in IL-2 expression potential, substantial enhancement of TCF1/TCF7 and CD62L central memory marker expression, and large reductions in exhaustion/terminal differentiation markers CD39, CD69, CTLA-4, PD-1, and TIM-3. Together, the findings disclosed herein strongly support the conclusion that adoptive T cell therapy—with either patient-derived tumor-specific T cells or with genetically engineered patient-derived or “off-the-shelf” universal donor T cells—will be more efficacious if the T cells are cultured and expanded with a PKA inhibitor, a combination of a PKA and a p38 inhibitor, or a combination of a PKA, p38, and PI3Kδ inhibitors.

It has been reported that p38 is downstream of PKA in a signaling cascade. See, for example, Lajevic M D, Suleiman S, Cohen R L, Chambers D A. Activation of p38 mitogen-activated protein kinase by norepinephrine in T-lineage cells. Immunology. 2011; 132(2):197-208. The PKA inhibitor the inventors employed herein, Rp-8-Br-cAMPS, exhibits low cell permeability and must be used at high concentrations to adequately inhibit PKA; thus, it remains formally possible that full PKA inhibition was not achieved in these experiments, and that complete PKA inhibition can render p38 inhibition unnecessary.

The inventors focused on IL-2 production as the primary readout for sustained memory T cell activity, as other cytokines that promote anti-tumor immune responses—such as IFN-γ and GM-CSF—are more associated with terminally differentiated T cells that lack a high capacity for self-renewal. Nevertheless, the inventors also measured IFN-γ, TNF, and GM-CSF production from the restimulated T cells and found that levels of these cytokines followed the trends observed for IL-2, although with smaller fold-changes and greater variability between experiments.

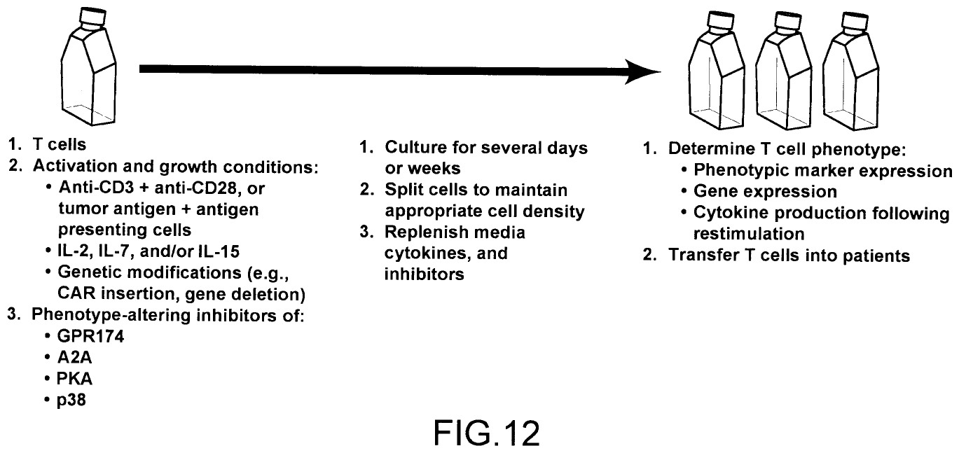

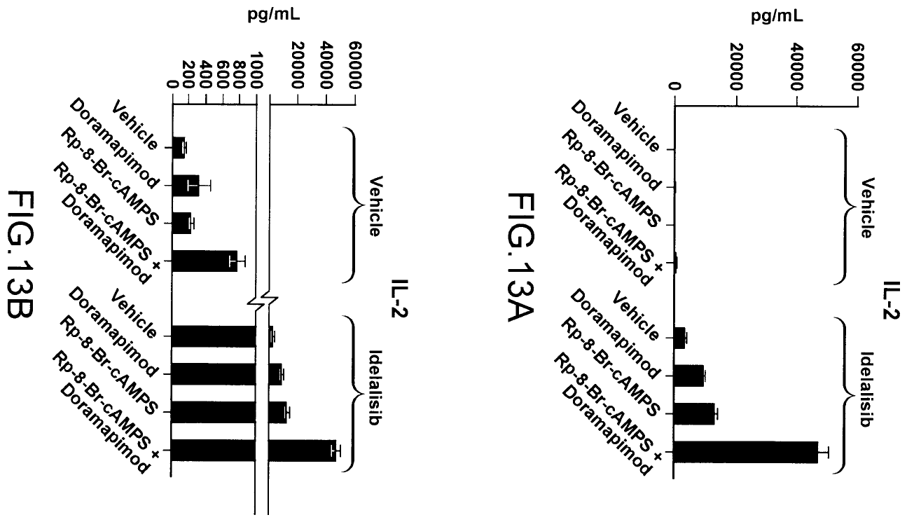

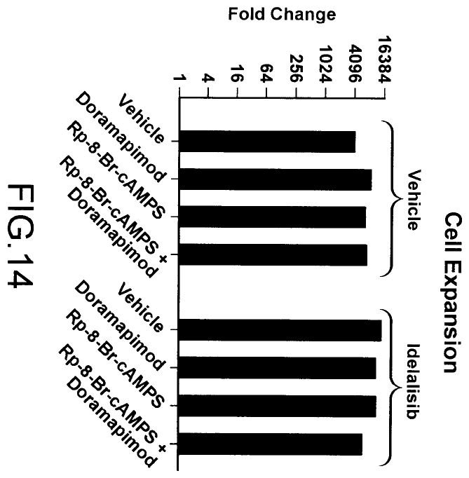

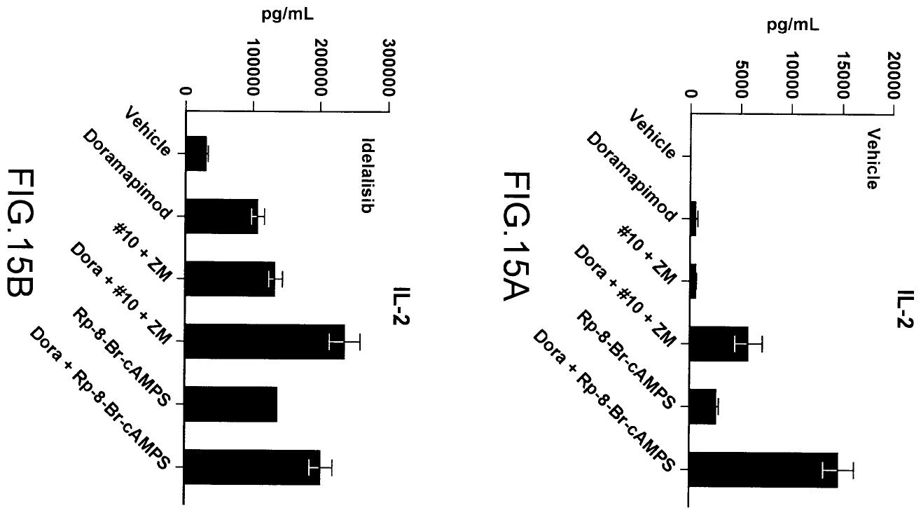

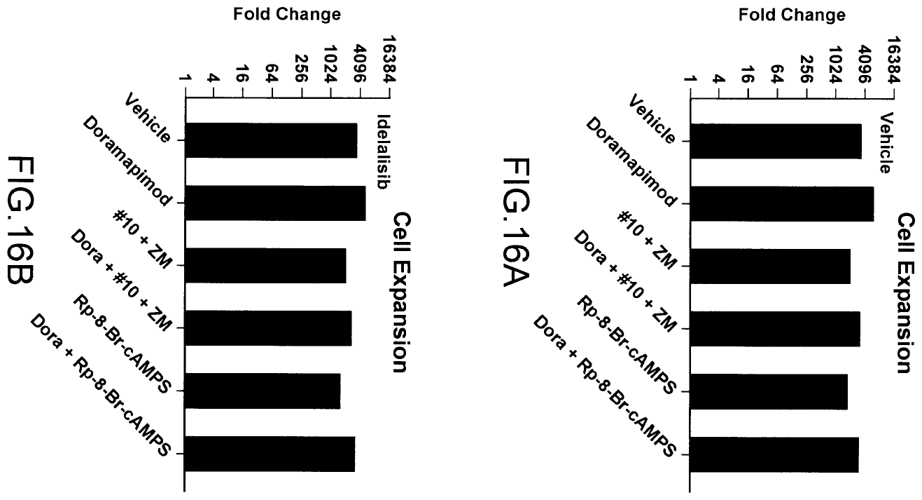

Thus, a population of therapeutic T cells comprising at least a sub-population of phenotype-altered T cells can be produced by the methods of the disclosure, for instance, as depicted in FIG. 12 schematically showing one such illustrative example. Such T cell populations can be used therapeutically, e.g., administered to a subject (e.g., a human patient with cancer) in need thereof to treat cancers treatable by adoptive T cell therapy.

In some embodiments, the methods include the following series of steps:
(i) culturing a population of T cells that are obtained from a subject in need of adoptive T cell therapy in the presence of a composition comprising a PKA inhibitor, an A2A inhibitor, a GPR174 inhibitor, or a combination thereof alone or optionally in combination with a p38 inhibitor and/or a PI3Kδ inhibitor, for a time period of at least 2 days (e.g., from 2 days and up to 40 days); and
(ii) washing or otherwise removing the composition (including all inhibitors comprised in the composition) from the T cells; thereby generating a population of T cells comprising phenotype-altered T cells, wherein said phenotype-altered T cells exhibit an increase in central memory T cell phenotypes, including an increased capacity for IL-2 production, and exhibit greater antitumor activity in an in vivo setting, and combinations thereof as compared to control T cells; and
(iii) optionally administering the T cell population generated according to step (ii) to a subject in need thereof.

The inventors thus have demonstrated that a combination of PKA and p38 inhibitors resulted in synergistic enhancement of IL-2 production and memory phenotype, as exemplified above. Addition of a PI3Kδ inhibitor, such as that used in the above-described examples, markedly amplified these effects without overriding the synergy between the PKA and p38 inhibitors.

All publications, patent applications, and patents mentioned in this specification are herein incorporated by reference.

Various modifications and variations of the described methods, compositions, and compounds, of the disclosure will be apparent to those skilled in the art without departing from the scope and spirit of the disclosure. Although the disclosure has been described in connection with specific desired embodiments, it should be understood that the disclosure as claimed should not be unduly limited to such specific embodiments. Indeed, various modifications of the described modes for carrying out the disclosure that are obvious to those skilled in the fields of medicine, immunology, pharmacology, oncology, or related fields are intended to be within the scope of the disclosure.

1. A method for treating a disease, comprising administering to a subject in need thereof a therapeutically effective amount of phenotype-altered T cells, wherein the phenotype-altered T cells are prepared by a method comprising culturing a population of T cells in vitro in the presence of a phenotype-altering composition comprising a phenotype-altering agent selected from the group consisting of a protein kinase A (PKA) inhibitor, an A2A adenosine receptor inhibitor, a GPR174 inhibitor, and combinations thereof for a time sufficient to alter a phenotype of at least a subpopulation of the population of T cells.

2. (canceled)

3. The method of claim 1, wherein the composition further comprises a p38 inhibitor, a PI3Kδ inhibitor, or a combination thereof.

4-5. (canceled)

6. The method of claim 1, wherein the population of T cells comprises genetically modified T cells.

7. (canceled)

8. The method of claim 6, wherein the genetically modified T cells comprise an exogenous nucleic acid encoding a T Cell Receptor (TCR), an exogenous nucleic acid encoding a Chimeric Antigen Receptor (CAR), or a combination thereof.

9. (canceled)

10. The method of claim 1, wherein the population of T cells comprises autologous T cells or allogenic T cells, including T cells isolated from a cancer patient that naturally express TCRs specific for antigens expressed by the patient's tumor.

11. (canceled)

12. The method of claim 1, wherein the disease is cancer.

13. (canceled)

14. The method of claim 1, wherein the phenotype altering agent is a GPR174 inhibitor.

15. (canceled)

16. The method of claim 14, wherein the GPR174 inhibitor is a small molecule represented by any one of Formulae I, II, III, IV, V, or VIII.

17. The method of claim 1, wherein the phenotype altering agent is a protein kinase A (PKA) inhibitor.

18. The method of claim 17, wherein the PKA inhibitor is a small molecule or a peptide inhibitor of PKA-C, or an antisense oligonucleotide targeting PKA-Cα and/or PKA-Cβ.

19. The method of claim 17, wherein the protein kinase A (PKA) inhibitor is selected from the group consisting of HA-100 dihydrochloride, Rp-cAMPS, H-89 dihydrochloride, PKI (5-24), Staurosporine, Calphostin C, KT 5720, Rp-8-Br-cAMPS, 5-Iodotubercidin, Piceatannol, Fasudil (monohydrochloride salt), ML-7 hydrochloride, CGP-74514A hydrochloride, ML-9, Daphnetin, Myricetin, PKC-412, A-674563, K-252a, H-7 dihydrochloride, bisindolylmaleimide IV, cGK1alpha inhibitor-cell permeable DT-3, TX-1123, Rp-8-PIP-cAMPS, 8-bromo2′-monobutyrladenosine-3′,5′-cyclic monophosphorothioate Rp-isomer, Bisindolylmaleimide III hydrochloride, Rp-adenosine 3′,5′-cyclic monophosphorothioate sodium salt, A-3 hydrochloride, H-7, H-8·2HCl, K252c, HA-1004 dihydrochloride, K-252b, HA-1077 dihydrochloride, MDL-27,032, H-9 hydrochloride, Rp-8-CPT-cAMPS, bisindolylmaleimide III, -lacetamido-4-cyano-3-methyllisoquinoline, Ilmofosine, Rp-8-hexylaminoadenosine 3′,5′-monophosphorothioate, HA-1004 hydrochloride, PKA Inhibitor IV, Adenosine 3′,5′-cyclic monophosphorothioate 8-chloro Rp-isomer sodium salt, adenosine 3′,5′cyclic monophosphorothioate 2′-O-monobutyryl Rp-isomer sodium salt, 4-cyano-3-methylisoquinoline, 8-hydroxyadenosine-3′,5′-monophosphorothioate Rp-isomer, PKI (6-22) amide, SB 218078, Rp-8-pCPT-cyclic GMPS sodium, Sp-8-pCPT-cAMPS, N[2-(p-Cinnamylamino)shyethyl]-5-isoquinolone sulfonamide, AT7867, GSK 690693, PKI (14-22) amide (myristoylated), Rp-8-bromo-cAMPS, or combinations thereof.

20. The method of claim 1, wherein the phenotype altering agent is an A2A adenosine receptor inhibitor.

21. The method of claim 20, wherein the A2A adenosine receptor inhibitor is selected from the group consisting of ZM 241385 (CAS 139180-30-6), istradefylline (CAS 155270-99-8), xanthine amine congener (CAS 96865-92-8), XCC (CAS 96865-83-7), ANR 94 (CAS 634924-89-3), PSB 1115 (CAS 409344-71-4), 3,7-dimethyl-1-propargylxanthine (CAS 14114-46-6), SCH 58261 (CAS 160098-96-4), SCH 442416 (CAS 316173-57-6), 8-(3-chlorostyryl)caffeine (CAS 147700-11-6), CGS 15943 (CAS 104615-18-1), ST4206 (CAS 246018-36-9), KF21213 (CAS 155271-17-3), regadenoson (CAS 313348-27-5), preladenant (CAS 377727-87-2), CGS 21680 (CAS 120225-54-9), tozadenant (CAS 870070-55-6), Sch412348 (CAS 377727-26-9), ST3932 (CAS 1246018-21-2), A2A receptor antagonist 1 (CPI-444 analog; CAS 443103-97-7), istradefylline (CAS 155270-99-8), AZD4635 (CAS 1321514-06-0), CGS 15943 (CAS 104615-18-1), vipadenant (CAS 442908-10-3), CPI-444 (CAS 1202402-40-1), TC-G 1004 (CAS 1061747-72-5), 4-desmethyl istradefylline (CAS 160434-48-0), PSB 0777 (CAS 2122196-16-9), or a combination thereof.

22. The method of claim 3, wherein the p38 inhibitor is selected from the group consisting of doramapimod (CAS 285983-48-4), losmapimod (CAS 585543-15-3), SX 011 (CAS 309913-42-6), SB202190 (CAS 350228-36-3), VX 702 (CAS 745833-23-2), JX-401 (CAS 349087-34-9), p38 MAP Kinase Inhibitor VIII (CAS 321351-00-2). SCIO 469 (CAS 309913-83-5), p38 MAP Kinase Inhibitor V (CAS 271576-77-3), p38 MAP Kinase Inhibitor IX (N-(isoazol-3-yl)-4-methyl-3-(1-phenyl-1H-pyrazolo[3,4-d]pyrimidin-4-ylamino)benzamide), PD 169316 (CAS 152121-53-4), p38 MAP Kinase Inhibitor III (CAS 581098-48-8), PH-797804 (CAS 586379-66-0), RWJ 67657 (CAS 215303-72-3), VX 745 (CAS 209410-46-8), LY 364947 (CAS 396129-53-6), p38 MAP Kinase Inhibitor (CAS 219138-24-6), SB 239063 (CAS 193551-21-2), SB 202190 (CAS 152121-30-7), SB 203580 (CAS 152121-47-6), p38 MAP Kinase Inhibitor IV (CAS 1638-41-1), SD-169 (CAS 1670-87-7), N-(5-Chloro-2-methylphenyl)-7-nitrobenzo[c][1,2,5]oxadiazol-4-amine (FGA-19), or a combination thereof.

23. The method of claim 3, wherein the PI3Kδ inhibitor is Acalisib (GS-9820, CAL-120), Dezapelisib (INCB040093), Idelalisib (CAL-101, GS-1101), Leniolisib (CDZ173), Inperlisib (YY-20394, PI3K(delta)-IN-2), Nemiralisib (GSK2269557), Parsaclisib (INCB050465, IBI-376), Puquitinib (XC-302), Seletalisib (UCB-5857), Zandelisib (ME-401, PWT143), ACP-319 (AMG 319), BGB-10188, GS-9901, GSK2292767, HMPL-689, IOA-244 (MSC236084), RV1729, or SHC014748M.

24. The method of claim 3, wherein the phenotype-altering composition comprises a PKA inhibitor and a p38 inhibitor.

25. The method of claim 3, wherein the phenotype-altering composition comprises a PKA inhibitor, a p38 inhibitor, and a PI3Kδ inhibitor.

26. The method of claim 25, wherein the PKA inhibitor is Rp-8-Br-cAMPS, the p38 inhibitor is doramapimod, and the PI3Kδ inhibitor is idelalisib.

27. The method of claim 1, wherein the phenotype of at least a subpopulation of the population of T cells is altered after the culture period and/or the phenotype of at least a subpopulation of the population of T cells is altered after transfer of the T cells into the subject.

28. The method of claim 27, wherein the phenotype altered after transfer into the subject is selected from the group consisting of greater persistence, prolonged survival, greater antitumor activity, and combinations thereof as compared to control T cells, wherein the control T cells are identical to the T cells cultured in the presence of the composition except that the control T cells are not cultured in the presence of the composition.

29-86. (canceled)

87. The method of claim 1, wherein the T cells are T cells obtained from the subject in need thereof, T cells isolated from a universal donor, or universal donor T cells derived from stem cells.
